(19) United States
(12) Patent Application Publication
(10) Pub. No.: US 2023/0335253 A1
(43) Pub. Date: Oct. 19, 2023

(54) Devices, Systems, and Methods, including Augmented Reality (AR) Eyewear, for Estimating Food Consumption and Providing Nutritional Coaching

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 18/121,841

(22) Filed: Mar. 15, 2023

Related U.S. Application Data
(63) Continuation-in-part of application No. 17/903,746, filed on Sep. 6, 2022.
(63) Continuation-in-part of application No. 17/239,960, filed on Apr. 26, 2021.
(63) Continuation-in-part of application No. 16/737,052, filed on Jan. 8, 2020.
(63) Continuation-in-part of application No. 16/568,580, filed on Sep. 12, 2019, now Pat. No. 11,478,158.
Application No. 16/737,052 is a continuation-in-part of application No. 16/568,580, filed on Sep. 12, 2019, now Pat. No. 11,478,158.
Application No. 16/737,052 is a continuation-in-part of application No. 15/963,061, filed on Apr. 25, 2018, now Pat. No. 10,772,559.
(63) Continuation-in-part of application No. 15/963,061, filed on Apr. 25, 2018, now Pat. No. 10,772,559.
Application No. 16/737,052 is a continuation-in-part of application No. 15/725,330, filed on Oct. 5, 2017, now Pat. No. 10,607,507.
(63) Continuation-in-part of application No. 15/725,330, filed on Oct. 5, 2017, now Pat. No. 10,607,507.
Application No. 16/737,052 is a continuation-in-part of application No. 15/431,769, filed on Feb. 14, 2017, now abandoned.
(63) Continuation-in-part of application No. 15/431,769, filed on Feb. 14, 2017, now abandoned.
(63) Continuation-in-part of application No. 15/418,620, filed on Jan. 27, 2017, now abandoned.
Application No. 16/737,052 is a continuation-in-part of application No. 15/294,746, filed on Oct. 16, 2016, now Pat. No. 10,627,861.
(63) Continuation-in-part of application No. 15/294,746, filed on Oct. 16, 2016, now Pat. No. 10,627,861.
(63) Continuation-in-part of application No. 15/206,215, filed on Jul. 8, 2016, now abandoned.
(63) Continuation-in-part of application No. 14/992,073, filed on Jan. 11, 2016, now abandoned.
(63) Continuation-in-part of application No. 14/951,475, filed on Nov. 24, 2015, now Pat. No. 10,314,492.
(63) Continuation-in-part of application No. 14/948,308, filed on Nov. 21, 2015, now abandoned.
(63) Continuation-in-part of application No. 14/550,953, filed on Nov. 22, 2014, now abandoned.
(63) Continuation-in-part of application No. 14/449,387, filed on Aug. 1, 2014, now abandoned.
(63) Continuation-in-part of application No. 14/330,649, filed on Jul. 14, 2014, now abandoned.
(63) Continuation-in-part of application No. 14/132,292, filed on Dec. 18, 2013, now Pat. No. 9,442,100.
(63) Continuation-in-part of application No. 13/901,131, filed on May 23, 2013, now Pat. No. 9,536,449.
(63) Continuation-in-part of application No. 13/901,099, filed on May 23, 2013, now Pat. No. 9,254,099.
(63) Continuation-in-part of application No. 13/616,238, filed on Sep. 14, 2012, now abandoned.
(63) Continuation-in-part of application No. 13/523,739, filed on Jun. 14, 2012, now Pat. No. 9,042,596.
(60) Provisional application No. 63/279,773, filed on Nov. 16, 2021.
(60) Provisional application No. 63/171,838, filed on Apr. 7, 2021.
(60) Provisional application No. 62/930,013, filed on Nov. 4, 2019.
(60) Provisional application No. 62/857,942, filed on Jun. 6, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/814,713, filed on Mar. 6, 2019.
(60) Provisional application No. 62/814,692, filed on Mar. 6, 2019.
(60) Provisional application No. 62/800,478, filed on Feb. 2, 2019.
(60) Provisional application No. 62/549,587, filed on Aug. 24, 2017.
(60) Provisional application No. 62/439,147, filed on Dec. 26, 2016.
(60) Provisional application No. 62/439,147, filed on Dec. 26, 2016.
(60) Provisional application No. 62/349,277, filed on Jun. 13, 2016.
(60) Provisional application No. 62/349,277, filed on Jun. 13, 2016.
(60) Provisional application No. 62/349,277, filed on Jun. 13, 2016.
(60) Provisional application No. 62/311,462, filed on Mar. 22, 2016.
(60) Provisional application No. 62/297,827, filed on Feb. 20, 2016.
(60) Provisional application No. 62/245,311, filed on Oct. 23, 2015.

(51) Int. Cl. G06F 1/16; G16H 20/60; G02B 27/01

(52) U.S. Cl. CPC G16H 20/60; G02B 27/0101; G02B 27/017; G06F 1/163; G02B 2027/0138; G02B 2027/014; G02B 2027/0178

(57) ABSTRACT

A device, system, or method for estimating a person’s food consumption comprises two types of wearable sensors. A first type of sensor collects data to detect when the person consumes food. A second type of sensor is activated when data from the first type of sensor indicates that the person is consuming food. Data from these sensors are analyzed to estimate the types and amounts of food consumed by the person. This device, system, or method then provides feedback and/or coaching to the person to improve their eating habits.

### CROSS-REFERENCE TO RELATED APPLICATIONS

This application is a continuation-in-part of U.S. Pat. Application 17903746 filed on 2022-09-06. This application is a continuation-in-part of U.S. Pat. Application 17239960 filed on 2021-04-26. This application is a continuation-in-part of U.S. Pat. Application 16737052 filed on 2020-01-08.

U.S. Pat.Application 17903746 was a continuation-in-part of U.S. Pat. Application 16568580 filed on 2019-09-12. U.S. Pat. Application 17903746 was a continuation-in-part of U.S. Patent Application 16737052 filed on 2020-01-08. U.S. Pat. Application 17903746 was a continuation-in-part of U.S. Pat. Application 17239960 filed on 2021-04-26. U.S. Pat. Application 17903746 claimed the priority benefit of U.S. Provisional Application 63279773 filed on 2021-11-16.

U.S. Pat. Application 17239960 claimed the priority benefit of U.S. Provisional Application 63171838 filed on 2021-04-07. U.S. Pat. Application 17239960 was a continuation-in-part of U.S. Pat. application 16737052 filed on 2020-01-08.

U.S. Pat. Application 16737052 claimed the priority benefit of U.S. Provisional Application 62930013 filed on 2019-11-04. U.S. Pat. Application 16737052 claimed the priority benefit of U.S. provisional application 62857942 filed on 2019-06-06. U.S. patent application 16737052 claimed the priority benefit of U.S. Provisional Application 62814713 filed on 2019-03-06. U.S. Pat. Application 16737052 claimed the priority benefit of U.S. Provisional Application 62814692 filed on 2019-03-06. U.S. Pat. Application 16737052 claimed the priority benefit of U.S. Provisional Application 62800478 filed on 2019-02-02. U.S. Pat. Application 16737052 was a continuation-in-part of U.S. Pat. Application 16568580 filed on 2019-09-12. U.S. Pat. Application 16737052 was a continuation-in-part of U.S. Pat. application 15963061 filed on 2018-04-25 which issued as U.S. Pat. 10772559 on 2020-09-15. U.S. Pat. Application 16737052 was a continuation-in-part of U.S. Pat. Application 15725330 filed on 2017-10-05 which issued as U.S. Pat. 10607507 on 2020-03-31. U.S. Pat. Application 16737052 was a continuation-in-part of U.S. Pat. Application 15431769 filed on 2017-02-14. U.S. Pat. Application 16737052 was a continuation-in-part of U.S. Pat. Application 15294746 filed on 2016-10-16 which issued as U.S. Pat. 10627861 on 2020-04-21.

U.S. Pat. Application 16568580 claimed the priority benefit of U.S. Provisional Application 62857942 filed on 2019-06-06. U.S. Pat. Application 16568580 claimed the priority benefit of U.S. Provisional Application 62814713 filed on 2019-03-06. U.S. Pat. Application 16568580 claimed the priority benefit of U.S. Provisional Application 62814692 filed on 2019-03-06. U.S. Pat. Application 16568580 was a continuation-in-part of U.S. Pat. Application 15963061 filed on 2018-04-25 which issued as U.S. Pat. 10772559 on 2020-09-15. U.S. Pat. Application 16568580 was a continuation-in-part of U.S. Pat. Application 15725330 filed on 2017-10-05 which issued as U.S. Pat. 10607507 on 2020-03-31. U.S. Pat. Application 16568580 was a continuation-in-part of U.S. Pat. Application 15431769 filed on 2017-02-14. U.S. Pat. Application 16568580 was a continuation-in-part of U.S. Pat. Application 15418620 filed on 2017-01-27. U.S. Pat. Application 16568580 was a continuation-in-part of U.S. Pat. Application 15294746 filed on 2016-10-16 which issued as U.S. Pat. 10627861 on 2020-04-21.

U.S. Pat. Application 15963061 was a continuation-in-part of U.S. Pat. Application 14992073 filed on 2016-01-11. U.S. Pat. Application 15963061 was a continuation-in-part of U.S. Pat. Application 14550953 filed on 2014-11-22.

U.S. Pat. Application 15725330 claimed the priority benefit of U.S. Provisional Application 62549587 filed on 2017-08-24. U.S. Pat. Application 15725330 claimed the priority benefit of U.S. Provisional Application 62439147 filed on 2016-12-26. U.S. Pat. Application 15725330 was a continuation-in-part of U.S. Pat. Application 15431769 filed on 2017-02-14. U.S. Pat. Application 15725330 was a continuation-in-part of U.S. Pat. Application 14951475 filed on 2015-11-24 which issued as U.S. Pat. 10314492 on 2019-06-11.

U.S. Pat. Application 15431769 claimed the priority benefit of U.S. Provisional Application 62439147 filed on 2016-12-26. U.S. Pat. Application 15431769 claimed the priority benefit of U.S. Provisional Application 62349277 filed on 2016-06-13. U.S. Pat. Application 15431769 claimed the priority benefit of U.S. Provisional Application 62311462 filed on 2016-03-22. U.S. Pat. Application 15431769 was a continuation-in-part of U.S. Pat. Application 15294746 filed on 2016-10-16 which issued as U.S. Pat. 10627861 on 2020-04-21. U.S. Pat. Application 15431769 was a continuation-in-part of U.S. Pat. Application 15206215 filed on 2016-07-08. U.S. Pat. Application 15431769 was a continuation-in-part of U.S. Pat. Application 14992073 filed on 2016-01-11. U.S. Pat. Application 15431769 was a continuation-in-part of U.S. Pat. Application 14330649 filed on 2014-07-14.

U.S. Pat. Application 15418620 claimed the priority benefit of U.S. Provisional Application 62297827 filed on 2016-02-20. U.S. Pat. Application 15418620 was a continuation-in-part of U.S. Pat. Application 14951475 filed on 2015-11-24 which issued as U.S. Pat. 10314492 on 2019-06-11.

U.S. Pat. Application 15294746 claimed the priority benefit of U.S. Provisional Application 62349277 filed on 2016-06-13. U.S. Pat. Application 15294746 claimed the priority benefit of U.S. Provisional Application 62245311 filed on 2015-10-23. U.S. Pat. Application 15294746 was a continuation-in-part of U.S. Pat. Application 14951475 filed on 2015-11-24 which issued as U.S. Pat. 10314492 on 2019-06-11.

U.S. Pat. Application 15206215 claimed the priority benefit of U.S. Provisional Application 62349277 filed on 2016-06-13. U.S. Pat. Application 15206215 was a continuation-in-part of U.S. Pat. Application 14951475 filed on 2015-11-24 which issued as U.S. Pat. 10314492 on 2019-06-11. U.S. Pat. Application 15206215 was a continuation-in-part of U.S. Pat. Application 14948308 filed on 2015-11-21.

U.S. Pat. Application 14992073 was a continuation-in-part of U.S. Pat. Application 14562719 filed on 2014-12-07 which issued as U.S. Pat. 10130277 on 2018-11-20. U.S. Pat. Application 14992073 was a continuation-in-part of U.S. Pat. Application 13616238 filed on 2012-09-14.

U.S. Pat. Application 14951475 was a continuation-in-part of U.S. Pat. Application 14071112 filed on 2013-11-04. U.S. Pat. Application 14951475 was a continuation-in-part of U.S. Pat. Application 13901131 filed on 2013-05-23 which issued as U.S. Pat. 9536449 on 2017-01-03.

U.S. Pat. Application 14948308 was a continuation-in-part of U.S. Pat. Application 14550953 filed on 2014-11-22. U.S. Pat. Application 14948308 was a continuation-in-part of U.S. Pat. Application 14449387 filed on 2014-08-01. U.S. Pat. Application 14948308 was a continuation-in-part of U.S. Pat. application 14132292 filed on 2013-12-18 which issued as U.S. pat. 9442100 on 2016-09-13. U.S. Pat. application 14948308 was a continuation-in-part of U.S. Pat. Application 13901099 filed on 2013-05-23 which issued as U.S. Pat. 9254099 on 2016-02-09.

U.S. Pat. Application 14562719 claimed the priority benefit of U.S. Provisional Application 61932517 filed on 2014-01-28.

U.S. Pat. Application 14330649 was a continuation-in-part of U.S. Pat. Application 13523739 filed on 2012-06-14 which issued as U.S. Pat. 9042596 on 2015-05-26.

The entire contents of these applications are incorporated herein by reference.

### FEDERALLY SPONSORED RESEARCH

Not Applicable

### SEQUENCE LISTING OR PROGRAM

Not Applicable

### BACKGROUND -- FIELD OF INVENTION

This invention relates to wearable devices for measuring food consumption.

### INTRODUCTION

Many health problems are caused by poor nutrition. Many people consume too much unhealthy food or not enough healthy food. Although there are complex behavioral reasons for poor dietary habits, better nutritional monitoring and awareness concerning the types and quantities of food consumed can help people to improve their dietary habits and health. Information concerning the types and quantities of food consumed can be part of a system that provides constructive feedback and/or incentives to help people improve their nutritional intake. People can try to track the types and quantities of food consumed without technical assistance. Their unassisted estimates of the types and quantities of consumed food can be translated into types and quantities of nutrients consumed. However, such unassisted tracking can be subjective. Also, such unassisted tracking can be particularly challenging for non-standardized food items such as food prepared in an ad hoc manner at restaurants or in homes. It would be useful to have a relatively-unobtrusive wearable device which can help people to accurately track the types and quantities of food which they consume and provide coaching to encourage them to have healthier eating habits.

### REVIEW OF THE RELEVANT ART

In the patent literature, U.S. Pat. 10901509 (Aimone et al., Jan. 26, 2021, “Wearable Computing Apparatus and Method”) discloses a wearable computing device comprising at least one brainwave sensor. U.S. Pat. 11222422 (Alshurafa et al., Jan. 11, 2022, “Hyperspectral Imaging Sensor”) discloses a hyperspectral imaging sensor system to identify item composition. U.S. Pat. Application 20160148535 (Ashby, May 26, 2016, “Tracking Nutritional Information about Consumed Food”) discloses an eating monitor which monitors swallowing and/or chewing. U.S. Pat. Application 20160148536 (Ashby, May 26, 2016, “Tracking Nutritional Information about Consumed Food with a Wearable Device”) discloses an eating monitor with a camera. U.S. Pat. 9146147 (Bakhsh, Sep. 29, 2015, “Dynamic Nutrition Tracking Utensils”) discloses nutritional intake tracking using a smart utensil.

U.S. Pat. Application 20210307677 (Bi et al., Oct. 7, 2021, “System for Detecting Eating with Sensor Mounted by the Ear”) discloses a wearable device for detecting eating episodes via a contact microphone. U.S. Pat. Application 20210307686 (Catani et al., Oct. 7, 2021, “Methods and Systems to Detect Eating”) discloses methods and systems for automated eating detection comprising a continuous glucose monitor (CGM) and an accelerometer. U.S. Pat. Application 20190213416 (Cho et al., Jul. 11, 2019, “Electronic Device and Method for Processing Information Associated with Food”) and patent 10803315 (Cho et al., Oct. 13, 2020, “Electronic Device and Method for Processing Information Associated with Food”) disclose analysis of food images to obtain nutritional information concerning food items and to recommend food consumption quantities. U.S. Pat. Application 20170061821 (Choi et al., Mar. 2, 2017, “Systems and Methods for Performing a Food Tracking Service for Tracking Consumption of Food Items”) discloses a food tracking service. U.S. Pat. Application 20190167190 (Choi et al., Jun. 6, 2019, “Healthcare Apparatus and Operating Method Thereof”) discloses a dietary monitoring device which emits light of different wavelengths.

U.S. Pat. Application 11478096 (Chung et al., Oct. 25, 2022, “Food Monitoring System”) discloses a serving receptacle, an information processor, and utensils which are used to estimate food quantity. U.S. Pat. Application 20220400195 (Churovich et al., Dec. 15, 2022, “Electronic Visual Food Probe”) and patent 11366305 (Churovich et al., Jun. 21, 2022, “Electronic Visual Food Probe”) disclose an electronic visual food probe to view inside food. U.S. Pat. 10143420 (Contant, Dec. 4, 2018, “Eating Utensil to Monitor and Regulate Dietary Intake”) discloses a dietary intake regulating device that also monitors physical activity. U.S. Pat. Application 20160163037 (Dehais et al., Jun. 9, 2016, “Estimation of Food Volume and Carbs”) discloses an image-based food identification system including a projected light pattern. U.S. Pat. Application 20170249445 (Devries et al., Aug. 31, 2017, “Portable Devices and Methods for Measuring Nutritional Intake”) discloses a nutritional intake monitoring system with biosensors.

U.S. Pat. Application 20180214077 (Dunki-Jacobs, Aug. 2, 2018, “Meal Detection Devices and Methods”) and patent 10791988 (Dunki-Jacobs, Aug. 2, 2018, “Meal Detection Devices and Methods”) disclose using biometric sensors to detect meal intake and control a therapeutic device. U.S. Pat. Application 20150294450 (Eyring, Oct. 15, 2015, “Systems and Methods for Measuring Calorie Intake”) discloses an image-based system for measuring caloric input. U.S. Pat. Application 20220301683 (Feilner, Sep. 22, 2022, “Detecting and Quantifying a Liquid and/or Food Intake of a User Wearing a Hearing Device”) discloses detecting and quantifying a food intake via a microphone.

U.S. Pat. Applications 20090012433 (Fernstrom et al., Jan. 8, 2009, “Method, Apparatus and System for Food Intake and Physical Activity Assessment”), 20130267794 (Fernstrom et al., Oct. 10, 2013, “Method, Apparatus and System for Food Intake and Physical Activity Assessment”), and 20180348187 (Fernstrom et al., Dec. 6, 2018, “Method, Apparatus and System for Food Intake and Physical Activity Assessment”), as well as U.S. Pats. 9198621 (Fernstrom et al., Dec. 1, 2015, “Method, Apparatus and System for Food Intake and Physical Activity Assessment”) and 10006896 (Fernstrom et al., Jun. 26, 2018, “Method, Apparatus and System for Food Intake and Physical Activity Assessment”), disclose wearable buttons and necklaces for monitoring eating with cameras. U.S. Pat. 10900943 (Fernstrom et al, Jan. 26, 2021, “Method, Apparatus and System for Food Intake and Physical Activity Assessment”) discloses monitoring food consumption using a wearable device with two video cameras and an infrared sensor. U.S. Pat. Application 20150325142 (Ghalavand, Nov. 12, 2015, “Calorie Balance System”) discloses a calorie balance system with smart utensils and/or food scales.

U.S. Pat. Applications 20160299061 (Goldring et al., Oct. 13, 2016, “Spectrometry Systems, Methods, and Applications”), 20170160131 (Goldring et al., Jun. 8, 2017, “Spectrometry Systems, Methods, and Applications”), 20180085003 (Goldring et al., Mar. 29, 2018, “Spectrometry Systems, Methods, and Applications”), 20180120155 (Rosen et al., May 3, 2018, “Spectrometry Systems, Methods, and Applications”), and 20180180478 (Goldring et al., Jun. 28, 2018, “Spectrometry Systems, Methods, and Applications”) disclose a handheld spectrometer to measure the spectra of objects. U.S. Pat. Application 20180136042 (Goldring et al., May 17, 2018, “Spectrometry System with Visible Aiming Beam”) discloses a handheld spectrometer with a visible aiming beam. U.S. Pat. Application 20180252580 (Goldring et al., Sep. 6, 2018, “Low-Cost Spectrometry System for End-User Food Analysis”) discloses a compact spectrometer that can be used in mobile devices such as smart phones. U.S. Pat. Application 20190033130 (Goldring et al., Jan. 31, 2019, “Spectrometry Systems, Methods, and Applications”) discloses a hand held spectrometer with wavelength multiplexing. U.S. Pat. Application 20190033132 (Goldring et al., Jan. 31, 2019, “Spectrometry System with Decreased Light Path”) discloses a spectrometer with a plurality of isolated optical channels. U.S. patent application 20190041265 (Rosen et al., Feb. 7, 2019, “Spatially Variable Filter Systems and Methods”) discloses a compact spectrometer system with a spatially variable filter.

U.S. Pat. Application 20190295440 (Hadad, Sep. 26, 2019, “Systems and Methods for Food Analysis, Personalized Recommendations and Health Management”) discloses a method for developing a food ontology. U.S. Pat. Applications 20190244541 (Hadad et al., Aug. 8, 2019, “Systems and Methods for Generating Personalized Nutritional Recommendations”), 20140255882 (Hadad et al., Sep. 11, 2014, “Interactive Engine to Provide Personal Recommendations for Nutrition, to Help the General Public to Live a Balanced Healthier Lifestyle”), and 20190290172 (Hadad et al., Sep. 26, 2019, “Systems and Methods for Food Analysis, Personalized Recommendations, and Health Management”) disclose methods to provide nutrition recommendations based on a person’s preferences, habits, medical and activity.

U.S. Pat. Application 20190272845 (Hasan et al., Sep. 5, 2019, “System and Method for Monitoring Dietary Activity”) discloses a system for monitoring dietary activity via a neck-worn device with an audio input unit. U.S. Pat. Application 20160103910 (Kim et al., Apr. 14, 2016, “System and Method for Food Categorization”) discloses a food categorization engine. U.S. Pat. Application 20190244704 (Kim et al., Aug. 8, 2019, “Dietary Habit Management Apparatus and Method”) discloses a dietary habit management apparatus using biometric measurements. U.S. Pat. Application 20200015697 (Kinreich, Apr. 15, 2021, “Method and System for Analyzing Neural and Muscle Activity in a Subject’s Head for the Detection of Mastication”) discloses a wearable apparatus to automatically monitor consumption by analyzing images captured of the environment. U.S. Pat. Application 20220012467 (Kuo et al., Jan. 13, 2022, “Multi-Sensor Analysis of Food”) discloses a method for estimating food composition by 3D imaging and millimeter-wave radar.

U.S. Pat. Application 20160140869 (Kuwahara et al., May 19, 2016, “Food Intake Controlling Devices and Methods”) discloses image-based technologies for controlling food intake. U.S. Pats. 10359381 (Lewis et al., Jul. 23, 2019, “Methods and Systems for Determining an Internal Property of a Food Product”) and 11313820 (Lewis et al., Apr. 26, 2022, “Methods and Systems for Determining an Internal Property of a Food Product”) disclose analyzing interior and external properties of food. U.S. Pat. application 20170156634 (Li et al., Jun. 8, 2017, “Wearable Device and Method for Monitoring Eating”) and patent 10499833 (Li et al., Dec. 10, 2019, “Wearable Device and Method for Monitoring Eating”) disclose a wearable device with an acceleration sensor to monitor eating. U.S. Pat. 11568760 (Meier, Jan. 31, 2023, “Augmented Reality Calorie Counter”) discloses using chewing noises and food images to estimate food volume. U.S. Pat. 10952670 (Mori et al., Mar. 23, 2021, “Meal Detection Method, Meal Detection System, and Storage Medium”) discloses meal detection by analyzing arm motion data and heart rate data.

U.S. Pat. Application 20150302160 (Muthukumar et al., Oct. 22, 2015, “Method and Apparatus for Monitoring Diet and Activity”) discloses a method and device for analyzing food with a camera and a spectroscopic sensor. U.S. Pats. 10249214 (Novotny et al., Apr. 2, 2019, “Personal Wellness Monitoring System”) and 11206980 (Novotny et al., Dec. 28, 2021, “Personal Wellness Monitoring System”) disclose a personal nutrition, health, wellness and fitness monitor that captures 3D images. U.S. Pat. Application 20160313241 (Ochi et al., Nov. 27, 2016, “Calorie Measurement Device”) disclose, Mar. 17, 2016, “Food Intake Monitor”) discloses a jaw motion sensor to measure food intake. U.S. Pat. Application 20210183493 (Oh et al., Jun. 17, 2021, “Systems and Methods for Automatic Activity Tracking”) discloses systems and methods for tracking activities (e.g., eating moments) from a plurality of multimodal inputs.

U.S. Pat. 9349297 (Ortiz et al., May 24, 2016, “System and Method for Nutrition Analysis Using Food Image Recognition”) discloses a system and method for determining the nutritional value of a food item. U.S. Pat. 9364106 (Ortiz, Jun. 14, 2016, “Apparatus and Method for Identifying, Measuring and Analyzing Food Nutritional Values and Consumer Eating Behaviors”) discloses a food container for determining the nutritional value of a food item. U.S. Pat. Application 20180005545 (Pathak et al., Jan. 4, 2018, “Assessment of Nutrition Intake Using a Handheld Tool”) discloses a smart food utensil for measuring food mass. U.S. Pat. Application 20210369187 (Raju et al., Dec. 2, 2021, “Non-Contact Chewing Sensor and Portion Estimator”) discloses an optical proximity sensor to monitor chewing. U.S. Pat. 10423045 (Roberts et al., Sep. 24, 2019, “Electro-Optical Diffractive Waveplate Beam Shaping System”) discloses optical beam shaping systems with a diffractive waveplate diffuser.

U.S. Pat. Application 20160073953 (Sazonov et al., Mar. 17, 2016, “Food Intake Monitor”) discloses monitoring food consumption using a wearable device with a jaw motion sensor and a hand gesture sensor. U.S. Pat. Application 20180242908 (Sazonov et al., Aug. 30, 2018, “Food Intake Monitor”) and U.S. Pat. 10736566 (Sazonov, Aug. 11, 2020, “Food Intake Monitor”) disclose monitoring food consumption using an ear-worn device or eyeglasses with a pressure sensor and accelerometer. U.S. Pat. Applications 20200337635 (Sazonov et al., Oct. 29, 2020, “Food Intake Monitor”) and 20210345959 (Sazonov et al., Nov. 11, 2021, “Food Intake Monitor”) and U.S. Pats. 11006896 (Sazonov et al., May 18, 2021, “Food Intake Monitor”) and 11564623 (Sazonov et al., Jan. 31, 2023, “Food Intake Monitor”) disclose an optical proximity sensor and/or temporalis muscle activity sensor to monitor chewing.

U.S. Pat. Application 20210110159 (Shashua et al., Apr. 15, 2021, “Systems and Methods for Monitoring Consumption”) and patent 11462006 (Shashua et al., Oct. 4, 2022, “Systems and Methods for Monitoring Consumption”) disclose a wearable apparatus to automatically monitor consumption by a user by analyzing images. U.S. Pat. 10952669 (Shi et al., Mar. 23, 2021, “System for Monitoring Eating Habit Using a Wearable Device”) discloses a wearable device for monitoring eating behavior with an imaging sensor and an electromyography (EMG) sensor. U.S. Pat. 11510610 (Tanimura et al., Nov. 29, 2022, “Eating Monitoring Method, Program, and Eating Monitoring Device”) discloses an eating monitoring method using a sensor to measure jaw movement. U.S. Pat. 11013430 (Tanriover et al., May 25, 2021, “Methods and Apparatus for Identifying Food Chewed and/or Beverage Drank”) discloses methods and apparatuses for identifying food consumption via a chewing analyzer that extracts vibration data.

U.S. Pat. Application 20190333634 (Vleugels et al., Oct. 31, 2019, “Method and Apparatus for Tracking of Food Intake and Other Behaviors and Providing Relevant Feedback”), 20170220772 (Vleugels et al., Aug. 3, 2017, “Method and Apparatus for Tracking of Food Intake and Other Behaviors and Providing Relevant Feedback”), and 20180300458 (Vleugels et al., Oct. 18, 2018, “Method and Apparatus for Tracking of Food Intake and Other Behaviors and Providing Relevant Feedback”), as well as U.S. Pats. 10102342 (Vleugels et al., Oct. 16, 2018, “Method and Apparatus for Tracking of Food Intake and Other Behaviors and Providing Relevant Feedback”) and 10373716 (Vleugels et al., Aug. 6, 2019, “Method and Apparatus for Tracking of Food Intake and Other Behaviors and Providing Relevant Feedback”), disclose a method for detecting, identifying, analyzing, quantifying, tracking, processing and/or influencing food consumption. U.S. Pat. Application 20190236465 (Vleugels, Aug. 1, 2019, “Activation of Ancillary Sensor Systems Based on Triggers from a Wearable Gesture Sensing Device”) discloses an eating monitor with gesture recognition.

U.S. Pat. Application 20200294645 (Vleugels, Sep. 17, 2020, “Gesture-Based Detection of a Physical Behavior Event Based on Gesture Sensor Data and Supplemental Information from at Least One External Source”) discloses an automated medication dispensing system which recognizes gestures. U.S. Pat. 10790054 (Vleugels et al., Sep. 29, 2020, “Method and Apparatus for Tracking of Food Intake and Other Behaviors and Providing Relevant Feedback”) discloses a computer-based method of detecting gestures. U.S. Pat. Application 20200381101 (Vleugels, Dec. 3, 2020, “Method and Apparatus for Tracking of Food Intake and Other Behaviors and Providing Relevant Feedback”) discloses methods for detecting, identifying, analyzing, quantifying, tracking, processing and/or influencing, related to the intake of food, eating habits, eating patterns, and/or triggers for food intake events, eating habits, or eating patterns.

U.S. Pat. Applications 20200381101 (Vleugels, Dec. 3, 2020, “Method and Apparatus for Tracking of Food Intake and Other Behaviors and Providing Relevant Feedback”) and 20210350920 (Vleugels et al., Nov. 11, 2021, “Method and Apparatus for Tracking of Food Intake and Other Behaviors and Providing Relevant Feedback”) disclose methods for detecting, identifying, analyzing, quantifying, tracking, processing and/or influencing the intake of food, eating habits, eating patterns, and/or triggers for food intake events, eating habits, or eating patterns.

U.S. Pat. Application 20160091419 (Watson et al., Mar. 31, 2016, “Analyzing and Correlating Spectra, Identifying Samples and Their Ingredients, and Displaying Related Personalized Information”) discloses a spectral analysis method for food analysis. U.S. Pat. Applications 20170292908 (Wilk et al., Oct. 12, 2017, “Spectrometry System Applications”) and 20180143073 (Goldring et al., May 24, 2018, “Spectrometry System Applications”) disclose a spectrometer system to determine spectra of an object. U.S. Pat. Application 20170193854 (Yuan et al., Jan. 5, 2016, “Smart Wearable Device and Health Monitoring Method”) discloses a wearable device with a camera to monitor eating. U.S. patent 10058283 (Zerick et al., Apr. 6, 2016, “Determining Food Identities with Intra-Oral Spectrometer Devices”) discloses an intra-oral device for food analysis.

In the non-patent literature, Amft et al., 2005 (“Detection of Eating and Drinking Arm Gestures Using Inertial Body-Worn Sensors”) discloses eating detection by analyzing arm gestures. Bedri et al., 2015 (“Detecting Mastication: A Wearable Approach”; access to abstract only) discloses eating detection using an ear-worn devices with a gyroscope and proximity sensors. Bedri et al., 2017 (“EarBit: Using Wearable Sensors to Detect Eating Episodes in Unconstrained Environments”) discloses eating detection using an ear-worn device with inertial, optical, and acoustic sensors. Bedri et al., 2020a (“FitByte: Automatic Diet Monitoring in Unconstrained Situations Using Multimodal Sensing on Eyeglasses”) discloses food consumption monitoring using a device with a motion sensor, an infrared sensor, and a camera which is attached to eyeglasses. Bell et al., 2020 (“Automatic, Wearable-Based, In-Field Eating Detection Approaches for Public Health Research: A Scoping Review”) reviews wearable sensors for eating detection.

Bi et al., 2016 (“AutoDietary: A Wearable Acoustic Sensor System for Food Intake Recognition in Daily Life”) discloses eating detection using a neck-worn device with sound sensors. Bi et al., 2017 (“Toward a Wearable Sensor for Eating Detection”) discloses eating detection using ear-worn and neck-worn devices with sound sensors and EMG sensors. Bi et al., 2018 (“Auracle: Detecting Eating Episodes with an Ear-Mounted Sensor”) discloses eating detection using an ear-worn device with a microphone. Borrell, 2011 (“Every Bite You Take”) discloses food consumption monitoring using a neck-worn device with GPS, a microphone, an accelerometer, and a camera. Brenna et al., 2019 (“A Survey of Automatic Methods for Nutritional Assessment) reviews automatic methods for nutritional assessment. Chun et al., 2018 (“Detecting Eating Episodes by Tracking Jawbone Movements with a Non-Contact Wearable Sensor”) discloses eating detection using a necklace with an accelerometer and range sensor.

Chung et al., 2017 (“A Glasses-Type Wearable Device for Monitoring the Patterns of Food Intake and Facial Activity”) discloses eating detection using a force-based chewing sensor on eyeglasses. Dimitratos et al., 2020 (“Wearable Technology to Quantify the Nutritional Intake of Adults: Validation Study”) discloses high variability in food consumption monitoring using only a wristband with a motion sensor. Dong et al., 2009 (“A Device for Detecting and Counting Bites of Food Taken by a Person During Eating”) discloses bite counting using a wrist-worn orientation sensor. Dong et al., 2011 (“Detecting Eating Using a Wrist Mounted Device During Normal Daily Activities”) discloses eating detection using a watch with a motion sensor. Dong et al., 2012b (“A New Method for Measuring Meal Intake in Humans via Automated Wrist Motion Tracking”) discloses bite counting using a wrist-worn gyroscope. Dong et al., 2014 (“Detecting Periods of Eating During Free-Living by Tracking Wrist Motion”) discloses eating detection using a wrist-worn device with motion sensors.

Farooq et al., 2016 (“A Novel Wearable Device for Food Intake and Physical Activity Recognition”) discloses eating detection using eyeglasses with a piezoelectric strain sensor and an accelerometer. Farooq et al., 2017 (“Segmentation and Characterization of Chewing Bouts by Monitoring Temporalis Muscle Using Smart Glasses With Piezoelectric Sensor”) discloses chew counting using eyeglasses with a piezoelectric strain sensor. Fontana et al., 2014 (“Automatic Ingestion Monitor: A Novel Wearable Device for Monitoring of Ingestive Behavior”) discloses food consumption monitoring using a device with a jaw motion sensor, a hand gesture sensor, and an accelerometer. Fontana et al., 2015 (“Energy Intake Estimation from Counts of Chews and Swallows”) discloses counting chews and swallows using wearable sensors and video analysis. Jasper et al., 2016 (“Effects of Bite Count Feedback from a Wearable Device and Goal-Setting on Consumption in Young Adults”) discloses the effect of feedback based on bite counting.

Liu et al., 2012 (“An Intelligent Food-Intake Monitoring System Using Wearable Sensors”) discloses food consumption monitoring using an ear-worn device with a microphone and camera. Magrini et al., 2017 (“Wearable Devices for Caloric Intake Assessment: State of Art and Future Developments”) reviews wearable devices for automatic recording of food consumption. Makeyev et al., 2012 (“Automatic Food Intake Detection Based on Swallowing Sounds”) discloses swallowing detection using wearable sound sensors. Merck et al., 2016 (“Multimodality Sensing for Eating Recognition”; access to abstract only) discloses eating detection using eyeglasses and smart watches on each wrist, combining motion and sound sensors.

Mirtchouk et al., 2016 (“Automated Estimation of Food Type and Amount Consumed from Body-Worn Audio and Motion Sensors”; access to abstract only) discloses food consumption monitoring using in-ear audio plus head and wrist motion. Mirtchouk et al., 2017 (“Recognizing Eating from Body-Worn Sensors: Combining Free-Living and Laboratory Data”) discloses eating detection using head-worn and wrist-worn motion sensors and sound sensors. O’Loughlin et al., 2013 (“Using a Wearable Camera to Increase the Accuracy of Dietary Analysis”) discloses food consumption monitoring using a combination of a wearable camera and self-reported logging. Prioleau et al., 2017 (“Unobtrusive and Wearable Systems for Automatic Dietary Monitoring”) reviews wearable and hand-held approaches to dietary monitoring. Rahman et al., 2015 (“Unintrusive Eating Recognition Using Google Glass”) discloses eating detection using eyeglasses with an inertial motion sensor.

Sazonov et al., 2008 (“Non-Invasive Monitoring of Chewing and Swallowing for Objective Quantification of Ingestive Behavior”) discloses counting chews and swallows using ear-worn and/or neck-worn strain and sound sensors. Sazonov et al., 2009 (“Toward Objective Monitoring of Ingestive Behavior in Free-Living Population”) discloses counting chews and swallows using strain sensors. Sazonov et al., 2010a (“The Energetics of Obesity: A Review: Monitoring Energy Intake and Energy Expenditure in Humans”) reviews devices for monitoring food consumption. Sazonov et al., 2010b (“Automatic Detection of Swallowing Events by Acoustical Means for Applications of Monitoring of Ingestive Behavior”) discloses swallowing detection using wearable sound sensors. Sazonov et al., 2012 (“A Sensor System for Automatic Detection of Food Intake Through Non-Invasive Monitoring of Chewing”) discloses eating detection using a wearable piezoelectric strain gauge.

Schiboni et al., 2018 (“Automatic Dietary Monitoring Using Wearable Accessories”) reviews wearable devices for dietary monitoring. Sen et al., 2018 (“Annapurna: Building a Real-World Smartwatch-Based Automated Food Journal”; access to abstract only) discloses food consumption monitoring using a smart watch with a motion sensor and a camera. Sun et al., 2010 (“A Wearable Electronic System for Objective Dietary Assessment”) discloses food consumption monitoring using a wearable circular device with earphones, microphones, accelerometers, or skin-surface electrodes. Tamura et al., 2016 (“Review of Monitoring Devices for Food Intake”) reviews wearable devices for eating detection and food consumption monitoring. Thomaz et al., 2013 (“Feasibility of Identifying Eating Moments from First-Person Images Leveraging Human Computation”) discloses eating detection through analysis of first-person images. Thomaz et al., 2015 (“A Practical Approach for Recognizing Eating Moments with Wrist-Mounted Inertial Sensing”) discloses eating detection using a smart watch with an accelerometer.

Vu et al., 2017 (“Wearable Food Intake Monitoring Technologies: A Comprehensive Review”) reviews sensing platforms and data analytic approaches to solve the challenges of food-intake monitoring, including ear-based chewing and swallowing detection systems and wearable cameras. Young, 2020 (“FitByte Uses Sensors on Eyeglasses to Automatically Monitor Diet: CMU Researchers Propose a Multimodal System to Track Foods, Liquid Intake”) discloses food consumption monitoring using a device with a motion sensor, an infrared sensor, and a camera which is attached to eyeglasses. Zhang et al., 2016 (“Diet Eyeglasses: Recognising Food Chewing Using EMG and Smart Eyeglasses”; access to abstract only) discloses eating detection using eyeglasses with EMG sensors. Zhang et al., 2018a (“Free-Living Eating Event Spotting Using EMG-Monitoring Eyeglasses”; access to abstract only) discloses eating detection using eyeglasses with EMG sensors. Zhang et al., 2018b (“Monitoring Chewing and Eating in Free-Living Using Smart Eyeglasses”) discloses eating detection using eyeglasses with EMG sensors.

### SUMMARY OF THE INVENTION

This invention can be embodied in a device, system, or method for estimating a person’s consumption of food and nutrients via two or more different types of sensors which are worn by the person. A first type of wearable sensor automatically collects data which is analyzed to detect when the person is consuming food. A second type of wearable sensor is triggered to collect additional data when analysis of data from the first type of sensor indicates that the person is consuming food. Data from the first and second types of sensors are jointly analyzed to estimate the types and amounts of food consumed by the person. This device, system, or method detects when a person consumes food, analyzes data from wearable sensors to estimate the types and amounts of food that the person consumes, and provides feedback and/or coaching to the person to improve their eating habits.

### BRIEF DESCRIPTION OF THE FIGURES

FIGS. 1 through 4 shows four views, at four different times, of an example of wearable device (e.g. Augmented Reality eyewear) with a first sensor (e.g. a microphone), a second sensor (e.g. a camera), and a feedback mechanism (e.g. a display) which detects when a person consumes food, estimates the types and amounts of food consumed by the person, and provides feedback and/or coaching to the person.

FIG. 1 shows this eyewear at a first point in time, before a person has begun consuming food.

FIG. 2 shows this eyewear at a second point in time, when the person is consuming food and this food consumption is detected by analysis of data from the first sensor (e.g. the microphone).

FIG. 3 shows this eyewear at a third point in time, when detection of food consumption has triggered activation of a second sensor (e.g. the camera).

FIG. 4 shows this eyewear at a fourth point in time, when data from the first sensor (e.g. the microphone) and data from the second sensor (e.g. the camera) have been analyzed to estimate the types and amounts of food consumed by the person and feedback is provided to the person (e.g. via a display in the AR eyewear).

### DETAILED DESCRIPTION OF THE FIGURES

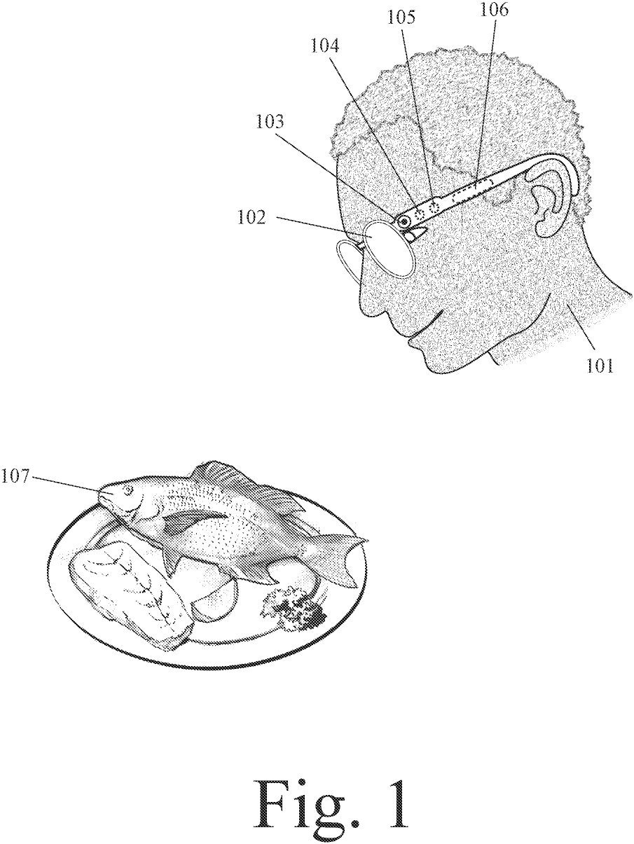

FIGS. 1 through 4 show four sequential views of an example of a device or system for estimating food consumption comprising: a first wearable sensor which is configured to be worn by a person, wherein a first set of data is recorded by the first sensor, and wherein the first set of data is analyzed to detect when the person is consuming food; a second wearable sensor which is configured to be worn by the person, wherein a second set of data is recorded by the second sensor, wherein the first set of data and the second set of data are jointly analyzed to estimate the types and amounts of food consumed by the person, and wherein (a) the second sensor is triggered to start recording the second set of data when analysis of the first set of data indicates that the person is consuming food or (b) wherein the second sensor is triggered to increase the amount, level, or scope of data in the second set of data when analysis of the first set of data indicates that the person is consuming food; a data processor, wherein the first set of data and the second set of data are analyzed by the data processor; and a feedback mechanism which provides feedback to the person based on the types and amounts of food consumed by the person.

In the example shown in FIGS. 1 through 4, the first wearable sensor is a microphone 105 which is part of Augmented Reality (AR) eyewear 102 worn by person 101, the second wearable sensor is a camera 103 which is also part of the eyewear, and the feedback mechanism is a light-emitting display 104 which displays information 108 concerning types and amounts of food 107 in the person’s field of view via the eyewear. FIGS. 1 through 4 also show data processor 106, wherein data from the wearable sensors is processed. In an example, data processing can be done by a separate and/or remote data processor. In this example, the sensors are integral parts of the eyewear. In another example, a device or system can be modular, wherein a device or component with sensors is attached to eyewear.

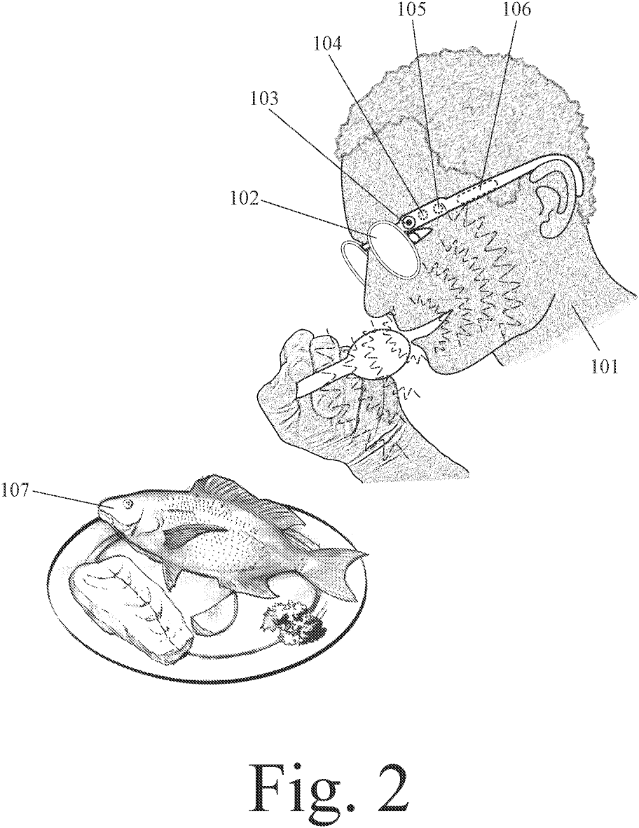

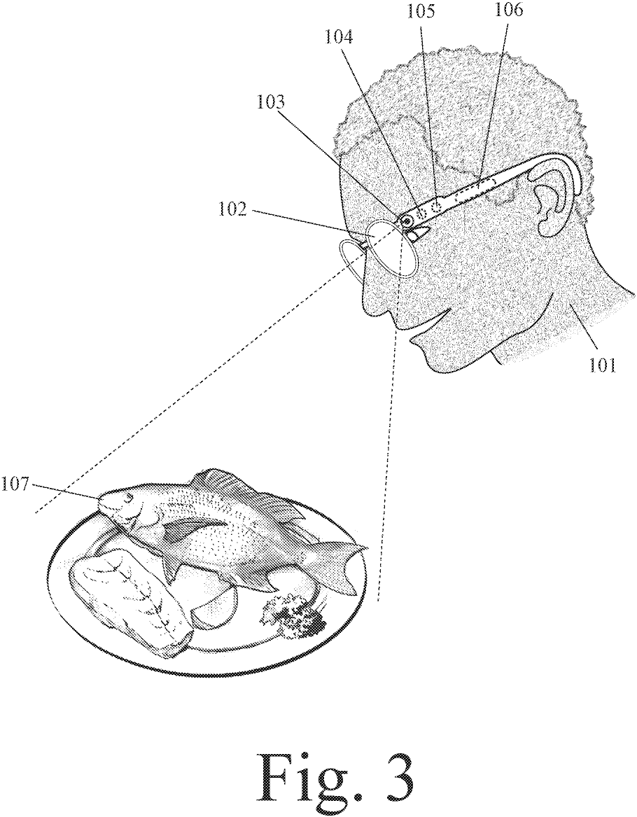

FIG. 1 shows this device or system at a first point in time before the person has begun consuming food. FIG. 2 shows this device or system at a second point in time in which the person is consuming food. In this example, the person’s food consumption is detected by analysis of a first set of data (e.g. chewing and/or swallowing sounds recorded by the microphone), wherein this analysis identifies chewing and/or swallowing sounds which are associated with food consumption. FIG. 3 shows this device or system at a third point in time when detection of food consumption has triggered collection of a second set of data (e.g. food images recorded by the camera, which is automatically triggered and/or activated by detection of food consumption).

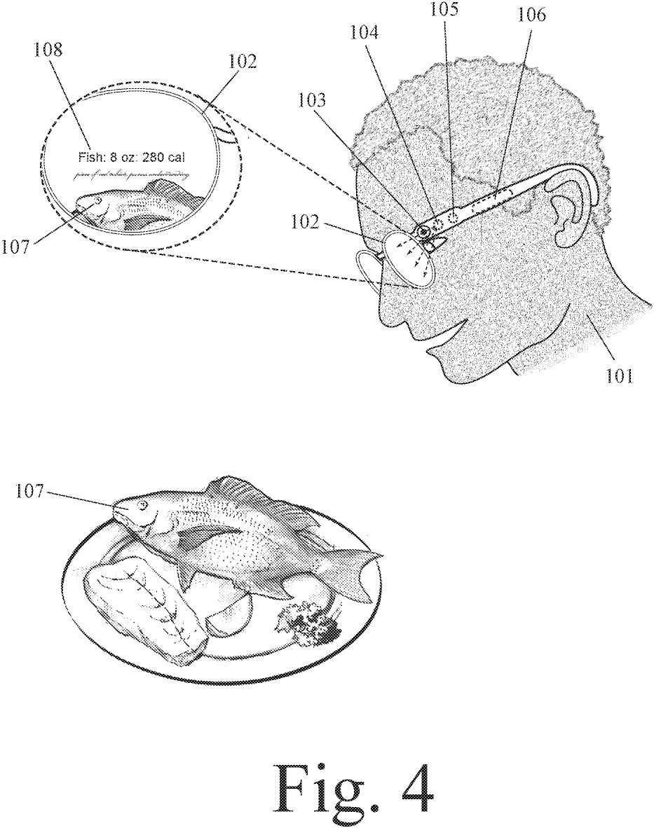

FIG. 4 shows this device or system at a fourth point in time when: the first and second sets of data have been analyzed to estimate the types and amounts of food being consumed by the person; and information concerning these types and amounts of food are being displayed in the person’s view by the Augmented Reality (AR) eyewear. The dotted-line oval in upper left portion of FIG. 4 shows an enlarged view of what the person sees as they look through the left lens of the eyewear. Example variations discussed elsewhere in this disclosure or in priority-linked disclosures can also be applied to this example where relevant.

In an example, a device, system, or method for estimating food consumption can comprise: a first wearable sensor which is configured to be worn by a person, wherein a first set of data is recorded by the first sensor, and wherein the first set of data is analyzed to detect when the person is consuming food; a second wearable sensor which is configured to be worn by the person, wherein a second set of data is recorded by the second sensor, wherein the first set of data and the second set of data are jointly analyzed to estimate the types and amounts of food consumed by the person, and wherein (a) the second sensor is triggered to start recording the second set of data when analysis of the first set of data indicates that the person is consuming food or (b) wherein the second sensor is triggered to increase the amount, level, or scope of data in the second set of data when analysis of the first set of data indicates that the person is consuming food; a data processor, wherein the first set of data and the second set of data are analyzed by the data processor; and a feedback mechanism which provides feedback to the person based on the types and amounts of food consumed by the person.

In an example, the first wearable sensor can be a microphone which is part of, or attached to, eyewear; the second wearable sensor can be a camera which is part of, or attached to, the eyewear; and the feedback mechanism communicates information concerning the types and amounts of food to the person via the eyewear. In an example, the first wearable sensor can be a microphone which is part of, or attached to, an ear-worn device; the second wearable sensor can be a camera which is part of, or attached to, the ear-worn device; and the feedback mechanism communicates information concerning the types and amounts of food to the person via the ear-worn device.

In an example, the first wearable sensor can be a microphone which is part of, or attached to, eyewear; the second wearable sensor can be a camera which is part of, or attached to, the eyewear; and the feedback mechanism changes the appearance of food in the person’s field of vision via eyewear to change the person’s food consumption behavior. In an example, the first wearable sensor can be a motion which is part of, or attached to, a wrist-worn device; the second wearable sensor can be a camera which is part of, or attached to, the wrist-worn device; and the feedback mechanism makes recommendations concerning food consumption to the person via the wrist-worn device.

In an example, the first wearable sensor can be a motion which is part of, or attached to, a wrist-worn device; the second wearable sensor can be a camera which is part of, or attached to, the wrist-worn device; and the feedback mechanism communicates information concerning the types and amounts of food to the person via the wrist-worn device. In an example, the first wearable sensor can be a reflective optical which is part of, or attached to, an ear-worn device; the second wearable sensor can be a camera which is part of, or attached to, the ear-worn device; and the feedback mechanism makes recommendations concerning food consumption to the person via the ear-worn device.

In an example, the first wearable sensor can be a reflective optical which is part of, or attached to, an ear-worn device; the second wearable sensor can be a camera which is part of, or attached to, the ear-worn device; and the feedback mechanism communicates information concerning the types and amounts of food to the person via the ear-worn device. In an example, the first wearable sensor can be a reflective optical which is part of, or attached to, eyewear; the second wearable sensor can be a camera which is part of, or attached to, the eyewear; and the feedback mechanism communicates information concerning the types and amounts of food to the person via the eyewear.

In an example, the first wearable sensor can be a reflective optical which is part of, or attached to, eyewear; the second wearable sensor can be a camera which is part of, or attached to, the eyewear; and the feedback mechanism changes the appearance of food in the person’s field of vision via eyewear to change the person’s food consumption behavior. In an example, the first wearable sensor can be a microphone which is part of, or attached to, an ear-worn device; the second wearable sensor can be a camera which is part of, or attached to, the ear-worn device; and the feedback mechanism makes recommendations concerning food consumption to the person via the ear-worn device.

In an example, the first wearable sensor can be a microphone which is part of, or attached to, an ear-worn device; the second wearable sensor can be a camera which is part of, or attached to, the ear-worn device; and the feedback mechanism makes recommendations concerning food consumption to the person via a phone. In an example, the first wearable sensor can be a microphone which is part of, or attached to, an ear-worn device; the second wearable sensor can be a camera which is part of, or attached to, the ear-worn device; and the feedback mechanism communicates information concerning the types and amounts of food to the person via a phone.

In an example, the first wearable sensor can be a microphone which is part of, or attached to, eyewear; the second wearable sensor can be a camera which is part of, or attached to, the eyewear; and the feedback mechanism makes recommendations concerning food consumption to the person via the eyewear. In an example, the first wearable sensor can be a motion which is part of, or attached to, a finger-worn device; the second wearable sensor can be a camera which is part of, or attached to, the finger-worn device; and the feedback mechanism makes recommendations concerning food consumption to the person via a phone.

In an example, the first wearable sensor can be a motion which is part of, or attached to, a finger-worn device; the second wearable sensor can be a camera which is part of, or attached to, the finger-worn device; and the feedback mechanism communicates information concerning the types and amounts of food to the person via a phone. In an example, the first wearable sensor can be a motion which is part of, or attached to, a wrist-worn device; the second wearable sensor can be a camera which is part of, or attached to, the wrist-worn device; and the feedback mechanism communicates information concerning the types and amounts of food to the person via a phone.

In an example, the first wearable sensor can be a optical which is part of, or attached to, an ear-worn device; the second wearable sensor can be a camera which is part of, or attached to, the ear-worn device; and the feedback mechanism makes recommendations concerning food consumption to the person via the ear-worn device. In an example, the first wearable sensor can be a reflective optical which is part of, or attached to, eyewear; the second wearable sensor can be a camera which is part of, or attached to, the eyewear; and the feedback mechanism makes recommendations concerning food consumption to the person via the eyewear.

In an example, the first wearable sensor can be a optical which is part of, or attached to, an ear-worn device; the second wearable sensor can be a camera which is part of, or attached to, the ear-worn device; and the feedback mechanism communicates information concerning the types and amounts of food to the person via the ear-worn device. In an example, the first wearable sensor can be a optical which is part of, or attached to, eyewear; the second wearable sensor can be a camera which is part of, or attached to, the eyewear; and the feedback mechanism makes recommendations concerning food consumption to the person via the eyewear.

In an example, the first wearable sensor can be a optical which is part of, or attached to, eyewear; the second wearable sensor can be a camera which is part of, or attached to, the eyewear; and the feedback mechanism communicates information concerning the types and amounts of food to the person via the eyewear. In an example, the first wearable sensor can be a optical which is part of, or attached to, eyewear; the second wearable sensor can be a camera which is part of, or attached to, the eyewear; and the feedback mechanism changes the appearance of food in the person’s field of vision via eyewear to change the person’s food consumption behavior.

In an example, the first wearable sensor can be a reflective optical which is part of, or attached to, an ear-worn device; the second wearable sensor can be a camera which is part of, or attached to, the ear-worn device; and the feedback mechanism makes recommendations concerning food consumption to the person via a phone. In an example, the first wearable sensor can be a reflective optical which is part of, or attached to, an ear-worn device; the second wearable sensor can be a camera which is part of, or attached to, the ear-worn device; and the feedback mechanism communicates information concerning the types and amounts of food to the person via a phone.

In an example, the first wearable sensor can be a EMG which is part of, or attached to, an ear-worn device; the second wearable sensor can be a camera which is part of, or attached to, the ear-worn device; and the feedback mechanism makes recommendations concerning food consumption to the person via the ear-worn device. In an example, the first wearable sensor can be a EMG which is part of, or attached to, an ear-worn device; the second wearable sensor can be a camera which is part of, or attached to, the ear-worn device; and the feedback mechanism communicates information concerning the types and amounts of food to the person via the ear-worn device.

In an example, the first wearable sensor can be a EMG which is part of, or attached to, a wrist-worn device; the second wearable sensor can be a camera which is part of, or attached to, the wrist-worn device; and the feedback mechanism makes recommendations concerning food consumption to the person via the wrist-worn device. In an example, the first wearable sensor can be a EMG which is part of, or attached to, a wrist-worn device; the second wearable sensor can be a camera which is part of, or attached to, the wrist-worn device; and the feedback mechanism communicates information concerning the types and amounts of food to the person via the wrist-worn device.

In an example, the first wearable sensor can be a microphone which is part of, or attached to, an ear-worn device; the second wearable sensor can be a camera which is part of, or attached to, the ear-worn device; and the feedback mechanism makes recommendations concerning food consumption to the person via eyewear. In an example, the first wearable sensor can be a microphone which is part of, or attached to, a wrist-worn device; the second wearable sensor can be a camera which is part of, or attached to, the wrist-worn device; and the feedback mechanism makes recommendations concerning food consumption to the person via the wrist-worn device.

In an example, the first wearable sensor can be a microphone which is part of, or attached to, a wrist-worn device; the second wearable sensor can be a camera which is part of, or attached to, the wrist-worn device; and the feedback mechanism makes recommendations concerning food consumption to the person via a phone. In an example, the first wearable sensor can be a microphone which is part of, or attached to, a wrist-worn device; the second wearable sensor can be a camera which is part of, or attached to, the wrist-worn device; and the feedback mechanism makes recommendations concerning food consumption to the person via eyewear.

In an example, the first wearable sensor can be a microphone which is part of, or attached to, a wrist-worn device; the second wearable sensor can be a camera which is part of, or attached to, the wrist-worn device; and the feedback mechanism communicates information concerning the types and amounts of food to the person via the wrist-worn device. In an example, the first wearable sensor can be a microphone which is part of, or attached to, a wrist-worn device; the second wearable sensor can be a camera which is part of, or attached to, the wrist-worn device; and the feedback mechanism communicates information concerning the types and amounts of food to the person via a phone.

In an example, the first wearable sensor can be a microphone which is part of, or attached to, a wrist-worn device; the second wearable sensor can be a camera which is part of, or attached to, the wrist-worn device; and the feedback mechanism communicates information concerning the types and amounts of food to the person via eyewear. In an example, the first wearable sensor can be a microphone which is part of, or attached to, a wrist-worn device; the second wearable sensor can be a camera which is part of, or attached to, the wrist-worn device; and the feedback mechanism changes the appearance of food in the person’s field of vision via eyewear to change the person’s food consumption behavior.

In an example, the first wearable sensor can be a motion which is part of, or attached to, a finger-worn device; the second wearable sensor can be a camera which is part of, or attached to, the finger-worn device; and the feedback mechanism makes recommendations concerning food consumption to the person via the finger-worn device. In an example, the first wearable sensor can be a motion which is part of, or attached to, a finger-worn device; the second wearable sensor can be a camera which is part of, or attached to, the finger-worn device; and the feedback mechanism makes recommendations concerning food consumption to the person via eyewear.

In an example, the first wearable sensor can be a motion which is part of, or attached to, a finger-worn device; the second wearable sensor can be a camera which is part of, or attached to, the finger-worn device; and the feedback mechanism communicates information concerning the types and amounts of food to the person via the finger-worn device. In an example, the first wearable sensor can be a motion which is part of, or attached to, a finger-worn device; the second wearable sensor can be a camera which is part of, or attached to, the finger-worn device; and the feedback mechanism communicates information concerning the types and amounts of food to the person via eyewear.

In an example, the first wearable sensor can be a motion which is part of, or attached to, a finger-worn device; the second wearable sensor can be a camera which is part of, or attached to, the finger-worn device; and the feedback mechanism changes the appearance of food in the person’s field of vision via eyewear to change the person’s food consumption behavior. In an example, the first wearable sensor can be a motion which is part of, or attached to, a wrist-worn device; the second wearable sensor can be a camera which is part of, or attached to, an eyewear; and the feedback mechanism makes recommendations concerning food consumption to the person via the eyewear.

In an example, the first wearable sensor can be a motion which is part of, or attached to, a wrist-worn device; the second wearable sensor can be a camera which is part of, or attached to, the wrist-worn device; and the feedback mechanism makes recommendations concerning food consumption to the person via a phone. In an example, the first wearable sensor can be a motion which is part of, or attached to, a wrist-worn device; the second wearable sensor can be a camera which is part of, or attached to, the wrist-worn device; and the feedback mechanism makes recommendations concerning food consumption to the person via the eyewear.

In an example, the first wearable sensor can be a motion which is part of, or attached to, a wrist-worn device; the second wearable sensor can be a camera which is part of, or attached to, eyewear; and the feedback mechanism communicates information concerning the types and amounts of food to the person via the eyewear. In an example, the first wearable sensor can be a motion which is part of, or attached to, a wrist-worn device; the second wearable sensor can be a camera which is part of, or attached to, the wrist-worn device; and the feedback mechanism communicates information concerning the types and amounts of food to the person via the eyewear.

In an example, the first wearable sensor can be a motion which is part of, or attached to, a wrist-worn device; the second wearable sensor can be a camera which is part of, or attached to, eyewear; and the feedback mechanism changes the appearance of food in the person’s field of vision via the eyewear to change the person’s food consumption behavior. In an example, the first wearable sensor can be a motion which is part of, or attached to, a wrist-worn device; the second wearable sensor can be a camera which is part of, or attached to, the wrist-worn device; and the feedback mechanism changes the appearance of food in the person’s field of vision via eyewear to change the person’s food consumption behavior.

In an example, the first wearable sensor can be a optical which is part of, or attached to, an ear-worn device; the second wearable sensor can be a camera which is part of, or attached to, the ear-worn device; and the feedback mechanism makes recommendations concerning food consumption to the person via a phone. In an example, the first wearable sensor can be a optical which is part of, or attached to, an ear-worn device; the second wearable sensor can be a camera which is part of, or attached to, the ear-worn device; and the feedback mechanism communicates information concerning the types and amounts of food to the person via a phone.

In an example, the first wearable sensor can be a reflective optical which is part of, or attached to, an ear-worn device; the second wearable sensor can be a camera which is part of, or attached to, the ear-worn device; and the feedback mechanism makes recommendations concerning food consumption to the person via eyewear. In an example, the first wearable sensor can be a reflective optical which is part of, or attached to, an ear-worn device; the second wearable sensor can be a camera which is part of, or attached to, the ear-worn device; and the feedback mechanism communicates information concerning the types and amounts of food to the person via eyewear.

In an example, the first wearable sensor can be a reflective optical which is part of, or attached to, an ear-worn device; the second wearable sensor can be a camera which is part of, or attached to, the ear-worn device; and the feedback mechanism changes the appearance of food in the person’s field of vision via eyewear to change the person’s food consumption behavior. In an example, the first wearable sensor can be a spectroscopic optical which is part of, or attached to, a wrist-worn device; the second wearable sensor can be a camera which is part of, or attached to, the wrist-worn device; and the feedback mechanism makes recommendations concerning food consumption to the person via the wrist-worn device.

In an example, the first wearable sensor can be a spectroscopic optical which is part of, or attached to, a wrist-worn device; the second wearable sensor can be a camera which is part of, or attached to, the wrist-worn device; and the feedback mechanism makes recommendations concerning food consumption to the person via a phone. In an example, the first wearable sensor can be a spectroscopic optical which is part of, or attached to, a wrist-worn device; the second wearable sensor can be a camera which is part of, or attached to, the wrist-worn device; and the feedback mechanism makes recommendations concerning food consumption to the person via eyewear.

In an example, the first wearable sensor can be a spectroscopic optical which is part of, or attached to, a wrist-worn device; the second wearable sensor can be a camera which is part of, or attached to, the wrist-worn device; and the feedback mechanism communicates information concerning the types and amounts of food to the person via the wrist-worn device. In an example, the first wearable sensor can be a spectroscopic optical which is part of, or attached to, a wrist-worn device; the second wearable sensor can be a camera which is part of, or attached to, the wrist-worn device; and the feedback mechanism communicates information concerning the types and amounts of food to the person via a phone.

In an example, the first wearable sensor can be a spectroscopic optical which is part of, or attached to, a wrist-worn device; the second wearable sensor can be a camera which is part of, or attached to, the wrist-worn device; and the feedback mechanism communicates information concerning the types and amounts of food to the person via eyewear. In an example, the first wearable sensor can be a spectroscopic optical which is part of, or attached to, a wrist-worn device; the second wearable sensor can be a camera which is part of, or attached to, the wrist-worn device; and the feedback mechanism changes the appearance of food in the person’s field of vision via eyewear to change the person’s food consumption behavior.

In an example, the first wearable sensor can be a EMG which is part of, or attached to, an ear-worn device; the second wearable sensor can be a camera which is part of, or attached to, the ear-worn device; and the feedback mechanism makes recommendations concerning food consumption to the person via a phone. In an example, the first wearable sensor can be a EMG which is part of, or attached to, an ear-worn device; the second wearable sensor can be a camera which is part of, or attached to, the ear-worn device; and the feedback mechanism makes recommendations concerning food consumption to the person via eyewear.

In an example, the first wearable sensor can be a EMG which is part of, or attached to, an ear-worn device; the second wearable sensor can be a camera which is part of, or attached to, the ear-worn device; and the feedback mechanism communicates information concerning the types and amounts of food to the person via a phone. In an example, the first wearable sensor can be a EMG which is part of, or attached to, a wrist-worn device; the second wearable sensor can be a camera which is part of, or attached to, the wrist-worn device; and the feedback mechanism communicates information concerning the types and amounts of food to the person via a phone.

In an example, the first wearable sensor can be a microphone which is part of, or attached to, an ear-worn device; the second wearable sensor can be a camera which is part of, or attached to, the ear-worn device; and the feedback mechanism communicates information concerning the types and amounts of food to the person via eyewear. In an example, the first wearable sensor can be a microphone which is part of, or attached to, an ear-worn device; the second wearable sensor can be a camera which is part of, or attached to, the ear-worn device; and the feedback mechanism changes the appearance of food in the person’s field of vision via eyewear to change the person’s food consumption behavior.

In an example, the first wearable sensor can be a optical which is part of, or attached to, an ear-worn device; the second wearable sensor can be a camera which is part of, or attached to, the ear-worn device; and the feedback mechanism makes recommendations concerning food consumption to the person via eyewear. In an example, the first wearable sensor can be a optical which is part of, or attached to, an ear-worn device; the second wearable sensor can be a camera which is part of, or attached to, the ear-worn device; and the feedback mechanism communicates information concerning the types and amounts of food to the person via eyewear.

In an example, the first wearable sensor can be a optical which is part of, or attached to, an ear-worn device; the second wearable sensor can be a camera which is part of, or attached to, the ear-worn device; and the feedback mechanism changes the appearance of food in the person’s field of vision via eyewear to change the person’s food consumption behavior. In an example, the first wearable sensor can be a EMG which is part of, or attached to, an ear-worn device; the second wearable sensor can be a camera which is part of, or attached to, the ear-worn device; and the feedback mechanism communicates information concerning the types and amounts of food to the person via eyewear.

In an example, the first wearable sensor can be a EMG which is part of, or attached to, an ear-worn device; the second wearable sensor can be a camera which is part of, or attached to, the ear-worn device; and the feedback mechanism changes the appearance of food in the person’s field of vision via eyewear to change the person’s food consumption behavior. In an example, the first wearable sensor can be a EMG which is part of, or attached to, a wrist-worn device; the second wearable sensor can be a camera which is part of, or attached to, the wrist-worn device; and the feedback mechanism makes recommendations concerning food consumption to the person via a phone.

In an example, the first wearable sensor can be a EMG which is part of, or attached to, a wrist-worn device; the second wearable sensor can be a camera which is part of, or attached to, the wrist-worn device; and the feedback mechanism makes recommendations concerning food consumption to the person via eyewear. In an example, the first wearable sensor can be a EMG which is part of, or attached to, a wrist-worn device; the second wearable sensor can be a camera which is part of, or attached to, the wrist-worn device; and the feedback mechanism communicates information concerning the types and amounts of food to the person via eyewear. In an example, the first wearable sensor can be a EMG which is part of, or attached to, a wrist-worn device; the second wearable sensor can be a camera which is part of, or attached to, the wrist-worn device; and the feedback mechanism changes the appearance of food in the person’s field of vision via eyewear to change the person’s food consumption behavior.

In an example, a device for measuring a person’s consumption of one or more selected types of foods, ingredients, and/or nutrients can measure one or more types selected from the group consisting of: a selected type of carbohydrate, a class of carbohydrates, or all carbohydrates; a selected type of sugar, a class of sugars, or all sugars; a selected type of fat, a class of fats, or all fats; a selected type of cholesterol, a class of cholesterols, or all cholesterols; a selected type of protein, a class of proteins, or all proteins; a selected type of fiber, a class of fiber, or all fibers; a specific sodium compound, a class of sodium compounds, or all sodium compounds; high-carbohydrate food, high-sugar food, high-fat food, fried food, high-cholesterol food, high-protein food, high-fiber food, and/or high-sodium food.

In an example, a device for measuring a person’s consumption of one or more selected types of foods, ingredients, and/or nutrients can measure one or more types selected from the group consisting of: simple carbohydrates, simple sugars, saturated fat, trans fat, Low Density Lipoprotein (LDL), and salt. In an example, a device for measuring consumption of a selected nutrient can measure a person’s consumption of simple carbohydrates. In an example, a device for measuring consumption of a selected nutrient can measure a person’s consumption of simple sugars. In an example, a device for measuring consumption of a selected nutrient can measure a person’s consumption of saturated fats. In an example, a device for measuring consumption of a selected nutrient can measure a person’s consumption of trans fats. In an example, a device for measuring consumption of a selected nutrient can measure a person’s consumption of Low Density Lipoprotein (LDL). In an example, a device for measuring consumption of a selected nutrient can measure a person’s consumption of sodium. In an example, a food-identifying sensor can detect one or more nutrients selected from the group consisting of: amino acid or protein (a selected type or general class), carbohydrate (a selected type or general class, such as single carbohydrates or complex carbohydrates), cholesterol (a selected type or class, such as HDL or LDL), dairy products (a selected type or general class), fat (a selected type or general class, such as unsaturated fat, saturated fat, or trans fat), fiber (a selected type or class, such as insoluble fiber or soluble fiber), mineral (a selected type), vitamin (a selected type), nuts (a selected type or general class, such as peanuts), sodium compounds (a selected type or general class), sugar (a selected type or general class, such as glucose), and water. In an example, food can be classified into general categories such as fruits, vegetables, or meat.

In an example, a device for measuring a person’s consumption of a selected nutrient can measure a person’s consumption of food that is high in simple carbohydrates. In an example, a device for measuring consumption of a selected nutrient can measure a person’s consumption of food that is high in simple sugars. In an example, a device for measuring consumption of a selected nutrient can measure a person’s consumption of food that is high in saturated fats. In an example, a device for measuring consumption of a selected nutrient can measure a person’s consumption of food that is high in trans fats. In an example, a device for measuring consumption of a selected nutrient can measure a person’s consumption of food that is high in Low Density Lipoprotein (LDL). In an example, a device for measuring consumption of a selected nutrient can measure a person’s consumption of food that is high in sodium.

In an example, a device for measuring a person’s consumption of a selected nutrient can measure a person’s consumption of food wherein a high proportion of its calories comes from simple carbohydrates. In an example, a device for measuring consumption of a selected nutrient can measure a person’s consumption of food wherein a high proportion of its calories comes from simple sugars. In an example, a device for measuring consumption of a selected nutrient can measure a person’s consumption of food wherein a high proportion of its calories comes from saturated fats. In an example, a device for measuring consumption of a selected nutrient can measure a person’s consumption of food wherein a high proportion of its calories comes from trans fats. In an example, a device for measuring consumption of a selected nutrient can measure a person’s consumption of food wherein a high proportion of its calories comes from Low Density Lipoprotein (LDL). In an example, a device for measuring consumption of a selected nutrient can measure a person’s consumption of food wherein a high proportion of its weight or volume is comprised of sodium compounds.

In an example, a device for measuring nutrient consumption can track the quantities of selected chemicals that a person consumes via food consumption. In various examples, these consumed chemicals can be selected from the group consisting of carbon, hydrogen, nitrogen, oxygen, phosphorus, and sulfur. In an example, a food-identifying device can selectively detect consumption of one or more types of unhealthy food, wherein unhealthy food is selected from the group consisting of: food that is high in simple carbohydrates; food that is high in simple sugars; food that is high in saturated or trans fat; fried food; food that is high in Low Density Lipoprotein (LDL); and food that is high in sodium.

In a broad range of examples, a food-identifying sensor can measure one or more types selected from the group consisting of: a selected food, ingredient, or nutrient that has been designated as unhealthy by a health care professional organization or by a specific health care provider for a specific person; a selected substance that has been identified as an allergen for a specific person; peanuts, shellfish, or dairy products; a selected substance that has been identified as being addictive for a specific person; alcohol; a vitamin or mineral; vitamin A, vitamin B1, thiamin, vitamin B12, cyanocobalamin, vitamin B2, riboflavin, vitamin C, ascorbic acid, vitamin D, vitamin E, calcium, copper, iodine, iron, magnesium, manganese, niacin, pantothenic acid, phosphorus, potassium, riboflavin, thiamin, and zinc; a selected type of carbohydrate, class of carbohydrates, or all carbohydrates; a selected type of sugar, class of sugars, or all sugars; simple carbohydrates, complex carbohydrates; simple sugars, complex sugars, monosaccharides, glucose, fructose, oligosaccharides, polysaccharides, starch, glycogen, disaccharides, sucrose, lactose, starch, sugar, dextrose, disaccharide, fructose, galactose, glucose, lactose, maltose, monosaccharide, processed sugars, raw sugars, and sucrose; a selected type of fat, class of fats, or all fats; fatty acids, monounsaturated fat, polyunsaturated fat, saturated fat, trans fat, and unsaturated fat; a selected type of cholesterol, a class of cholesterols, or all cholesterols; Low Density Lipoprotein (LDL), High Density Lipoprotein (HDL), Very Low Density Lipoprotein (VLDL), and triglycerides; a selected type of protein, a class of proteins, or all proteins; dairy protein, egg protein, fish protein, fruit protein, grain protein, legume protein, lipoprotein, meat protein, nut protein, poultry protein, tofu protein, vegetable protein, complete protein, incomplete protein, or other amino acids; a selected type of fiber, a class of fiber, or all fiber; dietary fiber, insoluble fiber, soluble fiber, and cellulose; a specific sodium compound, a class of sodium compounds, and all sodium compounds; salt; a selected type of meat, a class of meats, and all meats; a selected type of vegetable, a class of vegetables, and all vegetables; a selected type of fruit, a class of fruits, and all fruits; a selected type of grain, a class of grains, and all grains; high-carbohydrate food, high-sugar food, high-fat food, fried food, high-cholesterol food, high-protein food, high-fiber food, and high-sodium food.

In an example, a device for measuring a person’s consumption of at least one specific food, ingredient, and/or nutrient that can analyze food composition can also identify one or more potential food allergens, toxins, or other substances selected from the group consisting of: ground nuts, tree nuts, dairy products, shell fish, eggs, gluten, pesticides, animal hormones, and antibiotics. In an example, a device can analyze food composition to identify one or more types of food whose consumption is prohibited or discouraged for religious, moral, and/or cultural reasons, such as pork or meat products of any kind.

In an example, a camera can automatically shut off (e.g. stop recording images) after a defined period of time wherein no chewing and/or swallowing motions are detected. In an example, a camera can automatically shut off (e.g. stop recording images) after 1 to 5 minutes wherein no chewing and/or swallowing motion is detected. In an example, a method for food consumption monitoring can comprise: (a) using a chewing and/or swallowing sensor on a device worn by a person to record vibrations; (b) if analysis of data from the chewing and/or swallowing sensor indicates that the person is eating, then activating a camera on the device to start recording images of space and/or objects in front of the person to capture food images; (d) if analysis of data from the chewing and/or swallowing sensor indicates that the person is eating, then also analyzing chewing motions, swallowing motions, and/or food images to estimate the types and/or amounts of food that the person is eating; and (e) if analysis of data from the chewing and/or swallowing sensors indicates that the person has stopped eating for a period of time (e.g. between 3 and 15 minutes), then deactivating the camera so that it stops recording images.

In an example, a camera can be activated and/or triggered by (analysis of) signals from a sensor. In an example, a camera can be activated and/or triggered to record images of food in response to analysis of signals from a sensor which indicate consumption of food. In an example, a camera can be activated and/or triggered to record images of food in response to analysis of signals from a sensor which indicate that food is nearby. In an example, a camera can be activated to start recording images immediately when eating is detected by one or more sensors. In an example, a camera can be activated to start recording images immediately when eating is detected by analysis of data from one or more sensors.

In an example, a camera can be attached to eyewear via two components comprising a first component which is permanently attached to the eyewear and a second component which is easily and removably attached to the first component, where the camera is permanently attached to the second component. In an example, a camera can be located at the junction and/or connection between side and front portions of an eyewear frame. In an example, a camera can be mounted to eyewear by a clip, clamp, hook, snap, pin, plug, screw, bolt, elastic band, adhesive, or magnet. In an example, a camera which is attached to eyewear can have a focal vector which is directed downward and forward.

In an example, a camera can be located on the anterior third of the length of a sidepiece of an eyewear frame. In an example, a camera can be located on the central third of the length of a sidepiece of an eyewear frame. In an example, a camera can be located on the front portion of an eyewear frame. In an example, a camera can be located on the posterior third of the length of a sidepiece of an eyewear frame. In an example, a camera on eyewear can have a focal vector which is directed downward and forward. In an example, an eyewear-mounted camera can have a focal vector which faces forward and downward. In an example, an eyewear-mounted camera can have a focal vector which faces forward and downward at an angle between 5 and 20 degrees relative to a horizontal plane which is parallel and/or tangential to the horizon. In an example, an eyewear-mounted camera can have a focal vector which faces forward and downward at an angle between 5 and 20 degrees relative to a plane which is perpendicular to a vertical vector when a person is standing up. In an example, an eyewear-mounted camera can have a focal vector which faces forward and downward at an angle between 5 and 20 degrees relative to a plane which is horizontal when a person is standing up.

In an example, a camera can be mounted above a person’s ear. In an example, a camera can be mounted on a person’s ear. In an example, a camera can be part of an earpiece or ear bud. In an example, a camera can be embodied in an ear ring. In an example, a device and/or system can comprise a necklace or neckband with a camera. In an example, a device and/or system can comprise a necklace or neckband with a vibration sensor and a camera. In an example, a device and/or system can comprise a necklace or neckband with a motion sensor and a camera.

In an example, a camera can continually record images in front of a person in a loop, but these images can be automatically erased after a short period of time (e.g. less than 5 minutes) unless food is detected nearby. In an example, a camera can continually record images in front of a person in a loop, but these images can be automatically erased after a short period of time (e.g. less than 1 minute) unless food is detected nearby. In an example, a camera can continually record images in front of a person in a loop, but these images can be automatically erased after a short period of time (and not communicated externally) unless food is detected nearby. In an example, a camera can continually record images, but these images are automatically erased after 10 to 60 seconds if no nearby food is detected. In an example, a camera can continually record images, but these images are automatically erased after 1 to 5 minutes if no nearby food is detected. In an example, images recorded by a camera can be sent through one or more face-detection filters which obscure and/or erase images of people’s faces (in the interest of privacy) but still enable capturing food images. In an example, images recorded by a camera can be sent through one or more filters which obscure and/or erase images of people (in the interest of privacy) but still enable capturing food images.

In an example, a camera can record images of food near a person at two or more different times, wherein a change in the size and/or volume of the food between the images is used to help measure the amount of food consumed by the person. In an example, a camera can record images of food near a person at different times, wherein a change in the size and/or volume of the food between the images is used to help measure the amount of food consumed by the person. In an example, a camera can record images of food near a person at preset time intervals during a meal, wherein changes in the size and/or volume of the food between the images is used to help measure the amount of food consumed by the person.

In an example, a connector between an eyewear frame and a camera and an eyewear frame can have an electromagnetic actuator which adjusts the angle between eyewear frame and the camera. In an example, a connector between an eyewear frame and a camera and an eyewear frame can have an actuator which adjusts the angle between eyewear frame and the camera. In an example, a connector between an eyewear frame and a camera and an eyewear frame can have a rotating component which adjusts the angle between eyewear frame and the camera. In an example, a connector between an eyewear frame and a camera and an eyewear frame can be adjusted to adjust the angle between eyewear frame and the camera. In an example, a device can comprise a camera for recording food images which is clipped, hooked, and/or clamped onto a sidepiece (e.g. “temple”) of an eyewear frame. In an example, the focal vector of a camera attached to eyewear can be based on data from a motion sensor which is also attached to the eyewear.

In an example, a device and/or system can automatically trigger a camera worn by a person to begin recording food images when one or more sensors worn by the person detect that the person is eating. In an example, when a device and/or system detects that a person has started eating, the system automatically activates a camera to start recording food images. In an example, when a wearable device and/or system detects that a person has started eating, the system automatically activates a wearable camera to start recording food images. In an example, when a wearable device and/or system detects that a person has started eating, the system automatically activates a camera on the device and/or system to start recording food images.

In an example, a device and/or system can calibrate the spectral distribution of a food image, adjusting it to a standard spectral distribution before further image processing. In an example, the temperature of food in a food image can be calibrated using an infrared sensor. In an example, a device can project a laser beam into a camera’s field of vision to help calibrate the scale of food images captured by the camera. In an example, a device and/or system can calibrate the scale of a food image, adjusting it to a standard scale before further image processing. In an example, a device and/or system can calibrate the perspective angle of a food image, adjusting it to a standard perspective angle before further image processing. In an example, a device can project a laser beam into a camera’s field of vision to help calibrate the angular perspective of food images captured by the camera.

In an example, a device and/or system can comprise a chewing and/or swallowing sensor and a camera, wherein the camera is activated and/or triggered to record food images at multiple time intervals while eating is detected by the chewing and/or swallowing sensor. In an example, a device and/or system can comprise a chewing and/or swallowing sensor and a camera, wherein the camera is activated and/or triggered to record food images at multiple time intervals while eating is detected based on data from the chewing and/or swallowing sensor. In an example, a device and/or system can comprise a chewing and/or swallowing sensor and a camera, wherein the camera is activated and/or triggered to record food images at multiple time intervals while eating is detected based on data from the chewing and/or swallowing sensor. In an example, a device and/or system can comprise a wearable chewing and/or swallowing sensor and a wearable camera, wherein the camera is activated and/or triggered to record food images at multiple time intervals while eating is detected based on data from the chewing and/or swallowing sensor.

In an example, a device and/or system can comprise a chewing and/or swallowing sensor and a camera which are integrated into eyewear, wherein the camera is activated and/or triggered to record food images at multiple time intervals while eating is detected based on data from the chewing and/or swallowing sensor. In an example, a device and/or system can comprise a chewing and/or swallowing sensor and a camera which are integrated into eyewear, wherein the camera is activated and/or triggered to record food images at multiple time intervals while eating is detected based on data from the chewing and/or swallowing sensor.

In an example, a device and/or system can comprise a chewing and/or swallowing sensor and a camera which are integrated into a necklace, pendant, or neck band, wherein the camera is activated and/or triggered to record food images at multiple time intervals while eating is detected based on data from the chewing and/or swallowing sensor. In an example, a device and/or system can comprise a chewing and/or swallowing sensor and a camera which are integrated into an ear-worn device, wherein the camera is activated and/or triggered to record food images at multiple time intervals while eating is detected based on data from the chewing and/or swallowing sensor.

In an example, a device and/or system can comprise a chewing and/or swallowing sensor and a camera which are integrated into eyewear, wherein the camera is activated and/or triggered to record food images when chewing and/or swallowing is detected by analyzing data from the chewing and/or swallowing sensor. In an example, a device and/or system can comprise a chewing and/or swallowing sensor and a camera which are integrated into eyewear, wherein the camera is activated and/or triggered to record food images when eating is detected by analyzing data from the chewing and/or swallowing sensor.

In an example, a device and/or system can comprise a chewing and/or swallowing sensor and a camera in a smart band or smart watch, wherein the camera is activated and/or triggered to record food images when chewing and/or swallowing is detected by analyzing data from the chewing and/or swallowing sensor. In an example, a device and/or system can comprise a chewing and/or swallowing sensor and a camera in a smart band or smart watch, wherein the camera is activated and/or triggered to record food images when eating is detected by analyzing data from the chewing and/or swallowing sensor. In an example, a device and/or system can comprise a chewing and/or swallowing sensor and a camera in a smart band or smart watch, wherein the camera is activated and/or triggered to record food images at multiple time intervals while eating is detected based on data from the chewing and/or swallowing sensor.

In an example, a device can comprise a camera and a movable mirror, wherein the focal direction of the camera is adjusted to record food images from different perspectives by moving the mirror. In an example, a device can comprise a camera and a movable mirror, wherein the focal direction of the camera is adjusted by moving the mirror. In an example, a device can comprise one or more actuators which move a mirror so that a camera maintains focal direction toward nearby food. In an example, a device can comprise one or more actuators which move a camera to maintain a focal direction toward nearby food. In an example, a device can comprise one or more actuators which move a camera lens to maintain a focal direction toward nearby food. In an example, a device can comprise a camera for recording food images which is clipped onto the collar of an upper-body garment.

In an example, a device can comprise a motion sensor (e.g. accelerometer, gyroscope, and inclinometer) and a camera, wherein data from the motion sensor is used to determine the angular perspective of food images captured by the camera. In an example, a device can comprise a motion sensor (e.g. accelerometer, gyroscope, and inclinometer) and a camera, wherein data from the motion sensor is used to determine the distance of food images captured by the camera. In an example, a device can have a camera with a wide-angle and/or fish eye lens. In an example, a device can adjust a camera between recording wide angle images or narrow angle images.

In an example, a device can comprise one or more actuators which move a mirror so that a camera maintains focal direction toward a person’s hand in order to capture interactions between the person’s hand and nearby food. In an example, a device can comprise one or more actuators which move a camera to maintain a focal direction toward a person’s hand in order to capture interactions between the person’s hand and nearby food. In an example, a device can comprise one or more actuators which move a camera lens to maintain a focal direction toward a person’s hand in order to capture interactions between the person’s hand and nearby food. In an example, a device can comprise one or more electrical actuators which move a camera to maintain a focal direction toward nearby food.

In an example, a device can comprise one or more actuators which move a mirror so that a camera tracks a person’s hand in order to capture interactions between the person’s hand and nearby food. In an example, a device can comprise one or more actuators which move a camera to track nearby food. In an example, a device can comprise one or more actuators which move a camera to track a person’s hand in order to capture interactions between the person’s hand and nearby food. In an example, a device can comprise one or more actuators which move a camera lens to track nearby food.

In an example, a device can comprise one or more actuators which move a camera lens to track a person’s hand in order to capture interactions between the person’s hand and nearby food. In an example, a device can comprise one or more electrical actuators which move a mirror so that a camera tracks nearby food. In an example, a device can comprise one or more electrical actuators which move a mirror so that a camera tracks a person’s hand in order to capture interactions between the person’s hand and nearby food. In an example, a device can comprise one or more electrical actuators which move a mirror so that a camera maintains focal direction toward nearby food. In an example, a device can comprise one or more electrical actuators which move a camera lens to maintain a focal direction toward nearby food. In an example, a device can comprise one or more electrical actuators which move a camera to maintain a focal direction toward a person’s hand in order to capture interactions between the person’s hand and nearby food.

In an example, a device can comprise one or more electrical actuators which move a mirror so that a camera maintains focal direction toward a person’s hand in order to capture interactions between the person’s hand and nearby food. In an example, a device can comprise one or more electrical actuators which move a camera lens to maintain a focal direction toward a person’s hand in order to capture interactions between the person’s hand and nearby food. In an example, a device can have a camera with lens with an adjustable focal direction, wherein the focal direction is automatically adjusted to keep an object identified as food in the camera’s line of sight.

In an example, a device can comprise one or more electrical actuators which move a camera to track nearby food. In an example, a device can comprise one or more electrical actuators which move a camera to track a person’s hand in order to capture interactions between the person’s hand and nearby food. In an example, a device can comprise one or more electrical actuators which move a camera lens to track nearby food. In an example, a device can comprise one or more electrical actuators which move a camera lens to track a person’s hand in order to capture interactions between the person’s hand and nearby food. In an example, a device can comprise one or more actuators which move a mirror so that a camera tracks nearby food.

In an example, a device can comprise two cameras, wherein a first camera scans for nearby food at a first focal distance and a second camera scans for nearby food at a second focal distance. In an example, a device can comprise two cameras, wherein a first camera scans for nearby food in a first direction and a second camera scans for nearby food in a second direction. In an example, a device can transmit data from one or more sensors and/or cameras to a remote data processor through network. In an example, a device for measuring food consumption can comprise two or more cameras to capture images of food from different perspectives. In an example, a device for measuring food consumption can comprise two or more cameras to capture images of food from different perspectives to better estimate the size, volume, and/or amount of nearby food. In an example, a system can activate a plurality of cameras on a person’s smart watch to record food images when a motion sensor (e.g. accelerometer, gyroscope, and/or inclinometer) on the smart watch detects motion patterns which indicate that the person is eating.

In an example, a device can create three-dimensional virtual models of food by combining images from two cameras. In an example, a device can estimate food volume and/or amount by creating three-dimensional virtual models of food by combining images from two cameras. In an example, a device for measuring food consumption can comprise two or more cameras to create three-dimensional virtual models of nearby food. In an example, a three-dimensional virtual model of nearby food can be created by combining images from two cameras at two different locations relative to the food.

In an example, a device can have a camera with a downward and forward field of view. In an example, a device can comprise a camera with an achromatic lens. In an example, a device can have a camera with a diffractive lens. In an example, a device can comprise a camera and a mirror. In an example, a method for determining the amount of food consumed can comprise waving and/or moving a camera back and forth over food at periodic intervals during an eating event and/or meal. In an example, a camera can record images of food near a person during a meal when detected chews and/or swallows reach selected target numbers of chews and/or swallows, wherein changes in the size and/or volume of the food between the images is used to help measure the amount of food consumed by the person.

In an example, a device can have a camera with lens with an adjustable focal length, wherein the focal length is automatically adjusted to keep an object identified as food in focus. In an example, the focal length of a camera can be automatically varied to (scan to) identify the focal plane of nearby food. In an example, a camera can have a lens whose focal length can be automatically adjusted to maintain its focus on nearby food if there is a change in the distance between the camera and the food. In an example, a camera can have a lens whose focal length can be automatically adjusted to focus on nearby food and blur nearby people or non-food nearby objects. In an example, a device can identify (a unit of) standardized food by identifying a bar code, QR code, or other digital code on a food container, food packaging, food label, food menu, food shelf tab, or food display sign.

In an example, a device can project a laser beam into a camera’s field of vision to help calibrate images captured by the camera. In an example, the light intensity of a food image recorded by a wearable camera can be calibrated using an object with a known brightness (e.g. standard light bulb, light fixture, and/or LED) in the image. In an example, the light intensity of food images recorded by a wearable camera can be calibrated using an ambient light sensor. In an example, a device and/or system can calibrate the brightness of a food image, adjusting it to a standard level of brightness before further image processing.

In an example, a device can project a laser beam into a camera’s field of vision to help calibrate colors in food images captured by the camera. In an example, a device can project a laser beam into a camera’s field of vision to help calibrate the color spectrum of images captured by the camera. In an example, the color spectrum of food images recorded by a wearable camera can be calibrated using a spectroscopic sensor. In an example, a device and/or system can calibrate the color spectrum of a food image, adjusting it to a standard color spectrum before further image processing. In an example, the color spectrum of a food image recorded by a wearable camera can be calibrated using an object with a known color spectrum (e.g. standard brand food packing, box, bottle, can, and/or container) in the image. In an example, the color spectrum of a food image recorded by a wearable camera can be calibrated using an object with a known color spectrum (e.g. laser beam emitted by a smart watch) in the image.

In an example, a device can record images of food. In an example, a device can automatically adjust the exposure time of a food image. In an example, a device can automatically adjust the focal distance of a food image. In an example, a device can automatically adjust the focal width of a food image. In an example, a device can automatically adjust the resolution of a food image. In an example, a device can automatically crop a food image.

In an example, a device can: (a) continually record first images of space in front of a person to scan for nearby food; (b) if nearby food is detected, then detect if the person starts to eat within a certain period of time; (c) if eating by the person is not detected within that period of time, then delete the images; (d) if eating by the person is detected within that period of time, then record second images of space in front of the person when the person stops eating or is near finished eating based on consumption rate; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on hand and/or arm movements; (c) if eating by the person is not detected within that period of time, then delete the images; (d) if eating by the person is detected within that period of time, then record second images of space in front of the person when the person stops eating or is near finished eating based on changes in the rate of hand and/or arm movements; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on hand and/or arm movements; (c) if eating by the person is not detected within that period of time, then delete the images; (d) if eating by the person is detected within that period of time, then record second images of space in front of the person when the person stops eating or is near finished eating based on changes in hand and/or arm movements; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on hand and/or arm movements; (c) if eating by the person is not detected within that period of time, then delete the images; (d) if eating by the person is detected within that period of time, then record second images of space in front of the person when the person stops eating based on cessation of hand and/or arm movements; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on chewing or swallowing movements; (c) if eating by the person is not detected within that period of time, then delete the images; (d) if eating by the person is detected within that period of time, then record second images of space in front of the person when the person stops eating or is near finished eating based on changes in the rate of chewing and/or swallowing movements; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on chewing or swallowing movements; (c) if eating by the person is not detected within that period of time, then delete the images; (d) if eating by the person is detected within that period of time, then record second images of space in front of the person when the person stops eating or is near finished eating based on changes in chewing and/or swallowing movements; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on chewing or swallowing movements; (c) if eating by the person is not detected within that period of time, then delete the images; (d) if eating by the person is detected within that period of time, then record second images of space in front of the person when the person stops eating based on cessation of chewing and/or swallowing movements; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on (changes in the rate of) chewing or swallowing sounds; (c) if eating by the person is not detected within that period of time, then delete the images; (d) if eating by the person is detected within that period of time, then record second images of space in front of the person when the person stops eating based on (changes in the rate of) chewing and/or swallowing sounds; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on (changes in the rate of) hand and/or arm sounds; (c) if eating by the person is not detected within that period of time, then delete the images; (d) if eating by the person is detected within that period of time, then record second images of space in front of the person when the person stops eating based on (changes in the rate of) hand and/or arm sounds; (e) estimate the amount of food consumed by the person by analyzing the difference in food volume and/or size between the first and second images.

In an example, a device can: (a) detect when a person first starts to eat, (b) record a first image of nearby food, (c) detect when the person has finished eating or is near finished based on consumption rate, (d) record a second image of nearby food, and (e) estimate the amount of food consumed by the person by analyzing the difference in food volume and/or size between the first and second images. In an example, a device can: (a) detect when a person is near food, (b) record a first image of the nearby food, (c) detect when the person has started to eat, (d) detect when the person has finished eating or is near finished based on consumption rate, (e) record a second image of nearby food, and (f) estimate the amount of food consumed by the person by analyzing the difference in food volume and/or size between the first and second images.

In an example, a method for monitoring food consumption can comprise: (a) using an motion sensor worn by a person to measure tissue motion; (b) analyzing measured tissue motions to identify chewing and/or swallowing motions; (c) if chewing and/or swallowing motions have been identified during a period of time (e.g. the last 1-10 minutes), then activating a camera worn by the person to record images of the area in front of the person; (e) if chewing and/or swallowing motions have been identified during the period of time, then analyzing the recorded images to identify food; (f) if food is not identified in the recorded images, then erasing the recorded images; (g) if food is identified in the recorded images, then analyzing the tissue motions and the recorded images to estimate the types and/or amounts of food consumed by the person.

In an example, a system can activate a camera on a person’s smart watch to record food images when a motion sensor on the smart watch detects motion patterns which indicate that the person is eating. In an example, a device can activate a camera to record food images when a wrist-worn motion sensor detects motion patterns which indicate that a person is eating. In an example, a system can comprise a smart watch worn on one of a person’s non-dominant arm and a wrist-band with a motion sensor worn on the wrist of the person’s dominant arm, wherein eating motions detected by the motion sensor trigger a camera on the smart watch to record food images. In an example, a wrist-worn device for measuring a person’s food consumption can trigger a camera to start recording food images when eating is detected by analysis of the person’s arm motion and heart rate. In an example, system can comprise two wrist-worn devices, one on dominant and one on non-dominant, with motion sensors on both and camera on one. In an example, system can comprise two wrist-worn devices, one on dominant and one on non-dominant, with motion sensor and camera on each.

In an example, a system can analyze images recorded by a wearable camera to identify food items in those images. In an example, food images captured by a wearable camera can be analyzed to segment and/or identify different types of food in a meal. In an example, food images captured by a wearable camera can be analyzed to estimate the types and/or amounts of nearby food. In an example, food images captured by a wearable camera can be analyzed to estimate the amounts, sizes, and/or portions of different types of food in a meal. In an example, food images captured by a wearable camera can be analyzed to estimate the amounts of food portions in a meal.

In an example, a system can comprise a smart watch with a motion sensor and eyewear with a camera, wherein eating motions detected by the motion sensor trigger the camera to record images of nearby food and/or the person’s hand-to-mouth interactions. In an example, a system can comprise a wrist-band with a motion sensor worn on the wrist of the person’s dominant arm and eyewear with a camera, wherein eating motions detected by the motion sensor trigger the camera to record images of nearby food and/or the person’s hand-to-mouth interactions. In an example, a system can comprise a wrist-worn device (e.g. smart watch) and a head-worn device (e.g. eyewear) worn by a person, wherein the system monitors the distance between the wrist-worn device and the head-worn device, and wherein the system triggers and/or activates a camera on the head-worn device to record images (which may include food) when the distance is less than a minimum distance.

In an example, a system can comprises two wrist-worn devices with accelerometers, wherein detection of eating motion on either side triggers an eyewear camera to start recording food images. In an example, a system can comprises two wrist-worn devices, wherein detection of eating motion on either side triggers an eyewear camera to start recording food images. In an example, a system can activate a camera on a person’s eyewear to record food images when a wrist-worn motion sensor detects motion patterns which indicate that the person is eating. In an example, a system can activate a camera on a person’s eyewear to record food images when a motion sensor on a person’s smart watch detects motion patterns which indicate that the person is eating.

In an example, a wrist-worn device (e.g. smart watch) with a camera can record food images from different angular perspectives to create a three-dimensional virtual mode of food as a person waves (e.g. horizontally moves) the device over the food. In an example, a wrist-worn device (e.g. smart watch) with a camera can record food images from different angular perspectives to create a three-dimensional virtual model of food as a person waves (e.g. horizontally moves) the device over a meal. In an example, a device can have a camera with lens with an adjustable focal direction, wherein the focal direction is automatically moved to keep a wrist-worn device (e.g. smart watch or wrist band) on person’s hand in the camera’s line of sight. In an example, a device can have a camera with lens with an adjustable focal direction, wherein the focal direction is automatically adjusted to keep the person’s hand in the camera’s line of sight and wherein this adjustment is based, at least in part, on a motion sensor worn on the person’s hand or wrist. In an example, the focal vector of a camera on a wrist-worn device (e.g. smart watch or wrist band) can be automatically moved to continue to focus on nearby food as the device moves relative to the food.

In an example, a wrist-worn device for measuring a person’s food consumption can trigger a camera to start recording food images when eating is detected by analysis of data from a motion sensor and a spectroscopic sensor on the device. In an example, a wrist-worn device for measuring a person’s food consumption can trigger a camera to start recording food images when eating is detected by analysis of the person’s arm motion and heart rate based on data from a motion sensor and a spectroscopic sensor on the device. In an example, a device and/or system can comprise a necklace or neckband with a spectroscopic sensor and a camera.

In an example, an eyewear-based camera can detect utensils (e.g. fork, spoon, or knife) which are associated with eating. In an example, an eyewear-based camera can detect hand-to-mouth motions which are associated with eating. In an example, an eyewear-based camera can detect glasses, cans, or other beverage-holding containers which are associated with drinking. In an example, analysis of images recorded by an eyewear-based camera can detect utensils (e.g. fork, spoon, or knife) which are associated with eating. In an example, analysis of images recorded by an eyewear-based camera can detect hand-to-mouth motions which are associated with eating. In an example, analysis of images recorded by an eyewear-based camera can detect glasses, cans, or other beverage-holding containers which are associated with drinking. In an example, system can comprise two wrist-worn devices and eyewear, one on dominant and one on non-dominant, with motion sensors on both wrist-worn devices and a camera on the eyewear.

In an example, an eyewear-worn or eyewear integrated device can comprise right and left side cameras, to record food images from different angles to create 3D virtual models of food. In an example, an eyewear-worn or eyewear integrated device can comprise right and left side cameras, to record food images from different angles for three-dimensional analysis of food quantities and/or volumes. In an example, this device can be embodied in eyewear with a right-side camera and a left-side camera, wherein the focal vectors of the cameras are angled so as to converge at a distance between 1 and 4 feet in front of the device. In an example, this device can be embodied in eyewear with a right-side camera and a left-side camera, wherein the focal vectors of the cameras are angled inward so as to converge at a distance between 1 and 4 feet in front of the device.

In an example, analysis of food images recorded by a camera at different times during a meal can be compared to an initial food image recorded at the start of the meal to estimate (and display) the cumulative amount of food consumed after each time interval based on changes in the size and/or volume of the food. In an example, analysis of food images recorded by a camera at different times during a meal can be compared to an initial food image recorded at the start of the meal to estimate (and display) the cumulative amount of calories consumed after each time interval based on changes in the size and/or volume of the food. In an example, analysis of food images recorded by a camera at different times during a meal can be compared to an initial food image recorded at the start of the meal to estimate (and display) the cumulative amounts of different types of nutrients consumed after each time interval based on the type of food and changes in the size and/or volume of the food.

In an example, device can activate a camera to record images of food at different times during food consumption to better estimate the change in nearby food quantity and/or volume. In an example, device can activate a camera to record images of food at different times during food consumption to better estimate the amount of nearby food consumed by a person. In an example, a method for determining the amount of food consumed can comprise waving and/or moving a camera back and forth over food at multiple times during an eating event and/or meal.

In an example, face recognition can be used to narrow camera’s field of vision so as not to record images of nearby people. In an example, face recognition can be used to blur out faces of nearby people in camera’s field of vision. In an example, portions of images recorded by a wearable camera which include people can be automatically cropped out and/or blurred to exclude images of people or objects which are not food before those images are transmitted to a remote data processor. In an example, portions of images recorded by a wearable camera which include people can be automatically cropped out and/or blurred before those images are stored in long-term local memory and/or transmitted to a remote data processor. In an example, if images of other people are identified in images, then the field of view of the camera can be narrowed and/or redirected so that other people are no longer within the camera’s field of view.

In an example, if a camera on AR eyewear has a view of nearby food which is obscured and/or insufficient to determine food type and/or amount, then the eyewear can show the person how to move either the food and/or their head to obtain an un-obscured and/or sufficient view to determine food type and/or amount. In an example, if a camera on AR eyewear has a view of nearby food which is obscured and/or insufficient to determine food type and/or amount, then the eyewear can display virtual images which show the person how to move either the food and/or their head to obtain an un-obscured and/or sufficient view to determine food type and/or amount. In an example, if a camera on AR eyewear has a view of nearby food which is obscured and/or insufficient to determine food type and/or amount, then the eyewear can display virtual images which show the person how to where to move the food and/or how to move their head in order to obtain an un-obscured and/or sufficient view to determine food type and/or amount. In an example, a device can comprise a camera for recording food images which is clipped, hooked, and/or clamped onto eyeglasses.

In an example, images of food recorded by two cameras, on the right and left sides of the frontpiece of an eyeglass frame, respectively, are analyzed to create a three-dimensional virtual model of the food. In an example, images of food recorded by two cameras, on the right and left sidepieces (e.g. “temples”) of an eyeglass frame, respectively, are analyzed to create a three-dimensional virtual model of the food. In an example, images of food recorded by two cameras at different locations on an eyeglass frame are analyzed and/or merged to create a three-dimensional virtual model of the food. In an example, images of food recorded by two cameras at different locations are jointly analyzed and/or merged to create a three-dimensional virtual model of the food.

In an example, images recorded by a camera can be analyzed to identify food. In an example, images recorded by a camera can be analyzed to identify food, including types and amounts of food. In an example, images recorded by a camera can be analyzed to identify food, including types and amounts of food portions in a meal. In an example, a camera can capture multiple still-frame images of food. In an example, a camera can capture video images of food. In an example, a camera can record pictures and/or images of food. In an example, a camera can record video images of food. In an example, a device can include an image acquisition component, such as a camera.

In an example, the angle between a camera and an eyewear frame can be adjusted. In an example, a device can comprise a rotating component which adjusts the angle between eyewear frame and the camera. In an example, a device can comprise an actuator which adjusts the angle between eyewear frame and the camera. In an example, a device can comprise an electromagnetic actuator which adjusts the angle between eyewear frame and the camera. In an example, the focal direction and/or depth a camera on eyewear can be automatically changed (e.g. cyclically varied) to capture images of nearby food from different angles and/or focal distances. In an example, the focal direction of an eyewear-mounted camera can be automatically adjusted based on data from an eyewear-mounted IMU (e.g. accelerometer, gyroscope, and inclinometer).

In an example, the focal direction and/or distance of a wearable camera can be adjusted to include images of food but exclude images of people. In an example, the focal direction of a camera can be automatically narrowed and/or moved to capture images of nearby food, but not nearby people. In an example, the focal vector of a camera can be automatically varied to (scan to) identify the local direction of nearby food. In an example, the focal vector of a camera can be automatically pivoted and/or rotated to (scan to) identify the local direction of nearby food.

In an example, the focal vector of a camera can be adjusted based on data from a motion sensor. In an example, a device can automatically adjust the focal distance of a camera. In an example, a device can automatically adjust the focal direction of a camera. In an example, a device can comprise a camera and a motion sensor, wherein the focal vector of the camera is automatically changed based on data from the motion sensor. In an example, a device can have a camera with lens with an adjustable focal direction, wherein the focal direction is automatically moved to keep the person’s hand in the camera’s line of sight. In an example, the focal vector of a camera can be adjusted based on the orientation of a person’s head as measured by a motion sensor.

In an example, the focal vector of a camera on eyewear can be automatically moved to continue to track nearby food as the eyewear moves relative to the food. In an example, the focal vector of a camera on eyewear can be automatically moved to continue to focus on nearby food as the eyewear moves relative to the food. In an example, the focal vector of an eyewear-mounted camera can be automatically moved based on data from an eyewear-mounted IMU (e.g. accelerometer, gyroscope, and inclinometer) to maintain a downward angle between 5 and 20 degrees between a horizontal plane (e.g. parallel and/or tangential to the horizon) and the focal vector.

In an example, the focal vector of a camera worn on a person can be automatically moved to track nearby food as the person’s body moves. In an example, the focal vector of a camera can be automatically adjusted to capture images of nearby food, but not nearby people. In an example, the focal scope of a camera can be automatically adjusted to capture images of nearby food, but not nearby people. In an example, the focal range of a camera can be automatically adjusted to capture images of nearby food, but not nearby people. In an example, face recognition can be used to direct camera’s focal vector away from nearby people. In an example, a camera can have field and/or breadth of view which can be automatically adjusted to track nearby food.

In an example, there can be a delay between when eating is detected by analysis of data from one or more sensors and when a camera is activated to start recording images. In an example, there can be a 1 to 5 minute delay between when eating is detected by analysis of data from one or more sensors and when a camera is activated to start recording images. In an example, there can be a 5 to 60 second delay between when eating is detected by analysis of data from one or more sensors and when a camera is activated to start recording images. In an example, a camera can remain covered until activated by eating detection.

In an example, this device can be embodied in eyewear with a right-side camera and a left-side camera, wherein the focal vectors of the cameras are automatically adjusted to maintain focus on food which is identified nearby. In an example, this device can be embodied in eyewear with a right-side camera and a left-side camera, wherein the focal vectors of the cameras are automatically adjusted to create there-dimension images of nearby food. In an example, a three-dimensional virtual model of nearby food can be created by moving a single camera to different locations relative to the food. In an example, when a device and/or system detects that a person has started eating, the system automatically starts recording food images. In an example, when a device and/or system detects that a person has started eating, the system automatically starts recording food images unless the person takes action to block this recording.

In an example, a camera can have a conical field of vision which extends outward from the camera aperture and downwards toward a reachable food source. In an example, a reachable food source can be food on a plate. In an example, a reachable food source can be encompassed by the circular end of the conical field of vision. In an example, a device and method of taking pictures of both nearby food and the person’s mouth, while a person eats, can do a much better job of estimating the types and quantities of food actually consumed than one of the devices or methods in the prior art that only takes pictures of either nearby food or the person’s mouth.

In an example, a device and method comprise one or more cameras that automatically and collectively take pictures of a person’s mouth and pictures of nearby food as the person eats, without the need for human intervention to actively aim or focus a camera toward a person’s mouth or a nearby food. In an example, a device and method takes pictures of a person’s mouth and a food source automatically by eliminating the need for human intervention to aim a camera, such as a camera, towards the person’s mouth and the food source. A device and method includes cameras whose locations, and/or the movement of those locations while the person eats, enables the fields of vision of the cameras to automatically encompass the person’s mouth and a food source.

In an example, a device can comprise multiple cameras that take images along different imaging vectors so that the device takes pictures of nearby food and a person’s mouth simultaneously. In an example, a device comprises a camera with a wide-angle lens that takes pictures within a wide field of vision so that the device takes pictures of nearby food and a person’s mouth simultaneously. In an example, a device can include at least two cameras worn on a person’s body, wherein the field of vision from a first camera automatically encompasses the person’s mouth as the person eats, and wherein the field of vision from a second camera automatically encompasses nearby food as the person eats.

In an example, a device comprises a human-energy input measuring device and method that includes a wearable camera that identifies the types and quantities of food consumed based on images of food from a plurality of points along a food consumption pathway. In an example, a device and method takes pictures of a person’s mouth and nearby food from multiple angles, from a camera worn on a body member that moves as food travels along a food consumption pathway.

In an example, one or more cameras can be moved automatically, independently of movement of a body member to which the cameras are attached, in order to increase the probability of encompassing both a person’s mouth and a nearby food. In an example, the lenses of one or more cameras can be automatically and independently moved in order to increase the probability of encompassing both the person’s mouth and a nearby food. In various examples, a lens can be automatically shifted or rotated to change the direction or focal length of the camera’s field of vision. In an example, the lenses of one or more cameras can be automatically moved to track the person’s mouth and hand. In an example, the lenses of one or more cameras can be automatically moved to scan for reachable food sources.

In an example, the field of vision and the focal length of a wearable camera (such as a wearable digital video camera) can be adjusted automatically to track a particular object as the object moves, the sensor moves, or both the object and the sensor move. In an example, a wrist-worn camera may track the ends of the person’s fingers wherein a utensil or glass is engaged. In an example, a wrist-worn camera may track the person’s face and mouth even when the person is moves their arm and hand. In an example, a camera may continuously or periodically scan the space around the person’s hand and/or mouth to increase the probability of automatically detecting food consumption. In an example, the field of vision and/or focal length of a camera can be automatically adjusted based on the output of a motion sensor. In an example, a camera and a motion sensor can both be incorporated into a device that is worn on the person’s wrist. In an example, a camera can be worn on the person’s neck and a sound sensor can be worn on the person’s wrist.

In an example, the fields of vision from one or more cameras can collectively and automatically encompass a person’s mouth and a nearby food, when the person eats, without the need for human intervention, when the person eats, to manually aim a camera toward the person’s mouth or toward a reachable food source. In an example, the fields of vision from one or more cameras can be moved as a person moves their arm when the person eats; and wherein this movement causes the fields of vision from one or more cameras to collectively and automatically encompass the person’s mouth and a nearby food, when the person eats, without the need for human intervention, when the person eats, to manually aim a camera toward the person’s mouth or toward a reachable food source.

In an example, the fields of vision from one or more cameras in a device collectively and automatically encompass the person’s mouth and a nearby food, when the person eats, without the need for human intervention (when the person eats) to manually aim a camera toward the person’s mouth or toward a reachable food source. In an example, the cameras have wide-angle lenses that encompass nearby food and the person’s mouth without any need for aiming or moving the cameras. Alternatively, a camera may sequentially and iteratively focus on the food source, then on the person’s mouth, then back on the food source, and so forth.

In an example, there can be one camera that takes pictures of both a person’s mouth and a nearby food. In an example, there can be two or more cameras, worn on one or more locations on a person, that collectively and automatically take pictures of the person’s mouth when the person eats and pictures of nearby food when the person eats. In an example, this picture taking can occur in an automatic manner as the person eats. In various examples, a camera worn on a person’s body can take pictures of food at multiple points as it moves along a food consumption pathway. In various examples, a device can comprise a wearable, mobile, calorie-input-measuring device that automatically records and analyzes food images in order to detect and measure human caloric input. In various examples, a device comprises a wearable, mobile, energy-input-measuring device that automatically analyzes food images to measure human energy input.

In an example, a camera may be activated by a chewing sensor. In an example, a device and/or system can comprise a chewing and/or swallowing sensor and a camera, wherein the camera is activated and/or triggered to record food images when chewing and/or swallowing is detected by the chewing and/or swallowing sensor. In an example, a device and/or system can comprise a chewing and/or swallowing sensor and a camera, wherein the camera is activated and/or triggered to record food images when chewing and/or swallowing is detected by analyzing data from the chewing and/or swallowing sensor.

In an example, a device and/or system can comprise a chewing and/or swallowing sensor and a camera which are attached to eyewear, wherein the camera is activated and/or triggered to record food images at multiple time intervals while eating is detected based on data from the chewing and/or swallowing sensor. In an example, a device and/or system can comprise a chewing and/or swallowing sensor and a camera which are attached to eyewear, wherein the camera is activated and/or triggered to record food images at multiple time intervals while eating is detected based on data from the chewing and/or swallowing sensor. In an example, a device and/or system can comprise a chewing and/or swallowing sensor and a camera which are attached to eyewear, wherein the camera is activated and/or triggered to record food images at multiple time intervals while eating is detected based on data from the chewing and/or swallowing sensor.

In an example, a device and/or system can comprise a chewing and/or swallowing sensor and a camera, wherein the camera is activated and/or triggered to record food images when eating is detected by the chewing and/or swallowing sensor. In an example, a device and/or system can comprise a chewing and/or swallowing sensor and a camera, wherein the camera is activated and/or triggered to record food images when eating is detected by analyzing data from the chewing and/or swallowing sensor. In an example, a device and/or system can comprise a sound-based chewing sensor and a camera, wherein the camera is activated and/or triggered to record food images when chewing is detected by the chewing sensor.

In an example, a device and/or system can comprise a chewing and/or swallowing sensor and a camera which are attached to the frame of eyewear, wherein the camera is activated and/or triggered to record food images at multiple time intervals while eating is detected based on data from the chewing and/or swallowing sensor. In an example, a device and/or system can comprise a chewing and/or swallowing sensor and a camera which are attached to eyewear, wherein the camera is activated and/or triggered to record food images when chewing and/or swallowing is detected by analyzing data from the chewing and/or swallowing sensor. In an example, a device and/or system can comprise a chewing and/or swallowing sensor and a camera which are attached to eyewear, wherein the camera is activated and/or triggered to record food images when eating is detected by analyzing data from the chewing and/or swallowing sensor.

In an example, a device and/or system can comprise a wearable chewing and/or swallowing sensor and a wearable camera, wherein the camera is activated and/or triggered to record food images when chewing and/or swallowing is detected by analyzing data from the chewing and/or swallowing sensor. In an example, a device and/or system can comprise a wearable chewing and/or swallowing sensor and a wearable camera, wherein the camera is activated and/or triggered to record food images when eating is detected by analyzing data from the chewing and/or swallowing sensor.

In an example, a method for food consumption monitoring can comprise: (a) using an optical sensor on a device worn by a person to measure motions of the person’s body tissue on or near the person’s jaw; (b) using a data processor to analyze these tissue motions to detect chewing and/or swallowing motions which indicate that the person is eating; (c) if analysis of these tissue motions indicates that the person is eating, then activating a camera on the device to start recording images of space and/or objects in front of the person to capture food images; (d) if analysis of these tissue motions indicates that the person is eating, then also analyzing these tissue motions and these food images to estimate the types and/or amounts of food that the person is eating; and (e) if analysis of these tissue motions indicates that the person has not eaten (e.g. has stopped eating) during a recent period of time, then deactivating the camera so that it stops recording images.

In an example, a method for food consumption monitoring can comprise: (a) using a chewing and/or swallowing sensor on a device worn by a person to record vibrations; (b) if analysis of data from the chewing and/or swallowing sensor indicates that the person is eating, then activating a camera on the device to start recording images of space and/or objects in front of the person to capture food images; (c) if analysis of data from the chewing and/or swallowing sensor indicates that the person is eating, then also analyzing chewing motions, swallowing motions to estimate the amount of food that the person is eating; (d) if analysis of data from the chewing and/or swallowing sensor indicates that the person is eating, then also analyzing food images to estimate the types of food that the person is eating.

In an example, a method for monitoring food consumption can comprise: (a) using an optical sensor worn by a person to measure tissue motion; (b) analyzing measured tissue motions to identify chewing and/or swallowing motions; (c) if chewing and/or swallowing motions have been identified during a period of time (e.g. the last 1-10 minutes), then activating a camera worn by the person to record images of the area in front of the person; (e) if chewing and/or swallowing motions have been identified during the period of time, then analyzing the recorded images to identify food; (f) if food is not identified in the recorded images, then erasing the recorded images; (g) if food is identified in the recorded images, then analyzing the tissue motions and the recorded images to estimate the types and/or amounts of food consumed by the person.

In an example, a device can comprise one or more cameras that automatically and collectively take pictures of a person’s mouth and pictures of nearby food as the person eats, without the need for human intervention to initiate picture taking when the person starts to eat. In an example, a device can comprise one or more cameras that collectively and automatically take pictures of the person’s mouth and pictures of a nearby food, when the person eats, without the need for human intervention.

In an example, a method of estimating a person’s caloric intake can include the step of having the person wear one or more cameras, wherein these cameras collectively and automatically take pictures of nearby food and the person’s mouth. In an example, a device comprises a method of measuring a person’s caloric intake that includes having the person wear one or more cameras, at one or more locations on the person, from which locations these cameras are able to collectively and automatically take pictures of the person’s mouth as the person eats and take pictures of nearby food as the person eats. In an example, a noncontinuous camera in a round of passively-collected data collection can be a sound-activated camera. In an example, the camera (such as a wearable video camera) can be activated to take pictures by chewing, biting, or swallowing sounds that are detected by a wearable microphone.

In an example, a sensor of a generally more-intrusive type (that operates in a less-continuous manner) can collect data only when it is triggered by the results from a sensor of a generally less-intrusive type (that operates in more-continuous manner). For example, a generally more-intrusive camera can be activated to take pictures only when results from a generally less-intrusive motion sensor indicate that a person is probably eating. In an example, passively-collected data about food consumption can be received from one or more motion-triggered cameras that are worn on the person. In an example, these one or more cameras can be wearable video cameras.

In an example, cameras can start taking pictures only when sensors indicate that the person is probably eating. This can reduce privacy concerns as compared to a device and method that takes pictures all the time. In an example, a camera and method can automatically begin taking images when wearable sensors indicate that the person is probably consuming food. In an example, one or more cameras can collectively and automatically take pictures of a person’s mouth and pictures of a nearby food, when the person eats, without the need for human intervention, when the person eats, to activate picture taking.

In an example, a device and/or system can comprise a microphone and a camera, wherein the camera is activated and/or triggered to record food images when swallowing is detected by analyzing sounds recorded by the microphone. In an example, a device and/or system can comprise a microphone and a camera which are integrated into eyewear, wherein the camera is activated and/or triggered to record food images when chewing is detected by analyzing sounds recorded by the microphone. In an example, a device and/or system can comprise a microphone and a camera which are integrated into eyewear, wherein the camera is activated and/or triggered to record food images when swallowing is detected by analyzing sounds recorded by the microphone.

In an example, a device and/or system can comprise a microphone and a camera in a smart band or smart watch, wherein the camera is activated and/or triggered to record food images when swallowing is detected by analyzing sounds recorded by the microphone. In an example, a device worn on a person’s head or neck can comprise a proximity detector that detects when a person’s hand is in proximity with the person’s mouth, wherein detection of this hand-to-mouth proximity triggers and/or activates a camera and/or microphone on the person’s head or neck to record images and/or sounds for more accurate estimation of the types and/or amounts of food consumed by the person.

In an example, a device and/or system can comprise a microphone and a camera which are attached to eyewear, wherein the camera is activated and/or triggered to record food images when chewing is detected by analyzing sounds recorded by the microphone. In an example, a device and/or system can comprise a microphone and a camera which are attached to eyewear, wherein the camera is activated and/or triggered to record food images when swallowing is detected by analyzing sounds recorded by the microphone. In an example, a device and/or system can comprise a microphone and a camera in a smart band or smart watch, wherein the camera is activated and/or triggered to record food images when chewing is detected by analyzing sounds recorded by the microphone.

In an example, a device and/or system can comprise a necklace or neckband with a microphone and a camera. In an example, a device and/or system can comprise a wearable microphone and a wearable camera, wherein the camera is activated and/or triggered to record food images when chewing is detected by analyzing sounds recorded by the microphone. In an example, a device and/or system can comprise a wearable microphone and a wearable camera, wherein the camera is activated and/or triggered to record food images when swallowing is detected by analyzing sounds recorded by the microphone. In an example, a device and/or system can comprise a microphone and a camera, wherein the camera is activated and/or triggered to record food images when chewing is detected by analyzing sounds recorded by the microphone.

In an example, a device worn on a person’s head or neck can comprise an infrared sensor that detects when something (e.g. the person’s hand) is close to the person’s mouth, wherein this detection triggers and/or activates a camera and/or microphone on the person’s head or neck to record images and/or sounds for more accurate estimation of the types and/or amounts of food consumed by the person. In an example, a device worn on a person’s head or neck can comprise an infrared proximity detector that detects when a person’s hand is in proximity with the person’s mouth, wherein detection of this hand-to-mouth proximity triggers and/or activates a camera and/or microphone on the person’s head or neck to record images and/or sounds for more accurate estimation of the types and/or amounts of food consumed by the person.

In an example, a method for food consumption monitoring can comprise: (a) recording sounds from a microphone worn by a person; (b) analyzing the sounds to detect when the person is eating; (c) activating a camera worn by the person to record images when analysis of the sounds indicates that the person is eating; and (d) analyzing the sounds and images to estimate the types and/or amounts of food that the person is eating. In an example, a method for food consumption monitoring can comprise: (a) recording sounds from a microphone worn by a person; (b) analyzing the sounds to detect when the person is eating; (c) recording images from a camera when analysis of the sounds indicates that the person is eating; and (d) analyzing the sounds and images to estimate the types and/or amounts of food that the person is eating.

In an example, a method for food consumption monitoring can comprise: (a) recording sounds from a microphone on a necklace or neck band worn by a person; (b) analyzing the sounds to detect when the person is eating; (c) activating a camera on the necklace or neck band to record images when analysis of the sounds indicates that the person is eating; and (d) analyzing the sounds and images to estimate the types and/or amounts of food that the person is eating. In an example, a method for food consumption monitoring can comprise: (a) recording sounds from a microphone on eyewear worn by a person; (b) analyzing the sounds to detect when the person is eating; (c) activating a camera on the eyewear to record images when analysis of the sounds indicates that the person is eating; and (d) analyzing the sounds and images to estimate the types and/or amounts of food that the person is eating.

In an example, a method for food consumption monitoring can comprise: (a) recording sounds from a microphone on a device worn by a person; (b) analyzing the sounds to detect when the person is eating; (c) activating a camera on the device to record images when analysis of the sounds indicates that the person is eating; and (d) analyzing the sounds and images to estimate the types and/or amounts of food that the person is eating (or has eaten). In an example, a method for food consumption monitoring can comprise: (a) recording sounds from a microphone on a device worn by a person; (b) analyzing the sounds to detect when the person is eating; (c) activating a forward-facing camera on the device to record images of the space in front of the person when analysis of the sounds indicates that the person is eating; and (d) analyzing the sounds and images to estimate the types and/or amounts of food that the person is eating (or has eaten).

In an example, a method for food consumption monitoring can comprise: (a) recording sounds from a microphone on a device worn by a person; (b) analyzing the sounds to detect chewing and/or swallowing sounds which indicate that the person is eating; (c) activating a forward and downward facing camera on the device to record images of the space in front of the person when analysis of the sounds indicates that the person is eating; and (d) jointly analyzing the sounds and images to estimate the types and/or amounts of food that the person is eating (or has eaten). In an example, a method for food consumption monitoring can comprise: (a) recording sounds from a microphone on a device worn by a person; (b) analyzing the recorded sounds to detect chewing and/or swallowing sounds which indicate that the person is eating; (c) if analysis of the recorded sounds does not indicate that the person is eating, then deleting the recorded sounds within a specified period of time; (d) if analysis of the recorded sounds indicates that the person is eating, then activating a camera on the device to record images of the space in front of the person to record food images; and (e) if analysis of the recorded sounds indicates that the person is eating, then analyzing the sounds and the images to estimate the types and/or amounts of food that the person is eating (or has eaten).

In an example, a method for food consumption monitoring can comprise: (a) using a microphone on a device worn by a person to record sounds; (b) using a data processor to analyze the recorded sounds to detect chewing and/or swallowing sounds which indicate that the person is eating; (c) if analysis of the recorded sounds does not indicate that the person is eating, then deleting the recorded sounds a period of time (e.g. of a first length) after they were recorded; (d) if analysis of the recorded sounds indicates that the person is eating, then activating a camera on the device to start recording images of space and/or objects (including food) in front of the person; (e) if analysis of the recorded sounds indicates that the person is eating, then analyzing the sounds and the images to estimate the types and/or amounts of food that the person is eating; and (f) if analysis of the recorded sounds indicates that the person has not eaten (e.g. has stopped eating) during a recent period of time (e.g. of a second length), then deactivating the camera so that it stops recording images.

In an example, a method for food consumption monitoring can comprise: (a) using a microphone on a device worn by a person to record sounds; (b) using a data processor to analyze the recorded sounds to detect chewing and/or swallowing sounds which indicate that the person is eating; (c) if analysis of the recorded sounds does not indicate that the person is eating, then deleting the recorded sounds a period of time (e.g. between 1 and 10 minutes) after they were recorded; (d) if analysis of the recorded sounds indicates that the person is eating, then activating a camera on the device to start recording images of space and/or objects (including food) in front of the person; (e) if analysis of the recorded sounds indicates that the person is eating, then analyzing the sounds and the images to estimate the types and/or amounts of food that the person is eating; and (f) if analysis of the recorded sounds indicates that the person has not eaten (e.g. has stopped eating) during a recent period of time (e.g. between 1 and 10 minutes), then deactivating the camera so that it stops recording images.

In an example, a method for food consumption monitoring can comprise: (a) using a microphone on a device worn by a person to record sounds; (b) using a data processor to analyze the recorded sounds to detect chewing and/or swallowing sounds which indicate that the person is eating; (c) if analysis of the recorded sounds does not indicate that the person is eating, then deleting the recorded sounds a period of time (e.g. between 30 and 90 seconds) after they were recorded; (d) if analysis of the recorded sounds indicates that the person is eating, then activating a camera on the device to start recording images of space and/or objects (including food) in front of the person; (e) if analysis of the recorded sounds indicates that the person is eating, then analyzing the sounds and the images to estimate the types and/or amounts of food that the person is eating; and (f) if analysis of the recorded sounds indicates that the person has not eaten (e.g. has stopped eating) during a recent period of time (e.g. between 3 and 10 minutes), then deactivating the camera so that it stops recording images.

In an example, a method for food consumption monitoring can comprise: (a) using a microphone on a device worn by a person to record sounds; (b) using a data processor to analyze the recorded sounds to detect chewing and/or swallowing sounds which indicate that the person is eating; (c) if analysis of the recorded sounds does not indicate that the person is eating, then deleting the recorded sounds; (d) if analysis of the recorded sounds indicates that the person is eating, then activating a camera on the device to record food images; and (e) if analysis of the recorded sounds indicates that the person is eating, then analyzing the sounds and the images to estimate the types and/or amounts of food that the person is eating (or has eaten).

In an example, a method for food consumption monitoring can comprise: (a) using a microphone on a device worn by a person to record sounds; (b) using a data processor to analyze these sounds to detect chewing and/or swallowing which indicate that the person is eating; (c) if analysis of these sounds does not indicate that the person is eating, then deleting these sounds a period of time (e.g. between 30 seconds and 10 minutes) after they have been records; (d) if analysis of these sounds indicates that the person is eating, then activating a camera on the device to start recording images of space and/or objects in front of the person to capture food images; (e) if analysis of these muscle signals indicates that the person is eating, then also analyzing these sounds and these food images to estimate the types and/or amounts of food that the person is eating; and (f) if analysis of these sounds indicates that the person has stopped eating for a period of time (e.g. between 3 and 15 minutes), then deactivating the camera so that it stops recording images.

In an example, a method for food consumption monitoring can comprise: (a) using an acoustic sensor on a device worn by a person to record sounds; (b) using a data processor to analyze these sounds to detect chewing and/or swallowing which indicate that the person is eating; (c) if analysis of these sounds does not indicate that the person is eating, then deleting these sounds a period of time (e.g. between 30 seconds and 10 minutes) after they have been records; (d) if analysis of these sounds indicates that the person is eating, then activating a camera on the device to start recording images of space and/or objects in front of the person to capture food images; (e) if analysis of these muscle signals indicates that the person is eating, then also analyzing these sounds and these food images to estimate the types and/or amounts of food that the person is eating; and (f) if analysis of these sounds indicates that the person has stopped eating for a period of time (e.g. between 3 and 15 minutes), then deactivating the camera so that it stops recording images.

In an example, a method for food consumption monitoring can comprise: (a) recording sounds from a microphone worn by a person; (b) analyzing the sounds to detect when the person is eating; (c) activating a camera to record images when analysis of the sounds indicates that the person is eating; and (d) analyzing the sounds and images to estimate the types and/or amounts of food that the person is eating. In an example, a method for food consumption monitoring can comprise: (a) recording sounds from a microphone on a wrist-worn device worn by a person; (b) analyzing the sounds to detect when the person is eating; (c) activating a camera on the wrist-worn device to record images when analysis of the sounds indicates that the person is eating; and (d) analyzing the sounds and images to estimate the types and/or amounts of food that the person is eating.

In an example, a method for monitoring food consumption can comprise: (a) using a microphone worn by a person to record sounds; (b) analyzing the recorded sounds to identify chewing and/or swallowing sounds; (c) if chewing and/or swallowing sounds have not been identified during a period of time (e.g. the last 1-10 minutes), then erasing the recorded sounds; (d) if chewing and/or swallowing sounds have been identified during the period of time, then activating a camera worn by the person to record images of the area in front of the person; (e) if chewing and/or swallowing sounds have been identified during the period of time, then analyzing the recorded images to identify food; (f) if food is not identified in the recorded images, then erasing both the recorded sounds and the recorded images; (g) if food is identified in the recorded images, then analyzing the recorded sounds and the recorded images to estimate the types and/or amounts of food consumed by the person.

In an example, a method for monitoring food consumption can comprise: (a) using a microphone worn by a person to record sounds; (b) analyzing the recorded sounds to detect chewing and/or swallowing sounds; (c) if no chewing and/or swallowing sounds have been detected during the last X minutes (where X is between 1 and 10), then erasing the recorded sounds; (d) if chewing and/or swallowing sounds have been detected during the last X minutes, then activating a camera worn by the person to start recording images of the area in front of the person in order to capture food images; (e) if chewing and/or swallowing sounds have been detected during the last X minutes, then analyzing the recorded sounds and recorded images to estimate the types and/or amounts of food consumed by the person; (d) if chewing and/or swallowing sounds have not been detected during the last X minutes, then deactivating a camera worn by the person so that it stops recording images of the area in front of the person.

In an example, a method for monitoring food consumption can comprise: (a) using a microphone worn by a person to record sounds; (b) analyzing the recorded sounds to detect chewing and/or swallowing sounds; (c) if no chewing and/or swallowing sounds have been detected during the last X minutes (where X is between 1 and 10), then erasing the recorded sounds; (d) if chewing and/or swallowing sounds have been detected during the last X minutes, then using a camera worn by the person to record images of the area in front of the person in order to capture food images; and (e) if chewing and/or swallowing sounds have been detected during the last X minutes, then analyzing the recorded sounds and recorded images to estimate the types and/or amounts of food consumed by the person.

In an example, a necklace worn on a person’s neck can comprise an infrared proximity detector that detects when a person’s hand is in proximity with the person’s mouth, wherein detection of this hand-to-mouth proximity triggers and/or activates a camera and/or microphone on the necklace to record images and/or sounds for more accurate estimation of the types and/or amounts of food consumed by the person. In an example, a processor can compare sound patterns from a microphone to sound patterns associated with chewing to identify food consumption (and activate a camera). In an example, a system can comprise a smart watch and/or wrist band worn on a person’s wrist and eyewear worn on the person’s head, wherein the system tracks the distance between the smart watch and/or wrist band and the eyewear, and wherein a camera and/or microphone on the eyewear is triggered and/or activated to start recording images and/or sounds when this distance is below a selected minimum distance (which may indicate that the person is eating).

In an example, a system can comprise a wrist-worn device (e.g. smart watch) and a head-worn device (e.g. eyewear) worn by a person, wherein the system monitors the distance between the wrist-worn device and the head-worn device, and wherein the system triggers and/or activates a camera and/or a microphone on the head-worn device to record images and/or sounds when the distance goes below a minimum distance (which may indicate that the person is eating). In an example, a system can prompt a person to record food images with a camera when a wearable microphone detects that the person is eating.

In an example, an earpiece worn on a person’s head can comprise an infrared proximity detector that detects when a person’s hand is in proximity with the person’s mouth, wherein detection of this hand-to-mouth proximity triggers and/or activates a camera and/or microphone on the earpiece to record images and/or sounds for more accurate estimation of the types and/or amounts of food consumed by the person. In an example, eyewear worn on a person’s head can comprise an infrared proximity detector that detects when a person’s hand is in proximity with the person’s mouth, wherein detection of this hand-to-mouth proximity triggers and/or activates a camera and/or microphone on the eyewear to record images and/or sounds for more accurate estimation of the types and/or amounts of food consumed by the person.

In an example, a device and/or system can visually display estimated perimeters, borders, and/or outlines between different types of food in a meal and prompt a person to confirm or correct these estimated perimeters, border, and/or outlines. In an example, a device and/or system can visually display estimated perimeters, borders, and/or outlines between different types of food in a meal and prompt a person to confirm or correct these estimated perimeters, border, and/or outlines using a touch screen. In an example, a device and/or system can visually display estimated perimeters, borders, and/or outlines between different types of food in a meal and prompt a person to confirm or correct these estimated perimeters, border, and/or outlines via speech.

In an example, a system can identify the perimeters, borders, and/or outlines of different types of food in a multi-food meal. In an example, a system can identify the perimeters, borders, and/or outlines of different types of food in a multi-food meal by analyzing one or more characteristics of food selected from the group consisting of: shape, size, color, texture, temperature, position on a plate or other dish, and timing in a multi-course meal. In an example, a system can identify the perimeters, borders, and/or outlines of different portions of food in a meal by analyzing one or more characteristics of food selected from the group consisting of: shape, size, color, texture, temperature, position on a plate or other dish, and timing in a multi-course meal.

In an example, a device can further comprise a projected laser beam which forms a pattern which is used as a fiducial member to help estimate the scale and/or distance of food in an image. In an example, the size of a portion of food can be estimated relative to one of more fiducial objects near the food. In an example, a person’s hand can be used as a fiducial member to help estimate the scale and/or distance of food in an image. In an example, a plate, cup, utensil, and/or beverage container can be used as a fiducial member to help estimate the scale and/or distance of food in an image. In an example, the size of a portion of food can be estimated relative to a plate, bowl, utensil, cup, or other beverage-holding container.

In an example, a device can project one or more beams of light (in a portion of the light spectrum which is not visible to humans), wherein these light beams form a pattern of light on (or near) food, and wherein this pattern is used as a fiducial element to calibrate the distance, size, volume, and/or focal angle of the food relative to a camera in food images recorded by the camera. In an example, a device can project one or more beams of light (in a portion of the light spectrum which is not visible to humans), wherein these light beams form a checkerboard pattern of light on (or near) food, and wherein this pattern is used as a fiducial element to calibrate the distance, size, volume, and/or focal angle of the food relative to a camera in food images recorded by the camera. In an example, a device can project one or more beams of light (in a portion of the light spectrum which is not visible to humans), wherein these light beams form a geometric pattern of light on (or near) food, and wherein this pattern is used as a fiducial element to calibrate the distance, size, volume, and/or focal angle of the food relative to a camera in food images recorded by the camera.

In an example, a device can project one or more beams of light (in a portion of the light spectrum which is not visible to humans), wherein these light beams form a grid and/or matrix of light on (or near) food, and wherein this grid and/or matrix pattern is used as a fiducial element to calibrate the distance, size, volume, and/or focal angle of the food relative to a camera in food images recorded by the camera. In an example, a device can project one or more beams of light (in a portion of the light spectrum which is not visible to humans), wherein these light beams form a circle of light on (or near) food, and wherein this circle is used as a fiducial element to calibrate the distance, size, volume, and/or focal angle of the food relative to a camera in food images recorded by the camera. In an example, a device can project a light pattern (in a portion of the light spectrum which is not visible to humans) on or near food, wherein this light pattern is used as a fiducial element to calibrate the distance, size, and/or angle of the food relative to a camera in recorded images of the food.

In an example, estimation of the size, volume, and/or amount of food can be based in part on the size, volume, and/or amount of food relative to (e.g. as a percentage of) the size of a projected pattern of laser light. In an example, estimation of the size, volume, and/or amount of food can be based in part on the size, volume, and/or amount of food relative to (e.g. as a percentage of) the size of a projected pattern of laser light projected on, or near, the food. In an example, estimation of the size, volume, and/or amount of food can be informed by the known size of a dining-related object such as a plate, bowl, cup, glass, mug, can, knife, fork, spoon, chop stick, napkin, or placemat. In an example, estimation of the size, volume, and/or amount of food can be based in part on the size, volume, and/or amount of food relative to (e.g. as a percentage of) the size a known dining-related object such as a plate, bowl, cup, glass, mug, can, knife, fork, spoon, chop stick, napkin, or placemat. In an example, estimation of the size, volume, and/or amount of food can be based in part on the size, volume, and/or amount of food relative to (e.g. as a percentage of) the size of a person’s hand.

In an example, the distance and angle from a camera to food can be measured by analysis of the recorded apparent size and shape of an object in a food image (e.g. plate, cup, bowl, glass, can, knife, fork, spook, or napkin) with a known actual size and shape. In an example, the distance and angle from a camera to food can be measured by analysis of the apparent size and shape of an object in a food image with a known actual size and shape.

In an example, a chewing and/or swallowing sensor can be held in contact with a person’s head and/or neck by a spring on an eyewear frame. In an example, a chewing and/or swallowing sensor can be held in contact with a person’s head and/or neck by compressive foam on an eyewear frame. In an example, a chewing and/or swallowing sensor can be held in contact with a person’s head and/or neck by an inflatable chamber on an eyewear frame. In an example, a chewing and/or swallowing sensor can be held in contact with a person’s head and/or neck by a small-scale pneumatic mechanism on an eyewear frame. In an example, a chewing and/or swallowing sensor can be held in contact with a person’s head and/or neck by a small-scale hydraulic mechanism on an eyewear frame. In an example, a device can comprise eyewear with an EEG sensor on the nose bridge of the frame to detect chewing. In an example, a device can comprise eyewear with a vibration sensor on the nose bridge of the frame to detect chewing. In an example, a device can comprise eyewear with a piezoelectric sensor on the nose bridge of the frame to detect chewing.

In an example, a chewing and/or swallowing sensor can be held in contact with a person’s head and/or neck by compressive foam. In an example, a chewing and/or swallowing sensor can be held in contact with a person’s head and/or neck by an inflatable chamber. In an example, a chewing and/or swallowing sensor can be held in contact with a person’s head and/or neck by adhesion. In an example, a chewing and/or swallowing sensor can be held in contact with a person’s head and/or neck by a spring mechanism. In an example, a chewing and/or swallowing sensor can be held in contact with a person’s head and/or neck by a small-scale pneumatic mechanism. In an example, a chewing and/or swallowing sensor can be held in contact with a person’s head and/or neck by a small-scale hydraulic mechanism.

In an example, a chewing and/or swallowing sensor can have a layer of conductive elastomeric polymer. In an example, a chewing and/or swallowing sensor can comprise a conductive elastomeric polymer. In an example, a chewing can have a layer of conductive elastomeric polymer which measures stretching and/or deformation of tissue from jaw motion. In an example, a chewing can have a layer of conductive elastomeric polymer (e.g. a silicon-based polymer) which measures stretching and/or deformation of tissue from jaw motion. In an example, a chewing can have a layer of conductive elastomeric polymer (e.g. PDMS) which measures stretching and/or deformation of tissue from jaw motion. In an example, a chewing and/or swallowing sensor to detect eating can be made from PDMS which has been made impregnated with silver or aluminum. In an example, a chewing and/or swallowing sensor to detect eating can be made from PDMS which has been made impregnated with carbon nanotubes.

In an example, a chewing sensor can detect and measure jaw movement. In an example, a chewing sensor can detect and/or monitor mandible movement. In an example, a chewing sensor can detect and measure mandible movement. In an example, a sensor can detect upward, downward, and lateral motions of a person’s jaw, wherein specific patterns of upward, downward, and lateral motions are identified to detect eating (vs. other activities such as speaking). In an example, a sensor can detect upward and downward motions of a person’s jaw, wherein specific patterns of upward and downward motions are identified to detect eating (vs. other activities such as speaking). In an example, a sensor can be a strain and/or stretch sensor which measures movement of a person’s skin caused by chewing and/or swallowing. In an example, a sensor can measure motion of tissue in the laryngopharynx area.

In an example, a device can comprise a strain sensor which is located behind a person’s ear. In an example, a food consumption sensor can be worn around a person’s ear, spanning between 50% and 75% of the circumference of the outer ear. In an example, a food consumption sensor can be worn on, around, or in a person’s ear. In an example, a sensor can detect changes in air pressure in the ear canal. In an example, a sensor to detect and/or measure food consumption can be located in front of a person’s outer ear (e.g. auricle). In an example, a sensor to detect and/or measure food consumption can be located below a person’s outer ear (e.g. auricle). In an example, a sensor to detect and/or measure food consumption can be located behind a person’s outer ear (e.g. auricle).

In an example, a device can detect eating by monitoring and measuring electrical potentials from muscles associated with chewing and/or swallowing. In an example, a device can estimate food consumption by monitoring and measuring electrical potentials from muscles associated with chewing and/or swallowing. In an example, changes in the ratio of chewing muscle signals to swallowing muscle signals can be analyzed to help estimate the types and/or quantities of food consumed. In an example, the ratio of chewing muscle signals to swallowing muscle signals can be analyzed to help estimate the types and/or quantities of food consumed. In an example, a chewing and/or swallowing sensor can comprise an electrode which measures electrical and/or electromagnetic energy from muscles. In an example, a sensor can detect repeating and/or cyclical patterns in muscle activity which indicate eating. In an example, this device can measure temporalis muscle activity using an ultrasound sensor.

In an example, a device can: (a) continually record first images of space in front of a person to scan for nearby food; (b) if nearby food is detected, then detect if the person starts to eat within a certain period of time based on (changes in the rate of) hand and/or arm muscle signals; (c) if eating by the person is not detected within that period of time, then delete the images; (d) if eating by the person is detected within that period of time, then record second images of space in front of the person when the person stops eating based on (changes in the rate of) hand and/or arm muscle signals; (e) estimate the amount of food consumed by the person by analyzing the difference in food volume and/or size between the first and second images.

In an example, a device can: (a) continually record first images of space in front of a person to scan for nearby food; (b) if nearby food is detected, then detect if the person starts to eat within a certain period of time based on (changes in the rate of) chewing or swallowing muscle signals; (c) if eating by the person is not detected within that period of time, then delete the images; (d) if eating by the person is detected within that period of time, then record second images of space in front of the person when the person stops eating based on (changes in the rate of) chewing and/or swallowing muscle signals; (e) estimate the amount of food consumed by the person by analyzing the difference in food volume and/or size between the first and second images.

In an example, a motion and/or vibration sensor to detect and/or measure food consumption can be located in front of a person’s outer ear (e.g. auricle). In an example, a motion and/or vibration sensor to detect and/or measure food consumption can be located below a person’s outer ear (e.g. auricle). In an example, a motion and/or vibration sensor to detect and/or measure food consumption can be located behind a person’s outer ear (e.g. auricle). In an example, a motion and/or vibration sensor to detect and/or measure food consumption can be located above a person’s outer ear (e.g. auricle).

In an example, a sensor can be a chewing sensor. In an example, a sensor can be a swallow sensor. In an example, a sensor can be located in a person’s mouth. In an example, a sensor can be attached to a person’s teeth. In an example, a sensor can be attached to the upper palate of a person’s mouth. In an example, a sensor can detect opening and closing of a person’s mouth, wherein specific patterns of opening and closing indicate eating (vs. other activities such as speaking). In an example, a sensor can detect opening and closing of a person’s mouth, wherein specific patterns of opening and closing are identified to detect eating (vs. other activities such as speaking).

In an example, a sensor can be made with PDMS which has been made electroconductive by being impregnated with conductive particles and/or material. In an example, a sensor can be made with PDMS which has been made electroconductive by being impregnated with silver, aluminum, and/or carbon. In an example, a sensor can be made with PDMS which has been made electroconductive by being impregnated with carbon nanotubes. In an example, a strain sensor to detect eating can be made from PDMS which has been made impregnated with silver or aluminum. In an example, a strain sensor to detect eating can be made from PDMS which has been made impregnated with carbon nanotubes.

In an example, a sensor to detect and/or measure food consumption can be located above a person’s outer ear (e.g. auricle). In an example, a stretch and/or strain sensor to detect and/or measure food consumption can be located in front of a person’s outer ear (e.g. auricle). In an example, a stretch and/or strain sensor to detect and/or measure food consumption can be located below a person’s outer ear (e.g. auricle). In an example, a stretch and/or strain sensor to detect and/or measure food consumption can be located behind a person’s outer ear (e.g. auricle). In an example, a stretch and/or strain sensor to detect and/or measure food consumption can be located above a person’s outer ear (e.g. auricle).

In an example, a swallow sensor can be attached to a person’s ear. In an example, an acoustic sensor to detect and/or measure food consumption can be located in front of a person’s outer ear (e.g. auricle). In an example, an acoustic sensor to detect and/or measure food consumption can be located below a person’s outer ear (e.g. auricle). In an example, an acoustic sensor to detect and/or measure food consumption can be located behind a person’s outer ear (e.g. auricle). In an example, an acoustic sensor to detect and/or measure food consumption can be located above a person’s outer ear (e.g. auricle).

In an example, a system can analyze, estimate, track, and/or monitor the number of bites per meal and/or per interval of time. In an example, changes in the ratio of chewing sounds to swallowing sounds can be analyzed to help estimate the types and/or quantities of food consumed. In an example, the ratio of chewing sounds to swallowing sounds can be analyzed to help estimate the types and/or quantities of food consumed. In an example, a device can analyze the ratio of chewing motions to hand-to-mouth motions. In an example, a device can analyze the ratio of chewing motions to biting motions.

In an example, an optical sensor worn on a person’s eyeglasses can monitor, detect, and/or measure a person’s chewing motions associated with eating food. In an example, an optical sensor worn on a person’s eyeglasses which directs light beams toward the person’s temple area can monitor, detect, and/or measure a person’s chewing motions associated with eating food. In an example, an optical sensor worn on a person’s eyeglasses which directs light beams toward the person’s jaw can monitor, detect, and/or measure a person’s chewing motions associated with eating food. In an example, an optical sensor worn on a person’s eyeglasses which directs infrared light beams toward the person’s temple area can monitor, detect, and/or measure a person’s chewing motions associated with eating food.

In an example, an optical sensor worn on a person’s eyeglasses which directs infrared light beams toward the person’s jaw can monitor, detect, and/or measure a person’s chewing motions associated with eating food. In an example, an optical sensor on eyewear can be held into close contact with a person’s tissue by one of more mechanisms selected from the group consisting of: a solenoid; compressive foam; elastomeric material (e.g. PDMS); a magnet; a spring; adhesive; an inflatable compartment; a MEMS piston; and an elastic band. In an example, an optical chewing sensor can be integrated into a custom eyewear frame. In an example, chewing intensity can be analyzed to help estimate types and amounts of food consumed. In an example, a device can monitor deformation and/or stretching of the surface of a person’s body which is caused by chewing and/or swallowing.

In an example, the amounts and/or types of food consumed can be measure by the frequency and/or number of swallows. In an example, the number of swallowing sounds, rate of swallowing sounds, frequency of swallowing sounds, and/or magnitude of swallowing sounds can be analyzed to estimate the types and/or quantities of food consumed. In an example, the number of swallowing motions, frequency of swallowing motions, and/or magnitude of swallowing motions can be analyzed to estimate the types and/or quantities of food consumed. In an example, the amounts and/or types of food consumed can be measure by the frequency and/or number of chews.

In an example, the number of chewing sounds, rate of chewing sounds, frequency of chewing sounds, and/or magnitude of chewing sounds can be analyzed to estimate the types and/or quantities of food consumed. In an example, the number of chewing motions, frequency of chewing motions, and/or magnitude of chewing motions can be analyzed to estimate the types and/or quantities of food consumed. In an example, a system can analyze, estimate, track, and/or monitor the number of chewing motions per meal and/or per interval of time. In an example, this device can measure temporalis muscle activity based on analysis of reflected near-infrared light energy. In an example, this device can measure temporalis muscle activity based on analysis of reflected infrared light energy. In an example, this device can measure temporalis muscle activity based on light reflected from a person’s face.

In an example, a device can include a data processing and transmission unit which estimates the person’s caloric intake based on what the person says about what they are eating (actively-entered data), based on biting, chewing, and swallowing sounds (passively-collected data) or the combination of both of these data sources. In an example, a data processing and transmission unit can transmit these data to a remote computer wherein the person’s caloric intake is estimated.

In an example, a device can comprise a light emitter (e.g. LED) which emits light beams toward a person’s head and a light receiver (e.g. photodiode) which receives those light beams after they have been reflected back from the person’s head, wherein variation patterns in the reflected light beams over time is used to detect, monitor, and/or measure chewing motions by the person. In an example, a device can comprise a light emitter (e.g. LED) which emits infrared or near-infrared light beams toward a person’s head and a light receiver (e.g. photodiode) which receives those light beams after they have been reflected back from the person’s head, wherein variation in the magnitude of the reflected light beams is used to detect, monitor, and/or measure chewing motions by the person.

In an example, a device can comprise a light emitter array and a light detector array. In an example, a device can comprise a light emitter, an optical interferometer, and a light receiver. In an example, a device can comprise a light emitter (e.g. LED) which emits infrared or near-infrared light beams toward a person’s head and a light receiver (e.g. photodiode) which receives those light beams after they have been reflected back from the person’s head, wherein variation patterns in the reflected light beams over time is used to detect, monitor, and/or measure chewing motions by the person. In an example, a device can comprise a light emitter, a light receiver, and an opaque barrier between the light emitter and the light receiver.

In an example, a device can comprise a sensor with a light-emitter and a light-receiver which reflects light off the surface of a person’s jaw and/or face to detect movement of jaw muscles. In an example, a device can comprise a sensor with a light-emitter and a light-receiver which detects movement of jaw muscles. In an example, a device can comprise a sensor with a light-emitter and a light-receiver which detects jaw movement. In an example, a device can comprise a sensor with a light-emitter and a light-receiver which detects chewing and/or swallowing motion.

In an example, a device can comprise an optical sensor in proximity to the surface of a person’s head (e.g. jaw and/or side). In an example, an optical sensor to detect and/or measure food consumption can be located in front of a person’s outer ear (e.g. auricle). In an example, an optical sensor to detect and/or measure food consumption can be located below a person’s outer ear (e.g. auricle). In an example, an optical sensor to detect and/or measure food consumption can be located behind a person’s outer ear (e.g. auricle). In an example, an optical sensor to detect and/or measure food consumption can be located above a person’s outer ear (e.g. auricle).

In an example, a device can comprise an optical sensor which is worn on a person’s neck. In an example, a device can comprise an optical sensor which is worn on a person’s head. In an example, a device can comprise an optical sensor which is worn on a person’s ear. In an example, a device can comprise an optical sensor which is worn on a person’s arm. In an example, a device can comprise an optical sensor which is in contact with the surface of a person’s head (e.g. jaw and/or side). In an example, a device can comprise an optical sensor which detects movement of jaw muscles. In an example, a device can comprise an optical sensor which detects jaw movement. In an example, a device can comprise an optical sensor which detects chewing and/or swallowing motion. In an example, a device can comprise an optical sensor which is worn on a person’s wrist.

In an example, a device can comprise an optical sensor. In an example, a device can comprise and/or include an optical sensor. In an example, a device can comprise and/or include an infrared optical sensor. In an example, a device can comprise and/or include a near-infrared optical sensor. In an example, a device can comprise a light-reflecting sensor which detects movement of jaw muscles. In an example, a device can comprise a light-reflecting sensor which detects jaw movement. In an example, a device can comprise a light-reflecting sensor which detects chewing and/or swallowing motion. In an example, a device for measuring a person’s food consumption can include a time-of-flight sensor.

In an example, a device can comprise one or more light emitters which direct near-infrared, infrared, visible, and ultraviolet light beams toward a portion of a person’s body and one or more light receivers which receive these light beams after they have been reflected from that portion of the person’s body, wherein changes in the light beams caused by interaction with the portion of the person’s body are analyzed to detect and/or measure chewing and/or swallowing motions. In an example, a device can comprise one or more light emitters which direct near-infrared, infrared, visible, and ultraviolet light beams toward a person’s jaw and one or more light receivers which receive these light beams after they have been reflected from the person’s jaw, wherein changes in the light beams caused by interaction with the person’s jaw are analyzed to detect and/or measure chewing and/or swallowing motions.

In an example, a device can comprise one or more light emitters which direct near-infrared, infrared, visible, and ultraviolet light beams toward a person’s neck and one or more light receivers which receive these light beams after they have been reflected from the person’s neck, wherein changes in the light beams caused by interaction with the person’s neck are analyzed to detect and/or measure chewing and/or swallowing motions. In an example, a device can comprise a light emitter (e.g. LED) which emits light beams toward a person’s head and a light receiver (e.g. photodiode) which receives those light beams after they have been reflected back from the person’s head, wherein variation in the magnitude of the reflected light beams is used to detect, monitor, and/or measure chewing motions by the person.

In an example, a device can direct near-infrared, infrared, visible, and ultraviolet light toward a portion of a person’s body to monitor for chewing and/or swallowing motions. In an example, swallowing motions can be identified by identification of specific patterns of variation in light reflected from tissue on a person’s neck. In an example, a device can emit light beams toward a person’s jaw, wherein movement of the person’s jaw (e.g. by chewing) causes variation in reflection of those light beams back to the device.

In an example, a device can further comprise one or more optical components selected from the group consisting of: optical prism, concave lens, lens, mirror, optical filter, adjustable lens, laser diode, photodetector, parabolic reflector, optical diffuser, optical louver, convex lens, waveguide, curved mirror, half-mirror, beam splitter, light filter, Fresnel lens, and digital micromirror device. In an example, the distance from a device to food can be measured by reflection of infrared light. In an example, a device can scan food with ultraviolet light. In an example, a device can scan food with near infrared light. In an example, a device can comprise an array of photodetectors. In an example, a device can comprise a waveguide. In an example, a device can scan food with light in range of 300 nm to 1200 nm. In an example, a device can include a photoplethysmography (PPG) sensor. In an example, a device can include an oxygen saturation sensor. In an example, a device can comprise a heart rate sensor. In an example, a device can comprise a Lidar sensor. In an example, a device can include a sensor which detects scents, odors, and/or smells.

In an example, a device can project ultraviolet, visible, near infrared or infrared light toward food. In an example, a device can project ultraviolet, visible, near infrared or infrared light toward food and receive that light after it has been reflected from the food. In an example, a device worn by a person can comprise a light emitter (e.g. a diode) which projects (infrared or near-infrared) light beams onto the person’s neck to detect chewing and/or swallowing. In an example, a device worn by a person can comprise a light emitter (e.g. a diode) which projects (infrared or near-infrared) light beams onto the person’s jaw to detect chewing and/or swallowing. In an example, a device worn by a person can comprise a light emitter (e.g. a diode) which projects (infrared or near-infrared) light beams onto the person’s temple to detect chewing and/or swallowing. In an example, a device can include a near-infrared light emitter and a light receiver, wherein near-infrared light is directed toward and reflected from a portion of a person’s body which moves when the person chews and/or swallows and wherein the reflected light is received by the light receiver.

In an example, a sensor can comprise an infrared light emitter and infrared light receiver, wherein the sensor detects jaw motion. In an example, a sensor can comprise an infrared light emitter and infrared light receiver, wherein the light emitter emits light toward a portion of a person’s body which moves when the person chews and the light receiver receives this light after it has been reflected. In an example, a device can comprise a sensor with a light-emitter and a light-receiver which reflects light off the surface of a person’s jaw and/or face to detect jaw movement. In an example, a device can comprise a sensor with a light-emitter and a light-receiver which reflects light off the surface of a person’s jaw and/or face to detect chewing and/or swallowing motion.

In an example, an optical sensor worn on a person’s ear can monitor, detect, and/or measure a person’s chewing motions associated with eating food. In an example, an optical sensor worn on a person’s ear which directs infrared light beams toward the person’s jaw can monitor, detect, and/or measure a person’s chewing motions associated with eating food. In an example, an optical sensor worn on a person’s ear which directs infrared light beams toward the person’s temple area can monitor, detect, and/or measure a person’s chewing motions associated with eating food. In an example, an optical sensor worn on a person’s ear which directs light beams toward the person’s jaw can monitor, detect, and/or measure a person’s chewing motions associated with eating food. In an example, an optical sensor worn on a person’s ear which directs light beams toward the person’s temple area can monitor, detect, and/or measure a person’s chewing motions associated with eating food.

In an example, a device and system can comprise a camera to record food images and a spectroscopy sensor to scan the food, wherein identification of food types and amounts based on analysis of the food images and identification of the nutritional composition of the food based on analysis of data from the spectroscopy sensor are used to determine the types and amounts of food. In an example, a device and system can comprise a camera to record food images and a spectroscopy sensor to scan the food, wherein combined analysis of the food images and data from the spectroscopy sensor is used to determine the type and amount of food. In an example, a device and system can comprise a camera to record food images and a spectroscopy sensor to scan the food, wherein analysis of the food images and analysis of data from the spectroscopy sensor are used to determine the types and amounts of food.

In an example, a device and system can comprise a camera to record food images and a spectroscopy sensor to scan the food, wherein multivariate analysis of the food images and data from the spectroscopy sensor is used to determine the types and amounts of food. In an example, a device and system can comprise a camera to record food images and a spectroscopy sensor to scan the food, wherein identification of food types and amounts from the food images and identification of food types from the spectroscopy sensor are used to determine the types and amounts of food. In an example, a device can comprise a camera to capture multispectral images of food

In an example, a device can capture hyperspectral images of food in the NIR (Near Infrared) range of the light spectrum. In an example, a device can capture hyperspectral images of food in range of 300 nm to 1200 nm. In an example, a device for measuring a person’s nutritional consumption can comprise a hyperspectral imager. In an example, a device can identify the compositions of different foods based on their hyperspectral distribution signatures. In an example, a device can identify types of food based on their hyperspectral distribution signatures. In an example, a device can identify types of food based on their hyperspectral distribution signatures. In an example, a device can comprise a hyperspectral camera to capture hyperspectral images of food.

In an example, a device can comprise a handheld spectrometer. In an example, a handheld spectroscopic scanner can be used to evaluate the freshness of nearby food. In an example, a handheld spectroscopic scanner can be used to identify food allergens in nearby food. In an example, a handheld spectroscopic scanner can be used to scan nearby food to identify allergens. In an example, a handheld spectroscopic scanner can be used to scan nearby food to identify allergens, contaminants, and/or spoilage. In an example, a wearable device for measuring a person’s food consumption can be in wireless communication with a handheld spectroscopic sensor. In an example, a wearable device for measuring a person’s food consumption can be part of a system which also includes a handheld spectroscopic sensor. In an example, a wearable device for measuring a person’s food consumption can be in wireless communication with a handheld spectrometer. In an example, a wearable device for measuring a person’s food consumption can be part of a system which also includes a handheld spectrometer. In an example, a system for measuring a person’s food consumption can include a mobile phone and a handheld spectroscopic food scanner which are in wireless communication with each other.

In an example, a device can comprise a hyperspectral sensor which directs light beams in spectral ranges which vary over time toward food to detect different types of food ingredients by analyzing spectral changes caused by transmission of the light beams through the food. In an example, a device can comprise a hyperspectral sensor which directs light beams in spectral ranges which vary over time toward food to identify different types of ingredients in food by analyzing spectral changes caused by transmission of the light beams through the food. In an example, a device can comprise a hyperspectral sensor which directs light beams in different spectral ranges toward food to detect different types of food ingredients by analyzing spectral changes caused by transmission of the light beams through the food. In an example, a device can comprise a hyperspectral sensor which directs light beams in different spectral ranges toward food to identify different types of ingredients in food by analyzing spectral changes caused by transmission of the light beams through the food.

In an example, a device can comprise a laser light emitter and a spectroscopic sensor, wherein a light beam from the light emitter is used to aim the spectroscopic sensor toward food to scan the food. In an example, a device can comprise a laser light emitter and a spectrometer, wherein a light beam from the light emitter is used to aim the spectrometer toward food to scan the food. In an example, a device can comprise a light emitter (e.g. LED) which emits light beams toward a person’s head and a light receiver (e.g. photodiode) which receives those light beams after they have been reflected back from the person’s head, wherein variation in the magnitude, direction, and/or spectrum of the reflected light beams is used to detect, monitor, and/or measure chewing motions by the person.

In an example, a device can comprise a light emitter (e.g. LED) which emits infrared or near-infrared light beams toward a person’s head and a light receiver (e.g. photodiode) which receives those light beams after they have been reflected back from the person’s head, wherein variation in the magnitude, direction, and/or spectrum of the reflected light beams is used to detect, monitor, and/or measure chewing motions by the person. In an example, a device can comprise a light emitter (e.g. LED) which emits infrared or near-infrared light beams toward a person’s head and a light receiver (e.g. photodiode) which receives those light beams after they have been reflected back from the person’s head, wherein Fourier Transformation analysis of variation in the magnitude, direction, and/or spectrum of the reflected light beams is used to detect, monitor, and/or measure chewing motions by the person.

In an example, a device can comprise a spectroscopic sensor which directs light beams in different spectral ranges toward food to identify different types of ingredients in food by analyzing spectral changes caused by transmission of the light beams through the food. In an example, a device can direct light beams in different spectral ranges toward food to identify different types of ingredients in food by analyzing spectral changes caused by reflection of the light beams from the food. In an example, a device can direct light beams in near infrared and ultraviolet spectral ranges toward food to detect different types of food ingredients by analyzing spectral changes caused by transmission of the light beams through the food.

In an example, a device can comprise a spectroscopic sensor which is worn on a person’s wrist. In an example, a device can comprise a spectroscopic sensor which is worn on a person’s neck. In an example, a device can comprise a spectroscopic sensor which is worn on a person’s head. In an example, a device can comprise a spectroscopic sensor which is worn on a person’s ear. In an example, a device can comprise a spectroscopic sensor which is worn on a person’s arm.

In an example, a device can comprise a spectroscopic sensor. In an example, a system can include a spectroscopic scanner which is used to scan nearby food to identify allergens. In an example, a system can include a spectroscopic scanner which is used to scan nearby food to identify allergens, contaminants, and/or spoilage. In an example, a system can include a spectroscopic scanner which is used to identify food allergens in nearby food. In an example, a system can include a spectroscopic scanner which is used to evaluate the freshness of nearby food. In an example, a system can include a database with information on the average nutritional and/or molecular compositions of different types of food, wherein this information is adjusted (e.g. using Bayesian statistical methods) based on information on the nutritional and/or molecular composition of nearby food based on data from a spectroscopic scanner which is used to scan that food.

In an example, a device can comprise and/or include a spectroscopic optical sensor. In an example, a method for determining the nutritional composition of food can comprise waving and/or moving a spectroscopic scanner back and forth over food at periodic intervals during an eating event and/or meal. In an example, a method for determining the nutritional composition of food can comprise waving and/or moving a spectroscopic scanner back and forth over food at multiple times during an eating event and/or meal. In an example, a device can use a spectroscopic scan of a meal to segment the meal into portions of different types of food. In an example, a device can comprise a spectroscopic sensor which is used to identify a type of food and/or food ingredient to which a person is allergic. In an example, a device can comprise a spectroscopic sensor which detects movement of jaw muscles. In an example, a device can comprise a spectroscopic sensor which detects jaw movement.

In an example, a device can comprise one or more light emitters which direct near-infrared, infrared, visible, and ultraviolet light beams toward nearby food and one or more light receivers which receive these light beams after they have been reflected from the food, wherein changes in the light beams caused by interaction with the food are analyzed to identify the type of food. In an example, a device can comprise one or more light emitters which direct near-infrared, infrared, visible, and ultraviolet light beams toward nearby food and one or more light receivers which receive these light beams after they have been reflected from the food, wherein changes in the light beams caused by interaction with the food are analyzed to estimate the nutritional and/or molecular composition of the food.

In an example, a device can direct light beams in different spectral ranges toward food to detect different types of food ingredients. In an example, a device can scan food with light beams in different spectral ranges to detect different types of food ingredients. In an example, a device can direct light beams in different spectral ranges toward food to identify different types of ingredients in food. In an example, a device can scan food with light beams in different spectral ranges to identify different types of ingredients in food. In an example, a device can direct light beams in near infrared and ultraviolet spectral ranges toward food to identify different types of ingredients in food by analyzing spectral changes caused by transmission of the light beams through the food. In an example, a device can direct light beams in different spectral ranges toward food to identify different types of ingredients in food by analyzing spectral changes caused by transmission of the light beams through the food.

In an example, a device can direct light beams in different spectral ranges toward food to detect different types of food ingredients by analyzing spectral changes caused by transmission of the light beams through the food. In an example, a device can comprise a spectroscopic sensor which directs light beams in different spectral ranges toward food to detect different types of food ingredients by analyzing spectral changes caused by transmission of the light beams through the food. In an example, a device can direct light beams in different spectral ranges toward food to detect different types of food ingredients by analyzing spectral changes caused by reflection of the light beams from the food. In an example, food composition can be estimated by one or more methods selected from the group consisting of: analysis of food images; chemical analysis of a food sample; radar (e.g. In the millimeter frequency range); and spectroscopic analysis (e.g. analysis of changes in the spectral distribution of light reflect from, or transmitted through, food).

In an example, a device can direct light beams in spectral ranges which vary over time toward food to identify different types of ingredients in food. In an example, a device can direct light beams in spectral ranges which vary over time toward food to identify different types of ingredients in food by analyzing spectral changes caused by reflection of the light beams from the food. In an example, a device can direct light beams in spectral ranges which vary over time toward food to identify different types of ingredients in food by analyzing spectral changes caused by transmission of the light beams through the food. In an example, a device can comprise a spectroscopic sensor which directs light beams in spectral ranges which vary over time toward food to identify different types of ingredients in food by analyzing spectral changes caused by transmission of the light beams through the food.

In an example, a device can identify the compositions of different foods based on their hyperspectral signatures. In an example, a device can identify types of food based on their hyperspectral signatures. In an example, a device can identify types of food based on their hyperspectral signatures. In an example, a device can analyze food composition using hyperspectral imaging. In an example, hyperspectral imaging of nearby food can be used as part of measuring the types and/or amounts of nutrients that a person consumes. In an example, hyperspectral imaging can be used as part of measuring the types and/or amounts of nutrients that a person consumes.

In an example, a device can identify types of food based on their spectroscopic signatures. In an example, a device can identify the compositions of different foods based on their spectroscopic signatures. In an example, a device can comprise a spectroscopic sensor which projects ultraviolet, visible, near infrared or infrared light toward food and receives that light back after it has been reflected from the food. In an example, a device can comprise a spectroscopic sensor which is used to identify a type of food and/or food ingredient selected from the group consisting of: eggs, gluten, lactose, milk, peanuts, processed sugar, seafood, shellfish, soy, tree nuts, and wheat. In an example, a device can comprise a spectroscopic sensor which is used to identify a type of food and/or food ingredient to which a person is allergic, wherein the food or ingredient is selected from the group consisting of: selected from the group consisting of: eggs, gluten, lactose, milk, peanuts, processed sugar, seafood, shellfish, soy, tree nuts, and wheat.

In an example, a device can identify types of food based on their spectral frequency signatures. In an example, a device can identify types of food based on their spectral frequency signatures. In an example, a device can identify the compositions of different foods based on their spectral frequency signatures. In an example, a device can identify types of food based on their spectral distributions. In an example, a device can identify types of food based on their spectral distributions. In an example, a device can identify the compositions of different foods based on their spectral distributions. In an example, a device can automatically adjust the spectral distribution of a food image. In an example, a device can comprise a multispectral imaging component. In an example, a device can include a camera and a light emitter, wherein the device automatically emits light. In an example, a device can comprise an array of tunable light emitters.

In an example, a device can scan food with light beams in spectral ranges which vary over time to detect different types of food ingredients. In an example, a device can scan food with light beams in spectral ranges which vary over time to identify different types of ingredients in food. In an example, a device can direct light beams in spectral ranges which vary over time toward food to detect different types of food ingredients. In an example, a device can direct light beams in spectral ranges which vary over time toward food to detect different types of food ingredients by analyzing spectral changes caused by reflection of the light beams from the food. In an example, a device can direct light beams in spectral ranges which vary over time toward food to detect different types of food ingredients by analyzing spectral changes caused by transmission of the light beams through the food. In an example, a device can comprise a spectroscopic sensor which directs light beams in spectral ranges which vary over time toward food to detect different types of food ingredients by analyzing spectral changes caused by transmission of the light beams through the food.

In an example, a device can use Raman spectroscopy to identify the nutritional composition of food. In an example, a device can use Raman spectroscopy to identify the molecular composition of food. In an example, a device for measuring food composition can use radar spectroscopy. In an example, radar spectroscopy can be used to measure food composition. In an example, a device for measuring a person’s nutritional consumption can comprise a spectrometer. In an example, a device for measuring a person’s nutritional consumption can comprise a near-infrared spectrometer. In an example, a device for measuring a person’s nutritional consumption can comprise an ultrasonic spectrometer. In an example, signals from a sensor can be analyzed using power spectral methods.

In an example, a method for measuring a person’s food consumption can comprise: (a) recording data concerning a person’s arm and/or hand motion from a motion sensor on wrist-worn device; (b) recording data concerning the person’s heart rate from an optical (e.g. spectroscopic and/or PPG) sensor on the wrist-worn device; (c) analyzing the data concerning the person’s arm and/or hand motion and the data concerning the person’s heart rate; (d) activating a camera on the wrist-worn device to start recording food images if multivariate (e.g. joint, combined) analysis of the arm and/or hand motion data and heart rate data indicates that the person is eating; and (e) estimating the types and/or amounts of food that the person has consumed based on multivariate (e.g. joint, combined) analysis of the person’s arm and/or hand motion, the person’s heart rate, and the food images.

In an example, a method for measuring a person’s food consumption can comprise: (a) recording data concerning a person’s arm and/or hand motion from a motion sensor on wrist-worn device; (b) recording data concerning the person’s heart rate from an optical (e.g. spectroscopic and/or PPG) sensor on the wrist-worn device; (c) analyzing the data concerning the person’s arm and/or hand motion and the data concerning the person’s heart rate; (d) activating a camera on the wrist-worn device to start recording food images if analysis of the arm and/or hand motion data indicates eating-associated hand-to-mouth motions and heart rate data indicates an eating-associated increase in heart rate; and (e) estimating the types and/or amounts of food that the person has consumed based on multivariate (e.g. joint, combined) analysis of the person’s arm and/or hand motion, the person’s heart rate, and the food images.

In an example, a method for measuring a person’s food consumption can comprise: (a) recording data concerning a person’s arm and/or hand motion from a motion sensor on wrist-worn device; (b) recording data concerning the person’s heart rate from an optical (e.g. spectroscopic and/or PPG) sensor on the wrist-worn device; (c) analyzing the data concerning the person’s arm and/or hand motion and the data concerning the person’s heart rate using time-series analysis; (d) activating a camera on the wrist-worn device to start recording food images if analysis of the arm and/or hand motion data indicates that the person is eating; and (e) estimating the types and/or amounts of food that the person has consumed based on multivariate (e.g. joint, combined) analysis of the person’s arm and/or hand motion, the person’s heart rate, and the food images.

In an example, a method for measuring a person’s food consumption can comprise: (a) recording data concerning a person’s arm and/or hand motion from a motion sensor on wrist-worn device; (b) recording data concerning the person’s heart rate from an optical (e.g. spectroscopic and/or PPG) sensor on the wrist-worn device; (c) analyzing the data concerning the person’s arm and/or hand motion and the data concerning the person’s heart rate using Fourier Transformation; (d) activating a camera on the wrist-worn device to start recording food images if analysis of the arm and/or hand motion data indicates that the person is eating; and (e) estimating the types and/or amounts of food that the person has consumed based on multivariate (e.g. joint, combined) analysis of the person’s arm and/or hand motion, the person’s heart rate, and the food images.

In an example, a system can further comprise a utensil (e.g. a spoon or a fork) with an optical (e.g. spectroscopic) sensor which collects data to estimate the type of food in a spoonfull or forkfull. In an example, a system can further comprise a utensil (e.g. a spoon or a fork) with an optical (e.g. spectroscopic) sensor which collects data to estimate the nutritional composition of food in a spoonfull or forkfull. In an example, a spectroscopic sensor can be incorporated into a spoon in order to measure the nutritional composition of food in the spoon. In an example, a spectroscopic sensor can be incorporated into a spoon in order to measure the molecular composition of food in the spoon.

In an example, the amounts and/or types of food consumed can be measure by the spectral distribution of swallowing sounds. In an example, the amounts and/or types of food consumed can be measure by the spectral distribution of chewing sounds. In an example, information concerning the number of chewing motions by a person, the number of swallowing motions by a person, images of food near the person, and spectroscopic scans of that food can be jointly analyzed to determine the types and amounts of nutrients consumed by the person. In an example, information concerning the number (and frequency or rate) of chewing motions by a person, the number (and frequency or rate) of swallowing motions by a person, (changes in) images of food near the person (over time), and spectroscopic scans of that food can be jointly analyzed to determine the types and amounts of nutrients consumed by the person. In an example, a device can comprise a spectroscopic sensor which detects chewing and/or swallowing motion. In an example, information concerning chewing, swallowing, food images, and spectroscopic scans of food can be jointly analyzed to determine the types and amounts of nutrients consumed by a person.

In an example, a device can comprise a wristband and/or smartwatch worn by a person which further comprises a motion sensor and an EMG sensor, wherein data from the motion sensor and the EMG sensor are used to detect when the person is eating. In an example, a system can comprise two wrist-worn devices with EMG sensors, wherein detection of eating motion on either side triggers an eyewear camera to start recording food images. In an example, a system can comprise two wrist-worn devices with EMG sensors, one worn on the right wrist and one worn on the left wrist, to monitor for eating-related hand motions on either side.

In an example, a device can comprise an EMG sensor for recording the activity of chewing and/or swallowing related muscles which is clipped onto the collar of an upper-body garment. In an example, a device can comprise an EMG sensor for recording the activity of chewing and/or swallowing related muscles which is clipped, hooked, and/or clamped onto eyeglasses. In an example, a device can comprise an EMG sensor for recording the activity of chewing and/or swallowing related muscles which is clipped, hooked, and/or clamped onto a sidepiece (e.g. “temple”) of an eyewear frame. In an example, a device can comprise eyewear with an EMG sensor on the nose bridge of the frame. In an example, a device can comprise eyewear with an EMG sensor on the nose bridge of the frame to detect chewing. In an example, this device can be part of a system which includes a wrist band with an EMG sensor to detect and/or track eating-related hand-to-mouth motions. In an example, this device can be part of a system which includes an arm band with an EMG sensor to detect and/or track eating-related hand-to-mouth motions. In an example, a device can comprise an EMG sensor which is worn on a person’s wrist. In an example, a device can comprise an EMG sensor which is worn on a person’s arm.

In an example, a device can comprise an EMG sensor in proximity to the surface of a person’s head (e.g. jaw and/or side). In an example, a device can comprise an EMG sensor which is worn on a person’s neck. In an example, a device can comprise an EMG sensor which is worn on a person’s head. In an example, a device can comprise an EMG sensor which is worn on a person’s ear. In an example, a device can comprise an EMG sensor which is in contact with the surface of a person’s head (e.g. jaw and/or side). In an example, a device can comprise an EMG sensor which records electrical signals from one or more of the following muscles or the nerves which innervate those muscles: masseter, temporalis, trapezius, troglodytam fictus, hyoid, medial pterygoid, sphenomandibularis, sternomastoid, lateral pterygoid, and sternohyomastoid.

In an example, a device can detect eating by monitoring and measuring electrical potentials from muscles which move a person’s jaw and/or move food along the person’s GI tract by peristaltic motion. In an example, a device can estimate food consumption by monitoring and measuring electrical potentials from muscles which move a person’s jaw and/or move food along the person’s GI tract by peristaltic motion.

In an example, a device can include an EMG (electromyographic) sensor which measures electrical and/or electromagnetic energy from muscles and/or the nerves which innervate those muscles. In an example, a device can comprise an EMG sensor worn on a person’s head or neck which measures electrical activity of muscles associated with chewing and/or swallowing. In an example, the ratio of chewing-related EMG signals to swallowing-related EMG signals can be analyzed to help estimate the types and/or quantities of food consumed.

In an example, a device can include an EMG sensor for detecting eating based on electrical signals from one or more muscles selected from the group consisting of: masseter, temporalis, trapezius, troglodytam fictus, hyoid, medial pterygoid, sphenomandibularis, sternomastoid, lateral pterygoid, and sternohyomastoid. In an example, a device can include an EMG sensor that records signals from the sphenomandibularis muscle or the nerves which innervate that muscle. In an example, a device can include an EMG sensor that records signals from the medial pterygoid muscle or the nerves which innervate that muscle. In an example, a device can include an EMG sensor that records signals from the masseter muscle or the nerves which innervate that muscle. In an example, a device can include an EMG sensor that records signals from the lateral pterygoid muscle or the nerves which innervate that muscle.

In an example, a device can include an EMG sensor records signals from the hyoid muscle or the nerves which innervate that muscle. In an example, a device can include an EMG sensor records signals from the trapezius muscle or the nerves which innervate that muscle. In an example, a device can include an EMG sensor records signals from the temporalis muscle or the nerves which innervate that muscle. In an example, a device can include an EMG sensor records signals from the sternomastoid muscle or the nerves which innervate that muscle. In an example, a device can include an EMG sensor records signals from the sternohyomastoid muscle or the nerves which innervate that muscle. In an example, food consumption can be detected by monitoring activity of the temporalis muscle via EMG, sound, motion, or optical movement. In an example, this device can measure temporalis muscle activity using an EMG sensor.

In an example, a device or system for measuring a person’s food consumption can comprise: (a) using an EMG sensor to collect data concerning muscles which move a person’s jaw and/or throat for chewing and/or swallowing; (b) analyzing data from the EMG sensor to detect (chewing and/or swallowing which indicates) that the person is eating; (c) activating a camera to record food images when analysis of data from the EMG sensor indicates that the person is eating; (d) analyzing data from the EMG sensor and recorded food images to measure the types and/or amounts of food consumed by the person.

In an example, a device or system for measuring a person’s food consumption can comprise: (a) using an EMG sensor on eyewear (e.g. AR eyewear) to collect data concerning muscles which move a person’s jaw and/or throat for chewing and/or swallowing; (b) analyzing data from the EMG sensor to detect (chewing and/or swallowing which indicates) that the person is eating; (c) activating a camera on the eyewear to record food images when analysis of data from the EMG sensor indicates that the person is eating; (d) analyzing data from the EMG sensor and recorded food images to measure the types and/or amounts of food consumed by the person.

In an example, a device or system for measuring a person’s food consumption can comprise: (a) using an EMG sensor on a wrist-worn device (e.g. a smart watch) to collect data concerning muscles which move a person’s arm and/or hand; (b) analyzing data from the EMG sensor to detect (hand-to-mouth motions that indicate) that the person is eating; (c) activating a camera on the wrist-worn device to record food images when analysis of data from the EMG sensor indicates that the person is eating; (d) analyzing data from the EMG sensor and recorded food images to measure the types and/or amounts of food consumed by the person.

In an example, a method for food consumption monitoring can comprise: (a) using an EMG sensor on a device worn by a person to measure muscle signals on or near the person’s jaw; (b) using a data processor to analyze these muscle signals to detect chewing and/or swallowing motions which indicate that the person is eating; (c) if analysis of these muscle signals indicates that the person is eating, then activating a camera on the device to start recording images of space and/or objects in front of the person to capture food images; (d) if analysis of these muscle signals indicates that the person is eating, then also analyzing these muscle signals and these food images to estimate the types and/or amounts of food that the person is eating; and (e) if analysis of these muscle signals indicates that the person has not eaten (e.g. has stopped eating) during a recent period of time, then deactivating the camera so that it stops recording images.

In an example, a method for monitoring food consumption can comprise: (a) using an EMG sensor worn by a person to measure muscle activity; (b) analyzing muscle activity to identify chewing and/or swallowing; (c) if chewing and/or swallowing have been identified during a period of time (e.g. the last 1-10 minutes), then activating a camera worn by the person to record images of the area in front of the person; (e) if chewing and/or swallowing have been identified during the period of time, then analyzing the recorded images to identify food; (f) if food is not identified in the recorded images, then erasing the recorded images; (g) if food is identified in the recorded images, then analyzing the muscle activity and the recorded images to estimate the types and/or amounts of food consumed by the person. In an example, a sensor can detect repeating and/or cyclical patterns in neural impulses which indicate eating.

In an example, an EMG sensor can be incorporated into an adhesive patch which a person wears on their body. In an example, an EMG sensor can be incorporated into an adhesive patch to detect eating. In an example, an EMG sensor on eyewear can be held into close contact with a person’s tissue by one of more mechanisms selected from the group consisting of: a solenoid; compressive foam; elastomeric material (e.g. PDMS); a magnet; a spring; adhesive; an inflatable compartment; a MEMS piston; and an elastic band. In an example, an EMG sensor which is attached to eyewear can be held into close contact with a person’s tissue by one of more mechanisms selected from the group consisting of: a solenoid; compressive foam; elastomeric material (e.g. PDMS); a magnet; a spring; adhesive; an inflatable compartment; a MEMS piston; and an elastic band.

In an example, changes in the ratio of chewing-related EMG signals to swallowing-related EMG signals can be analyzed to help estimate the types and/or quantities of food consumed. In an example, a system can prompt a person to record food images with a camera when a wearable EMG sensor detects that the person is eating. In an example, a system can prompt a person to verbally identify food when a wearable EMG sensor detects that the person is eating. In an example, a device can comprise an EMG-based user interface.

In an example, a device can comprise and/or include an EEG sensor. In an example, a system can identify correlations between a person’s brainwave patterns recorded by EEG sensors worn by the person with the person’s consumption of selected types and/or amounts of food. In an example, a system can use these identified correlations to predict and/or estimate the person’s future consumption of selected types and/or amounts of food. In an example, a system can correlate patterns of electromagnetic signals received by EEG sensors worn by a person with a person’s hunger level and/or satiety level. In an example, a system can analyze patterns of electromagnetic signals received by EEG sensors worn by a person to identify associations between these patterns and a person’s hunger level and/or satiety level. In an example, a system can analyze patterns of electromagnetic signals received by EEG sensors worn by a person to evaluate a person’s hunger level and/or satiety level.

In an example, a device can include a capacitance sensor. In an example, a device can include a capacitance sensor which measures changes in tissue capacitance to detect eating. In an example, a device can comprise a capacitance sensor which is in contact with the surface of a person’s head (e.g. jaw and/or side). In an example, a device can include a capacitance sensor to detect eating. In an example, a device can comprise a capacitance sensor in proximity to the surface of a person’s head (e.g. jaw and/or side). In an example, a device can comprise a magnetic sensor. In an example, a device can comprise a galvanic skin response (GSR) sensor worn by a person which activates a camera to record food images when analysis of data from the sensor indicates that the person is eating.

In an example, a device can include a permittivity sensor. In an example, a device can include a permittivity sensor which measures changes in tissue permittivity to detect eating. In an example, a device can include a permittivity sensor to detect eating. In an example, a device can include a magnetic sensor. In an example, a device can include a magnetic energy sensor. In an example, a device can include an electromagnetic energy sensor which measures changes in tissue electromagnetism to detect eating. In an example, a device can include an electromagnetic energy sensor which measures changes in an electromagnetic field to detect eating. In an example, a device can comprise an electromagnetic energy sensor which is in contact with the surface of a person’s head (e.g. jaw and/or side). In an example, a device can include an electromagnetic energy sensor to detect eating. In an example, a device can comprise an electromagnetic energy sensor in proximity to the surface of a person’s head (e.g. jaw and/or side).

In an example, a device can include an impedance sensor. In an example, a device can include an impedance sensor which measures changes in tissue impedance to detect eating. In an example, a device can comprise an impedance sensor which is in contact with the surface of a person’s head (e.g. jaw and/or side). In an example, a device can include an impedance sensor to detect eating. In an example, a device can comprise an impedance sensor in proximity to the surface of a person’s head (e.g. jaw and/or side).

In an example, a sensor can detect repeating and/or cyclical patterns in body-generated electromagnetic signals which indicate eating. In an example, a device can comprise an electromagnetic field sensor which is in contact with the surface of a person’s head (e.g. jaw and/or side). In an example, a device can comprise an electromagnetic field sensor in proximity to the surface of a person’s head (e.g. jaw and/or side). In an example, a device can include an electromagnetic energy sensor. In an example, a sensor can detect repeating and/or cyclical patterns in body-generated electrical signals which indicate eating. In an example, a device can comprise an electrical resistance sensor. In an example, a device can comprise an electrical inductance sensor. In an example, a device can comprise an electrical capacitance sensor. In an example, a device can include an electrogastrography (EGG) sensor. In an example, a device can comprise a piezoelectric sensor.

In an example, a chewing and/or swallowing sensor can be a piezoelectric bone-conduction microphone. In an example, a device can comprise a bone conduction microphone. In an example, a device can comprise a condenser microphone, a dynamic microphone, a silicon microphone, a sound sensor, a vibration sensor, an acoustic sensor, and/or an audio input device. In an example, a device can comprise a microphone. In an example, a device can comprise a sound recording sensor (such as a microphone). In an example, a device can have a microphone which records sounds from a person’s stomach tract to detect eating.

In an example, a device can comprise a microphone as part of a smart watch. In an example, a device can comprise a microphone attached to a necklace. In an example, a device can comprise a microphone attached to or inserted into ear. In an example, a device can comprise a microphone for recording eating sounds which is clipped, hooked, and/or clamped onto eyeglasses. In an example, a device can comprise a microphone for recording eating sounds which is clipped, hooked, and/or clamped onto a sidepiece (e.g. “temple”) of an eyewear frame. In an example, a device can comprise a microphone for recording eating sounds which is clipped onto the collar of an upper-body garment.

In an example, a device can comprise a microphone which is place on a person’s tissue which covers their mastoid bone. In an example, a device can comprise a microphone which is place on a person’s tissue over their mastoid bone. In an example, a device can comprise a microphone which is located behind a person’s ear. In an example, a device can have a microphone which records sounds from a person’s GI tract to detect eating. In an example, a device for measuring a person’s food consumption can comprise a microphone which is worn on or around the person’s neck. In an example, a microphone can be attached to a person’s skin. In an example, a microphone can be attached to a person’s ear. In an example, a microphone can be attached to a person’s abdomen. In an example, a microphone can be placed behind a person’s ear. In an example, this device can measure temporalis muscle activity using a microphone.

In an example, a device can comprise a microphone which is worn on a person’s wrist. In an example, a device can comprise a microphone which is worn on a person’s neck. In an example, a device can comprise a microphone which is worn on a person’s head. In an example, a device can comprise a microphone which is worn on a person’s ear. In an example, a device can comprise a microphone which is worn on a person’s arm. In an example, a device can comprise an adhesive patch with a microphone which records sounds from a person’s GI tract to detect eating.

In an example, a device can comprise a microphone, wherein speech automatically recognized and blurred out. In an example, a microphone can continually record sounds, but these sounds are automatically erased after 1 to 5 minutes if no eating-related sounds are detected. In an example, a microphone can continually record sounds, but these sounds are automatically erased after 10 to 60 seconds if no eating-related sounds are detected. In an example, analysis of acoustic signals (sound) from a microphone can be used to detect chewing, the rate of chewing, the amount of food consumed. In an example, cyclical variation of amplitude of sounds recorded by a wearable microphone can be analyzed using to identify chewing and/or swallowing.

In an example, a device can comprise an adhesive patch with a microphone to detect eating. In an example, a microphone can be attached to an article of clothing. In an example, a microphone can be attached to eyeglasses or eyewear. In an example, a microphone can be attached to eyewear. In an example, a system can comprise a wrist-worn device (e.g. smart watch) and a head-worn device (e.g. eyewear) worn by a person, wherein the system monitors the distance between the wrist-worn device and the head-worn device, and wherein the system triggers and/or activates a microphone on the head-worn device to record sounds (which may include chewing and/or swallowing sounds) is less than a minimum distance. In an example, a wearable microphone can be incorporated into an adhesive patch which a person wears on their body.

In an example, a device can comprise and/or include an acoustic sensor. In an example, a device can comprise and/or include an ultrasonic sensor. In an example, a sensor can be an acoustic sensor. In an example, a device can include a carbon microphone to record sounds which are analyzed to detect eating. In an example, a device can include a condenser microphone to record sounds which are analyzed to detect eating. In an example, a device can include a contact microphone. In an example, a device can include a dynamic microphone to record sounds which are analyzed to detect eating. In an example, a device can include a laser microphone to record sounds which are analyzed to detect eating. In an example, a device can include a liquid microphone to record sounds which are analyzed to detect eating. In an example, a device can include a MEMS microphone to record sounds which are analyzed to detect eating.

In an example, a device can include a piezoelectric microphone to record sounds which are analyzed to detect eating. In an example, a device can include a ribbon microphone to record sounds which are analyzed to detect eating. In an example, a device can prompt a person to provide information on the food types and/or amounts that they are eating when their eating is detected by microphone that the person wears. In an example, a microphone can be a contact microphone. In an example, a microphone can be a piezoelectric microphone. In an example, a microphone can comprise a piezoelectric film. In an example, a microphone can convert sound into electrical signals. In an example, a sensor can be a microphone. In an example, a system can prompt a person to verbally identify food when a wearable microphone detects that the person is eating.

In an example, a sensor can detect repeating and/or cyclical patterns in body-generated sounds which indicate eating. In an example, a device and/or system can comprise a sound-based swallowing sensor and a camera, wherein the camera is activated and/or triggered to record food images when swallowing is detected by the swallowing sensor. In an example, a sound-based sensor can be worn on a person’s neck as part of a necklace or neck band. In an example, a device can comprise a sound sensor which is in contact with the surface of a person’s head (e.g. jaw and/or side). In an example, a device can comprise a sound sensor in proximity to the surface of a person’s head (e.g. jaw and/or side). In an example, a system can prompt a person to verbally identify food when a wearable sound sensor detects that the person is eating. In an example, sound recorded by a chewing and/or swallowing sensor can be sent through low pass filter. In an example, an acoustic sensor can convert sound into electrical signals.

In an example, an microphone on eyewear can be held into close contact with a person’s tissue by one of more mechanisms selected from the group consisting of: a solenoid; compressive foam; elastomeric material (e.g. PDMS); a magnet; a spring; adhesive; an inflatable compartment; a MEMS piston; and an elastic band. In an example, an microphone which is attached to eyewear can be held into close contact with a person’s tissue by one of more mechanisms selected from the group consisting of: a solenoid; compressive foam; elastomeric material (e.g. PDMS); a magnet; a spring; adhesive; an inflatable compartment; a MEMS piston; and an elastic band.

In an example, cyclical variation of amplitude of sounds recorded by a wearable microphone can be analyzed using to identify chewing and/or swallowing which, in turn, is used to estimate the amount of food consumed by a person. In an example, cyclical variation of amplitude of sounds recorded by a wearable microphone can be analyzed using Fourier Transformation to identify chewing and/or swallowing. In an example, cyclical variation of amplitude of sounds recorded by a wearable microphone can be analyzed using Fourier Transformation to identify chewing and/or swallowing which, in turn, is used to estimate the amount of food consumed by a person. In an example, sounds recorded by a microphone can be sent through one or more filters which obscure and/or erase speech (in the interest of privacy) but still enable capturing chewing and/or swallow sounds. In an example, this device can measure temporalis muscle activity based on analysis of ultrasonic sound energy. In an example, this device can measure temporalis muscle activity based on analysis of sound. In an example, this device can measure temporalis muscle activity based on analysis of reflected ultrasonic sound energy.

In an example, cyclical variation of sounds recorded by a wearable microphone can be analyzed using Fourier Transformation to identify chewing and/or swallowing. In an example, cyclical variation of sounds recorded by a wearable microphone can be analyzed using Fourier Transformation to identify chewing and/or swallowing which, in turn, is used to estimate the amount of food consumed by a person. In an example, eating can be detected by analyzing undulating and/or sinusoid patterns in sounds recorded by a microphone which are caused by of chewing and/or swallowing food. In an example, sounds recorded by a wearable microphone can be analyzed using Fourier Transformation to identify chewing and/or swallowing. In an example, sounds recorded by a wearable microphone can be analyzed to identify chewing and/or swallowing. In an example, the frequencies and amplitudes of sounds recorded by a wearable microphone can be analyzed to identify chewing and/or swallowing.

In an example, a sound sensor can be used for this continuous, but less-intrusive, monitoring function. A sound sensor can continually monitor for eating events by monitoring for biting, chewing, and swallowing sounds. In an example, a non-continuous camera can be activated by the output of wearable sound sensor or a wearable motion sensor. In an example, a sound sensor can be worn on the person’s neck in a manner like a necklace. In an example, a sound sensor can be worn behind the person’s ear in a manner like a bluetooth communication device. In an example, when the sound sensor detects chewing, biting, or swallowing sounds, then the sound sensor activates a camera which can better determine what, if anything, the person is eating.

In an example, operation of a camera can be triggered by sound. In an example, a device can detect biting, chewing, or swallowing sounds as a person eats. In an example, a wearable sound sensor can be worn around the neck like a necklace. In an example, a wearable sound sensor can detect chewing, biting, or swallowing sounds that indicate a probable eating activity. This detection can be through direct contact with the body or through chewing, biting, or swallowing sounds traveling through the air. In an example, a sound sensor can be worn behind the ear. In an example, a wearable sound sensor can be worn under clothing in a manner that is less conspicuous than a wearable camera. In an example, a “level 1” sensor can be a wearable motion sensor or sound sensor and a “level 2” sensor can be a wearable camera or other image-creating sensor.

In an example, a device can be part of a system which analyzes the number, frequency, and amplitude of swallowing sounds. In an example, a device can be part of a system which analyzes the number, frequency, and amplitude of chewing sounds. In an example, a device can be part of a system which analyzes the number and frequency of swallowing sounds. In an example, a device can be part of a system which analyzes the number and frequency of chewing sounds. In an example, a device can monitor sounds transmitted through the mastoid bone.

In an example, a device can transform and/or convert an analog sound into a digital signal. In an example, a type of food can be identified by the amplitude and frequency distribution of chewing and/or swallowing sounds when a person eats that type of food. In an example, a type of food can be identified by the sound signature of chewing and/or swallowing sounds when a person eats that type of food. In an example, chewing sound patterns can be analyzed to identify the amounts and types of food being eaten. In an example, chewing sound patterns can be analyzed to identify the (general) type of food being eaten. In an example, chewing sounds can be analyzed to identify the (general) type of food being eaten.

In an example, eating can be detected by analyzing the amplitude and frequency of chewing and/or swallowing sounds. In an example, temporal analysis of chewing sound patterns can be used identify the amounts and types of food being eaten. In an example, the amounts and/or types of food consumed can be estimated by analyzing the relationship between chewing sounds and swallowing sounds. In an example, the amounts and/or types of food consumed can be estimated by comparing chewing sounds and swallowing sounds during an eating event. In an example, the amounts and/or types of food consumed can be measure by the amplitude of chewing sounds. In an example, the amounts and/or types of food consumed can be measure by the amplitude of swallowing sounds. In an example, the amounts and/or types of food consumed can be measure by the pitch of chewing sounds.

In an example, the amounts and/or types of food consumed can be measure by the pitch of swallowing sounds. In an example, the amounts and/or types of food consumed can be measure by the temporal distribution of chewing sounds. In an example, the amounts and/or types of food consumed can be measure by the temporal distribution of swallowing sounds. In an example, the frequency and pitch of chewing sounds can be analyzed to identify the (general) type of food being eaten.

In an example, a microphone and speaker unit can continually monitor sounds for biting, chewing, or swallowing sounds that indicate that the person is probably eating something. As a person inserts food into their mouth and begins to bite, chew, and swallow, these sounds can be detected by the microphone and speaker unit. In an example, chewing, biting, and swallowing sounds can be conducted through the person’s body to the microphone and speaker unit, instead of (or in addition to) being conducted through the air. These sounds can be analyzed directly in the microphone and speaker unit or they can be transmitted for analysis in a data processing and transmission unit. In an example, analysis of these sounds can indicate probable eating.

In an example, a device can analyze the relationship between hand-to-mouth motions and chewing motions. In an example, a device can analyze the relationship between biting motions and chewing motions. In an example, a device can be part of a system which analyzes the number, frequency, and amplitude of swallowing motions. In an example, a device can be part of a system which analyzes the number, frequency, and amplitude of chewing motions. In an example, a device can be part of a system which analyzes the number and frequency of swallowing motions. In an example, a device can be part of a system which analyzes the number and frequency of chewing motions.

In an example, a device can comprise an inertial motion unit (IMU). In an example, a device can comprise an inertial motion unit (IMU) which is worn on a person’s neck. In an example, a device can comprise an inertial motion unit (IMU) which is worn on a person’s head. In an example, a device can comprise an inertial motion unit (IMU) which is worn on a person’s ear. In an example, a device can comprise an inertial motion unit (IMU) which is worn on a person’s arm. In an example, a device can comprise and/or include an accelerometer. In an example, a device can detect eating by monitoring and measuring mechanical motion of a person’s jaw and/or nearby tissue. In an example, a device can estimate food consumption by monitoring and measuring mechanical motion of a person’s jaw and/or nearby tissue. In an example, a device can track and count biting motions. In an example, a device can track and count chewing motions. In an example, a device can track and count hand-to-mouth motions.

In an example, a motion sensor can comprise an accelerometer, a gyroscope, and an inclinometer. In an example, a motion sensor can comprise an accelerometer and a gyroscope. In an example, a system can further comprise a utensil (e.g. a spoon or a fork) with a motion sensor which collects data to estimate the amount (e.g. quantity and/or weight) of food in a spoonfull or forkfull. In an example, a system can integrate data from a person’s recent pattern of movements and/or locations with data from wearable sensors to enhance estimation of the types and/or amounts of food consumed by the person. In an example, a chewing sensor can be an IMU (Inertial motion sensor) comprising an accelerometer and a gyroscope (and an inclinometer).

In an example, an eating-related hand-to-mouth movement can comprise the following sequence of movements: upward movement of a hand; rotation of the hand; a pause; counter-rotation of the hand; and downward movement of the hand. In an example, chewing motions can be detected and/or measured by cyclical changes in the angle and/or amplitude of light reflected from tissue near a person’s jaw. In an example, chewing motions can be detected and/or measured by changes in the angle of light reflected from a person’s jaw. In an example, chewing motions can be detected and/or measured by changes in the angle of light reflected from tissue near a person’s jaw. In an example, chewing motions can be detected and/or measured by changes in the angle and/or amplitude of light reflected from a person’s jaw. In an example, chewing motions can be identified by identification of specific patterns of variation in light reflected from tissue near a person’s jaw. In an example, data from sensors can be analyzed to group chewing and/or swallowing motions together into an eating event. In an example, data on a person’s hand movements and chewing movements can be analyzed to identify relationships between those hand movements and chewing movements. In an example, data on a person’s hand movements and chewing movements can be jointly analyzed to better estimate the types and amounts of foods consumed by a person.

In an example, a “level 1” sensor can be a wearable camera with a narrow field of view and a short-range focus and a “level 2” sensor can be a wearable camera with a wide field of view and a variable-range focus. In an example, a “level 1” sensor can be a wearable camera that only takes pictures when a motion or sound sensor suggests that an eating event is occurring and a “level 2” sensor can be a wearable camera that takes pictures continuously. In an example, a “level 1” sensor can be a wearable camera that only takes pictures at a certain time of day, or in a certain GPS-indicated location, that suggests that the person can be eating, and a “level 2” camera can take pictures continuously. In an example, a device can monitor a person’s food consumption and estimates the person’s caloric intake when a person tilts their hand upwards to bring glass up to their mouth to drink. This tilting movement, especially when followed by a pause and then a reverse tilting movement, can be detected and recognized by a motion sensor as indicating a probable eating event.

In an example, a device can comprise a wristband and/or smartwatch worn by a person which further comprises one or more motion sensors and one or more EMG sensors, wherein data from the motion sensor and the EMG sensor are used to detect eating-related gestures and/or hand-to-mouth motions. In an example, a device can comprise a wristband and/or smartwatch worn by a person can further comprises one or more motion sensors and one or more EMG sensors, wherein data from the motion sensor and the EMG sensor are used to track and/or count eating-related gestures and/or hand-to-mouth motions. In an example, a device can detect eating by monitoring hand gestures using one or more EMG sensors. In an example, a device can estimate food consumption by monitoring hand gestures using one or more EMG sensors. In an example, this device can be part of a system which includes a wrist band with an EMG sensor to detect and/or track eating-related hand gestures. In an example, this device can be part of a system which includes an arm band with an EMG sensor to detect and/or track eating-related hand gestures.

In an example, a device can detect eating by monitoring hand gestures using one or more motion sensors. In an example, a device can detect eating by monitoring hand gestures using one or more IMU sensors. In an example, a device can detect eating by monitoring hand gestures using a smart watch. In an example, a device can detect eating by monitoring hand gestures using a camera. In an example, a device can estimate food consumption by monitoring hand gestures using one or more motion sensors. In an example, a device can estimate food consumption by monitoring hand gestures using one or more IMU sensors.

In an example, a device can estimate food consumption by monitoring hand gestures using a smart watch. In an example, a device can estimate food consumption by monitoring hand gestures using a camera. In an example, an eyewear-based camera can detect hand gestures which are associated with eating. In an example, analysis of images recorded by an eyewear-based camera can detect hand gestures which are associated with eating. In an example, food consumption can be detected and/or measured based on joint analysis of data from motion sensors, sound sensors, and hand gesture sensors. In an example, this device can be part of a system which includes a wrist band with a motion sensor to detect and/or track eating-related hand gestures. In an example, a device can comprise a gesture-based user interface.

In an example, a method for food consumption monitoring can comprise: (a) analyzing hand gesture and/or hand motion data from a wrist-worn device (e.g. smart watch or wrist band) worn by a person; (b) if analysis of this data indicates that the person is eating, then activating a camera on eyewear worn by the person to start recording images to capture food images; (c) if analysis of this data indicates that the person is eating, then also analyzing this hand gesture and/or hand motion data and the food images to estimate the types and/or amounts of food that the person is eating; and (e) if analysis of this data indicates that the person has stopped eating for a period of time (e.g. between 3 and 15 minutes), then deactivating the camera so that it stops recording images.

In an example, a method for food consumption monitoring can comprise: (a) analyzing hand gesture and/or hand motion data from a device (e.g. smart watch or wrist band) worn by a person; (b) if analysis of this data indicates that the person is eating, then activating a camera on the device to start recording images to capture food images; (c) if analysis of this data indicates that the person is eating, then also analyzing this hand gesture and/or hand motion data and the food images to estimate the types and/or amounts of food that the person is eating; and (e) if analysis of this data indicates that the person has stopped eating for a period of time (e.g. between 3 and 15 minutes), then deactivating the camera so that it stops recording images.

In an example, a method for measuring a person’s food consumption can comprise: (a) recording data concerning a person’s arm and/or hand motion from a motion sensor on wrist-worn device; (b) recording data concerning the person’s heart rate from an optical (e.g. spectroscopic and/or PPG) sensor on the wrist-worn device; (c) analyzing the data concerning the person’s arm and/or hand motion and the data concerning the person’s heart rate; (d) activating a camera on eyewear to start recording food images if multivariate (e.g. joint, combined) analysis of the arm and/or hand motion data and heart rate data indicates that the person is eating; and (e) estimating the types and/or amounts of food that the person has consumed based on multivariate (e.g. joint, combined) analysis of the person’s arm and/or hand motion, the person’s heart rate, and the food images.

In an example, a method for monitoring food consumption can comprise: (a) using camera on a person’s eyewear to record images; (b) analyzing the recorded images to identify hand gestures and/or motions related to eating food; (c) if hand gestures and/or motions related to eating food have not been identified during a period of time (e.g. the last 1-10 minutes), then deleting the recorded images; (d) if hand gestures and/or motions related to eating food have been identified during the period of time, analyzing the hand gestures and/or motions and the recorded images to estimate the types and/or amounts of food consumed by the person.

In an example, a method for monitoring food consumption can comprise: (a) monitoring a person’s hand gestures and/or motions; (b) analyzing the hand gestures and/or motions to identify eating motions; (c) if eating motions have been identified during a period of time (e.g. the last 1-10 minutes), then activating a camera worn by the person to record images of the area in front of the person; (e) if eating motions have been identified during the period of time, then analyzing the recorded images to identify food; (f) if food is not identified in the recorded images, then erasing the recorded images; (g) if food is identified in the recorded images, then analyzing the hand gestures and motions and the recorded images to estimate the types and/or amounts of food consumed by the person.

In an example, a method for monitoring food consumption can comprise: (a) using a wrist-worn device to monitor a person’s hand gestures and/or motions; (b) analyzing the hand gestures and/or motions to identify eating motions; (c) if eating motions have been identified during a period of time (e.g. the last 1-10 minutes), then activating an eyewear-mounted camera worn by the person to record images of the area in front of the person; (e) if eating motions have been identified during the period of time, then analyzing the recorded images to identify food; (f) if food is not identified in the recorded images, then erasing the recorded images; (g) if food is identified in the recorded images, then analyzing the hand gestures and motions and the recorded images to estimate the types and/or amounts of food consumed by the person.

In an example, a method for monitoring food consumption can comprise: (a) using a wrist-worn device to monitor a person’s hand gestures and/or motions; (b) analyzing the hand gestures and/or motions to identify eating motions; (c) if eating motions have been identified during a period of time (e.g. the last 1-10 minutes), then activating a camera on the wrist-worn device to record images; (e) if eating motions have been identified during the period of time, then analyzing the recorded images to identify food; (f) if food is not identified in the recorded images, then erasing the recorded images; (g) if food is identified in the recorded images, then analyzing the hand gestures and motions and the recorded images to estimate the types and/or amounts of food consumed by the person.

In an example, a smart shirt for measuring a person’s food intake can have two or more motion sensors attached to, woven into, and/or otherwise integrated into both sleeves and/or cuffs, respectively. In an example, a smart shirt for measuring a person’s food intake can have two or more motion sensors attached to, woven into, and/or otherwise integrated into both sleeves and/or cuffs, respectively, wherein the motion sensors detect eating-related arm and/or hand motions. In an example, a smart shirt for measuring a person’s food intake can have a motion sensor attached to, woven into, and/or otherwise integrated into a sleeve and/or cuff. In an example, a smart shirt for measuring a person’s food intake can have a motion sensor attached to, woven into, and/or otherwise integrated into a sleeve and/or cuff, wherein the motion sensor detects eating-related arm and/or hand motions. In an example, a device for measuring a person’s food intake can comprise a motion sensor which is attached to and/or integrated into the sleeve and/or cuff of a shirt. In an example, a device for measuring a person’s food intake can comprise a motion sensor which is attached to and/or integrated into the sleeve and/or cuff of a shirt, wherein the motion sensor detects eating-related arm and/or hand motions.

In an example, a system can analyze, estimate, track, and/or monitor the number of hand-to-mouth motions per meal and/or per interval of time. In an example, a system can comprise a smart watch worn on one of a person’s non-dominant arm and a wrist-band with a motion sensor worn on the wrist of the person’s dominant arm. In an example, a system can comprise a smart watch worn on one of a person’s non-dominant arm and a wrist-band with a motion sensor worn on the wrist of the person’s dominant arm, wherein eating motions detected by the motion sensor trigger two or more cameras on the smart watch to record images of nearby food and/or the person’s hand-to-mouth interactions. In an example, a system can comprise a smart watch worn on one of a person’s wrists (e.g. right wrist) and a wrist-band with a motion sensor worn on the other of the person’s wrists (e.g. left wrist). In an example, this device can be part of a system which includes a wrist band with a motion sensor to detect and/or track eating-related hand-to-mouth motions.

In an example, a motion sensor can detect movement patterns of the person’s hand that indicate that the person is probably eating. In an example, these movements can include reaching for food, grasping food (or a glass or utensil for transporting food), raising food up to the mouth, tilting the hand to move food into the mouth, pausing to chew or swallow food, and then lowering the hand. In an example, these movements may also include the back-and-forth hand movements that are involved when a person cuts food on a plate. In an example, a motion sensor is categorized as a relatively less-intrusive sensor, even though it operates continually to monitor possible eating events.

In an example, a video camera can scan in a spiral, radial, or back-and-forth pattern in order to monitor activity near both the person’s fingers and the person’s mouth. This requires that the device keep track of where the person’s fingers and mouth are, in three-dimensional space, relative to the camera as the person moves their arm, hand, and head. In an example, face recognition software can help the device to track the person’s mouth and gesture recognition software can help the device to track the person’s fingers.

In an example, a wearable motion sensor can be a three-dimensional accelerometer that is incorporated into a device that a person wears on their wrist, in a manner like a wrist watch. In an example, this three-dimensional accelerometer may detect probable eating events based on monitoring and analysis of the three-dimensional movement of the person’s arm and hand. Eating activity can be indicated by particular patterns of up and down, rolling and pitching, movements. Arm and hand movement can include movement of the person’s shoulder, elbow, wrist, and finger joints. In an example, a miniature video camera can track a food-transporting member (such as glass) as it is lifted upwards towards the person’s mouth, paused during consumption of the food, and then lowered back down.

In an example, an iterative method for measuring a person’s food consumption and caloric intake can comprise receiving passively-collected data about the person’s food consumption, in an automatic manner, from one or more sensors that are worn in or on the person. Passively-collected data about food consumption is defined herein as data about food consumption that is received in an automatic manner that does not require any voluntary action by the person in association with an eating event, other than the actual action of eating. For example, if a camera worn on a person’s wrist automatically records and analyzes images that indicate that the person is eating, then this would be passively-collected data about food consumption. In an example, if a motion sensor worn on a person’s wrist automatically records and analyzes motion data that indicates that the person is eating, then this would also be passively-collected data about food consumption.

In an example, in a first round of passively-collected data collection, an automatic sensor is a motion-activated camera (such as a wearable camera) that only collects information on food consumption when the person’s movements suggest that the person is eating. This is less intrusive on the person’s privacy than a camera that takes pictures continuously.

In an example, a device can measure food composition using radar. In an example, a device can measure food composition using radar with a frequency in the millimeter range. In an example, a device for measuring a person’s food consumption can transmit radar signals toward food, receive radar signals reflected back from the food, and analyze changes in those signals caused by interaction with the food. In an example, a device for measuring a person’s food consumption can transmit radar signals toward food, receive radar signals transmitted through the food, and analyze changes in those signals caused by interaction with the food.

In an example, a device for measuring a person’s food consumption can transmit radar signals toward food, receive radar signals reflected back from the food, and estimate the composition of the food based on changes in those signals caused by interaction with the food. In an example, a device for measuring a person’s food consumption can transmit radar signals toward food, receive radar signals transmitted through the food, and estimate the composition of the food based on changes in those signals caused by interaction with the food.

In an example, a device for measuring the types and/amounts of nutrients consumed by a person can include a radar emitter and radar receiver, wherein the radar emitter emits radar signals at different frequencies. In an example, a device for measuring the types and/amounts of nutrients consumed by a person can include a radar emitter and radar receiver, wherein the radar emitter emits radar signals whose frequencies vary in a cyclical and/or iterative manner. In an example, a method for measuring a person’s nutritional consumption can include: analyzing food images to estimate an amount of food; and analyzing radar waves reflected from the food to estimate the nutritional composition of the food. In an example, a method for measuring the types and amounts of nutrients consumed by a person can include: (a) analyzing food images to estimate an amount of food; and (b) analyzing radar waves reflected from the food to estimate the nutritional composition of the food.

In an example, a device can identify types and/or amounts of food in an image based on a person’s geographic location. In an example, a device can identify types and/or amounts of food in an image based on a person’s being in a particular restaurant. In an example, a system for measuring a person’s food consumption can include a GPS or other geographic location component, wherein the types and/or amounts of food available at a person’s geographic location (e.g. a particular restaurant) is included in multivariate estimation of the types and/or amounts of food consumed by that person at that location.

In an example, a system can analyze the relationship between ambient light level and a person’s consumption of different types and/or amounts of food. In an example, a system can analyze the relationship between ambient noise level and a person’s consumption of different types and/or amounts of food. In an example, a system can analyze the relationship between ambient temperature and a person’s consumption of different types and/or amounts of food. In an example, a system can integrate data from a person’s recent (internet) search pattern with data from wearable sensors to enhance estimation of the types and/or amounts of food consumed by the person.

In an example, a system can integrate data from a person’s recent pattern of movements and/or locations to enhance estimation of the types and/or amounts of food consumed by the person. In an example, a system can integrate data from a person’s recent (geographic) travel pattern to enhance estimation of the types and/or amounts of food consumed by the person. In an example, a system can analyze the relationship between a person’s location and the person’s consumption of different types and/or amounts of food. In an example, a system for managing nutritional intake can include analyzing data from a wearable food intake monitor to identify geographic locations which trigger a person to eat unhealthy types and/or amounts of food. In an example, a system can integrate data from a person’s recent (geographic) travel pattern with data from wearable sensors to enhance estimation of the types and/or amounts of food consumed by the person.

In an example, a system for managing nutritional intake can include analyzing data from a wearable food intake monitor to identify environmental conditions which trigger a person to eat unhealthy types and/or amounts of food. In an example, a system for managing nutritional intake can include analyzing data from a wearable food intake monitor to identify environmental conditions which prompt a person to eat unhealthy types and/or amounts of food. In an example, a system for managing nutritional intake can include analyzing data from a wearable food intake monitor to identify environmental conditions which trigger a person to eat. In an example, a device and/or system can analyze environmental conditions and eating behavior to identify environmental conditions which trigger a person to be more susceptible to eating unhealthy types and/or amounts of food.

In an example, a device can comprise and/or include a pressure sensor. In an example, a device can comprise a pressure sensor in proximity to the surface of a person’s head (e.g. jaw and/or side). In an example, a device can comprise a pressure sensor which is in contact with the surface of a person’s head (e.g. jaw and/or side). In an example, a device can comprise a strain sensor. In an example, a device can comprise and/or include a vibration sensor. In an example, a device can comprise a vibration sensor. In an example, a device can comprise a vibration sensor which is in contact with the surface of a person’s head (e.g. jaw and/or side). In an example, a device can comprise a vibration sensor in proximity to the surface of a person’s head (e.g. jaw and/or side).

In an example, a device can confirm an eating event based on confirming results from at least two of the following: chewing motions; chewing sounds; eye movements; hand gestures associated with food consumption; hand-to-mouth movements; identification of food in images; location in a restaurant identified by GPS or other location-identification system; swallowing motions; and swallowing sounds. In an example, a device can confirm an eating event based on confirming results from at least three of the following: chewing motions; chewing sounds; eye movements; hand gestures associated with food consumption; hand-to-mouth movements; identification of food in images; location in a restaurant identified by GPS or other location-identification system; swallowing motions; and swallowing sounds.

In an example, a device can detect eating and/or measure food consumption by multivariate analysis of data concerning two or more of the following actions: hand-to-mouth movements; hand gestures; chewing movements; swallowing movements; and eye movements. In an example, a device can detect eating and/or measure food consumption by multivariate analysis of data concerning two or more of the following: chewing motions; chewing sounds; eye movements; hand gestures associated with food consumption; hand-to-mouth movements; identification of food in images; location in a restaurant identified by GPS or other location-identification system; swallowing motions; and swallowing sounds. In an example, a system can use data from wearable sensors to estimate the types and/or amounts of food consumed by a person wherein this data can include one or more variables selected from the group consisting of: EEG sensor data, EGG sensor data, EMG sensor data, wearable spectroscopic sensor data, optical sensor data, strain sensor data, wearable microphone data, and stretch sensor data.

In an example, a device can prompt a person to provide information via a touch screen, voice command an speech recognition, keypad, or hand gesture concerning the food types and/or amounts that they are eating when their eating is detected by one or more wearable sensors selected from the group consisting of: microphone, motion sensor, optical sensor, spectroscopic sensor, EMG sensor, chewing sensor, swallowing sensor, heart rate sensor, and EEG sensor. In an example, a method for food consumption monitoring can comprise: (a) using a vibration sensor on a device worn by a person to record vibrations; (b) using a data processor to analyze these vibrations to detect chewing and/or swallowing which indicate that the person is eating; (c) if analysis of these vibrations indicates that the person is eating, then activating a camera on the device to start recording images of space and/or objects in front of the person to capture food images; (d) if analysis of these vibrations indicates that the person is eating, then also analyzing these vibrations and these food images to estimate the types and/or amounts of food that the person is eating; and (e) if analysis of these vibrations indicates that the person has stopped eating for a period of time (e.g. between 3 and 15 minutes), then deactivating the camera so that it stops recording images.

In an example, a method for monitoring food consumption can comprise: (a) using an strain and/or stretch sensor worn by a person to measure tissue motion; (b) analyzing measured tissue motions to identify chewing and/or swallowing motions; (c) if chewing and/or swallowing motions have been identified during a period of time (e.g. the last 1-10 minutes), then activating a camera worn by the person to record images of the area in front of the person; (e) if chewing and/or swallowing motions have been identified during the period of time, then analyzing the recorded images to identify food; (f) if food is not identified in the recorded images, then erasing the recorded images; (g) if food is identified in the recorded images, then analyzing the tissue motions and the recorded images to estimate the types and/or amounts of food consumed by the person.

In an example, a system can analyze the relationship between a person’s biometric parameters and the person’s consumption of different types and/or amounts of food. In an example, a device can analyze the relationship between a person’s stress level (e.g. measured by analysis of the person’s voice) and the types and/or amounts of food consumed by that person. In an example, a system can analyze the relationship between a person’s stress level and the person’s consumption of different types and/or amounts of food. In an example, a system can analyze the relationship between a person’s blood pressure and the person’s consumption of different types and/or amounts of food. In an example, a system can analyze the relationship between a person’s sleep and the person’s consumption of different types and/or amounts of food.

In an example, a system can estimate a type and/or amount of food by analyzing one or more characteristics of the food selected from the group consisting of: shape, size, color, texture, temperature, spectral distribution of light reflected from the food, position on a plate or other dish, timing in a multi-course meal, time of day, and geographic location. In an example, a system can estimate a type and/or amount of food by analyzing one or more characteristics of the food selected from the group consisting of: shape, size, color, texture, temperature, position on a plate or other dish, and timing in a multi-course meal. In an example, a system can identify a type of food by analyzing one or more characteristics of the food selected from the group consisting of: shape, size, color, texture, temperature, position on a plate or other dish, and timing in a multi-course meal. In an example, changes in the size and/or volume of food near a person can be used to estimate the amount of food consumed by the person.

In an example, a system can estimate the probability that a person is consuming food using multivariate analysis, wherein a first set of some variables, factors, and/or conditions in the multivariate analysis are associated with a higher probability that the person is consuming food, and a second set of variables, factors, and/or conditions in the multivariate analysis are associated with a lower probability that the person is consuming food. In an example, the first set can include one or more of the following variables, factors, and/or conditions: hand-to-mouth motions associated with eating, identification of food in recorded images, identification of chewing and/or swallowing sounds in recorded sounds, geographic location at a restaurant, identification of chewing and/or swallowing motions by a motion, stretch, and/or vibration sensor, identification of muscle activity associated with chewing and/or swallowing motions by an EMG sensor, identification of EEG patterns associated with food and/or eating by an EEG sensor, and time of day associated with eating (a meal) in eating history. In an example, the second set can include one or more of the following variables, factors, and/or conditions: hand-to-mouth motions associated with hand-to-mouth actions other than eating (e.g. brushing teeth, coughing, sneezing, yawning, smoking), identification of handheld objects other than food (e.g. pen, tool, clothing) in recorded images, and identification of coughing and/or yawing sounds in recorded sounds.

In an example, a system can integrate data from a person’s recent (internet) search pattern to enhance estimation of the types and/or amounts of food consumed by the person. In an example, a system can use data from demographic factors to estimate the types and/or amounts of food consumed by a person wherein this data can include one or more variables selected from the group consisting of: age, gender, socioeconomic status, and home location. In an example, a system can use data from environmental factors to estimate the types and/or amounts of food consumed by a person wherein this data can include one or more variables selected from the group consisting of: ambient humidity level, ambient light level, ambient scent analysis, ambient sound level, food purchase and/or meal order information, ambient speech analysis, current geographic location, and type of restaurant where the person is currently.

In an example, a system can use multivariate analysis of data from wearable sensors, demographic factors, biometric parameters, and environmental conditions to estimate the types and/or amounts of food consumed by a person, wherein this data can include one or more variables selected from the group consisting of: time of day, day of the week, season of the year, recent sleep history, EGG sensor data, ambient light level, EMG sensor data, gender, geographic location, type of restaurant wherein person is, blood oxygenation level, heart rate, heart rate variability, hydration level, stress level, food purchase and/or meal order information, socioeconomic status, blood pressure, body temperature, age, tissue impedance level, ambient scent analysis, EEG sensor data, ambient sound level, blood glucose level, recent speech history, ambient humidity level, weight, medical condition, recent food consumption history, ambient speech analysis, and recent exercise history.

In an example, a system for managing nutritional intake can include analyzing data from a wearable food intake monitor to identify values of biometric parameters which trigger a person to eat unhealthy types and/or amounts of food. In an example, a system for managing nutritional intake can include analyzing data from a wearable food intake monitor to identify physiological conditions which trigger a person to eat unhealthy types and/or amounts of food. In an example, a system for managing nutritional intake can include analyzing data from a wearable food intake monitor to identify times and/or events which trigger a person to eat unhealthy types and/or amounts of food.

In an example, a system for measuring a person’s food and/or nutritional consumption can include a hand-held device with one or more of the following types of food sensors: a camera; a chemical sensor; a light emitter and receiver; a radar emitter and receiver; and an optical scanner. In an example, a system for measuring a person’s food and/or nutritional consumption can include a wrist-worn device with one or more of the following types of food sensors: a camera; a chemical sensor; a light emitter and receiver; a radar emitter and receiver; and an optical scanner. In an example, food consumption can be detected and/or measured based on joint analysis of data from motion sensors, sound sensors, and food identification in recorded images.

In an example, changes in volume of nearby food and number of chewing and/or swallowing motions can be analyzed using multivariate methods to estimate amount of food consumed by person. In an example, changes in volume of nearby food and number of chewing and/or swallowing motions can be jointly analyzed using multivariate methods to estimate amount of food consumed by person. In an example, changes in volume of nearby food and number of chewing and/or swallowing motions can be analyzed in a multivariate manner to estimate amount of food consumed by person. In an example, a system can use multivariate analysis of data from wearable sensors, demographic factors, biometric parameters, and environmental conditions to estimate the types and/or amounts of food consumed by a person. In an example, the amounts and/or types of food consumed can be estimated by multivariate analysis of chewing sounds and swallowing sounds during an eating event. In an example, signals from a sensor can be analyzed using artificial intelligence.

In an example, data from a device to measure a person’s food consumption can be analyzed using one or more methods selected from the group consisting of: artificial neural network; Bayesian analysis; linear discriminant analysis; machine learning; multivariate linear regression; principle components analysis; carlavian curve analysis, random forest analysis; Fourier Transformation; and time-series analysis. In an example, images recorded by a device to measure a person’s food consumption can be analyzed using one or more methods selected from the group consisting of: artificial neural network; Bayesian analysis; linear discriminant analysis; machine learning; multivariate linear regression; principle components analysis; random forest analysis; Fourier Transformation; and time-series analysis.

In an example, movement a person’s temple can be monitored to detect and measure food consumption. In an example, a device can monitor movement of the mastoid bone. In an example, a device can monitor deformation and/or stretching of the surface of a person’s body which is caused by food consumption. In an example, a device can identify types and/or amounts of food in an image based on food temperature. In an example, a device can comprise two cameras, wherein a first camera scans for nearby food using a first spectral range and a second camera scans for nearby food using a second spectral range.

In an example, one or more of the following sensors can be incorporated into a hand-held device for estimating food composition: a camera; a chemical sensor; a light emitter and receiver; a radar emitter and receiver; and an optical scanner. In an example, one or more of the following sensors can be incorporated into a wrist-worn device for estimating food composition: a camera; a chemical sensor; a light emitter and receiver; a radar emitter and receiver; and an optical scanner. In an example, tissue motions recorded by a device to measure a person’s food consumption can be analyzed using one or more methods selected from the group consisting of: artificial neural network; Bayesian analysis; linear discriminant analysis; machine learning; multivariate linear regression; principle components analysis; random forest analysis; Fourier Transformation; and time-series analysis.

In an example, “level 1” wearable sensors can be relatively less-intrusive because of their low-level modality (e.g. motion and sound), non-continuous operation (e.g. only when triggered by a probable eating event), low-profile placement (e.g. under clothing), and/or flexible timing (e.g. delayed data acquisition after multiple eating events). In an example, “level 2” wearable sensors can be more-intrusive because of their high-level modality (e.g. images), continuous operation, high-profile placement (e.g. around the person’s neck), and/or immediate timing (e.g. real time data acquisition during eating events).

In an example, a camera can constantly maintain a line of sight to one or both of a person’s hands. In an example, this camera can scan for (and identify and maintain a line of sight to) a person’s hand only when a sensor indicates that the person is eating. In an example, this camera can scan for, acquire, and maintain a line of sight to nearby food only when a sensor indicates that the person is probably eating. In various examples, the sensors used to activate one or more of these cameras can be selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, a device and method can comprise at least one camera (or other camera) that takes pictures along an imaging vector which points toward the person’s mouth and/or face, during certain body configurations, while the person eats. In an example, a device and camera uses face recognition methods to adjust the direction and/or focal length of its field of vision in order to stay focused on the person’s mouth and/or face. Face recognition methods and/or gesture recognition methods may also be used to detect and measure hand-to-mouth proximity and interaction. In an example, one or more cameras automatically stay focused on the person’s mouth, even if the device moves, by the use of face recognition methods. In an example, the fields of vision from one or more cameras collectively encompass the person’s mouth and a nearby food, when the person eats, without the need for human intervention, when the person eats, because the cameras remain automatically directed toward the person’s mouth, toward a reachable food source, or both.

In an example, a device and method can take pictures of the person’s mouth and scan for nearby food only when a wearable sensor, such as an accelerometer, indicates that the person is (probably) eating. In various examples, one or more sensors that detect when the person is (probably) eating can be selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, a device can be embodied in a device for measuring a person’s food consumption and/or caloric intake comprising: (a) a first sensor and/or user interface that collects a first set of data concerning what the person eats; (b) a data processor that calculates a first estimate of the person’s caloric intake based on the first set of data, uses this first estimate of the person’s caloric intake to estimate predicted weight change for the person during a period of time, and compares predicted to actual weight change to determine whether predicted and actual weight change meet criteria for similarity and/or convergence; and (c) a second sensor and/or user interface that collects a second set of data concerning what the person eats if the criteria for similarity and/or convergence of predicted and actual weight change are not met. In an example, at least one of the sensors and/or user interfaces can be worn by the person.

In an example, a device can be embodied in a device for measuring a person’s food consumption and/or caloric intake comprising: (a) a first set of sensors and/or user interfaces that collect a first set of data concerning what the person eats, wherein this first set includes passively-collected data that is collected in a manner that does not require voluntary actions by the person associated with particular eating events other than the actions of eating, and wherein this first set also includes actively-entered data that is collected in a manner that requires voluntary actions by the person associated with particular eating events other than the actions of eating; (b) a data processor that calculates a first estimate of the person’s caloric intake based on the first set of data, uses this first estimate of the person’s caloric intake to estimate predicted weight change for the person during a period of time, and compares predicted to actual weight change to determine whether predicted and actual weight change meet criteria for similarity and/or convergence; and (c) a second set of sensors and/or user interfaces that collect a second set of data concerning what the person eats if the criteria for similarity and/or convergence of predicted and actual weight change are not met.

In an example, a device can be embodied in a device for measuring a person’s food consumption and/or caloric intake comprising: (a) a first set of sensors and/or user interfaces that receive a first set of data concerning what the person eats, wherein this first set includes passively-collected data that is received in a manner that does not require voluntary actions by the person associated with particular eating events other than the actions of eating, and wherein this first set also includes actively-entered data that is received in a manner that requires voluntary actions by the person associated with particular eating events other than the actions of eating; (b) a data processor that calculates a first estimate of the person’s caloric intake based on the first set of data, uses this first estimate of the person’s caloric intake to estimate predicted weight change for the person during a period of time, and compares predicted to actual weight change to determine whether predicted and actual weight change meet criteria for similarity and/or convergence; and (c) a second set of sensors and/or user interfaces that receive a second set of data concerning what the person eats if the criteria for similarity and/or convergence of predicted and actual weight change are not met.

In an example, a device can comprise one or more cameras which are automatically activated to take pictures when a person eats based on a sensor selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, a device can include a tamper-resisting member that comprises a sensor that detects and responds if the line of sight from one or more cameras to the person’s mouth or to a food source is impaired when a person is probably eating based on a sensor, wherein this sensor is selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, a device can include one or more cameras that collectively and automatically take pictures of the person’s mouth and pictures of a nearby food, when the person eats, without the need for human intervention, when the person eats, to activate picture taking. In an example, these one or more cameras take pictures continually. In an example, these one or more cameras are automatically activated to take pictures when a person eats based on a sensor selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor.

In an example, a device that automatically monitors caloric intake can comprise: one or more cameras that are worn on one or more locations on a person from which these cameras: collectively and automatically take pictures of the person’s mouth when the person eats and take pictures of nearby food when the person eats; wherein nearby food is a food source that the person can reach by moving their arm; wherein food can include liquid nourishment as well as solid food; wherein one or more cameras collectively and automatically take pictures of the person’s mouth and pictures of a nearby food, when the person eats, without the need for human intervention, when the person eats, to activate picture taking; and wherein the fields of vision from one or more cameras collectively and automatically encompass the person’s mouth and a nearby food, when the person eats, without the need for human intervention, when the person eats, to manually aim a camera toward the person’s mouth or toward a reachable food source; a tamper-resisting mechanism which detects and responds if the operation of the one or more cameras is impaired; wherein a tamper-resisting member comprises a sensor that detects and responds if the line of sight from one or more cameras to the person’s mouth or to a food source is impaired when a person is probably eating based on a sensor, wherein this sensor is selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor; and an image-analyzing member which automatically analyzes pictures of the person’s mouth and pictures of a reachable food source in order to estimate not just what food is at a reachable food source, but the types and quantities of food that are actually consumed by the person; and wherein the image-analyzing member uses one or more methods selected from the group consisting of: pattern recognition or identification; human motion recognition or identification; face recognition or identification; gesture recognition or identification; food recognition or identification; word recognition or identification; logo recognition or identification; bar code recognition or identification; and 3D modeling.

In an example, a device that automatically monitors caloric intake can comprise: (a) one or more cameras that are worn on one or more locations on a person from which these cameras: collectively and automatically take pictures of the person’s mouth when the person eats and pictures of nearby food when the person eats; wherein nearby food is a food source that the person can reach by moving their arm; and wherein food can include liquid nourishment as well as solid food; (b) a tamper-resisting mechanism which detects and responds if the operation of the one or more cameras is impaired; and (c) an image-analyzing member which automatically analyzes pictures of the person’s mouth and pictures of a reachable food source in order to estimate the types and quantities of food that are consumed by the person.

In an example, a first set of data can comprise sound data, motion data, or both sound and motion data, and a second set of data can comprise image data. In an example, a method can include the use of a first set of data comprising image data whose collection requires voluntary actions by the person associated with particular eating events other than the actions of eating and a second set of data comprising image data whose collection is more continuous than that of the first set of data. In an example, a method can include use of a first set of data comprising image data whose collection is triggered by sounds, motions, or sounds and motions indicating an eating event, not requiring voluntary actions by the person associated with particular eating events other than the actions of eating, and a second set of data comprising image data whose collection is more continuous than that of the first set of data. In an example, a method can provide a person with multiple incentives to provide both accurate and timely actively-entered food consumption data. One such incentive is the avoidance of increasingly-intrusive data collection methods. As the person becomes more engaged and accurate with respect to voluntary reporting of caloric intake, passively-collected data collection becomes less necessary and less intrusive.

In an example, a method for measuring a person’s caloric intake can comprise: (a) receiving a first set of data concerning what the person eats from a first source and receiving a second set of data concerning what the person eats from a second source; (b) calculating a first estimate of the person’s caloric intake based on the first set of data, calculating a second estimate of the person’s caloric intake based on the second set of data, and comparing these first and second estimates of caloric intake to determine whether these estimates meet criteria for similarity and/or convergence; and (c) if the first and second estimates of caloric intake do not meet the criteria for similarity and/or convergence, then receiving a third set of data concerning what the person eats and calculating one or more new estimates of caloric intake using this third set of data.

In an example, a method for measuring a person’s caloric intake can comprise: (a) receiving a first set of data concerning what the person eats; (b) calculating a first estimate of the person’s caloric intake based on the first set of data, using this first estimate of the person’s caloric intake to estimate predicted weight change for the person during a period of time, and comparing predicted weight change to actual weight change to determine whether predicted weight change and actual weight change meet criteria for similarity and/or convergence; and (c) if predicted weight change and actual weight change do not meet the criteria for similarity and/or convergence, then receiving a second set of data concerning what the person eats and calculating a second estimate of caloric intake using this second set of data.

In an example, a method for measuring a person’s caloric intake can comprise: receiving a first set of data concerning what the person eats from a first source and receiving a second set of data concerning what the person eats from a second source; calculating a first estimate of the person’s caloric intake based on the first set of data, calculating a second estimate of the person’s caloric intake based on the second set of data, and comparing these first and second estimates of caloric intake to determine whether these estimates meet criteria for similarity and/or convergence; and if the first and second estimates of caloric intake do not meet the criteria for similarity and/or convergence, then receiving a third set of data concerning what the person eats and calculating one or more new estimates of caloric intake using this third set of data.

In an example, a method for measuring a person’s caloric intake can comprise: receiving a first set of data concerning what the person eats; calculating a first estimate of the person’s caloric intake based on the first set of data, using this first estimate of the person’s caloric intake to estimate predicted weight change for the person during a period of time, and comparing predicted weight change to actual weight change to determine whether predicted weight change and actual weight change meet criteria for similarity and/or convergence; and if predicted weight change and actual weight change do not meet the criteria for similarity and/or convergence, then receiving a second set of data concerning what the person eats and calculating a second estimate of caloric intake using this second set of data.

In an example, a method for measuring the types and quantities of food consumed by a person can comprise: (a) receiving a first set of data from a first source concerning what the person eats and receiving a second set of data from a second source concerning what the person eats; (b) calculating a first estimate of the types and quantities of food consumed based on the first set of data, calculating a second estimate of the types and quantities of food consumed based on the second set of data, and comparing these first and second estimates of caloric intake to determine whether these estimates meet criteria for similarity and/or convergence; and then (c) if the first and second estimates of caloric intake do not meet the criteria for similarity and/or convergence, then receiving a third set of data concerning what the person eats and calculating a third estimate of the types and quantities of food consumed using this third set of data.

In an example, a system and/or method measuring caloric intake can escalate from collecting data on food consumption from “level 1” (less intrusive) automatic sensors to collecting data on food consumption from “level 2” (more intrusive) automatic sensors if the “level 1” sensors do not achieve a desired level of measurement accuracy. In an example, “level 1” sensors can be less-intrusive because of their low-level modality (e.g. motion or sound), non-continuous operation (e.g. only when triggered by an eating event), low-profile placement (e.g. under clothing), and/or flexible timing (e.g. delayed data acquisition at the end of the day). In an example, “level 2” can be more-intrusive because of their high-level modality (e.g. images), continuous operation, high-profile placement (e.g. around the neck), and/or immediate timing (e.g. real time data acquisition while eating).

In an example, a video camera can be activated to take pictures when other components of a device indicate that the person is probably eating. In an example, the operation of a video camera can be triggered when a motion sensor indicates that the person is probably eating. In an example, the operation of a video camera can be triggered when a sound sensor indicates that the person is probably eating. In an example, the operation of a video camera can be triggered when actively-entered data received from the person, such as through a microphone and a speaker unit, indicates that the person is eating.

In an example, data about food consumption can be used to estimate a person’s caloric intake. In an example, data can be in a raw format that does not explicitly identify the types and quantities of food consumed. Raw data can be analyzed to identify the types and quantities of food consumed as well as total caloric intake. In various examples, this analysis can include one or more methods selected from the group consisting of: food recognition or identification; visual pattern recognition or identification; human motion recognition or identification; chemical recognition or identification; smell recognition or identification; sound pattern recognition; word recognition or identification; logo recognition or identification; bar code recognition or identification; and 3D modeling.

In an example, data can be analyzed to extract information about the types and quantities of food consumed. In various examples, data about food consumption can be analyzed by one or more methods selected from the group consisting of: pattern recognition or identification; human motion recognition or identification; facial recognition or identification; gesture recognition or identification; food recognition or identification; sound pattern recognition; Fourier transformation; chemical recognition or identification; smell recognition or identification; word recognition or identification; logo recognition or identification; bar code recognition or identification; and 3D modeling.

In an example, first and second level passively-collected data sensors can be motion sensors and cameras, respectively. In an example, in a method for measuring caloric intake, the intrusiveness of data collection can be escalated only as far as is required to achieve a desired level of accuracy. In this respect, this method could be called “minimally-intrusive” yet highly-accurate caloric intake monitoring. This method can achieve accuracy that is superior to methods of estimating caloric intake in the prior art that rely on actively-entered data collection only -- especially voluntary methods with no empirically-validated methods to encourage compliance and accuracy. This method can be better (greater accuracy and/or greater privacy) than methods of estimating caloric intake in the prior art that rely on passively-collected data collection through a static configuration of sensors, especially a static configuration of more-intrusive sensors.

In an example, one or more sensors can continually monitor a person to collect data about the person’s food consumption. In various examples, one or more sensors can monitor sounds, motion, images, speed, geographic location, or other parameters. In other examples, one or more sensors can monitor parameters periodically, intermittently, or randomly. In other examples, the output of one type of sensor can be used to trigger operation of another type of sensor. In an example, a relatively less-intrusive sensor (such as a motion sensor) can be used to continually monitor the person and this less-intrusive sensor can trigger operation of a more-intrusive sensor (such as a camera) only when probable food consumption is detected by the less-intrusive sensor.

In an example, passively-collected data about a person’s food consumption can be collected automatically through one or more sensors. These one or more sensors can be selected from the group consisting of: accelerometer, inclinometer, other motion sensor, sound sensor, smell or olfactory sensor, blood pressure sensor, heart rate sensor, EEG sensor, ECG sensor, EMG sensor, electrical sensor, chemical sensor, gastric activity sensor, GPS sensor, camera or other image-creating sensor or device, optical sensor, piezoelectric sensor, respiration sensor, strain gauge, electrogoniometer, chewing sensor, swallow sensor, temperature sensor, and pressure sensor.

In an example, the first set of data can comprise image data whose collection is triggered by sounds, motions, or sounds and motions indicating an eating event, not requiring voluntary actions by the person associated with particular eating events other than the actions of eating, and the second set of data can comprise image data whose collection is more continuous than that of the first set of data. In an example, the first set of data comprises sound data, motion data, or both sound and motion data, and wherein the second set of data comprises image data. In an example, a three-cycle method for collecting food consumption data can escalates, as needed, from motion sensors to sound sensors to cameras. If similarity and/or convergence criteria are met in any given cycle, then the method comes to a stop. If the similarity and/or convergence criteria are not met, then the method escalates to the next data collection cycle, up to a maximum of three cycles.

In various examples, indications that a person is probably eating can be selected from the group consisting of: acceleration, inclination, twisting, or rolling of the person’s hand, wrist, or arm; acceleration or inclination of the person’s lower arm or upper arm; bending of the person’s shoulder, elbow, wrist, or finger joints; movement of the person’s jaw, such as bending of the jaw joint; smells suggesting food that are detected by an artificial olfactory sensor; detection of chewing, swallowing, or other eating sounds by one or more microphones; electromagnetic waves from the person’s stomach, heart, brain, or other organs; GPS or other location-based indications that a person is in an eating establishment (such as a restaurant) or food source location (such as a kitchen).

In various examples, passively-collected data about food consumption can include information selected from the group consisting of: the types and volumes of food sources within view and/or reach of the person; changes in the volumes of these food sources over time; the number of times that the person brings their hand (with food) to their mouth; the sizes or portions of food that the person brings to their mouth; and the number, frequency, speed, or magnitude of chewing, biting, or swallowing movements.

Some types of sensors and some modes of operation are more intrusive with respect to a person’s privacy and/or time than other types of sensors and modes of operation. In an example, wearable motion sensors and sound sensors can be less intrusive than wearable cameras. In an example, a wearable camera that records images within a narrow field of vision and a shorter focal length can be less intrusive than a wearable camera that records images with a wide field of vision and longer focal length. In an example, wearable sensors that operate only when triggered by a probable eating event are less intrusive than sensors than operate continuously. In an example, sensors that are worn under clothing or on less-prominent parts of the body are less intrusive than sensors that are worn on highly-visible portions of clothing or the body. In an example, sensors that allow a person to enter food consumption data a considerable time after a meal (delayed diet logging) are less intrusive than sensors that actively prompt a person to enter food consumption data right in the middle of a meal (real-time diet logging).

The “food consumption pathway” is defined as a path in space that is traveled by (a piece of) food from nearby food to a person’s mouth as the person eats. The distal endpoint of a food consumption pathway is a reachable food source and the proximal endpoint of a food consumption pathway is the person’s mouth. In various examples, food can be moved along a food consumption pathway by contact with a member selected from the group consisting of: a utensil; a beverage container; the person’s fingers; and the person’s hand.

In an example, a device and/or system can comprise a pair of eyeglasses, goggles, visor, or other eyewear. In an example, a device can be embodied in eyewear (e.g. a pair of eyeglasses). In an example, a device can be integrated into eyewear (e.g. a pair of eyeglasses). In an example, this device can be embodied in eyewear and/or eyeglasses. In an example, a device can be attached to the posterior third of the anterior-to-posterior length of the sidepiece of eyewear. In an example, a device can be attached to the central third of the anterior-to-posterior length of the sidepiece of eyewear. In an example, a device can be attached to the anterior third of the anterior-to-posterior length of the sidepiece of eyewear. In an example, a device can be clipped to the sidepiece of eyewear at two locations.

In an example, a device can be attached to eyewear by a clip, clasp, hook, snap, hook-and-eye material, button, pin, or magnet. In an example, a device can be attached to eyeglasses by a clip, clasp, pin, hook, snap, magnet, adhesive, or button. In an example, a device can be removably attached to eyewear (e.g. a pair of eyeglasses). In an example, a device can further comprise a hook-and-eye (e.g. Velcro) component which is attached to the sidepiece of eyewear. In an example, a device can further comprise an adhesive component which is attached to the sidepiece of eyewear. In an example, a device can further comprise a magnet which is used to attach the device to eyewear. In an example, a device can further comprise a channel and/or opening through which the sidepiece of eyewear is slid to attach the device to eyewear. In an example, a device can have two clips which are used to fasten it to the sidepiece of eyewear. In an example, this device can be embodied in an eyewear accessory or attachment.

In an example, a device can comprise eyewear with a vibration sensor on the nose bridge of the frame. In an example, a device can comprise eyewear with a piezoelectric sensor on the nose bridge of the frame. In an example, a device can comprise eyewear with an EEG sensor on the nose bridge of the frame. In an example, an EEG sensor on eyewear can be held into close contact with a person’s tissue by one of more mechanisms selected from the group consisting of: a solenoid; compressive foam; elastomeric material (e.g. PDMS); a magnet; a spring; adhesive; an inflatable compartment; a MEMS piston; and an elastic band. In an example, a sensor can be slipped over the sidepiece (e.g. temple of eyeglasses and/or eyewear).

In an example, a sensor can be clipped to eyeglasses and/or eyewear. In an example, an EEG sensor which is attached to eyewear can be held into close contact with a person’s tissue by one of more mechanisms selected from the group consisting of: a solenoid; compressive foam; elastomeric material (e.g. PDMS); a magnet; a spring; adhesive; an inflatable compartment; a MEMS piston; and an elastic band. In an example, an optical chewing sensor can be attached to a standard eyewear frame. In an example, an optical sensor which is attached to eyewear can be held into close contact with a person’s tissue by one of more mechanisms selected from the group consisting of: a solenoid; compressive foam; elastomeric material (e.g. PDMS); a magnet; a spring; adhesive; an inflatable compartment; a MEMS piston; and an elastic band.

In an example, a device or system for monitoring a person’s food consumption can be embodied in AR (Augmented Reality) eyewear. In an example, AR eyewear for measuring a person’s food consumption can display virtual objects (e.g. Information about food) in a person’s field of vision using one or more optical components selected from the group consisting of: active matrix organic light-emitting diode array, projector, or display; collimated light projector or display; digital micro-mirror array, projector, or display; digital pixel array or matrix; diode laser array, projector, or display; ferroelectric liquid crystal on silicon array, projector, or display; holographic optical element array or matrix; holographic projector or display; laser array or matrix; Light Emitting Diode (LED) array or matrix; light emitting diode array, projector, or display; liquid crystal display array, projector, or display; low-power (e.g. nano-watt) laser projector or display; microdisplay and/or microprojector; micro-display array or matrix; optoelectronic display; organic light emitting diode (OLED) array or matrix; passive matrix light-emitting diode array or matrix; photoelectric display; and transmission holographic optical element array or matrix.

In an example, a system for measuring a person’s food consumption can include AR eyewear, a smart watch (or wrist band), and a mobile phone which are in wireless communication with each other. In an example, a system for measuring a person’s food consumption can include AR eyewear and a smart watch (or wrist band) which are in wireless communication with each other. In an example, a system for measuring a person’s food consumption can include AR eyewear and a handheld spectroscopic food scanner which are in wireless communication with each other.

In an example, a device and/or system can comprise an earpiece, ear ring, or ear bud. In an example, a device can be attached to a person’s, worn on a person’s ear, worn around a person’s ear, and/or worn in a person’s ear. In an example, a device can be embodied in a hearing aid, earpiece, and/or ear bud. In an example, a device can be embodied in an ear bud. In an example, a device can be integrated into a hearing aid, earpiece, and/or ear bud. In an example, a device can be located behind a person’s ear. In an example, a device can be removably attached to a hearing aid, earpiece, and/or ear bud.

In an example, a device can have an arcuate and/or curved portion which loops over the top of a person’s ear. In an example, a device can have an arcuate and/or curved portion which loops around the back of a person’s ear. In an example, a device can have an arcuate and/or curved posterior portion which loops around the back of a person’s ear and an anterior portion which extends upward and forward from the person’s ear. In an example, a device can have an arcuate and/or curved posterior portion which loops over the top of a person’s ear and an anterior portion which extends upward and forward from the person’s ear.

In an example, a device can have an arcuate and/or curved posterior portion which loops around the back of a person’s ear and an anterior portion which extends upward and forward from the person’s ear over a portion of the person’s forehead. In an example, a device can have an arcuate and/or curved posterior portion which loops over the top of a person’s ear and an anterior portion which extends upward and forward from the person’s ear over a portion of the person’s forehead. In an example, a device can have an arcuate and/or curved posterior portion which loops around the back of a person’s ear and an anterior portion which extends upward and forward from the person’s ear over the person’s temple area.

In an example, a device can have an arcuate and/or curved posterior portion which loops over the top of a person’s ear and an anterior portion which extends upward and forward from the person’s ear over the person’s temple area. In an example, a device can have an arcuate and/or curved posterior portion which loops around a person’s ear and an anterior portion which extends upward and forward from the person’s ear over a portion of the person’s forehead. In an example, a device can have an arcuate and/or curved posterior portion which loops around a person’s ear and an anterior portion which extends upward and forward from the person’s ear over the person’s temple area. In an example, a device can hook around the rear of a person’s ear to attach it to a person’s head.

In an example, eating can be detected by measuring changes in air pressure in a person’s ear canal. In an example, food consumption can be detected by an ear bud by measuring changes in electromagnetic energy from the ear. In an example, food consumption can be detected by vibrations in the ear canal. In an example, this device can be embodied in a hearing aid. In an example, this device can be embodied in a hearing aid. In an example, this device can be embodied in an ear bud. In an example, this device can be embodied in an ear ring. In an example, this device can be embodied in an earpiece.

In an example, a system can comprise two wrist worn devices, one worn on each wrist, where one device have more sensors than the other. In an example, a wrist-worn device can count the number of hand-up-to-mouth, then tilt, then hand-back-from-mouth movements. In an example, a wrist-worn device can count the number of hand-to-mouth movements. In an example, a device can comprise an inertial motion unit (IMU) which is worn on a person’s wrist. In an example, device can be in communication with a smart watch via a data transmitter and receiver. In an example, a wrist-worn device for measuring a person’s food consumption can detect whether the person is eating by analyzing the person’s arm motion and heart rate.

In an example, this device can be embodied in a wrist band or bracelet. In an example, this device can be embodied in a smart watch. In an example, a device and/or system can comprise a smart watch, wristband, bracelet, gauntlet, or sleeve. In an example, a system can comprises two wrist-worn devices, one worn on the right wrist and one worn on the left wrist, to monitor for eating-related hand motions on either side. In an example, a system can comprises two wrist-worn devices with accelerometers, one worn on the right wrist and one worn on the left wrist, to monitor for eating-related hand motions on either side. In an example, system can comprise two wrist-worn devices, one on dominant and one on non-dominant.

In an example of this invention, the fields of vision from one or more wrist-worn cameras are shifted by movement of the person’s arm and hand while the person eats. This shifting causes the fields of vision from the one or more cameras to collectively and automatically encompass the person’s mouth and nearby food while the person is eating. This encompassing imaging occurs without the need for human intervention when the person eats. This eliminates the need for a person to manually aim a camera (or other camera) toward their mouth or toward a nearby food. In an example, a camera can be positioned on a person’s wrist at a location from which it takes pictures along an imaging vector that is directed generally upward from the camera toward the person’s mouth as the person eats. In an example, a camera can be positioned on the person’s wrist at a location from which it takes pictures along an imaging vector that is directed generally downward from the camera toward nearby food as the person eats.

In an example, a camera can be worn like a wrist watch, with a camera instead of a watch face, which has been rotated 180 degrees around a person’s wrist. In an example, the field of vision from this camera can points generally downward in a manner that would be likely to encompass nearby food which the person would engage with a utensil. In an example, this field of vision is rotated upward towards the person’s mouth by the rotation of the person’s wrist as the person brings a utensil up to their mouth. In this manner, a single wrist-worn camera can take pictures of both nearby food and a person’s mouth, due to the rolling motion of a person’s wrist as food is moved along a food consumption pathway.

In an example, a device and method can comprise a camera with a single wide-angle camera that is worn on the narrow side of a person’s wrist or upper arm, in a manner similar to wearing a watch or bracelet that is rotated approximately 90 degrees. This camera can automatically take pictures of the person’s mouth, a nearby food, or both as the person moves their arm and hand as the person eats. In an example, a device and method can comprise a camera with a single wide-angle camera that is worn on the anterior surface of a person’s wrist or upper arm, in a manner similar to wearing a watch or bracelet that is rotated approximately 180 degrees. This camera automatically takes pictures of the person’s mouth, a nearby food, or both as the person moves their arm and hand as the person eats. In an example, a device and method can comprise a camera that is worn on a person’s finger in a manner similar to wearing a finger ring, such that the camera automatically takes pictures of the person’s mouth, a nearby food, or both as the person moves their arm and hand as the person eats.

In an example, a device and method can comprise a device that is worn on a person so as to take images of food, or pieces of food, at multiple locations as food travels along a food consumption pathway. In an example, a device and method comprise a device that takes a series of pictures of a portion of food as it moves along a food consumption pathway between nearby food and the person’s mouth. In an example, a device and method comprise a wearable camera that takes pictures upwards toward a person’s face as the person’s arm bends when the person eats. In an example, a device comprises a camera that captures images of the person’s mouth when the person’s elbow is bent at an angle between 40-140 degrees as the person brings food to their mouth. In various examples, a device and method automatically takes pictures of food at a plurality of positions as food moves along a food consumption pathway. In an example, a device and method estimates the type and quantity of food consumed based, at least partially, on pattern analysis of images of the proximal and distal endpoints of a food consumption pathway.

In an example, a device and method comprise a camera with a single camera that takes pictures along shifting imaging vectors, as food travels along a food consumption pathway, so that it take pictures of a food source and the person’s mouth sequentially. In an example, a device and method takes pictures of a food source and a person’s mouth from different positions as food moves along a food consumption pathway. In an example, a device and method comprise a camera that scans for, locates, and takes pictures of the distal and proximal endpoints of a food consumption pathway. In an example, a device can be embodied in a wristband that is worn around the person’s wrist in a manner like a wrist-watch strap or a bracelet. In an example, a device can be attached to wristband, including one or more sensors that collect information concerning food consumption and a data processing and transmission unit. In an example, this device can include: data processing and transmission unit; a motion sensor; a microphone; a speaker; and a miniature video camera. In an example, the person can wear one such device on each wrist.

In an example, a device can comprise at least two cameras worn on a person’s body: wherein a first camera is worn on a body member selected from the group consisting of the person’s wrist, hand, lower arm, and finger; wherein the field of vision from the first camera automatically encompasses the person’s mouth as the person eats; wherein a second camera is worn on a body member selected from the group consisting of the person’s neck, head, torso, and upper arm; and wherein the field of vision from the second camera automatically encompasses nearby food as the person eats.

In an example, a device can comprise one or more of cameras which are worn on a location on the human body that provides at least one line of sight from the device to the person’s mouth and at least one line of sight to a nearby food, as food travels along a food consumption pathway. In various examples, these one or more cameras simultaneously or sequentially record images along at least two different vectors, one which points toward the mouth during at least some portion of a food consumption pathway and one which points toward the food source during at least some portion of a food consumption pathway. In various examples, a device and method comprise multiple cameras that are worn on a person’s wrist, hand, arm, or finger -- with some imaging elements pointed toward the person’s mouth from certain locations along a food consumption pathway and some imaging elements pointed toward nearby food from certain locations along a food consumption pathway.

In an example, a device can comprise two opposite-facing cameras that are worn on band around a person’s wrist. In an example, two wrist-worn cameras can take pictures of nearby food and the person’s mouth. These pictures are used to estimate, in an automatic and tamper-resistant manner, the types and quantities of food consumed by the person. Information on food consumed, in turn, is used to estimate the person’s caloric intake. As the person eats, these two cameras of the camera take pictures of nearby food and the person’s mouth. These pictures are analyzed, using pattern recognition or other image-analyzing methods, to estimate the types and quantities of food that the person consumes. In an example, these pictures are motion pictures (e.g. videos). In an example, these pictures can be still-frame pictures.

In an example, a device comprises two or more cameras wherein a first camera is pointed toward the person’s mouth most of the time, as the person moves their arm to move food along a food consumption pathway, and wherein a second camera is pointed toward nearby food most of the time, as the person moves their arm to move food along a food consumption pathway. In an example, a device comprises one or more cameras wherein: a first camera points toward the person’s mouth at least once as the person brings a piece (or portion) of food to their mouth from a nearby food; and a second camera points toward a reachable food source at least once as the person brings a piece (or portion) of food to their mouth from a reachable food source.

In an example, a more-intrusive (and presumably more-accurate) “level 2” sensor can be a wearable camera. In an example, a wearable camera can be a camera that is part of a device that the person wears on their wrist, in a manner like a wrist watch. In an example, this camera can be directed toward the person’s fingers to identify food which the person reaches for, grasps, holds, and consumes during eating activities. In various examples, this food can be on a plate, on a shelf, in a bag, in a glass, on a table, or otherwise within viewing (and reaching) distance of the person.

In an example, a person can wear a camera comprised of a wrist band to which are attached two cameras on the opposite (narrow) sides of the person’s wrist. In an example, a camera can be worn around a person’s wrist. Accordingly, the camera moves as food travels along a food consumption pathway. In an example, the fields of vision from two cameras on an automatic-camera can automatically and collectively encompass a person’s mouth and a nearby food, from at least some locations, as the camera moves when food travels along a food consumption pathway. In an example, this movement allows the camera to take pictures of both the person’s mouth and a reachable food source, as the person eats, without the need for human intervention to manually aim cameras toward either the person’s mouth or a nearby food, when the person eats.

In an example, a single wrist-mounted camera can be linked to a mechanism that shifts the camera’s imaging vector (and field of vision) automatically as food moves along a food consumption pathway. This shifting imaging vector allows a single camera to encompass nearby food and the person’s mouth, sequentially, from different locations along a food consumption pathway. In an example, a wearable camera can recognize that its line of sight to a person’s mouth is unobstructed because it recognizes the person’s mouth using face recognition methods. In other examples, a camera can recognize that its line of sight to the person’s mouth is unobstructed by using other pattern recognition or imaging-analyzing means. As long as a line of sight from the camera to the person’s mouth is maintained (unobstructed), the device and method can detect if the person starts eating and, in conjunction with images of a reachable food source, it can estimate caloric intake based on quantities and types of food consumed.

In an example, an invention can include an accelerometer that is worn on the person’s wrist and linked to the imaging vector of a camera. The accelerometer detects arm and hand motion as food moves along a food consumption pathway. Information concerning this arm and hand movement is used to automatically shift the imaging vector of a camera such that the field of vision of the camera sequentially captures images of a reachable food source and the person’s mouth from different positions along a food consumption pathway. In an example, when the accelerometer indicates that the person’s arm is in the downward phase of a food consumption pathway (in proximity to a reachable food source) then the imaging vector of the camera is directed upwards to get a good picture of the person’s mouth interacting with food. Then, when the accelerometer indicates that the person’s arm is in the upward phase of a food consumption pathway (in proximity to the person’s mouth), the imaging vector of the camera is directed downwards to get a good picture of a reachable food source.

In an example, having two cameras mounted on opposite sides of a person’s wrist increases the probability of encompassing both the person’s mouth and nearby food as the person rolls their wrist and bends their arm to move food along a food consumption pathway. In an example, more than two cameras can be attached on a band around the person’s wrist to further increase the probability of encompassing both the person’s mouth and a reachable food source.

In an example, multiple cameras can be worn on the same body member. In an example, multiple cameras can be worn on different body members. In an example, a camera can be worn on each of a person’s wrists or each of a person’s hands. In an example, one or more cameras can be worn on a body member and a supplemental camera can be located in a non-wearable device that is in proximity to the person. In an example, wearable and non-wearable cameras can be in wireless communication with each other. In an example, wearable and non-wearable cameras can be in wireless communication with an image-analyzing member. In an example, one camera can take pictures of both a person’s mouth and nearby food with only a single field of vision. In an example, this single wrist-mounted camera has a wide-angle lens that allows it to take pictures of the person’s mouth when a piece of food is at a first location along a food consumption pathway and allows it to take pictures of nearby food when a piece food is at a second location along a food consumption pathway.

In an example, one or more cameras can be worn on one or more body members selected from the group consisting of the person’s wrist, hand, arm, and finger; wherein the fields of vision from one or more cameras are moved as the person moves their arm when the person eats; and wherein this movement causes the fields of vision from one or more cameras to collectively and automatically encompass the person’s mouth and a nearby food, when the person eats, without the need for human intervention, when the person eats, to manually aim a camera toward the person’s mouth or toward a reachable food source.

In an example, one or more cameras can be worn on one or more body members selected from the group consisting of the person’s wrist, hand, arm, and finger; wherein the fields of vision from one or more cameras are moved as the person moves their arm when the person eats; and wherein this movement causes the fields of vision from one or more cameras to collectively and automatically encompass the person’s mouth and a nearby food, when the person eats, without the need for human intervention, when the person eats, to manually aim a camera toward the person’s mouth or toward a reachable food source.

In an example, pictures of a person’s mouth taken by a camera are particularly useful for estimating the quantities of food actually consumed by the person. Static or moving pictures of the person inserting pieces of food into their mouth, refined by counting the number or speed of chewing motions and the number of cycles of a food consumption pathway, can be used to estimate the quantity of food consumed. In an example, the fields of vision from one or more cameras can collectively and automatically encompass a person’s mouth and nearby food, when the person eats, without the need for human intervention, when the person eats, to manually aim a camera toward the person’s mouth or toward a reachable food source. In an example, the fields of vision from one or more cameras are moved as the person moves their arm when the person eats; and wherein this movement causes the fields of vision from one or more cameras to collectively and automatically encompass the person’s mouth and a nearby food, when the person eats, without the need for human intervention, when the person eats, to manually aim a camera toward the person’s mouth or toward a reachable food source.

In an example, there can be one miniature video camera in a device and it can be located on the outer portion of a person’s wrist where the main body of a wrist watch would generally be located. In an example, such a device can have one video camera located on the opposite side of the person’s wrist. In other examples, there can be two or more video cameras mounted on different locations around the person’s wrist. In an example with two or more video cameras, different cameras may track different objects. For example, one camera may track the person’s fingers and the other camera may track the person’s mouth. In various examples, different cameras may operate at different times and/or with different focal lengths.

In an example, two cameras can be worn on the narrow sides of a person’s wrist, between the posterior and anterior surfaces of the wrist, such that the moving field of vision from the first of these cameras automatically encompasses the person’s mouth (as the person moves their arm when they eat) and the moving field of vision from the second of these cameras automatically encompasses a reachable food source (as the person moves their arm when they eat). This embodiment of the invention is comparable to a wrist-watch that has been rotated 90 degrees around the person’s wrist, with a first camera located where the watch face would be and a second camera located on the opposite side of the wrist. In various examples of a device and method, the fields of vision from one or more cameras worn on the person’s wrist, hand, finger, or arm are shifted by movement of the person’s arm bringing food to their mouth along a food consumption pathway. In an example, this movement causes the fields of vision from these one or more cameras to collectively and automatically encompass the person’s mouth and a nearby food.

In various examples, movement of one or more wrist-worn cameras allows their fields of vision to automatically and collectively capture images of the person’s mouth and nearby food without the need for human intervention when the person eats. In an example, a device and method includes a camera that is worn on the person’s wrist, hand, finger, or arm, such that this camera automatically takes pictures of the person’s mouth, a nearby food, or both as the person moves their arm and hand when they eat. This movement causes the fields of vision from one or more cameras to collectively and automatically encompass the person’s mouth and nearby food as the person eats. Accordingly, there is no need for human intervention, when the person starts eating, to manually aim a camera (or other camera) toward the person’s mouth or toward a nearby food. Picture taking of the person’s mouth and the food source is automatic and virtually passive.

In an example, a device can comprise an EMG sensor which is worn on a person’s finger. In an example, a device can comprise a microphone which is worn on a person’s finger. In an example, a device can comprise a spectroscopic sensor which is worn on a person’s finger. In an example, a device can comprise an optical sensor which is worn on a person’s finger. In an example, a system for managing a person’s blood glucose level can include an insulin pump on a person and a finger ring with sensors which collect data which is analyzed to measure a person’s food consumption. In an example, a system for managing a person’s blood glucose level can include an insulin pump on a person and a finger ring with acoustic sensors which collect data which is analyzed to measure a person’s food consumption. In an example, a system for managing a person’s blood glucose level can include an insulin pump on a person and a finger ring with a microphone which collects data which is analyzed to measure a person’s food consumption.

In an example, a system for managing a person’s blood glucose level can include an insulin pump on a person and a finger ring with a microphone and a camera which collect record sounds and images which are analyzed to measure a person’s food consumption. In an example, a system for managing a person’s blood glucose level can include an insulin pump on a person and a finger ring with a microphone and a camera which collect record sounds and images which are analyzed to measure the types and amounts of food consumed by the person. In an example, a system for managing a person’s blood glucose level can include an insulin pump on a person and a finger ring with a microphone and a camera which collect data which is analyzed to measure a person’s food consumption. In an example, a finger ring with a camera can record food images from different angular perspectives as a person waves (e.g. horizontally moves) the ring over food. In an example, a finger ring with a camera can record food images from different angular perspectives as a person waves (e.g. horizontally moves) the ring over a meal.

In an example, this device can be embodied in a finger ring. In an example, a device can be embodied in one or more finger rings. In an example, a device can be part of a system comprising one or more finger rings. In an example, a device can comprise an inertial motion unit (IMU) which is worn on a person’s finger. In an example, a device can detect eating by monitoring hand gestures using one or more finger rings. In an example, a device can estimate food consumption by monitoring hand gestures using one or more finger rings.

In an example, a caloric-input measuring device can automatically estimate a person’s caloric intake based on analysis of pictures taken by one or more cameras worn on the person’s wrist, hand, finger, or arm. In various examples, a device can include one or more cameras worn on a body member selected from the group consisting of: wrist, hand, finger, upper arm, and lower arm. In various examples, a device includes one or more cameras that are worn in a manner similar to a wearable member selected from the group consisting of: wrist watch; bracelet; arm band; and finger ring.

In an example, a device can be attached to an article of clothing. In an example, a device can be attached to an article of clothing by a clip, clasp, pin, hook, snap, magnet, or button. In an example, a device can be clipped or otherwise attached to the collar of a garment. In an example, a device for measuring a person’s food consumption can be embodied in a necklace, neckband, pendant, neck-worn patch, or garment collar. In an example, a device for measuring a person’s food consumption can be incorporated into the collar of an article of clothing. In an example, a device for measuring a person’s food consumption can be attached to the collar of an article of clothing. In an example, a device can be attached to the collar of a garment by a clip, clasp, hook, snap, hook-and-eye material, button, pin, or magnet.

In an example, a radar emitter can be incorporated into a spoon in order to measure the nutritional composition of food in the spoon. In an example, a system can further comprise a utensil (e.g. a spoon or a fork) with a strain and/or pressure sensor which collects data to estimate the amount (e.g. quantity and/or weight) of food in a spoonfull or forkfull. In an example, a system can further comprise a utensil (e.g. a spoon or a fork) with a sensor which collects data to estimate the amount (e.g. quantity and/or weight) of food in a spoonfull or forkfull.

In an example, food is broadly defined to include liquid beverages, as well as solid and semi-solid food. In an example, the food-transporting member is a glass that contains a drinkable beverage. In various examples, the food-transporting member can be a cup, mug, or other beverage container. In an example, the food-transporting member can be a fork, spoon, chop stick, or other utensil that transports solid or semi-solid food up to the person’s mouth. In an example, the person can transport food directly to their mouth by grasping it with their fingers, without the need for a food-transporting member as an intermediary. For example, the person can grasp a piece of food (such as a potato chip or a peanut or an apple) directly with their fingers and bring it up to their mouth to consume it.

In an example, a device can comprise a probe which is inserted into food, wherein the probe further comprises a sound emitter and sound receiver. In an example, a device can comprise a probe which is inserted into food, wherein the probe further comprises an ultrasonic sound emitter and an ultrasonic sound receiver. In an example, a device can comprise a probe which is inserted into food, wherein the probe further comprises an electrical impedance sensor. In an example, a device can comprise a probe which is inserted into food, wherein the probe further comprises an electrical capacitance sensor. In an example, a device can comprise a probe which is inserted into food, wherein the probe further comprises an electromagnetic energy emitter and an electromagnetic energy receiver. In an example, a food probe that is inserted into food can include a thermal sensor (e.g. a thermistor). In an example, a food probe that is inserted into food can include a camera.

In an example, a device can comprise a probe which is inserted into food, wherein the probe further comprises an infrared light emitter and an infrared light receiver. In an example, a device can comprise a probe which is inserted into food, wherein the probe further comprises a light emitter and light receiver. In an example, a food probe that is inserted into food can include an optical lens. In an example, a food probe that is inserted into food can include a light emitter and a light receiver. In an example, a system for measuring a person’s food and/or nutritional consumption can include a food probe with one or more of the following types of food sensors: a camera; a chemical sensor; a light emitter and receiver; a radar emitter and receiver; and an optical scanner. In an example, a system for measuring a person’s food and/or nutritional consumption can include a food probe which is inserted into food, wherein the probe includes one or more of the following types of food sensors: a camera; a chemical sensor; a light emitter and receiver; a radar emitter and receiver; and an optical scanner.

In an example, a food probe that is inserted into food can include a light emitter which illuminates the interior of the food. In an example, a system can include a food probe which is removably inserted into and extract from a wearable device. In an example, a food probe can be held in a wearable device until it is removed for use. In an example, a food probe can be longitudinal with a length between one half inch and two inches. In an example, a food probe can be longitudinal with a length between 2″ and 5″. In an example, a food probe can be longitudinal with a length between 1″ and 3″.

In an example, a system can include a longitudinal food probe with a plurality of spectroscopic sensor along the length of the probe, wherein the probe is inserted into food to measure the composition of the food at multiple locations and/or depths in the food. In an example, a food probe that is inserted into food can include a spectroscopic sensor. In an example, a system can include a spectroscopic food probe which is inserted into food to measure the nutritional composition of the food. In an example, a system can include a spectroscopic food probe which is inserted into food to measure the molecular composition of the food. In an example, a system can include a spectroscopic food probe which is detached from a wearable device and inserted into food to measure the molecular composition of the food.

In an example, a device and/or system can comprise a button, pin, pip, patch, or pendant. In an example, a device and/or system can comprise a headband, partial-circumference headband, hair comb, or tiara. In an example, a device and/or system can further comprise headphones. In an example, a device can be attached to a person’s jaw or chin (e.g. adhered?) In an example, a device can be embodied in a nose ring. In an example, a device can be embodied in a smart band and/or bracelet. In an example, a device can be embodied in a tongue ring. In an example, a device can be embodied in eyeglass frames which can hold (prescription) lenses. In an example, a device can be part of a system comprising a smart necklace and/or pendant. In an example, a device can be worn on and/or around a person’s neck. In an example, a device can be worn on the back of a person’s neck to be less obtrusive. In an example, a device can be worn on the back of a person’s head to be less obtrusive.

In an example, a device can comprise a bone conduction headphone. In an example, a device can comprise an EEG-based (e.g. BCI) user interface. In an example, a device for measuring a person’s food consumption can be worn on or around the person’s neck. In an example, this device can be embodied in a headband. In an example, a device for measuring a person’s food consumption can comprise a heart rate monitor, wherein analysis of data from the heart rate monitor is used to identify changes in the speed, rate, frequency, amplitude, and/or waveform of the heart rate.

In an example, a system can use data from biometric parameters to estimate the types and/or amounts of food consumed by a person wherein this data can include one or more variables selected from the group consisting of: blood glucose level, blood oxygenation level, blood pressure, body temperature, heart rate, heart rate variability, hydration level, recent speech history, and tissue impedance level. In an example, a system can analyze the relationship between a person’s pulse rate and the person’s consumption of different types and/or amounts of food. In an example, a system can analyze the relationship between a person’s heart rate and the person’s consumption of different types and/or amounts of food.

In an example, a camera can be worn on a person’s wrist, neck, head, or torso. In an example, this camera can continuously track the location of the person’s mouth and take continuous video images of the person’s mouth to detect and identify food consumption. In an example, this camera can continuously track the location of the person’s hands and take continuous video images of the space near the person’s fingers to detect and identify food consumption. In an example, a camera can be worn on the person’s neck in a manner like a necklace, incorporated into a button worn on clothing, or incorporated into a finger or ear ring.

In an example, a device and method can comprise at least two cameras that are worn on a person’s body. One of these cameras can be worn on a body member selected from the group consisting of the person’s wrist, hand, lower arm, and finger, wherein the field of vision from this camera automatically encompasses the person’s mouth as the person eats. A second of these cameras can be worn on a body member selected from the group consisting of the person’s neck, head, torso, and upper arm, wherein the field of vision from the second camera automatically encompasses nearby food as the person eats.

In an example, a device and method can estimate the types and quantities of food actually consumed by a person based on pictures of both nearby food and the person’s mouth. Having both such images provides better information than either separately. Pictures of nearby food can be particularly useful for identifying the types of food available to the person for potential consumption. Pictures of the person’s mouth (including food traveling a food consumption pathway and food-mouth interaction such as chewing and swallowing) can be particularly useful for identifying the quantity of food consumed by the person. Combining both images in an integrated analysis provides more accurate estimation of the types and quantities of food actually consumed by the person. This information, in turn, provides better estimation of caloric intake by the person.

In an example, a device and method comprise one or more cameras that take pictures of: food at a food source; a person’s mouth; and interaction between food and the person’s mouth. The interaction between the person’s mouth and food can include biting, chewing, and swallowing. In an example, utensils or beverage-holding members can be used as intermediaries between the person’s hand and food. In an example, a device comprises a camera that automatically takes pictures of the interaction between food and the person’s mouth as the person eats. In an example, a device comprises a wearable device that takes pictures of nearby food that is located in front of the person.

In an example, a device can be on a necklace. In an example, a device can be worn on a person’s clothing as a button or a brooch instead of being worn like a pendant on a necklace. In an example, such a device can be worn on a person’s ear like an ear ring. In an example, such a device can be incorporated into a person’s eyeglasses. In an example, a device can comprise an ornamental chain that is worn around a person’s neck like a necklace. In an example, a device can further comprise an oval member that is worn on the necklace like a pendant. In an example, this oval device can include three parts: (a) a wireless data processing and transmission unit that communicates with a remote scale; (b) a microphone and speaker unit with voice recognition capability; and (c) a miniature video camera that automatically takes pictures of food as it is transported by the person’s hand up to their mouth.

In an example, a device can comprise at least two cameras or other cameras. A first camera can be worn on a location on the human body from which it takes pictures along an imaging vector which points toward the person’s mouth while the person eats. A second camera can be worn on a location on the human body from which it takes pictures along an imaging vector which points toward a nearby food. In an example, a device may include: (a) a camera that is worn on the person’s wrist, hand, arm, or finger such that the field of vision from this camera automatically encompasses the person’s mouth as the person eats; and (b) a camera that is worn on the person’s neck, head, or torso such that the field of vision from this camera automatically encompasses nearby food as the person eats.

In an example, a device can have an unobtrusive, or even attractive, design like a piece of jewelry. In various examples, a device can look similar to an attractive wrist watch, bracelet, finger ring, necklace, or ear ring. In an example, a device can increase the accuracy and compliance of caloric intake monitoring and estimation. In an example, a device can include at least two cameras worn on a person’s body: wherein a first camera is worn on a body member selected from the group consisting of the person’s wrist, hand, lower arm, and finger; wherein the field of vision from the first camera automatically encompasses the person’s mouth as the person eats; wherein a second camera is worn on a body member selected from the group consisting of the person’s neck, head, torso, and upper arm; and wherein the field of vision from the second camera automatically encompasses nearby food as the person eats.

In an example, a miniature video camera can take pictures of a person’s hand and fingers, and also the space surrounding the person’s hand and fingers, in order to detect and identify food. In an example, a video camera need not operate continuously. In an example, a miniature video camera that is attached like a pendant to a necklace can track a food-transporting member (such as a fork) as it transports food towards a person’s mouth. In an example, the field of vision that is seen by the miniature video camera can comprise a radially-extending circle that encompasses the space surrounding the person’s fingers, including food.

In an example, a person can wear a camera around their neck. In an example, this camera can be worn like a central pendant on the front of a necklace. From this location, this camera can have a forward-and-downward facing field of vision that encompasses a person’s hand and nearby food. In an example, this camera can uses gesture recognition, or other pattern recognition methods, to shift its focus so as to always maintain a line of sight to the person’s hand and/or to scan for potential reachable food sources.

In an example, a video camera can have a fixed focal direction and focal length. In an example, the focal direction of the video camera may always point toward the person’s fingers and the space surrounding the person’s fingers. In an example, the video camera can have a focal direction or focal length that is automatically adjusted while the camera is in operation. In an example, when it is in operation, the video camera can scan back and forth through the space near the person’s hand and fingers to search for food. In an example, the video camera can use pattern recognition to track the relative location of the person’s fingers. In an example, the camera can automatically adjust its focal direction and/or focal length to monitor and identify eating-related objects (such as a fork or glass) that come into contact with the person’s fingers.

In an example, a wearable camera can be worn on the person’s body and a non-wearable camera can be positioned in proximity to the person’s body. In various examples, a device and method can include one or more cameras that are worn on the person’s neck, head, or torso and one or more cameras that are positioned on a table, counter, or other surface in front of the person in order to simultaneously, or sequentially, take pictures of nearby food and the person’s mouth as the person eats.

In an example, a wearable device can have advantages over a non-wearable (e.g. handheld) device. It is possible to have a non-wearable camera that can be manually positioned (on a table or other surface) to be aimed toward an eating person, such that its field of vision includes both a food source and the person’s mouth. In theory, every time the person eats a meal or takes a snack, the person could: take out a camera (such as a smart phone); place the device on a nearby surface (such as a table, bar, or chair); manually point the device toward them so that both the food source and their mouth are in the field of vision; and manually push a button to initiate picture taking before they start eating. However, this manual process with a non-wearable device is highly dependent on the person’s compliance with this labor-intensive and possibly-embarrassing process.

In an example, a wearable device can measure a person’s caloric intake. In an example, a device can be worn on a person’s wrist in a manner similar to a wrist watch. In an example, a device can include: (a) a motion sensor that automatically analyzes movement of the person’s wrist to detect when the person is probably eating; (b) a microphone and speaker unit, with voice recognition capability, which enables two-way vocal communication between the device and the person; (c) a wireless data processing and transmission unit that communicates with a remote scale; and (d) a miniature video camera that automatically takes pictures of food as it is transported to the person’s mouth by the person’s hand. In various examples, one or more sensors can be implanted within the person’s body and may internally monitor chewing, swallowing, biting, other muscle activity, enzyme secretion, neural signals, or other ingestion-related processes or activities.

In an example, one or more cameras are worn on a body member that moves as food travels along a food consumption pathway. In this manner, these one or more cameras have lines of sight to the person’s mouth and to the food source during at least some points along a food consumption pathway. In various examples, this movement is caused by bending of the person’s shoulder, elbow, and wrist joints. In an example, a camera is worn on the wrist, arm, or hand of a dominant arm, wherein the person uses this arm to move food along a food consumption pathway. In an example, a camera can be worn on the wrist, arm, or hand of a non-dominant arm, wherein this other arm is generally stationery and not used to move food along a food consumption pathway. In an example, cameras can be worn on both arms.

In an example, these one or more sensors can be worn on the person’s body, either directly or worn on clothing. In various examples, these one or more sensors can be worn on the person’s wrist, neck, ear, head, arm, finger, mouth or other locations on the person’s body. In various examples, these one or more sensors can be worn in manner similar to that of a wrist watch, bracelet, necklace, pendant, button, belt, hearing aid, bluetooth device, ear ring, and/or finger ring. In various examples, a device can be worn on the person’s wrist, neck, ear, head, arm, finger, mouth, or torso. In various examples, a device can be worn in a manner similar to a wrist watch, bracelet, necklace, pendant, button, belt, hearing aid, ear plug, headband, eyeglasses, bluetooth device, ear ring, or finger ring. In various examples, a device includes one or more cameras that are worn on a body member selected from the group consisting of: neck; head; and torso. In various examples, a device includes one or more cameras that are worn in a manner similar to a wearable member selected from the group consisting of: necklace; pendant, dog tags; brooch; cuff link; ear ring; eyeglasses; wearable mouth microphone; and hearing aid.

In various examples, one or more cameras can be integrated into one or more wearable members that appear similar to a wrist watch, wrist band, bracelet, arm band, necklace, pendant, brooch, collar, eyeglasses, ear ring, headband, or ear-mounted bluetooth device. In an example, a device can comprise two cameras, or two cameras mounted on a single member, which are generally perpendicular to the longitudinal bones of the upper arm. In an example, one of these cameras can have an imaging vector that points toward a food source at different times while food travels along a food consumption pathway. In an example, another one of these cameras can have an imaging vector that points toward the person’s mouth at different times while food travels along a food consumption pathway. In an example, these different imaging vectors may occur simultaneously as food travels along a food consumption pathway. In an example, these different imaging vectors may occur sequentially as food travels along a food consumption pathway. A device and method may provide images from multiple imaging vectors, such that these images from multiple perspectives are automatically and collectively analyzed to identify the types and quantities of food consumed by the person.

In an example, a device and/or system will prompt a person to activate a sequence of sensors (e.g. motion sensor, microphone, camera, and spectroscopic sensor) to provide additional information about nearby food until the type and/or amount of food is identified by the device and/or system with a target minimum level of certainty, accuracy, and/or confidence. In an example, a device and/or system will prompt a person to activate an increasing number of sensors (e.g. motion sensor, microphone, camera, and spectroscopic sensor) to provide additional information about nearby food until the type and/or amount of food is identified by the device and/or system with a target minimum level of certainty, accuracy, and/or confidence. In an example, a device can prompt a person to do a spectroscopic scan of nearby food (and integrate this information via speech recognition software) in order to supplement and/or correct automatic classification of food types and/or amounts by the device.

In an example, a device can prompt a person to speak descriptions of food items (and integrate this information via speech recognition software) in order to supplement and/or correct automatic classification of food types and/or amounts by the device. In an example, a device and/or system will prompt a person to provide additional information about food until the type and/or amount of food is identified with a target minimum level of certainty, accuracy, and/or confidence. In an example, when a device and/or system detects that a person has started eating, the system prompts a person to take action to record food images.

“Level 2” actions are more intrusive into a person’s privacy and/or time than are “level 1” actions. Level 2 actions give the person less flexibility with respect to data entry structure, timing, and precision; and are less discrete with respect to the data entry interface. For these reasons, the person has an incentive to be engaged and record timely and accurate data concerning food consumption in “level 1” data collection in order to avoid the intrusiveness of “level 2” data collection. As discussed earlier in the analogy to road ridges (which can be annoying, but can save lives), the escalating intrusiveness of “level 2” actions could also be viewed as “annoying.” However, in the long-run, it can be far less “annoying” than the many negative health outcomes of obesity or the health risks of invasive stomach-altering surgery.

Actively-entered data about food consumption is received from voluntary actions that are performed by the person in association with eating events, other than the actions of eating itself. After passively-collected data and actively-entered data concerning food consumption are received, a method can then progress to estimation of the person’s caloric intake. In an example, this single estimate can be created by combining, weighting, and/or merging passively-collected data and actively-entered data concerning food consumption.

In an example, a “level 1” action can comprise manually entering a phrase to describe food into a mobile device and a “level 2” action can comprise manually taking (e.g. “pointing and shooting”) a picture of food. In an example, a “level 1” action can be manually entering (at the end of the day) information on food that was consumed during the day at a private moment, but a “level 2” action can be entering information about food consumed in real time during each eating event. In an example, a “level 1” action can be responding to a quiet vibration from a wearable device by entering data on food consumed, but a “level 2” action can be responding to full-volume voice inquiry from a wearable device.

In an example, a device can estimate the person’s caloric intake based on both passively-collected data and actively-entered data concerning food consumption. In an example, a device can escalate data collection to a more-accurate, but also more-intrusive, level of passively-collected data collection if the estimate of caloric intake from a less-intrusive level is not sufficiently accurate. In an example, the accuracy of estimates of caloric intake can be tested by comparing predicted weight gain or loss to actual weight gain or loss. In an example, if predicted and actual weight gain or loss do not meet the criteria for similarity and/or convergence, then the device can activate a level of automatic monitoring (and passively-collected data collection) which is more-accurate, but also more-intrusive into the person’s privacy and/or time.

In an example, a device on a wristband can be used to collect passively-collected data concerning the person’s food consumption, without requiring any voluntary actions by the person other than the actual actions of eating. In an example, a device can be used to collect actively-entered data concerning the person’s food consumption through voluntary actions by the person other than the actual actions of eating. In an example, the same device can be used to collect both passively-collected data and actively-entered data concerning the person’s food consumption. In an example, collection of passively-collected data vs. actively-entered data concerning food consumption can be independent of each other. In an example, collection of passive and actively-entered data can be causally linked. In an example, when passively-collected data from a sensor indicates that the person is probably eating, then the device can prompt the person to provide actively-entered data concerning what they are eating.

In an example, a first set of data can be received concerning what the person eats, wherein this first set includes passively-collected data that is collected in a manner that does not require voluntary actions by the person associated with particular eating events other than the actions of eating, and wherein this first set also includes actively-entered data that is collected in a manner that requires voluntary actions by the person associated with particular eating events other than the actions of eating. In an example, a method can include collection of both passively-collected data and actively-entered data concerning food consumption, calculating and comparing estimates of caloric intake from these two data sources, and collecting additional information if the criteria for similarity and/or convergence of these two estimates are not met.

In an example, a method for collecting eating-related data can comprise: (a) a first step in which passively-collected data about food consumption is received from motion sensors that are worn in or on the person and this data is used to estimate caloric intake; (b) a second step in which actively-entered data about food consumption is received from “level 1” actions performed by the person and this data used to estimate caloric intake; and (c) a third step in which these two estimates are compared to determined whether they meet the criteria for similarity and/or convergence. If these criteria are met, then the method stops and the person is only engaged with less-intrusive motion sensors and “level 1” actions.

In an example, a method for measuring a person’s caloric intake can comprise: (a) receiving a first set of data concerning what a person eats in a manner that does not require voluntary actions by the person associated with particular eating events other than the actions of eating; (b) receiving a second set of data concerning what the person eats in a manner that requires voluntary actions by the person associated with particular eating events other than the actions of eating; (c) calculating a first estimate of the person’s caloric intake based on the first set of data, calculating a second estimate of the person’s caloric intake based on the second set of data, and comparing these first and second estimates of caloric intake to determine whether these estimates meet criteria for similarity and/or convergence; and (d) if the first and second estimates of caloric intake do not meet the criteria for similarity and/or convergence, then receiving a third set of data concerning what the person eats and calculating one or more new estimates of caloric intake using this third set of data.

In an example, a person can provide actively-entered data first, before the automated detection of a likely eating event. In an example, accurate actively-entered data can be provided when such data is prompted by automated detection of a likely eating event. In an example, if accurate and timely data concerning food consumption is not provided at first, then the person will be prompted to provide it when the estimates of caloric intake from passive and actively-entered data fail to converge. In an example, a set of data can be received concerning what the person eats, wherein this set includes passively-collected data that is collected in a manner that does not require voluntary actions by the person associated with particular eating events other than the actions of eating, and wherein this set also includes actively-entered data that is collected in a manner that requires voluntary actions by the person associated with particular eating events other than the actions of eating.

In an example, a system and/or device can give a person an incentive to provide timely and accurate actively-entered data concerning food consumption in order to avoid potentially more-intrusive sensor monitoring and passively-collected data collection. Such a device and method can engage the person in their own energy balance and weight management to a greater degree than an entirely-passive device for automatic monitoring. Such a device and method can also ensure greater compliance and accuracy than an entirely-voluntary device for diet logging.

In an example, a system in which a person is prompted by a motion sensor to enter what they eat (each time that they eat) can provide a more accurate measurement of caloric intake than either passively-collected data from a motion sensor alone or actively-entered data from manual diet logging alone. Additional actively-entered data can be collected until different estimates of caloric intake meet the criteria for similarity and/or convergence. This provides the person with incentives for both timeliness and accuracy in actively-entered data reporting of food consumption. It also actively engages the person in managing their own energy balance.

In an example, actively-entered data about food consumption can be received from voluntary action performed by a person. Actively-entered data about a person’s food consumption is data that is received from voluntary actions performed by the person in association with eating events, other than the actual actions of eating. In various examples, voluntary action for recording food consumption can be selected from the group consisting of: writing on paper, typing on a keyboard, touching a touch screen, moving a cursor, speaking into a device with voice recognition capability, gesturing to a device with gesture recognition capability, manually scanning a bar code or other food code; and manually initiating the taking of a picture of food that will be consumed.

In an example, actively-entered data about food consumption can include precise information concerning the types and quantities of food consumed. In an example, actively-entered data about food consumption received can only include indirect raw data such as a picture of food, or general food categories, which must be subsequently analyzed in order to identify the types and quantities of food consumed. In an example, this actively-entered data can be received by a computer and stored therein. In an example, actively-entered data can be in a relatively raw form that requires analysis in order to identify the types and quantities of food consumed. For example, actively-entered data can comprise images of food consumed, without any accompanying explanation from the person. In various examples, analysis of actively-entered data can include one or more methods selected from the group consisting of: food image recognition or identification; word recognition or identification; logo recognition or identification; bar code recognition or identification; and 3D modeling.

In an example, actively-entered data can be independently initiated by the person before an eating event, during the eating event, or after the eating event. In an example, actively-entered data collection can be prompted or solicited based on the results of passively-collected data collection. For example, passively-collected data may suggest a high probability that the person is eating, which could trigger a request for actively-entered data entry. In an example of how this prompt can be operationalized, a wearable sensor can detect a probable eating event and this detection may prompt the person to enter actively-entered data about the eating event, if any.

In an example, actively-entered data provided by the person can include information about the types and quantities of food consumed. In various examples, this data can be provided before, during, or after eating. In an example, actively-entered data collection can be prompted or solicited in real time, when the microphone and speaker unit first detects probable eating. In an example, actively-entered data collection can be prompted or solicited at the end of the day and can be associated with multiple eating events detected by the microphone and speaker throughout the day. In an example, actively-entered data collection can be entirely independent; it may not be prompted or solicited at all.

In an example, an iterative method to measure caloric intake can have two caloric intake estimates based on passive and actively-entered data, respectively, wherein these estimates are compared and additional actively-entered data is only collected if these estimates do not meet criteria for similarity and/or convergence. In an example, collecting a first set of data does not require voluntary actions by the person associated with particular eating events other than the actions of eating, but collecting a second set of data does require voluntary actions by the person associated with particular eating events other than the actions of eating.

In an example, collection of a first set of data does not require voluntary actions by the person associated with particular eating events other than the actions of eating, but collection of a second set of data requires voluntary actions by the person associated with particular eating events other than the actions of eating, or vice versa. In an example, receiving the first set of data does not require voluntary actions by the person associated with particular eating events other than the actions of eating, but receiving the second set of data requires voluntary actions by the person associated with particular eating events other than the actions of eating, or vice versa.

In an example, collection of first and third sets of data does not require voluntary actions by the person associated with particular eating events other than the actions of eating, but collection of the second set of data does require voluntary actions by the person associated with particular eating events other than the actions of eating. In an example, collection of the first and second sets of data does not require voluntary actions by the person associated with particular eating events other than the actions of eating, but collection of the third set of data does require voluntary actions by the person associated with particular eating events other than the actions of eating.

In an example, collection of the first set of data does not require voluntary actions by the person associated with particular eating events other than the actions of eating, but collection of the second and third sets of data does require voluntary actions by the person associated with particular eating events other than the actions of eating. In an example, at least one of the first set of data and the second set of data comprises image data whose collection requires voluntary actions by the person associated with particular eating events other than the actions of eating, and the third set of data comprises image data whose collection is more continuous than the collection of at least one of the first and second sets of data.

In an example, solicitation or prompting of actively-entered data collection concerning food consumption can occur in real time when the motion sensor first detects a possible eating event. In an example, solicitation of actively-entered data can be delayed until after an eating event is finished. In an example, the device can keep a record of multiple eating events throughout the day and inquire about each during a cumulative data collection session at the end of the day. The latter is less intrusive with respect to eating events, but risks imprecision due to imperfect recall and “caloric amnesia.”

In an example, the collection and use of passively-collected data and actively-entered data can be done in parallel. In an example, collection of these two types of data can be done in an alternating manner or in a series. In an example, the collection of additional passively-collected data and additional actively-entered data can be done in a one-to-one correspondence. In an example, it can be done a many-to-one correspondence. In an example, the type of data (passive or voluntary) whose latest augmentation most contributes to the similarity and/or convergence of caloric intake estimates can be disproportionately selected for additional data collection.

In various examples, a person can be prompted to provide data information concerning food consumption using one or more methods selected from the group consisting of: a ring tone, a voice prompt, a musical prompt, an alarm, some other sound prompt, a text message, a phone call, a vibration or other tactile prompt, a mild electromagnetic stimulus, an image prompt, or activation of one or more lights. In various examples, some of these prompts are less intrusive with respect to the person’s privacy and/or time, while other prompts are more intrusive with respect to the person’s privacy and/or time -- especially in social eating situations. In various examples, prompts that are less easily detected by other people are generally less intrusive in social eating settings.

In various examples, actively-entered data concerning food consumption can be received before, concurrently with, or after passively-collected data is received. If the person initiates actively-entered data about food consumption before an eating event is detected via passively-collected data collection, then prompting of actively-entered data collection is not needed. In an example, a person’s initiating actively-entered data about food consumption prior to an eating event, wherein this submission comprises accurate reporting of food to be consumed, is rewarded by enabling the person to avoid a more intrusive prompt for data during the eating event.

In various examples, collection of “level 2” actively-entered data can be more-intrusive, offer less flexibility, and be more time-consuming than collection of “level 1” data. In an example, “level 1” actively-entered data collection may allow considerable flexibility in terms of whether food consumption entries are made before, during, or after eating. Level 2 actively-entered data collection may offer less flexibility in timing. For example, “level 2” data collection may require real-time reporting for maximum accuracy. If the person wants to eat in peace without dealing with real-time data prompts, then they have a strong incentive to provide accurate actively-entered data concerning food consumption in the first data collection cycle.

In various examples, the actively-entered data about food consumption can be obtained from one or more actions selected from the group consisting of: having the person enter the types and portions of food consumed on paper or into an electronic device, having the person manually calculate or estimate calories consumed and record or enter them on paper or into an electronic device. In various examples, human-computer interface options can be selected from the group consisting of: touch screen, keypad, mouse and/or other cursor-moving device, speech or voice recognition, gesture recognition, scanning a bar code or other food code, and taking a picture of food or food packaging.

Passively-collected data and actively-entered data generally have different strengths and weaknesses with respect to estimating caloric intake. Passively-collected data in which sensors automatically monitor a person’s behavior and surrounding space for eating events can be great for compliance and automated analysis of portion size, but can be intrusive. Actively-entered data in which a person uses all of their senses to identify food consumed can be great for accuracy and privacy when the person is 100% compliant with reporting, but 100% long-term compliance with manual diet logging is rare. Combining both passive and actively-entered data collection, in an optimal manner driven by empirical convergence of estimates can provide more accurate measurement of caloric intake than either passively-collected data alone or actively-entered data alone.

In an example, a device and/or system can visually display estimated perimeters, borders, and/or outlines between different types of food in a meal in a person’s field of vision via AR eyewear and prompt a person to confirm or correct these estimated perimeters, border, and/or outlines. In an example, a device and/or system can visually display estimated perimeters, borders, and/or outlines between different types of food in a meal in a person’s field of vision via AR eyewear and prompt a person to confirm or correct these estimated perimeters, border, and/or outlines using a touch screen. In an example, a device and/or system can visually display estimated perimeters, borders, and/or outlines between different types of food in a meal in a person’s field of vision via AR eyewear and prompt a person to confirm or correct these estimated perimeters, border, and/or outlines via speech.

In an example, a device and/or system will prompt a person to activate a sequence of increasingly resource-intensive sensors to provide additional information about nearby food until the type and/or amount of food is identified by the device and/or system with a target minimum level of certainty, accuracy, and/or confidence. In an example, a device and/or system will prompt a person to activate a sequence of increasingly time-consuming sensors to provide additional information about nearby food until the type and/or amount of food is identified by the device and/or system with a target minimum level of certainty, accuracy, and/or confidence.

In an example, a device and/or system will prompt a person to activate a sequence of sensors to provide additional information about nearby food until the type and/or amount of food is identified by the device and/or system with a target minimum level of certainty, accuracy, and/or confidence. In an example, a device and/or system will prompt a person to activate a sequence of increasingly-intrusive sensors to provide additional information about nearby food until the type and/or amount of food is identified by the device and/or system with a target minimum level of certainty, accuracy, and/or confidence.

In an example, a device for measuring a person’s food consumption can use the following feedback method to improve the estimation of types and/or amounts of food consumed by the person: (a) the device analyzes data from one or more sensors (e.g. microphone, optical sensor, and/or camera) worn by the person to determine preliminary values for the types and/or amounts of nearby food; (b) the device communicates these preliminary values to the person via a display screen, computer-generated speech, or the virtual display in AR eyewear; (c) the device receives confirmation or correction of these preliminary values from the person via a touch screen, speech recognition, or a keypad; and (d) the device adjusts these preliminary values to final values as needed based on any corrections of the preliminary values by the person. In an example, this feedback method can occur at the start of a meal, at the end of a meal, or multiple times during a meal.

In an example, a wearable device for measuring a person’s food consumption can further comprise a feedback mechanism involving the following steps: the device makes a preliminary identification of food type based on data from one or more wearable sensors (e.g. a microphone and camera); the device informs the person of the preliminary identification of food type (e.g. via computer-generated speech, a display screen, or a virtual object displayed in AR eyewear); and the device receives confirmation of this food type or correction of this food type from the person (e.g. via speech recognition, touch screen, or keypad).

In an example, a wearable device for measuring a person’s food consumption can further comprise a feedback mechanism involving the following steps: the device makes a preliminary identification of food type based on data from one or more wearable sensors (e.g. an optical sensor and s camera); the device informs the person of the preliminary identification of food type (e.g. via computer-generated speech, a screen, or a virtual object displayed in AR eyewear); and the device receives confirmation of this food type or correction of this food type from the person (e.g. via speech recognition, touch screen, or keypad).

In an example, information from a food-consumption monitoring device that measures a person’s consumption of at least one selected type of food, ingredient, and/or nutrient can be combined with a computer-to-human interface that provides feedback to encourage the person to eat healthy foods and to limit excess consumption of unhealthy foods. In an example, a food-consumption monitoring device can be in wireless communication with a separate feedback device that modifies the person’s eating behavior. In an example, capability for monitoring food consumption can be combined with capability for providing behavior-modifying feedback within a single device. In an example, a single device can be used to measure the selected types and amounts of foods, ingredients, and/or nutrients that a person consumes and to provide visual, auditory, tactile, or other feedback to encourage the person to eat in a healthier manner.

In an example, a device and/or system can notify a person that a camera worn by the person will be automatically activated to begin recording food images when one or more sensors worn by the person detect that the person is eating unless the person takes action to stop this activation. In an example, a device and/or system can notify a person that a camera worn by the person will be automatically activated to begin recording food images when one or more sensors worn by the person detect that the person is eating unless the person takes action to stop this activation within a selected time interval. In an example, recording of images by a camera can stopped by one or more of the following: no eating detected during a recent interval of time (e.g. during the last 3-15 minutes); lack of confirmation that a person is eating in response to a prompt to the person from the device; and/or activation of a stop command and/or button by a person.

In an example, when a device and/or system detects that a person has started eating, the system automatically activates a camera to start recording food images unless the person takes action with a selected interval of time to block this recording. In an example, when a wearable device and/or system detects that a person has started eating, the system automatically activates a wearable camera to start recording food images unless the person takes action with a selected interval of time to block this recording. In an example, when a wearable device and/or system detects that a person has started eating, the system automatically activates a camera on the device and/or system to start recording food images unless the person takes action with a selected interval of time to block this recording.

In an example, when a device with one or more sensors worn by a person detects that the person is eating, then the device can notify the person that a camera worn by the person will be automatically activated to begin recording food images unless the person takes action to stop this activation. In an example, when a device with one or more sensors worn by a person detects that the person is eating, then the device can notify the person that a camera worn by the person will be automatically activated to begin recording food images unless the person takes action to stop this activation within a selected time interval.

In an example, a system can prompt a person to record food images with a camera when a wearable sound sensor detects that the person is eating. In an example, a system can prompt a person to record food images with a camera when a wearable optical sensor detects that the person is eating. In an example, a system can prompt a person to record food images with a camera when a wearable motion sensor detects that the person is eating. In an example, when a device and/or system detects that a person has started eating, the system prompts a person to use a camera to record food images. In an example, when a wearable device and/or system detects that a person has started eating, the system prompts a person to use a wearable camera to record food images. In an example, when a wearable device and/or system detects that a person has started eating, the system prompts a person to use a camera on the device and/or system to record food images. In an example, a device and/or system can prompt a person to activate a camera worn by the person to record food images when one or more sensors worn by the person detect that the person is eating.

In an example, if a camera on a device has a view of nearby food which is obscured and/or insufficient to determine food type and/or amount, then the device can prompt a person to move either the food and/or the device to obtain an un-obscured and/or sufficient view to determine food type and/or amount. In an example, if a camera’s view of nearby food is obscured and/or insufficient to determine food type and/or amount, then the device can prompt a person to move the food and/or their body to obtain an un-obscured and/or sufficient view to determine food type and/or amount. In an example, if a camera on a device has a view of nearby food which is obscured and/or insufficient to determine food type and/or amount, then the device can guide a person to move either the food and/or the device to obtain an un-obscured and/or sufficient view to determine food type and/or amount.

In an example, a “level 2” action can comprise having a person manually focus and trigger (“point and shoot”) a camera toward food that will be consumed. In an example, “level 2” action can comprise having the person respond to a series of menu-driven prompts on a mobile touch screen device in order to precisely identify food that will be, is being, or has been consumed. In an example, a “level 2” action can comprise having the person answer an automatically-generated phone call and give a series of response to voice-prompts in order to precisely identify consumed food.

In an example, a first set of data can comprise image data whose collection requires voluntary actions by the person associated with particular eating events other than the actions of eating and a second set of data can comprise image data whose collection is more continuous than that of the first set of data. In an example, the first set of data comprises image data whose collection is intermittent, periodic, or random, not requiring voluntary actions by the person associated with particular eating events other than the actions of eating, and the second set of data comprises image data whose collection is more continuous than that of the first set of data. In an example, the first set of data comprises image data whose collection is triggered by sounds, motions, or sounds and motions indicating an eating event, not requiring voluntary actions by the person associated with particular eating events other than the actions of eating, and the second set of data comprises image data whose collection is more continuous than that of the first set of data. In an example, the first set of data comprises sound data, motion data, or both sound and motion data and the second set of data comprises image data.

In an example, a method can include use of data sets selected from the group consisting of: (a) at least one of a first set of data and a second set of data comprising image data whose collection requires voluntary actions by the person associated with particular eating events other than the actions of eating and the third set of data comprises image data whose collection is more continuous than the collection of at least one of the first and second sets of data; (b) at least one of the first set of data and the second set of data comprises image data whose collection is intermittent, periodic, or random, not requiring voluntary actions by the person associated with particular eating events other than the actions of eating, and the third set of data comprises image data whose collection is more continuous than the collection of at least one of the first and second sets of data; and (c) at least one of the first set of data and the second set of data comprises image data whose collection is triggered by sounds, motions, or sounds and motions indicating an eating event, this collection does not require voluntary actions by the person associated with particular eating events other than the actions of eating, and the third set of data comprises image data whose collection is more continuous than the collection of at least one of the first and second sets of data.

In an example, a method can include using a first set of data which comprises image data whose collection requires voluntary actions by the person associated with particular eating events other than the actions of eating and a second set of data which comprises image data whose collection is more continuous than that of the first set of data. In an example, initial automatic data collection can be periodic, short-focal-length images from a camera worn by the person, but additional data collection can be continuous, variable-focal-length images from the camera. In an example, the nature of the additional passively-collected data can be different than that of the original passively-collected data. For example, the original passively-collected data can be motion patterns collected from a motion sensor worn by the person, but the additional passively-collected data can be sound patterns from a sound sensor worn by the person. In an example, a first camera can constantly maintain a line of sight to a person’s mouth by constantly shifting the direction and/or focal length of its field of vision. In an example, this first camera can scan and acquire a line of sight to the person’s mouth only when a sensor indicates that the person is eating. In an example, this scanning function can comprise changing the direction and/or focal length of the camera’s field of vision.

In an example, a device can comprise a laser light emitter and a camera, wherein a light beam from the light emitter is used to aim the camera toward food to record food images. In an example, a camera on smart (e.g. AR) eyewear can track a projected laser beam which a person uses to trace boundaries between different types of food in a meal in order to segment an image of the meal into different food portions. In an example, a device can comprise a laser light emitter which emits a light beam which is used to aim the device to capture food images.

In an example, a device can comprise an aiming light beam which projects a square of light. In an example, a device can comprise an aiming light beam which projects a single point of light. In an example, a device can comprise an aiming light beam which projects a grid of light. In an example, a device can comprise an aiming light beam which projects a geometric pattern of light. In an example, a device can comprise an aiming light beam which projects a circle of light. In an example, a device can track a projected laser beam which a person uses to trace boundaries between different types of food in a meal in order to segment an image of the meal into different food portions.

In an example, a device and method can comprise one or more cameras that scan nearby space in order to identify a person’s mouth, hand, and/or reachable food source in response to sensors indicating that the person is probably eating. In an example, one of these cameras: (a) scans space surrounding the camera in order to identify the person’s hand and acquire a line of sight to the person’s hand when a sensor indicates that the person is eating; and then (b) scans space surrounding the person’s hand in order to identify and acquire a line of sight to any reachable food source near the person’s hand. In an example, the device and method may concentrate scanning efforts on the person’s hand at the distal endpoint of a food consumption pathway to detect and identify a nearby food. If the line of sight from this camera to the person’s hand and/or nearby food is subsequently obstructed or otherwise impaired, then a device and method detects and responds as part of its tamper-resisting features. In an example, this response is designed to restore imaging functionality to enable proper automatic monitoring and estimation of caloric intake.

In an example, a device can automatically adjust the imaging vectors or focal lengths of one or more cameras so that these cameras stay focused on a food source and/or the person’s mouth. Even if the line of sight from a camera to a food source, or to the person’s mouth, becomes temporarily obscured, the device can track the last-known location of the food source, or the person’s mouth, and search near that location in space to re-identify the food source, or mouth, to re-establish imaging contact. In an example, the device can track movement of the food source, or the person’s mouth, relative to the camera. In an example, the device can extrapolate expected movement of the food source, or the person’s mouth, and search in the expected projected of the food source, or the person’s mouth, in order to re-establish imaging contact. In various examples, a device and method may use face recognition and/or gesture recognition methods to track the location of the person’s face and/or hand relative to a wearable camera.

In an example, a camera on smart (e.g. AR) eyewear can track the location of the end of a person’s finger as the person traces boundaries between different types of food in a meal in order to segment an image of the meal into different food portions. In an example, a device can track the location of the end of a person’s finger as the person traces boundaries between different types of food in a meal in order to segment an image of the meal into different food portions. In an example, an eyewear-mounted camera can track the location of the end of a person’s finger as the person traces boundaries between different types of food in a meal in order to segment an image of the meal into different food portions.

In an example, a device and/or system can visually display estimated perimeters, borders, and/or outlines between different types of food in a meal and prompt a person to confirm or correct these estimated perimeters, border, and/or outlines by pointing with their finger. In an example, a device and/or system can visually display estimated perimeters, borders, and/or outlines between different types of food in a meal in a person’s field of vision via AR eyewear and prompt a person to confirm or correct these estimated perimeters, border, and/or outlines by pointing with their finger. In an example, an eyewear-mounted camera can track a projected laser beam which a person uses to trace boundaries between different types of food in a meal in order to segment an image of the meal into different food portions.

In an example, a device can comprise a hand and/or finger motion user interface. In an example, a device worn by a person can comprise a camera and a microphone, wherein the system prompts the person to sequentially point at different types of food, wherein the system prompts the person to sequentially vocally identify the different types of food as the person points to them, wherein images recorded by the camera are analyzed to track the location of the person’s finger, wherein sounds recorded by the microphone are analyzed to track the vocal identifications of food types by the person, and wherein the system associates areas of the images with vocal identifications of different food types as part of measuring the types and/or amounts of food in a meal. In an example, a device worn by a person can comprise a camera and a microphone, wherein the system tracks the location a person’s finger as the person points at different types of food in a meal, wherein the system tracks the person’s speech-based identification of the different types of food in the meal as the person’s points at them, and wherein the system uses this information in estimating the types and/or amounts of different foods in a meal.

In an example, a device worn by a person can comprise a camera and a microphone, wherein the system uses the camera to track the location a person’s finger as the person points at different types of food in a meal, wherein the system uses the microphone to record the person’s speech-based identification of the different types of food in the meal as the person’s points at them, and wherein the system associates different areas of a food image with different speech-based food identifications to estimate the types and/or amounts of different foods in a meal. In an example, a wearable device can comprise a camera and a microphone, wherein the system analyzes images recorded by the camera to track the location of a person’s finger, wherein the person sequentially points to different types of food in a meal, wherein the person verbally identifies the different types of food in the meal as they point at them, wherein the records the person’s verbal identifications of food types using the microphone, wherein the system associates the person’s verbal identification of different types of food with wherein those types of food are in the recorded images.

There are open-ended or menu-driven human-computer interfaces which can be used for actively-entered data collection. In various examples, an interface can comprise a touch screen, voice commands and recognition, key pad, gesture recognition, eye movements, or a variety of other modes of human-computer interaction.

In an example, a device can comprise a voice-based user interface. In an example, a device can comprise a speech recognition user interface. In an example, a device can comprise a speech and/or voice recognition system. In an example, a device can evaluate a person’s stress level by analysis of the person’s voice. In an example, a system can prompt a person to verbally identify food when a wearable motion sensor detects that the person is eating.

In an example, a device can comprise a microphone and a speaker which function as a two-way voice-based user interface. In an example, a microphone and speaker unit can emit voice-based messages that are heard by the person wearing the device and this unit also receives voice-based messages from this person. In an example, data processing and transmission unit can includes voice generation and voice recognition software. In an example, a microphone and speaker unit can be used to prompt the person wearing the device to enter actively-entered data concerning food consumption. In an example, microphone and speaker unit can be used to receive actively-entered data (in voice form) concerning food consumption from this person. In an example, a device can send messages to the person in voice form, but receive data from the person in another form such as through a keypad or touch screen. In other examples, a device can send messages to the person in non-voice form, such as a display screen, but receive messages from the person in voice form.

In an example, a device can include a data processing and transmission unit which estimates a person’s caloric intake. This estimation of caloric intake can be based on passively-collected data concerning food consumption collected by a motion sensor, actively-entered data concerning food consumption received via the person’s voice, or both passively-collected data and actively-entered data. In an example, an estimate of the person’s caloric expenditure is subtracted from the estimate of the person’s caloric intake in order to calculate the person’s net energy balance and to predict the person’s weight gain or loss. In an example, the estimate of caloric expenditure can come from a different device and be transmitted wirelessly to a data processing and transmission unit.

In an example, a device can prompt a person with clarifying questions concerning the types and quantities of food that person has consumed. These questions can be asked in real time, as a person eats, at a subsequent time, or periodically. In an example, a device and method may prompt the person with queries to refine initial automatically-generated estimates of the types and quantities of food consumed. Automatic estimates can be refined by interaction between the device and the person.

In an example, a method can comprise a first cycle of data collection in which actively-entered data about the person’s food consumption is received from “level 1” action performed by the person. In an example, a “level 1” action can comprise having the person make a voice entry into a device, wherein this voice entry briefly describes food that will be, is being, or has been consumed. In an example, a “level 1” action can comprise having the person enter data about food consumption via a menu-driven, touch-screen-activated user interface. In an example, a “level 1” action can comprise having the person scan a bar code (or other identifying code) on the packaging of food that will be, is being, or has been consumed. In an example, a “level 1” action can comprise pattern recognition of a logo, other design, or wording on the food packaging.

In an example, a motion sensor can detect a possible eating event (i.e. a glass being tilted up to the mouth and then back down) and this event can trigger a voice-based inquiry from the device to the person via a microphone and speaker unit. In an example, the device, upon detection of a probable eating event, can ask the person a question such as -- “If you are eating something, please identify it.” In an example, a device can solicits actively-entered data concerning food consumption from the person through a voice-based message. In other examples, a device can solicit actively-entered data via other means such as a display screen, buzzing or ring tone, vibration, or text message.

In an example, a person can respond to a voice-based prompt with voice-based data concerning what the person is eating. In an example, the person might respond to a device inquiry with the statement --“I am drinking a large glass of apple juice.” The person’s voice-based response can be received by a microphone and speaker unit. In an example, the voice-based response that is received by the microphone and speaker unit can be analyzed and understood by a data processing and transmission unit. In an example, the voice-based response can be transmitted to, and analyzed by, a remote computer. In an example, voice recognition or speech recognition software can be used to analyze the voice-based response.

In an example, a device can further comprise a button and/or vibration sensor which a person taps to activate a sensor and/or a camera to record eating data. In an example, a device can further comprise a button and/or vibration sensor which a person taps to activate a sensor and/or a camera to record and analyze eating data. In an example, a device can further comprise a button and/or vibration sensor which a person taps to activate a sensor and/or a camera to record and analyze data concerning food consumption. In an example, a device can further comprise a button and/or vibration sensor which a person taps to activate a sensor and/or a camera to record and analyze data concerning a meal. In an example, a device can further comprise a button and/or vibration sensor which a person taps to activate a sensor and/or a camera to record an eating event.

In an example, if images of other people are identified in images, these images can be deleted after food information has been extracted from the images. In an example, if images of other people are identified in images, then the portions of those images which show other people can be blurred, blacked out, or cropped out. In an example, recorded sounds and/or recorded images from a wearable device are erased with a selected time interval (e.g. 1-10 minutes) if no eating is detected and/or if eating stops.

In an example, recorded sounds and/or images from a wearable device are erased with a selected time interval (e.g. 1-10 minutes) after a person stops eating unless the person gives permission for those recorded sounds and/or images to be retained in memory. In an example, recorded sounds and/or recorded images from a wearable device are erased with a selected time interval (e.g. 1-10 minutes) after a person stops eating. In an example, device can continuously record sounds, but automatically erase sounds after short period of time if no eating detected. In an example, device can continuously record sounds, but automatically erase sounds after short period of time (e.g. less than 2 minutes) for privacy purposes if no eating detected.

In an example, recorded sounds and/or recorded images from a wearable device are only transmitted to a remote data processor if permission is given by the person wearing the device. In an example, data from one or more chewing and/or swallowing sensors can go through a high-pass filter. In an example, signals from a chewing and/or swallowing sensor can be sent through low pass filter. In an example, signals from a sensor can be amplified. In an example, signals from a sensor can be filtered. In an example, signals from a sensor can be truncated.

Continuous video imaging of the space surrounding a person, especially space near the person’s mouth and hands, is likely to provide relatively accurate monitoring of food consumption. However, continuous video imaging of the space surrounding a person, including whatever or whoever enters that space, can be relatively intrusive. Some approaches in the prior art that rely on continuous video imaging seek to address privacy concerns by having automated screening mechanisms that screen out images of people or things that would infringe on privacy. Devices and methods described herein can include automated screening mechanisms to enhance privacy. Having a wearable device that takes pictures all the time can raise privacy concerns. Having a device that continually takes pictures of a person’s mouth and continually scans space surrounding the person for potential food sources can be undesirable in terms of privacy, excessive energy use, or both.

In an example, a method for measuring a person’s caloric intake can comprise: (a) receiving a first set of data from a first source concerning what the person eats, wherein collecting this first set of data requires a first level of intrusion into the person’s privacy or time, and receiving a second set of data from a second source concerning what the person eats, wherein collecting this second set of data requires a second level of intrusion into the person’s privacy or time; (b) calculating a first estimate of the person’s caloric intake based on the first set of data, calculating a second estimate of the person’s caloric intake based on the second set of data, and comparing these first and second estimates of caloric intake to determine whether these estimates meet criteria for similarity and/or convergence; and (c) if the first and second estimates of caloric intake do not meet the criteria for similarity and/or convergence, then receiving a third set of data concerning what the person eats, wherein collection of this third set of data requires a third level of intrusion into the person’s privacy or time, and wherein this third level is greater than the first level and is greater than the second level, and calculating a third estimate of caloric intake based, in whole or in part, on this third set of data.

In an example, a method for measuring a person’s caloric intake can comprise: (a) receiving a first set of data concerning what the person eats, wherein collecting this first set of data requires a first level of intrusion into the person’s privacy or time; (b) calculating a first estimate of the person’s caloric intake based on the first set of data, using this first estimate of the person’s caloric intake to estimate predicted weight change for the person during a period of time, and comparing predicted weight change to actual weight change to determine whether predicted weight change and actual weight change meet criteria for similarity and/or convergence; and (c) if predicted weight change and actual weight change do not meet the criteria for similarity and/or convergence, then receiving a second set of data concerning what the person eats, wherein collection of this second set of data requires a second level of intrusion into the person’s privacy or time that is greater than the first level, and calculating a second estimate of caloric intake based, in whole or in part, on this second set of data.

In an example, a sound sensor can be more intrusive than a motion sensor with respect to possibly recording conversations or other sounds that could intrude on privacy, but is not as intrusive as a camera with respect to possibly recording people or other images that could intrude on privacy. In an example, speech recognition software could be used to explicitly filter out the recording of human speech in the interest of privacy, while still recording chewing, biting, and swallowing sounds in order to identify the types and quantities of food consumed.

In an example, one way to design a device and method to take pictures when a person eats without the need for human intervention is to simply have the device take pictures continuously. If the device is never turned off and takes pictures all the time, then it necessarily takes pictures when a person eats. In an example, such a device and method can: continually track the location of, and take pictures of, the person’s mouth; continually track the location of, and take pictures of, the person’s hands; and continually scan for, and take pictures of, any reachable food sources nearby. However, this can cause privacy problems.

In an example, the focal direction and focal range of such a wearable camera can be chosen so as to capture images of food consumed, while minimizing privacy-intruding images. In an example, pattern recognition can be used to automatically blur out privacy-intruding images (such as images of other people) by adjusting focal range in real time. In an example, a first set of data can comprise image data whose collection is triggered by sounds, motions, or sounds and motions indicating an eating event, not requiring voluntary actions by the person associated with particular eating events other than the actions of eating, and a second set of data can comprise image data whose collection is more continuous than that of the first set of data.

In an example, a device can be part of a system which identifies causal relationships between a person’s consumption of particular types and/or quantities of food and subsequent changes in the person’s body weight and/or shape. In an example, a system can analyze the relationship between a person’s caloric expenditure and the person’s consumption of different types and/or amounts of food. In an example, a system can analyze the relationship between a person’s exercise level and the person’s consumption of different types and/or amounts of food. In an example, a system can analyze the relationship between a person’s posture and the person’s consumption of different types and/or amounts of food. In an example, a system can analyze the relationship between a person’s internet activity and the person’s consumption of different types and/or amounts of food.

In an example, a device can store information concerning food (preference) rankings. In an example, a system can comprise a database which links types of foods to types of nutrients. In an example, a system can comprise a database which links types of foods to nutritional compositions. In an example, a system can comprise a database with information on the average ratios of different types of nutrients in different types of food. In an example, a system can comprise a database with information on the average nutritional compositions of different types of food. In an example, a system for measuring the types and/or amounts of nutrients consumed by a person can include a database which associates types of foods with types of nutrients.

In an example, a system for measuring the types and/or amounts of nutrients consumed by a person can include a database which includes standard, average, and/or baseline nutritional compositions for different types of foods. In an example, information concerning the types and amounts of food consumed by a person can be converted into types and amounts of nutrients using a database which associates specific foods with specific nutrients. In an example, a system for measuring the types and/or amounts of nutrients consumed by a person can include a database which associates different types of food with different nutritional compositions. In an example, information concerning the types and amounts of food consumed by a person can be converted into types and amounts of nutrients using a database concerning the nutritional composition of different types of food.

In an example, an estimation process can include automated pattern recognition and analysis of voluntarily-entered images in order to identify food types and quantities. In an example, a database of types of food (and portions) and their associated calories can be used to convert types and quantities of food into calories. In an example, estimation of caloric intake can be done by a data processing device such as a computer. In an example, estimation of the number of calories consumed by a person can be done, in whole or in part, by using a standardized database that associates certain types and quantities of food with certain calorie values. In an example, estimation of the number of calories consumed by the person can be done, in whole or in part, by predicting the calories associated with particular foods or meals based on the person’s historical eating patterns. For example, if the person tends to consume large portions of a particular food, then this is taken into account when estimating calories.

In an example, identification of the types and quantities of food consumed by a person can be done, in whole or in part, by using a standardized database that associates certain patterns of output from passively-collected data sensors with consumption of certain types and quantities of food. In an example, estimation of the number of calories consumed by the person can be done, in whole or in part, by using a standardized database that associates certain types and quantities of food with certain calorie values.

In an example, information concerning the types and quantities of food consumed is used to estimate caloric intake. In an example, a standard database of the calories associated with various types of food, and portions thereof, can be used to convert information about the types and quantities of food consumed into an estimate of caloric intake. In an example, a customized database specific to an individual can be created based on the person’s past eating habits. In an example, an estimate of caloric intake can be estimated directly from raw passively-collected data received without the need for an intermediate step involving identifying specific types and quantities of food consumed. In an example, an estimate of caloric intake can be for a particular eating event, such as a specific meal or snack. In an example, an estimate of caloric intake can be for a specific period of time such as a day, week, or month.

In an example, a device can identify consumption of at least one selected type of food. In such an example, selected types of ingredients or nutrients can be estimated indirectly using a database that links common types and amounts of food with common types and amounts of ingredients or nutrients. In an example, a device can directly identify consumption of at least one selected type of ingredient or nutrient. The latter does not rely on estimates from a database, but does require more complex ingredient-specific or nutrient-specific sensors. Various embodiments of the device and method disclosed herein can identify specific nutrients indirectly (through food identification and use of a database) or directly (through the use of nutrient-specific sensors).

In an example, a device can identify types and/or amounts of food in an image based on the size and/or shape of a container, package, or dish in which (or on which) the food is located and/or served. In an example, a device can identify types and/or amounts of food in an image based on the design of a container, package, or dish in which (or on which) the food is located and/or served. In an example, a device can analyze food images to identify logos, brands, or other product-identifying images and/or words on food packaging, labels, boxes, bottles, and/or containers.

In an example, a device can identify types and/or amounts of food in an image based on a logo, wording, picture, or digital code on a container, package, or dish in which (or on which) the food is located and/or served. In an example, estimation of the types and/or amounts of food in an image can be informed by identification of labels and/or logos of standardized (e.g. brand) packaged foods. In an example, estimation of the types and/or amounts of food in an image can be informed by identification of labels and/or logos of foods sold in standardized containers (e.g. standard size boxes, bottles, and cans). In an example, a device can identify types and/or amounts of food in an image based on food texture. In an example, a device can identify types and/or amounts of food in an image based on food size. In an example, a device can identify types and/or amounts of food in an image based on food shape. In an example, a device can identify types and/or amounts of food in an image based on food color. In an example, a device can transform and/or convert an analog image into a digital signal.

In an example, a system can analyze the color, texture, shape, size, temperature, and/or molecular composition of food items in images recorded by a wearable camera to identify the types and/or amounts of foods in those images. In an example, a system can analyze the color, texture, shape, size, temperature, and/or infrared spectral distribution of food items in images recorded by a wearable camera to identify the types and/or amounts of foods in those images. In an example, a system can analyze the color, texture, shape, size, and/or temperature of food items in images recorded by a wearable camera to identify the types and/or amounts of foods in those images. In an example, a system can analyze the color, texture, shape, and/or size of food items in images recorded by a wearable camera to identify the types and/or amounts of foods in those images.

In an example, a system can analyze the color, texture, shape, size, volume, temperature, moisture content, infrared spectral distribution, and/or geographic context of food items in images recorded by a wearable camera to identify the types and/or amounts of foods in those images. In an example, a system can analyze the color, texture, shape, size, volume, temperature, moisture content, and/or infrared spectral distribution of food items in images recorded by a wearable camera to identify the types and/or amounts of foods in those images. In an example, a system can analyze the color, texture, shape, size, volume, temperature, and/or infrared spectral distribution of food items in images recorded by a wearable camera to identify the types and/or amounts of foods in those images.

In an example, food images can be analyzed to estimate the amount of food per spoonfull. In an example, food images can be analyzed to estimate the amount of food per hand-to-mouth motion. In an example, food images can be analyzed to estimate the amount of food per forkfull. In an example, food images can be analyzed to estimate the amount of food per bite. In an example, data from sensors at different times and food images at different times can be analyzed to group chewing and/or swallowing motions together into an eating event. In an example, a food image can be assigned a time stamp.

A device and method that incorporates pictures of both a food source and the person’s mouth, while a person eats, can provide much more accurate than prior art that takes pictures of only a food source or only the person’s mouth. The wearable nature of a device makes it less reliant on manual activation, and much more automatic in its imaging operation, than non-wearable devices. In an example, a device need not depend on properly placing, aiming, and activating a camera every time a person eats. It operates in an automatic manner and is tamper resistant. All of these features combine to make a device a more accurate and dependable device and method of monitoring and measuring human caloric intake than devices and methods in the prior art.

Food is broadly defined herein to include beverages as well as solid and semi-solid food. In an example, images of food or food containers can be automatically analyzed to estimate the types and quantities of food consumed. In addition to the color, texture, and volume of food itself, features of food containers and packages may also be analyzed to identify food. For example, if a hand is holding a beverage can, then the image analysis might recognize a logo on the can in order to identify the beverage. In an example, a device and method can include at least one image-analyzing member. This image-analyzing member automatically analyzes pictures of a person’s mouth and pictures of nearby food in order to estimate the types and quantities of food consumed by this person. This is superior to prior art that only analyzes pictures of nearby food because the person might not actually consume all of the food at this food source.

In an example, a device and method for measuring caloric intake can comprise one or more cameras that are worn on a person at one or more locations from which these cameras automatically take (still or motion) pictures of the person’s mouth as the person eats and automatically take (still or motion) pictures of nearby food as the person eats. In an example, these images are automatically analyzed to estimate the types and quantities of food actually consumed by the person. In an example, a device can be entirely automatic for both food imaging and food identification. In an example, a device and method can automatically and comprehensively analyze images of food sources and a person’s mouth in order to provide final estimates of the types and quantities of food consumed. In an example, the food identification and quantification process performed by a device and method does not require any manual entry of information, any manual initiation of picture taking, or any manual aiming of a camera when a person eats. In an example, a device and method automatically analyzes images to estimate the types and quantities of food consumed without the need for real-time or subsequent human evaluation.

In an example, a device can for automatically monitoring caloric intake can comprise: one or more cameras that are worn on one or more locations on a person from which these cameras: collectively and automatically take pictures of the person’s mouth when the person eats and pictures of nearby food when the person eats; wherein nearby food is a food source that the person can reach by moving their arm; and wherein food can include liquid nourishment as well as solid food; a tamper-resisting mechanism which detects and responds if the operation of the one or more cameras is impaired; and an image-analyzing member which automatically analyzes pictures of the person’s mouth and pictures of a reachable food source in order to estimate the types and quantities of food that are consumed by the person.

In an example, a device can for automatically monitoring caloric intake can comprise: having a person wear one or more cameras at one or more locations on the person from which these cameras collectively and automatically take pictures of the person’s mouth when the person eats and pictures of nearby food when the person eats; wherein nearby food is a food source that the person can reach by moving their arm; and wherein food can include liquid nourishment as well as solid food; detecting and responding if the operation of the one or more cameras is impaired; and automatically analyzing pictures of the person’s mouth and pictures of a reachable food source in order to estimate the types and quantities of food that are consumed by the person.

In an example, a device can identify the types and quantities of food consumed based on: pattern recognition of food at a nearby food; changes in food at that source; analysis of images of food traveling along a food consumption pathway from a food source to the person’s mouth; and/or the number of cycles of food moving along a food consumption pathway. In various examples, food can be identified by pattern recognition of food itself, by recognition of words on food packaging or containers, by recognition of food brand images and logos, or by recognition of product identification codes (such as “bar codes”). In an example, analysis of images by a device and method occurs in real time, as the person is consuming food. In an example, analysis of images by a device and method occurs after the person has consumed food.

In an example, a device includes an image-analyzing member that analyzes one or more factors selected from the group consisting of: number of reachable food sources; types of reachable food sources; changes in the volume of food at a nearby food; number of times that the person brings food to their mouth; sizes of portions of food that the person brings to their mouth; number of chewing movements; frequency or speed of chewing movements; and number of swallowing movements.

In an example, a device that automatically monitors caloric intake can comprise: one or more cameras that are worn on one or more locations on a person from which these cameras: collectively and automatically take pictures of the person’s mouth when the person eats and pictures of nearby food when the person eats; wherein nearby food is a food source that the person can reach by moving their arm; and wherein food can include liquid nourishment as well as solid food; a tamper-resisting mechanism which detects and responds if the operation of the one or more cameras is impaired; and an image-analyzing member which automatically analyzes pictures of the person’s mouth and pictures of a reachable food source in order to estimate the types and quantities of food that are consumed by the person.

In an example, a method for automatically monitoring caloric intake can comprise: (a) having a person wear one or more cameras at one or more locations on the person from which these cameras collectively and automatically take pictures of the person’s mouth when the person eats and pictures of nearby food when the person eats; wherein nearby food is a food source that the person can reach by moving their arm; and wherein food can include liquid nourishment as well as solid food; (b) detecting and responding if the operation of the one or more cameras is impaired; and (c) automatically analyzing pictures of the person’s mouth and pictures of a reachable food source in order to estimate the types and quantities of food that are consumed by the person.

In an example, a method for automatically monitoring caloric intake can comprise: having a person wear one or more cameras at one or more locations on the person from which these cameras: collectively and automatically take pictures of the person’s mouth when the person eats and pictures of nearby food when the person eats; wherein nearby food is a food source that the person can reach by moving their arm; and wherein food can include liquid nourishment as well as solid food; detecting and responding if the operation of the one or more cameras is impaired; and automatically analyzing pictures of the person’s mouth and pictures of a reachable food source in order to estimate the types and quantities of food that are consumed by the person.

In an example, a reachable food source can be food in a bowl. In other examples, a reachable food source can be selected from the group consisting of: food on a plate, food in a bowl, food in a glass, food in a cup, food in a bottle, food in a can, food in a package, food in a container, food in a wrapper, food in a bag, food in a box, food on a table, food on a counter, food on a shelf, and food in a refrigerator. In an example, a system can comprise wireless communication from a first wearable member (that takes pictures of nearby food and a person’s mouth) to a second wearable member (that analyzes these pictures to estimate the types and quantities of food consumed by the person). In an example, a device can include wireless communication from a wearable member (that takes pictures of nearby food and a person’s mouth) to a non-wearable member (that analyzes these pictures to estimate the types and quantities of food consumed by the person). In an example, a device can include a single wearable member that takes and analyzes pictures, of nearby food and a person’s mouth, to estimate the types and quantities of food consumed by the person.

In an example, an image-analyzing member can use one or more methods selected from the group consisting of: pattern recognition or identification; human motion recognition or identification; face recognition or identification; gesture recognition or identification; food recognition or identification; word recognition or identification; logo recognition or identification; bar code recognition or identification; and 3D modeling. In an example, images of food can be automatically analyzed to estimate the types and quantities of food consumed. In various examples, analysis and identification of food and/or food packaging can include one or more methods selected from the group consisting of: food recognition or identification; visual pattern recognition or identification; chemical recognition or identification; smell recognition or identification; word recognition or identification; logo recognition or identification; bar code recognition or identification; and 3D modeling. The results of this new video data and analysis are then used to improve the accuracy of caloric intake estimation. In an example, pattern recognition software can identify the type of food at nearby food by: analyzing the food’s shape, color, texture, and volume; or by analyzing the food’s packaging.

In an example, when a is sitting at a table with many other diners wherein the table is set with food in family-style communal serving dishes, these family-style dishes can be passed around to serve food to everyone around the table. It would be challenging for a “source-only” camera to automatically differentiate between these communal serving dishes and a person’s individual plate. What happens when the person’s plate is removed or replaced? What happens when the person does not eat all of the food on their plate? These examples highlight the limitations of a device and method that only takes pictures of a nearby food, without also taking pictures of the person’s mouth.

In various examples, a device comprises one or more image-analyzing members that analyze one or more factors selected from the group consisting of: number and type of reachable food sources; changes in the volume of food observed at a nearby food; number and size of chewing movements; number and size of swallowing movements; number of times that pieces (or portions) of food travel along a food consumption pathway; and size of pieces (or portions) of food traveling along a food consumption pathway. In various examples, one or more of these factors can be used to analyze images to estimate the types and quantities of food consumed by a person. In various examples, analysis and identification of food or food packaging can include one or more methods selected from the group consisting of: food recognition or identification; visual pattern recognition or identification; chemical recognition or identification; smell recognition or identification; word recognition or identification; logo recognition or identification; bar code recognition or identification; and 3D modeling.

In various examples, one or more methods to analyze pictures, in order to estimate the types and quantities of food consumed, can be selected from the group consisting of: pattern recognition; food recognition; word recognition; logo recognition; bar code recognition; face recognition; gesture recognition; and human motion recognition. In various examples, a picture of the person’s mouth and/or nearby food can be analyzed with one or more methods selected from the group consisting of: pattern recognition or identification; human motion recognition or identification; face recognition or identification; gesture recognition or identification; food recognition or identification; word recognition or identification; logo recognition or identification; bar code recognition or identification; and 3D modeling. In an example, images of a person’s mouth and nearby food can be taken from at least two different perspectives in order to enable the creation of three-dimensional models of food.

Integrated analysis of pictures of both the food source and the person’s mouth can provide a relatively accurate estimate of the types and quantities of food actually consumed by this person, even in situations with multiple food sources and multiple diners. Integrated analysis can compare estimates of food quantity consumed based on changes in observed food volume at the food source to estimates of food quantity consumed based on mouth-food interaction and food consumption pathway cycles. With images of both nearby food and the person’s mouth, as the person eats, a device and method can determine not only what food the person has access to, but how much of that food the person actually eats.

In an example, a device can segment an image of a meal into portions of different types of food. In an example, a device can use pattern recognition to segment an image of a meal into portions of different types of food. In an example, a device can identify a food in an image of a meal based (at least in part) on identification of other foods in the meal. In an example, a device can identify a type of food in an image of a meal based (at least in part) on identification of other types of food in the meal because certain types of food tend to be served together in the same meal.

In an example, a device can evaluate a person’s stress level by analysis of the person’s voice and correlate their stress level with the types and/or amounts of food consumed by that person. In an example, the amounts and/or types of food consumed can be estimated by analyzing the correlation between chewing sounds and swallowing sounds during an eating event. In an example, a system for measuring a person’s food and/or nutritional consumption can use Bayesian statistical methods to estimate types and/or amounts of food consumed by a person, prompt the person to confirm or correct those estimated types and/or amounts, and then update future estimates based on the person’s response. In an example, a device worn by a person can comprise sensors wherein data from the sensors is analyzed using Principle Component analysis (PCA) to estimate the types and/or amounts of food and/or nutrients consumed by the person.

In an example, a system can provide a person with recommendations concerning food and/or activities based on a correlation (or other statistical relationship) between biometric indicators of a person’s stress level and the types and/or amounts of food that the person’ consumes which is identified by the system. In an example, a system can identify a correlation (or other statistical relationship) between biometric indicators of a person’s stress level and the types and/or amounts of food that the person’ consumes. In an example, a series of cyclical jaw motions can be grouped using statistical methods in order to identify a meal and/or eating event. In an example, food images can be analyzed using Principal Component Analysis (PCA).

In an example, caloric intake can be estimated by combining passively-collected data and actively-entered data using weights from a multivariate linear estimation model; using a Bayesian statistical model; using linear or non-linear mathematical programming; or using other multivariate statistical methods. In an example, weights can be standardized based on empirical evidence from a large population. In an example, weights can be customized to a specific individual based on the individual’s own eating habits, sensor output patterns, and diet logging behavior.

In an example, caloric intake can be estimated by combining passively-collected data and actively-entered data concerning food consumption: using weights from a multivariate linear estimation model; using weights from a Bayesian statistical model; using linear or non-linear mathematical programming; or using other multivariate statistical methods. In an example, these weights can be standardized, based on empirical evidence from many people over multiple time periods. In an example, these weights can be customized to a particular individual, based on the individual’s unique history of eating habits, sensor monitoring, and diet logging. In an example, criteria for similarity and/or convergence between two estimates are selected from the group consisting of: raw difference between two values is not greater than a target value; percentage difference between two values is not greater than a target value; mathematical analysis of paired variables predicts convergence between them; and statistical analysis of two variables does not show a statistically-significant difference between them.

In an example, the criteria for similarity and/or convergence of two estimates of caloric intake can be based on projected mathematical and/or statistical models. For example, mathematical convergence can be identified based on a series of paired estimates from passive and actively-entered data over time. In an example, paired estimates from passive and actively-entered data over time can come from a series of cycles. In an example, convergence criteria can be based on projected mathematical and/or statistical convergence of these two estimates. In an example, criteria for similarity and/or convergence of these two estimates can be based on overlapping statistical confidence intervals or statistical hypothesis testing.

In an example, the relative weights given to different data elements in an estimation process or the structure of the estimation model can be modified. In an example, the relative weights given to historical vs. current data in an estimation model can be adjusted. In an example, modification of the estimation process may use Bayesian statistical methods. In an example, modification of the estimation process may use nonlinear mathematical programming or optimization methods. In an example, modification of the estimation process can include goal-directed changes. In an example, modification of the estimation process can include randomized, non-goal-directed changes.

In an example, a device can be part of a system which uses time-series statistical methods to identify a time-lagged relationship between a person’s net caloric balance (e.g. cumulative caloric intake minus cumulative caloric expenditure) and subsequent changes in the person’s weight. In an example, a device can be part of a system which uses time-series statistical methods to identify a time-lagged relationship between a person’s net caloric balance (e.g. cumulative caloric intake minus cumulative caloric expenditure) and subsequent changes in the person’s body shape. In an example, a device can be part of a system which uses time-series statistical methods to identify a time-lagged relationship between a person’s net caloric balance (e.g. cumulative caloric intake minus cumulative caloric expenditure) and subsequent changes in the person’s health status. In an example, signals from a sensor can be analyzed using time-frequency methods. In an example, signals from a sensor can be analyzed using time-frequency decomposition methods.

In an example, a system can analyze the relationship between the time of day (or night) and a person’s consumption of different types and/or amounts of food. In an example, a system can analyze the relationship between lengths of time between meals and a person’s consumption of different types and/or amounts of food. In an example, a system can analyze the relationship between annual seasons and a person’s consumption of different types and/or amounts of food. In an example, a system can analyze the relationship between a person’s number of hours worked and the person’s consumption of different types and/or amounts of food.

In an example, a system can use data from temporal factors to estimate the types and/or amounts of food consumed by a person wherein this data can include one or more variables selected from the group consisting of: day of the week, season of the year, and time of day. In an example, a device and/or system can analyze eating behavior to identify specific times of the year when a person is more susceptible to eating unhealthy types and/or amounts of food. In an example, a device and/or system can analyze eating behavior to identify specific times of the week when a person is more susceptible to eating unhealthy types and/or amounts of food. In an example, a device and/or system can analyze eating behavior to identify specific times of the week when a person is more susceptible to eating unhealthy types and/or amounts of food. In an example, a device and/or system can analyze eating behavior to identify specific times of the day when a person is more susceptible to eating unhealthy types and/or amounts of food. In an example, a device can be part of a system which tracks cumulative consumption of different types of nutrients during a period of time (e.g. day, week, or month).

In an example, a method can include a first set of data comprising image data whose collection is intermittent, periodic, or random, not requiring voluntary actions by the person associated with particular eating events other than the actions of eating, and a second set of data comprising image data whose collection is more continuous than that of the first set of data. In an example, a motion-triggered camera can take video images for a set interval of time after analysis of output from the continually-operating motion sensor suggests that the person is eating. In an example, a motion-triggered camera may start taking pictures based on output from a motion sensor and may continue operation for as long as eating continues, wherein eating is determined based on the results of the motion sensor, the camera, or both. If analysis of images from the camera shows that the indication of probable eating by the motion sensor was a false alarm, then the camera can stop taking pictures. In an example, if the camera determines that a food source within view or reach of the person remains unfinished, then the camera may continue to take pictures even if motion stops for a period of time.

In an example, identification of the types and quantities of food consumed by the person can be done, in whole or in part, by predicting a person’s current eating patterns based on the person’s historical eating patterns. For example, if the person tends to eat a particular type of food at a particular time of day in a particular location, then this can be taken into account when identifying food consumed. In an example, estimation of the number of calories consumed by the person can be done, in whole or in part, by predicting the calories associated with particular foods or meals based on the person’s historical eating patterns. For example, if the person tends to consume larger-than-standard portions of a particular food, then this can be taken into account when estimating calories. In an example, the duration of imaging by a camera can depend on the strength of the probability indication that eating is occurring. If the results from one or more sensors indicate, with a high level of certainty, that eating is occurring, then the camera may operate for a longer period of time. If the results from one or more sensors are less certain with respect to whether the person is eating, then the camera may operate for a shorter period of time.

In an example, a device can comprise a light emitter (e.g. LED) which emits infrared or near-infrared light beams toward a person’s head and a light receiver (e.g. photodiode) which receives those light beams after they have been reflected back from the person’s head, wherein Fourier Transformation analysis of variation in the reflected light beams over time is used to detect, monitor, and/or measure chewing motions by the person. In an example, a device can comprise a light emitter (e.g. LED) which emits infrared or near-infrared light beams toward a person’s head and a light receiver (e.g. photodiode) which receives those light beams after they have been reflected back from the person’s head, wherein Fourier Transformation analysis of variation in the magnitude of the reflected light beams is used to detect, monitor, and/or measure chewing motions by the person.

In an example, a device can track changes and/or temporal variation in the speed, rate, frequency, and/or pace of eating-related motions using Fourier Transformation. In an example, a device can track changes and/or temporal variation in the speed, rate, frequency, and/or pace of hand-to-mouth motions related to eating using Fourier Transformation. In an example, a device can track changes and/or temporal variation in the speed, rate, frequency, and/or pace of chewing and/or swallowing motions using Fourier Transformation. In an example, a device can track the speed, rate, frequency, and/or pace of eating-related motions using Fourier Transformation. In an example, a device can track the speed, rate, frequency, and/or pace of hand-to-mouth motions related to eating using Fourier Transformation. In an example, a device can track the speed, rate, frequency, and/or pace of chewing and/or swallowing motions using Fourier Transformation. In an example, changes in the rate and/or frequency of jaw motion can be analyzed using Fourier Transformation.

In an example, a device can use Fourier Transformation to analyze a person’s hand-to-mouth motions during an eating event and provide feedback to the person. In an example, a device can use Fourier Transformation to analyze a person’s food consumption during an eating event and provide feedback to the person. In an example, a device can use Fourier Transformation to analyze a person’s chewing and/or swallowing motions during an eating event and provide feedback to the person. In an example, a sensor can detect repeating and/or cyclical patterns in skin deformation and/or stretching which indicate eating. In an example, a sensor can detect repeating and/or cyclical patterns which indicate eating.

In an example, Fourier analysis of chewing sound patterns can be used identify the amounts and types of food being eaten. In an example, a device can be part of a system which analyzes the number and frequency of chewing motions using Fourier Transformation. In an example, a device can be part of a system which analyzes the number and frequency of chewing sounds using Fourier Transformation. In an example, a device can be part of a system which analyzes the number and frequency of swallowing motions using Fourier Transformation. In an example, a device can be part of a system which analyzes the number and frequency of swallowing sounds using Fourier Transformation. In an example, a device can measure the nutritional composition of food by applying Fourier Transformation to radar signals reflected from food.

In an example, signals from a sensor can be analyzed using Fourier-based methods. In an example, signals from a sensor can be analyzed using Fourier Transformation. In an example, signals from one or more eating detection sensors can be analyzed using Fourier Transformation. In an example, signals from a sensor can be tracked over time, using Fourier Transformation. In an example, deformation of tissue on or near a person’s jaw can be analyzed using Fourier Transformation to identify chewing and/or swallowing motions. In an example, the rate and/or frequency of jaw motion can be analyzed using Fourier Transformation. In an example, deformation of tissue on or near a person’s jaw measured by an optical sensor can be analyzed using Fourier Transformation to identify chewing and/or swallowing motions. In an example, deformation of tissue on or near a person’s jaw measured by a strain or stretch sensor can be analyzed using Fourier Transformation to identify chewing and/or swallowing motions.

In an example, a method and/or system can comprise AI (artificial intelligence) messages, feedback, and/or coaching to help a person manage their food consumption. In an example, a method and/or system can comprise AI (artificial intelligence) messages, feedback, and/or coaching to help a person manage their net energy balance. In an example, a method and/or system can comprise AI (artificial intelligence) messages, feedback, and/or coaching to help a person manage the types and/quantities of food which they consume. In an example, a method and/or system can comprise AI (artificial intelligence) messages, feedback, and/or coaching to help a person manage their nutritional intake.

In an example, a method and/or system can comprise AI (artificial intelligence) messages, feedback, and/or coaching to help a person manage the types and/quantities of nutrients which they consume. In an example, a method and/or system can comprise AI (artificial intelligence) messages, feedback, and/or coaching to help a person manage the types and/quantities of food which the person consumes. In an example, a method and/or system can comprise AI (artificial intelligence) messages, feedback, and/or coaching to help a person manage the types and/quantities of nutrients which the person consumes.

In an example, a system can use AI to analyze images recorded by a wearable camera to identify food items in those images. In an example, a system can use AI to analyze the color, texture, shape, size, volume, temperature, moisture content, infrared spectral distribution, and/or geographic context of food items in images recorded by a wearable camera to identify the types and/or amounts of foods in those images. In an example, a system can use AI to analyze the color, texture, shape, size, volume, temperature, and/or infrared spectral distribution of food items in images recorded by a wearable camera to identify the types and/or amounts of foods in those images. In an example, a system can use AI to analyze the color, texture, shape, size, volume, temperature, moisture content, and/or infrared spectral distribution of food items in images recorded by a wearable camera to identify the types and/or amounts of foods in those images.

In an example, a system can use AI to analyze the color, texture, shape, size, temperature, and/or molecular composition of food items in images recorded by a wearable camera to identify the types and/or amounts of foods in those images. In an example, a system can use AI to analyze the color, texture, shape, size, temperature, and/or infrared spectral distribution of food items in images recorded by a wearable camera to identify the types and/or amounts of foods in those images. In an example, a system can use AI to analyze the color, texture, shape, size, and/or temperature of food items in images recorded by a wearable camera to identify the types and/or amounts of foods in those images. In an example, a system can use AI to analyze the color, texture, shape, and/or size of food items in images recorded by a wearable camera to identify the types and/or amounts of foods in those images.

In an example, a system for measuring a person’s food and/or nutritional consumption can improve the accurate of estimated types and/or amounts of food and/or nutrients consumed by the person by repeating the following steps in an iterative manner: (a) use Artificial Intelligence (AI) to estimate types and/or amounts of food consumed by a person, (b) prompt the person to confirm or correct those estimated types and/or amounts, and (c) train, refine, and/or update the AI based on the person’s response. In an example, images of food recorded by a camera can be analyzed using machine learning.

In an example, a system for measuring a person’s food and/or nutritional consumption can use a neural network to estimate types and/or amounts of food consumed by a person, prompt the person to confirm or correct those estimated types and/or amounts, and then refine, train, and/or update the neural network based on the person’s response. In an example, a system for measuring a person’s food and/or nutritional consumption can improve the accurate of estimated types and/or amounts of food and/or nutrients consumed by the person by repeating the following steps in an iterative manner: (a) use a neural network to estimate types and/or amounts of food consumed by a person, (b) prompt the person to confirm or correct those estimated types and/or amounts, and (c) train, refine, and/or update the neural network based on the person’s response. In an example, signals from one or more eating detection sensors can be analyzed using an Artificial Neural Network. In an example, signals from a sensor can be analyzed using a neural network.

In an example, food images recorded by a device can be analyzed using AI (Artificial Intelligence). In an example, a method and/or system can comprise AI (artificial intelligence) to help monitor and manage a person’s food consumption. In an example, a method and/or system can comprise AI (artificial intelligence) to help monitor and manage a person’s net energy balance. In an example, a method and/or system can comprise AI (artificial intelligence) to help monitor and manage a person’s nutritional intake.

In an example, signals from a sensor can be analyzed using machine learning. In an example, a system for measuring a person’s food and/or nutritional consumption can improve the accurate of estimated types and/or amounts of food and/or nutrients consumed by the person by repeating the following steps in an iterative manner: (a) use machine learning to estimate types and/or amounts of food consumed by a person, (b) prompt the person to confirm or correct those estimated types and/or amounts, and (c) train, refine, and/or update the machine learning based on the person’s response. In an example, signals from one or more eating detection sensors can be analyzed using a Support Vector Machine. In an example, a device worn by a person can comprise sensors wherein data from the sensors is analyzed using a Support Vector Machine (SVM) to estimate the types and/or amounts of food and/or nutrients consumed by the person.

In an example, a system can provide two stages of food and/or eating evaluation: a first stage of estimation of the types and/or amounts of food consumed which is provided by machine-based analysis (e.g. artificial intelligence) of food and/or eating information (e.g. eating motions, eating sounds, and/or food images) in a first timeframe; and a second stage of estimation of the types and/or amounts of food consumed which is provided by human analysis of food and/or eating information (e.g. eating motions, eating sounds, and/or food images) in a second timeframe, wherein the second timeframe is longer than the first timeframe. In an example, a system can provide two stages of food and/or eating evaluation: a first stage of food identification which is provided by machine-based analysis (e.g. artificial intelligence) in a first timeframe and a second stage of food identification which is provided by human analysis in a second timeframe, wherein the second timeframe is longer than the first timeframe.

In an example, a system can provide two stages of food and/or eating evaluation: a first stage of estimation of the types and/or amounts of food consumed which is provided by machine-based analysis (e.g. artificial intelligence) of food and/or eating information (e.g. eating motions, eating sounds, and/or food images) in a first timeframe (e.g. within 1 to 10 minutes); and a second stage of estimation of the types and/or amounts of food consumed which is provided by human analysis of food and/or eating information (e.g. eating motions, eating sounds, and/or food images) in a second timeframe (e.g. within 1 to 24 hours).

In an example, a system can provide two stages of food and/or eating evaluation: a first stage of estimation of the types and/or amounts of food consumed which is provided by machine-based analysis (e.g. artificial intelligence) of food and/or eating sensor data (e.g. eating motions, eating sounds, and/or food images) in a first timeframe (e.g. less than 5 minutes after data collection); and a second stage of estimation of the types and/or amounts of food consumed which is provided by human analysis of food and/or eating sensor data (e.g. eating motions, eating sounds, and/or food images) in a second timeframe (e.g. more than 5 hours after data collection).

In an example, a system can provide two stages of food and/or eating evaluation: a first stage of estimation of the types and/or amounts of food consumed which is provided by machine-based analysis (e.g. artificial intelligence) of food and/or eating information (e.g. eating motions, eating sounds, and/or food images) in a first timeframe (e.g. less than 10 minutes); and a second stage of estimation of the types and/or amounts of food consumed which is provided by human analysis of food and/or eating information (e.g. eating motions, eating sounds, and/or food images) in a second timeframe (e.g. more than 5 hours).

In an example, a device can include an image-analyzing member that provides an initial estimate of the types and quantities of food consumed by the person and this initial estimate is then refined by human interaction and/or evaluation. In an example, a method can start with the collection of passively-collected data concerning food consumption from “level 1” sensors that are worn in, or on, a person. Next, actively-entered data can be collected concerning food consumption. Then, the person’s caloric intake is estimated based on a combination, weighting, or merging of passively-collected data and actively-entered data. Data is collected and received concerning the person’s caloric expenditure. Then, predicted weight gain or loss for the person is estimated based on the person’s caloric intake minus the person’s caloric expenditure (i.e. net energy balance).

In an example, a system or device for estimating food and/or nutrition consumption can be partially automatic and partially refined by human evaluation or interaction. In an example, a device and method comprise a device and method that automatically analyzes images of food sources and a person’s mouth in order to provide initial estimates of the types and quantities of food consumed. These initial estimates are then refined by human evaluation and/or interaction. In an example, estimation of the types and quantities of food consumed is refined or enhanced by human interaction and/or evaluation.

In an example, analysis of food images and estimation of food consumed by a device and method can be entirely automatic or can be a mixture of automated estimates plus human refinement. Even a partially-automated device and method for calorie monitoring and estimation is superior to prior art that relies completely on manual calorie counting or manual entry of food items consumed. In an example, the estimates of the types and quantities of food consumed that are produced by a device are used to estimate human caloric intake. In an example, images of a person’s mouth, a nearby food, and the interaction between the person’s mouth and food can be automatically, or semi-automatically, analyzed to estimate the types of quantities of food that the person eats.

In an example, if two estimates from a first cycle of data collection do not meet criteria for similarity and/or convergence, then a method escalates to a second cycle of more-intrusive data collection. This second cycle begins with passively-collected data about a person’s food consumption received from a camera that is worn on the person and takes pictures in a more continuous manner than in the first cycle. In an example, this camera can be a miniature video camera that is worn by the person and which continuously takes video images of the space surrounding the person in this second cycle of more-intrusive data collection.

In an example, similarity and/or convergence of caloric intake estimates from passively-collected data and actively-entered data functions can serve as a proxy for data accuracy. Even in the case wherein estimates of caloric intake based on passively-collected data are consistently more accurate than estimates of caloric intake based on actively-entered data, this method can be superior to estimating caloric intake based on passively-collected data alone. Even if estimates from actively-entered data are redundant or inferior to estimates from passively-collected data, there are psychological and motivational benefits to engaging someone in managing their own energy balance and weight.

In an example, a device and/or system can estimate and display the number of each type of nutrient in each type of food in a meal. In an example, a device and/or system can estimate and display the number of calories in each type of food in a meal. In an example, a device and/or system for energy balance management can compare a person’s caloric intake to the person’s caloric expenditure to calculate the person’s net energy balance and display this information in graphical form to help the person manage this balance. In an example, a device and/or system for energy balance management can compare a person’s caloric intake to the person’s caloric expenditure to calculate the person’s net energy balance and display this information in graphical form to help the person correct an imbalance.

In an example, a device and/or system for energy balance management can compare a person’s cumulative caloric intake to the person’s cumulative caloric expenditure during a period of time to calculate the person’s cumulative net energy balance for that period of time and display this information in graphical form to help the person correct a net energy imbalance. In an example, a device and/or system for energy balance management can compare a person’s cumulative caloric intake to the person’s cumulative caloric expenditure during a period of time to calculate the person’s cumulative net energy balance for that period of time and display this information in graphical form to help the person correct a net energy surplus or deficit.

In an example, a combined device and system for measuring and modifying caloric intake and caloric expenditure can be a useful part of an overall approach for good nutrition, energy balance, fitness, weight management, and good health. As part of such an overall system, a device that measures a person’s consumption of at least one selected type of food, ingredient, and/or nutrient can play a key role in helping that person to achieve their goals with respect to proper nutrition, food consumption modification, energy balance, weight management, and good health outcomes.

In an example, information from a food-consumption monitoring device can be combined with information from a caloric expenditure monitoring device to comprise an overall system for energy balance, fitness, weight management, and health improvement. In an example, a food-consumption monitoring device can be in wireless communication with a separate fitness monitoring device. In an example, capability for monitoring food consumption can be combined with capability for monitoring caloric expenditure within a single device. In an example, a single device can be used to measure the types and amounts of food, ingredients, and/or nutrients that a person consumes as well as the types and durations of the calorie-expending activities in which the person engages.

In an example, a device and/or system for nutritional management can track a person’s cumulative intake of a selected nutrient during a period of time and display this information in graphical form to help the person correct a surplus or deficit in the person’s cumulative intake of that nutrient during that period of time. In an example, a device and/or system for nutritional management can track a person’s water consumption during a period of time and display this information in graphical form to help the person correct a surplus or deficit in the person’s water consumption during that period of time. In an example, a device can recommend a diet based on analysis of a data from one or more biometric sensors worn by the person and the person’s food consumption history.

In an example, a system can identify which types of feedback are most effective in changing a person’s food consumption behavior and can increase use of those types of feedback. In an example, a device can provide feedback to a person concerning where the person can go to find food which is healthier than nearby food in a food image recorded by the device. In an example, a device and/or system can provide feedback based on the types and/or amounts of food consumed. In an example, a device and/or system can provide feedback based on the location of food consumption. In an example, feedback can be designed to discourage eating prior to sleep or during the night.

In an example, feedback can comprise giving a person information about the nutritional composition of different types of food in a meal. In an example, feedback can comprise giving a person information about the molecular and/or nutritional composition of different types of available food. In an example, feedback can comprise giving a person information about the molecular and/or nutritional composition of different types of nearby food. In an example, feedback can comprise giving a person information about the molecular composition of different types of food in a meal. In an example, a device and/or system can comprise a touchscreen which is used to provide feedback to a person concerning the types and/or amounts of food which they have consumed.

In an example, feedback can include suggesting when a person stop eating during a meal. In an example, feedback can comprise giving a person suggestions about what portions of a meal to eat and what portions of a meal to skip. In an example, feedback can comprise giving a person suggestions about desired sizes of portions of different foods in a meal. In an example, a wearable device for measuring a person’s food consumption can further comprise a feedback mechanism involving the following steps: the device makes a preliminary identification of food type based on data from one or more wearable sensors; the device informs the person of the preliminary identification of food type; and the device receives confirmation of this food type or correction of this food type from the person.

In an example, a device can make recommendations concerning a person’s food consumption based on one or more factors selected from the group consisting of: the amounts of exercise that the person has done during a recent period of time; the person’s body mass index; the person’s history of food consumption; the person’s body shape; the person’s demographic characteristics; the person’s cumulative caloric expenditure; the person’s cumulative caloric intake; the types of sports in which the person participates; the person’s health status; the results of genetic testing; the person’s food allergies or religious requirements; the person’s history of exercise; the geographic availability of specific types of cuisine; the amounts of foods that the person has consumed during a recent period of time; the prices and/or cost of specific types of food; the types of exercise that the person has done during a recent period of time; the geographic availability of specific types of food; and the types of foods that the person has consumed during a recent period of time.

In an example, a device can recommend a dietary goal and/or exercise goal for a person based on one or more factors selected from the group consisting of: the amounts of exercise that the person has done during a recent period of time; the person’s body mass index; the person’s history of food consumption; the person’s body shape; the person’s demographic characteristics; the person’s cumulative caloric expenditure; the person’s cumulative caloric intake; the types of sports in which the person participates; the person’s health status; the results of genetic testing; the person’s food allergies or religious requirements; the person’s history of exercise; the geographic availability of specific types of cuisine; the amounts of foods that the person has consumed during a recent period of time; the prices and/or cost of specific types of food; the types of exercise that the person has done during a recent period of time; the geographic availability of specific types of food; and the types of foods that the person has consumed during a recent period of time.

In an example, a device can recommend a nutritional intake amount for a person based on one or more factors selected from the group consisting of: the amounts of exercise that the person has done during a recent period of time; the person’s body mass index; the person’s history of food consumption; the person’s body shape; the person’s demographic characteristics; the person’s cumulative caloric expenditure; the person’s cumulative caloric intake; the types of sports in which the person participates; the person’s health status; the results of genetic testing; the person’s food allergies or religious requirements; the person’s history of exercise; the geographic availability of specific types of cuisine; the amounts of foods that the person has consumed during a recent period of time; the prices and/or cost of specific types of food; the types of exercise that the person has done during a recent period of time; the geographic availability of specific types of food; and the types of foods that the person has consumed during a recent period of time.

In an example, a device can recommend a particular diet and/or exercise regimen for a person based on one or more factors selected from the group consisting of: the amounts of exercise that the person has done during a recent period of time; the person’s body mass index; the person’s history of food consumption; the person’s body shape; the person’s demographic characteristics; the person’s cumulative caloric expenditure; the person’s cumulative caloric intake; the types of sports in which the person participates; the person’s health status; the results of genetic testing; the person’s food allergies or religious requirements; the person’s history of exercise; the geographic availability of specific types of cuisine; the amounts of foods that the person has consumed during a recent period of time; the prices and/or cost of specific types of food; the types of exercise that the person has done during a recent period of time; the geographic availability of specific types of food; and the types of foods that the person has consumed during a recent period of time.

In an example, a device can recommend a particular diet for a person based on one or more factors selected from the group consisting of: the amounts of exercise that the person has done during a recent period of time; the person’s body mass index; the person’s history of food consumption; the person’s body shape; the person’s demographic characteristics; the person’s cumulative caloric expenditure; the person’s cumulative caloric intake; the types of sports in which the person participates; the person’s health status; the results of genetic testing; the person’s food allergies or religious requirements; the person’s history of exercise; the geographic availability of specific types of cuisine; the amounts of foods that the person has consumed during a recent period of time; the prices and/or cost of specific types of food; the types of exercise that the person has done during a recent period of time; the geographic availability of specific types of food; and the types of foods that the person has consumed during a recent period of time.

In an example, a device can recommend a type of meal based on analysis of a person’s food consumption history. In an example, a device can recommend a type of food based on analysis of a person’s food consumption history. In an example, a device can recommend a diet based on analysis of a person’s health and food consumption history. In an example, a device can recommend a diet based on analysis of a person’s food consumption history. In an example, a device can recommend an exercise regimen based on the person’s food consumption history. In an example, a device can recommend an exercise regimen for a person based on the types and/or amounts of food which the person has consumed.

In an example, a device worn by a person can analyze a meal in front of the person and recommend which foods in that meal the person should or should not eat. In an example, a device worn by a person can analyze a meal in front of the person and recommend how much (e.g. what fraction or percentage) of each of the foods in that meal the person should eat. In an example, a system can analyze the nutritional content of an image of a meal in front of a person and can recommend what parts and/or percentages of the meal a person should eat and/or what parts and/or percentages of the meal the person should skip. In an example, a system can analyze the nutritional content of a meal in front of a person and can recommend what parts and/or percentages of the meal a person should eat and/or what parts and/or percentages of the meal the person should skip.

In an example, a system can analyze an image of a menu and recommend which dishes would be best for a person. In an example, a system can analyze an image of a menu and recommend what at a person should order. In an example, a system can analyze options on a menu and recommend which dishes would be best for a person. In an example, a system can analyze options on a menu and recommend what at a person should order. In an example, a system can create a meal plan for a person. In an example, a system can create a meal plan for a person based in part on the types and/or amounts of food which the person has consumed. In an example, a system can create a dietary and/or nutritional plan for a person. In an example, a system can create a dietary and/or nutritional plan for a person based in part on the types and/or amounts of food which the person has consumed. In an example, a system can be integrated with a social network.

In an example, a system can provide a person with dietary and/or nutritional recommendations. In an example, a system can provide a person with meal recommendations. In an example, a system can provide a person with meal recommendations to help the person achieve their nutritional, dietary, and/or weight management goals. In an example, a system can recommend what portion, percentage, or parts of a meal a person should eat. In an example, a wearable device and/or system can recommend selected travel paths (or routes) for a person to follow to help the person to avoid exposure to sources of food which would be unhealthy for that person. In an example, a system can provide a person with recommendations for types of food to purchase.

In an example, a system can provide a person with recommendations for types of food to purchase based in part on the types and/or amounts of food which the person has consumed. In an example, a system can provide a person with recommendations for types of food to consume more frequently. In an example, a system can provide a person with recommendations for types of food to consume more frequently based in part on the types and/or amounts of food which the person has consumed. In an example, a system can provide a person with recommendations for types of food to consume less frequently.

In an example, a system can provide a person with recommendations for types of food to consume less frequently based in part on the types and/or amounts of food which the person has consumed. In an example, a system can provide a person with recommendations for types of food to avoid. In an example, a system can provide a person with recommendations for types of food to avoid based in part on the types and/or amounts of food which the person has consumed. In an example, a system can provide a person with dietary and/or nutritional recommendations based in part on the types and/or amounts of food which the person has consumed.

In an example, a system can provide a person with recommendations for types and/or amounts of food that the person should eat. In an example, a system can provide a person with recommendations for types and/or amounts of food that the person should eat to achieve their nutritional, dietary, and/or weight management goals. In an example, a system can provide a person with recommendations for types and/or amounts of food that the person should eat to achieve their nutritional, dietary, and/or weight management goals in light of the types and/or amounts of food that the person has consumed.

In an example, a system can provide a person with recommendations for types and/or amounts of food that the person should eat in light of the types and/or amounts of food that the person has consumed. In an example, a system can provide a person with recommendations for types and/or amounts of food that the person should buy. In an example, a system can provide a person with recommendations for types and/or amounts of food that the person should buy in light of the types and/or amounts of food that the person has consumed. In an example, a system can provide a person with meal recommendations to help the person achieve their nutritional, dietary, and/or weight management goals in light of the types and/or amounts of food that the person has consumed. In an example, a system can provide a person with meal recommendations in light of the types and/or amounts of food that the person has consumed. In an example, a system can provide a person with restaurant recommendations. In an example, a system can provide a person with restaurant recommendations in light of the types and/or amounts of food that the person has consumed.

In an example, a wearable device and/or system can recommend selected travel paths for a person to following when going through a food store to help the person to avoid exposure to unhealthy types of food. In an example, a wearable device and/or system can recommend selected travel paths (or routes) for a person to follow to help the person to avoid exposure to sources of unhealthy types of food. In an example, a wearable device and/or system can recommend selected travel paths for a person to following when going through a food store to help the person to avoid exposure to types of food which would be unhealthy for that person.

In an example, a device and/or system can analyze eating behavior to identify snacking behavior and provide a person with feedback to reduce consumption of unhealthy types and/or amounts of food. In an example, a device and/or system can analyze eating behavior to identify unhealthy eating patterns and provide a person with feedback to reduce consumption of unhealthy types and/or amounts of food. In an example, a system can analyze patterns of electromagnetic signals received by EEG sensors worn by a person to identify associations between these patterns tailor feedback to the person to decrease their consumption of unhealthy types and/or amounts of food. In an example, a system can track changes in a person’s consumption of unhealthy food following different types of feedback from the system in order to identify and increase use of the most effective types of feedback for that person.

In an example, a device and/or system can analyze eating behavior to identify conditions which cause a person to be more susceptible to consuming unhealthy types and/or amounts of food and provide the person with feedback to reduce consumption of unhealthy types and/or amounts of food. In an example, a device and/or system can analyze environmental conditions and eating behavior to identify environmental conditions which trigger a person to be more susceptible to consuming unhealthy types and/or amounts of food and provide the person with feedback to reduce consumption of unhealthy types and/or amounts of food. In an example, a device and/or system can analyze environmental conditions and eating behavior to identify environmental conditions which cause a person to be more susceptible to consuming unhealthy types and/or amounts of food and provide the person with feedback to reduce consumption of unhealthy types and/or amounts of food.

In an example, a device and/or system can analyze eating behavior to identify conditions which cause a person to be more susceptible to eating unhealthy types and/or amounts of food. In an example, a wearable device and/or system can guide a person through a food store along a path which helps the person to avoid exposure to types of food which would be unhealthy for that person. In an example, a wearable device and/or system can guide a person along travel paths (or routes) which help the person to avoid exposure to sources of food which would be unhealthy for that person. In an example, a device and/or system can analyze eating behavior to identify unhealthy eating patterns.

In an example, a device and/or system for nutritional intake management can trigger unpleasant feedback when a person eats a type and/or amount of food which is unhealthy. In an example, feedback in response to detection of consumption of unhealthy types and/or amounts of food can comprise displaying images of an overweight person. In an example, feedback in response to detection of consumption of unhealthy types and/or amounts of food by a person can comprise displaying images of that person with computer-simulated weight gain. In an example, feedback in response to detection of consumption of unhealthy types and/or amounts of food by a person can comprise displaying computer-simulated images of that person after weight gain. In an example, feedback in response to detection of consumption of unhealthy types and/or amounts of food by a person can comprise displaying computer-simulated images of that person after projected weight gain based on continued consumption of those types and/or amounts of food.

In an example, a device and/or system for nutritional intake management can trigger an unpleasant physiological response when a person eats a type and/or amount of food which is unhealthy. In an example, a device and/or system for nutritional intake management can trigger and/or induce a feeling of satiety when a person eats a type and/or amount of food which is unhealthy. In an example, a device and/or system for nutritional intake management can trigger and/or induce nausea when a person eats a type and/or amount of food which is unhealthy. In an example, a device and/or system for nutritional intake management can trigger and/or induce an upset stomach when a person eats a type and/or amount of food which is unhealthy. In an example, a device can play an unpleasant sound in a person’s hearing in response to nearby food with undesirable nutritional characteristics. In an example, a device can play an unpleasant sound in a person’s hearing in response to nearby food whose consumption would have negative health effects on the person.

In an example, a device can display an unpleasant virtual object in a person’s field of view in response to nearby food with undesirable nutritional characteristics. In an example, a device can display an unpleasant virtual object in a person’s field of view in response to nearby food whose consumption would have negative health effects on the person. In an example, a device and/or system for nutritional intake management can trigger unpleasant olfactory, acoustic, tactile, or visual sensation when a person eats a type and/or amount of food which is unhealthy. In an example, a device can activate an unpleasant visual, auditory, and/or tactile cue in response to image-based detection of unhealthy food (nearby or consumed).In an example, a device can activate an unpleasant visual, auditory, and/or tactile cue in response to detection of unhealthy food (nearby or consumed).

In an example, a wearable device and/or system can guide a person through a food store along a path which helps the person to avoid exposure to unhealthy types of food. In an example, a wearable device and/or system can guide a person along travel paths (or routes) which help the person to avoid exposure to sources of unhealthy types of food. In an example, a device and/or system can analyze environmental conditions and eating behavior to identify environmental conditions which cause a person to be more susceptible to eating unhealthy types and/or amounts of food.

In an example, feedback can be designed to discourage an unhealthy pattern of under-eating and over-eating. In an example, feedback can be designed to discourage an unhealthy cycle of under-eating and over-eating. In an example, feedback in response to unhealthy food consumption can be progressively more intrusive until the person responses but limiting further consumption. In an example, the frequency of feedback in response to unhealthy food consumption can escalate until the person responses but limiting further consumption. In an example, the magnitude of feedback in response to unhealthy food consumption can escalate until the person responses but limiting further consumption. In an example, the modes of feedback in response to unhealthy food consumption can escalate until the person responses but limiting further consumption.

In an example, a device and/or system can provide a person with nutritional and/or dietary coaching based on the types and/or amounts of food which the person has consumed. In an example, a device and/or system can provide a person with nutritional and/or dietary coaching based on the relative amounts of different type of nutrients which the person has consumed. In an example, a device and/or system can provide a person with nutritional and/or dietary coaching based on the proportions and/or ratios between different types of nutrients which the person has consumed. In an example, a device and/or system can provide a person with nutritional and/or dietary coaching based on the percentages, proportions, and/or ratios of actual amounts of different types of nutrients which the person has consumed vs. target amounts for those types of nutrients.

In an example, a device and/or system can provide a person with nutritional and/or dietary coaching based on the percentages, proportions, and/or ratios of actual amounts of different types of nutrients which the person has consumed during a period of time vs. target amounts for those types of nutrients for that period of time. In an example, a device and/or system can provide a person with nutritional and/or dietary coaching based on the percentages, proportions, and/or ratios of cumulative amounts of different types of nutrients which the person has consumed during a period of time vs. target amounts for those types of nutrients for that period of time. In an example, a device and/or system can provide a person with nutritional and/or dietary coaching based on the amounts of different type of nutrients which the person has consumed.

In an example, a device can analyze the relationship between a person’s stress level (e.g. measured by analysis of the person’s voice) and the types and/or amounts of food consumed by that person in order to help (e.g. guide or coach) the person to have healthier eating behavior. In an example, a device can analyze the relationship between a person’s stress level (e.g. measured by analysis of the person’s voice) and the types and/or amounts of food consumed by that person in order to help (e.g. guide or coach) the person avoid unhealthy stress-induced eating behavior. In an example, a system can provide a person with nutritional, dietary, and/or weight-management coaching.

In an example, a device can be part of a system which tracks a person’s cumulative caloric intake (during a period of time), the person’s caloric expenditure (during the period of time), and provides feedback and/or recommendations for behavior modification if the person’s net caloric balance goes below a target minimum amount or above a target maximum amount. In an example, a device can be part of a system which tracks a person’s cumulative caloric intake (during a period of time), the person’s caloric expenditure (during the period of time), and provides feedback and/or recommendations concerning the person’s eating and/or exercise if the person’s net caloric balance goes below a target minimum amount or above a target maximum amount.

In an example, a system can provide a person with feedback, guidance, recommendations, and/or messages concerning food and/or activities based on a correlation (or other statistical relationship) between biometric indicators of a person’s stress level and the types and/or amounts of food that the person’ consumes which is identified by the system. In an example, a device can be part of a system which tracks a person’s cumulative caloric intake (during a period of time), the person’s caloric expenditure (during the period of time), and provides feedback and/or recommendations for modification of the person’s eating and/or exercise behavior (during the period of time) if the person’s net caloric balance goes below a target minimum amount or above a target maximum amount.

In an example, a system can provide a person with nutritional, dietary, and/or weight-management coaching in light of the types and/or amounts of food that the person has consumed. In an example, a system can provide a person with nutritional and/or dietary coaching based on a correlation (or other statistical relationship) between biometric indicators of a person’s stress level and the types and/or amounts of food that the person’ consumes which is identified by the system. In an example, a system worn by a person can be in communication with dietary professionals who provide coaching to help the person reach their nutritional and/or weight management goals.

In an example, a system can provide feedback to a person, wherein this feedback includes nutritional and/or dietary coaching. In an example, a device or system can provide feedback including dietary coaching. In an example, a system can provide feedback to a person, wherein this feedback includes nutritional and/or dietary coaching in light of the types and/or amounts of food that the person has consumed. In an example, a device or system can provide feedback including dietary coaching from a nutritionist and/or dietician.

In an example, a system worn by a person can be in communication with other people so that other people can provide peer support and/or coaching to help the person meet their nutritional and/or weight management goals. In an example, a system worn by a person can be in communication with other people so that other people can provide peer support and/or coaching for the person to improve their nutritional health. In an example, feedback can include providing a person with a recommendation for substituting a more healthy type and/or amount for food for a less healthy type and/or amount food. In an example, feedback can include providing a person with a recommendation for eating a more healthy type and/or amount for food instead of a less healthy type and/or amount food near the person. In an example, feedback can include providing a person with a recommendation for eating a more healthy type and/or amount for food instead of a less healthy type and/or amount food in an image of nearby food. In an example, food images recorded by a device can be reviewed and analyzed by a dietician and/or nutritionist.

In an example, a device can be part of a system for maintaining caloric energy balance. In an example, a device can be part of a system for managing caloric energy balance. In an example, a device and/or system for energy balance management can compare a person’s caloric intake to the person’s caloric expenditure to calculate the person’s net energy balance. In an example, a device can be part of a multicomponent system which tracks a person’s caloric intake, the person’s caloric expenditure, and the cumulative net balance thereof. In an example, a device can be part of a system comprising a wearable device which measures nutritional intake and an exercise device, wherein the system tracks a person’s energy balance between caloric intake and caloric expenditure.

In an example, a device can be part of a system which analyzes the time-lagged relationship between a person’s net caloric balance (e.g. cumulative caloric intake minus cumulative caloric expenditure) and subsequent changes in the person’s weight. In an example, a device can be part of a system which analyzes the time-lagged relationship between a person’s net caloric balance (e.g. cumulative caloric intake minus cumulative caloric expenditure) and subsequent changes in the person’s body shape. In an example, a device can be part of a system which analyzes the time-lagged relationship between a person’s net caloric balance (e.g. cumulative caloric intake minus cumulative caloric expenditure) and subsequent changes in the person’s health status.

In an example, a device can be part of a system which analyzes the relationship between a person’s net caloric balance (e.g. cumulative caloric intake minus cumulative caloric expenditure) and changes in the person’s weight. In an example, a device can be part of a system which analyzes the relationship between a person’s net caloric balance (e.g. cumulative caloric intake minus cumulative caloric expenditure) and changes in the person’s body shape. In an example, a device can be part of a system which analyzes the relationship between a person’s net caloric balance (e.g. cumulative caloric intake minus cumulative caloric expenditure) and changes in the person’s health status.

In an example, feedback can include an estimated of the amount of exercise than a person would have to perform in order to balance out calories consumed by eating food. In an example, feedback can include an estimate of the number of calories which a person would have to expend in order to balance out calories consumed by eating food. In an example, a device can give a person feedback when they have consumed a target amount of calories during an eating event and/or meal. In an example, a device and/or system for energy balance management can compare a person’s caloric intake to the person’s caloric expenditure to calculate the person’s net energy balance and provide feedback to help the person manage this balance. In an example, a device and/or system for energy balance management can compare a person’s caloric intake to the person’s caloric expenditure to calculate the person’s net energy balance and provide feedback to help the person correct an imbalance.

A device can include an optional analytic component that analyzes and compares human caloric input vs. human caloric output for a particular person as part of an overall device, system, and method for overall energy balance and weight management. This overall device, system, and method can be used to help a person to lose weight or to maintain a desirable weight. In an example, a device and method can be used as part of a system with a human-energy input measuring component and a human-energy output measuring component. In an example, a device is part of an overall system for energy balance and weight management.

A person’s weight gain or loss can be predicted because: net energy balance is caloric intake minus caloric expenditure; and weight gain or loss follows directly from net energy balance. Predicted weight gain or loss can then be compared to actual weight gain or loss. If estimated caloric intake is inaccurate, then predicted weight gain or loss will be significantly different than actual weight gain or loss. If estimated caloric intake is accurate (and caloric expenditure is also accurate), then predicted weight gain or loss will be close to actual weight gain or loss.

In an example, a device and method for estimating human caloric intake can be used in conjunction with a device and method for estimating human caloric output and/or human energy expenditure. In an example, this present invention can be used in combination with a wearable and mobile energy-output-measuring component that automatically records and analyses images in order to detect activity and energy expenditure. In an example, this present invention can be used in combination with a wearable and mobile device that estimates human energy output based on patterns of acceleration and movement of body members. In an example, a device can be used in combination with an energy-output-measuring component that estimates energy output by measuring changes in the position and configuration of a person’s body.

In an example, a device and method includes an alarm that is triggered if a wearable camera is covered up. In various examples, a device comprises one or more cameras which detect and respond if their direct line of sight with the person’s mouth or nearby food is impaired. In an example, a device includes a tamper-resisting member that monitors a person’s mouth using face recognition methods and responds if the line of sight from a camera to the person’s mouth is impaired when a person eats. In an example, a device includes a tamper-resisting member that detects and responds if the person’s actual weight gain or loss is inconsistent with predicted weight gain or loss. Weight gain or loss can be predicted by the net balance of estimated caloric intake and estimated caloric expenditure.

In an example, a device and/or method for measuring a person’s caloric intake can comprise: (a) a first sensor and/or user interface that collects a first set of data concerning what the person eats; (b) a data processor that calculates a first estimate of the person’s caloric intake based on the first set of data, uses this first estimate of the person’s caloric intake to estimate predicted weight change for the person during a period of time, and compares predicted to actual weight change to determine whether predicted and actual weight change meet criteria for similarity and/or convergence; and (c) a second sensor and/or user interface that collects a second set of data concerning what the person eats if the criteria for similarity and/or convergence of predicted and actual weight change are not met.

In an example, a device can be embodied in a method for measuring a person’s caloric intake comprising: (a) receiving a first set of data concerning what a person eats, wherein this first set includes passively-collected data that is collected in a manner that does not require voluntary actions by the person associated with particular eating events other than the actions of eating, and wherein this first set also includes actively-entered data that is collected in a manner that requires voluntary actions by the person associated with particular eating events other than the actions of eating and; (b) calculating a first estimate of the person’s caloric intake based on this first set of data, using this first estimate of the person’s caloric intake to estimate predicted weight change for the person during a period of time, and comparing predicted weight change to actual weight change to determine whether predicted weight change and actual weight change meet criteria for similarity and/or convergence; and (c) if predicted weight change and actual weight change do not meet the criteria for similarity and/or convergence, then receiving a second set of data concerning what the person eats and calculating a second estimate of caloric intake using this second set of data.

In an example, a device can be incorporated into an overall device, system, and method for human energy balance and weight management. In an example, the estimates of the types and quantities of food consumed that are provided by this present invention are used to estimate human caloric intake. These estimates of human caloric intake are then, in turn, used in combination with estimates of human caloric expenditure as part of an overall system for human energy balance and weight management. In an example, estimates of the types and quantities of food consumed are used to estimate human caloric intake and wherein these estimates of human caloric intake are used in combination with estimates of human caloric expenditure as part of an overall system for human energy balance and human weight management.

In an example, a device for measuring a person’s caloric intake comprising: a first sensor and/or user interface that collects a first set of data concerning what the person eats; a data processor that calculates a first estimate of the person’s caloric intake based on the first set of data, uses this first estimate of the person’s caloric intake to estimate predicted weight change for the person during a period of time, and compares predicted to actual weight change to determine whether predicted and actual weight change meet criteria for similarity and/or convergence; and a second sensor and/or user interface that collects a second set of data concerning what the person eats if the criteria for similarity and/or convergence of predicted and actual weight change are not met.

In an example, a method for measuring the types and quantities of food consumed by a person can comprise: (a) receiving a first set of data concerning what the person eats; (b) calculating a first estimate of the types and quantities of food consumed based on the first set of data, using this first estimate of the types and quantities of food consumed to estimate predicted weight change for the person during a period of time, and comparing predicted to actual weight change to determine whether predicted and actual weight change meet criteria for similarity and/or convergence; and then (c) if predicted weight change and actual weight change do not meet the criteria for similarity and/or convergence, then receiving a second set of data concerning what the person eats and calculating a second estimate of the types and quantities of food consumed using this second set of data.

In an example, a person’s actual weight (gain or loss) can be measured by having the person stand on a scale and having the scale wirelessly transmit the person’s current weight to the same computing unit that performs caloric intake estimation. This computing unit can compare the person’s current weight to the person’s previous weight in order to calculate actual weight gain or loss. In an example, the person can manually enter current weight information from the scale via a human-computer interface such as touch screen, voice recognition, or keypad. In an example, the person can be prompted to stand on the scale periodically (e.g. each day, week, or month).

In an example, a person’s actual weight (gain or loss) can be monitored and estimated in a passive manner. For example, a camera can be placed in a location from which it can take pictures of the person in an automatic manner on a regular basis. In an example, these pictures can be automatically analyzed by three-dimensional image analysis in order to estimate the person’s weight (gain or loss). In an example, pressure or weight sensors can be placed in locations where the person walks, sits, or reclines on a regular basis. Data from these pressure or weight sensors can be analyzed to estimate the person’s weight (gain or loss).

In an example, a person’s actual weight can be measured when that person stands on scale. This weight value can then be transmitted wirelessly to data processing and transmission unit of a wearable device. Actual weight gain or loss is determined by changes in weight measurements between different times. The predicted weight gain or loss (based on estimated caloric intake and caloric expenditure) for the person can be compared to actual weight gain or loss for the person. If the predicted and actual weight gain or loss meet the criteria for similarity and/or convergence, then the device does not need to gather more information about food consumption. In an example, an estimate of a person’s caloric expenditure can be subtracted from an estimate of the person’s caloric intake in order to calculate the person’s net energy balance and to predict the person’s weight gain or loss for a given period of time.

In an example, caloric intake estimation provided by a device and method can become the energy-input measuring component of an overall system for energy balance and weight management. In an example, a device and method can estimate the energy-input component of energy balance. In an example, data concerning a person’s current weight on a scale can be adjusted to reflect differences in what the person is wearing, the time of day, the proximity to an eating event, or other factors which may temporarily distort the person’s weight. In an example, information concerning these factors can be voluntarily recorded by the person or automatically identified by one or more sensors. In an example, a camera in association with a scale may recognize the types of clothing currently worn by the person and adjust estimation of the person’s current weight accordingly.

In an example, estimates of the types and quantities of food consumed can be used to estimate human caloric intake and these estimates of human caloric intake can be used in combination with estimates of human caloric expenditure as part of an overall system for human energy balance and human weight management. In an example, if predicted weight gain or loss and the actual weight gain or loss for the person meet the criteria for similarity and/or convergence, then a miniature video camera is never activated. In an example, a video camera only operates when predicted and actual weight gain or loss do not meet the criteria for similarity and/or convergence. In this manner, a device provides the person with an incentive to provide timely and accurate actively-entered data concerning food consumption in order to avoid more-intrusive (image-based) monitoring. A device thus engages the person in their own energy balance and weight management more so than an entirely-passively-collected data collection device. It also provides greater compliance and accuracy than an entirely-voluntary data collection device.

In an example, a device and/or system can provide feedback based on the time of day of food consumption. In an example, feedback can be designed to discourage snacking (e.g. between meal times). In an example, a device and/or system can analyze eating behavior to identify specific times of the year when a person is more susceptible to consuming unhealthy types and/or amounts of food and provide the person with feedback to reduce consumption of unhealthy types and/or amounts of food. In an example, a device and/or system can analyze eating behavior to identify specific times of the week when a person is more susceptible to consuming unhealthy types and/or amounts of food and provide the person with feedback to reduce consumption of unhealthy types and/or amounts of food. In an example, a device and/or system can analyze eating behavior to identify specific times of the day when a person is more susceptible to consuming unhealthy types and/or amounts of food and provide the person with feedback to reduce consumption of unhealthy types and/or amounts of food.

In an example, a device and/or system for energy balance management can compare a person’s cumulative caloric intake to the person’s cumulative caloric expenditure during a period of time to calculate the person’s cumulative net energy balance for that period of time and provide feedback to help the person correct a net energy surplus or deficit. In an example, a device and/or system for energy balance management can compare a person’s cumulative caloric intake to the person’s cumulative caloric expenditure during a period of time to calculate the person’s cumulative net energy balance for that period of time and provide feedback to help the person correct a net energy imbalance.

In an example, a device can selectively provide feedback when a person consumes food during selected time (periods) of a day. In an example, a device can selectively provide feedback when a person consumes food at time (periods) of a day other than designated meal times. In an example, a person’s cumulative consumption of food can be tracked during a period of time (e.g. a day) and the person can receive feedback when a maximum target amount has been reached. In an example, a person’s cumulative consumption of food can be tracked during a period of time (e.g. a day or week) and the person can receive feedback when a maximum target amount for the period of time has been reached. In an example, a device can selectively provide feedback when a person consumes food with a selected amount of time before the person usually goes to bed and/or sleep.

In an example, feedback can comprise informing a person of the amount of food they have consumed within a period of time at different time intervals within this period of time. In an example, feedback can comprise informing a person of the amount of food they have consumed within a period of time as different amounts are reached. In an example, a device and/or system for nutritional management can track a person’s cumulative intake of a selected nutrient during a period of time and provide feedback to help the person correct a surplus or deficit in the person’s cumulative intake of that nutrient during that period of time. In an example, a device and/or system for nutritional management can track a person’s water consumption during a period of time and provide feedback to help the person correct a surplus or deficit in the person’s water consumption during that period of time.

In an example, feedback can include an estimated of the quantity or duration of a specific exercise (e.g. steps, running, other) than a person would have to do in order to expand the amount of calories consumed by eating food during a defined period of time or during a meal. In an example, feedback can include an estimated of the amount of exercise than a person would have to perform in order to balance out calories consumed by eating food during a defined period of time or during a meal. In an example, feedback can include an estimated of the amount of exercise than a person would have to perform in order to expand the amount of calories consumed by eating food during a defined period of time or during a meal.

In an example, feedback to a person can include a graph of consumption of different types of food over time. In an example, feedback to a person can include a graph of consumption of different types of nutrients over time. In an example, feedback to a person can include a graph of chewing and/or swallowing motions over time. In an example, feedback to a person can include a graph of caloric intake over time. In an example, feedback can include graphical display of a person’s fat consumption over time. In an example, feedback can include graphical display of a person’s carbohydrate consumption over time. In an example, feedback can include graphical display of a person’s calories consumed over time.

In an example, the key variables of this model (caloric intake, caloric expenditure, predicted weight gain or loss, and actual weight gain or loss) can be estimated for fixed duration, non-overlapping periods of time -- such as individual days, weeks, months, or years. In an example, these key variables can be estimated for a rolling time period, such as a rolling 7-day period wherein, each day, one day is dropped from the beginning of the rolling time period and one day is added to the end of the rolling time period. In an example, the key variables of this model can be estimated for variable-length periods whose variable lengths are defined empirically by clustering together multiple eating and/or physical activity events.

In an example, a device can analyze changes in the rate of a person’s food consumption during an eating event and provide feedback to the person. In an example, a device can analyze the rate of a person’s chewing and/or swallowing motions during an eating event and provide feedback to the person. In an example, a device can analyze changes in the rate of a person’s chewing and/or swallowing motions during an eating event and provide feedback to the person. In an example, there can be an optimal progression and/or variation of chewing and/or swallowing rate during the course of a meal, wherein a person receives feedback if their actual chewing and/or swallowing rate significantly deviates from this optical progression.

In an example, a device can track changes and/or temporal variation in the speed, rate, frequency, and/or pace of eating-related motions. In an example, a device can track changes and/or temporal variation in the speed, rate, frequency, and/or pace of hand-to-mouth motions related to eating. In an example, a device can track changes and/or temporal variation in the speed, rate, frequency, and/or pace of chewing and/or swallowing motions. In an example, a device can track the speed, rate, frequency, and/or pace of eating-related motions. In an example, a device can track the speed, rate, frequency, and/or pace of hand-to-mouth motions related to eating. In an example, a device can track the speed, rate, frequency, and/or pace of chewing and/or swallowing motions.

In an example, a system can analyze, estimate, track, and/or monitor the frequency, rate, pace, speed, and/or variability of bites per meal and/or per interval of time. In an example, a system can analyze, estimate, track, and/or monitor the frequency, rate, pace, speed, and/or variability of chewing motions per meal and/or per interval of time. In an example, a system can analyze, estimate, track, and/or monitor the frequency, rate, pace, speed, and/or variability of hand-to-mouth motions per meal and/or per interval of time. In an example, the amounts and/or types of food consumed can be measure by the rate of swallowing sounds. In an example, the amounts and/or types of food consumed can be measure by the rate of chewing sounds.

In an example, an increase in the rate of a person’s jaw motions during an eating event can be analyzed to provide feedback to the person. In an example, a decrease in the rate of a person’s jaw motions during an eating event can be analyzed to provide feedback to the person. In an example, a device can analyze the rate of a person’s hand-to-mouth motions during an eating event and provide feedback to the person. In an example, a device can analyze changes in the rate of a person’s hand-to-mouth motions during an eating event and provide feedback to the person. In an example, a device can analyze the rate of a person’s food consumption during an eating event and provide feedback to the person.

In an example, rate of food consumption can be analyzed and feedback provided on the rate of consumption. In an example, a person can receive feedback to chew more slowly if the rate of chewing sounds exceeds a maximum value. In an example, a person can receive feedback to chew more slowly if the rate of chewing motions exceeds a maximum value. In an example, a person can receive feedback to eat more slowly if the rate of chewing and/or swallowing sounds exceeds a maximum value. In an example, a person can receive feedback to eat more slowly if the rate of chewing and/or swallowing motions exceeds a maximum value. In an example, variation in the rate of a person’s jaw motions during an eating event can be analyzed to provide feedback to the person.

In an example, there can be an optimal increase and then decrease in chewing and/or swallowing rate during the course of a meal, wherein a person receives feedback if their actual chewing and/or swallowing rate significantly deviates from this optical progression. In an example, there can be an optimal decrease in chewing and/or swallowing rate during the course of a meal, wherein a person receives feedback if their actual chewing and/or swallowing rate significantly deviates from this optical progression. In an example, feedback can include suggesting when a person slow their eating pace and/or rate during a meal. In an example, a device and/or system can provide feedback based on the rate and/or speed of food consumption.

In an example, a device can project a laser beam which is automatically scanned and/or moved around the border, perimeter, and/or outline of nearby food. In an example, a device can project a laser beam which is automatically scanned and/or moved around the border, perimeter, and/or outline of only a subset of nearby food, wherein the device recommends that the person only consume the subset of the nearby food. In an example, a device can project a laser beam which is automatically scanned and/or moved around the border, perimeter, and/or outline of only a subset, percentage, and/or fraction of nearby portion food, wherein the device recommends that the person only consume the subset of the nearby food.

In an example, a system can display a virtual border, perimeter, and/or outline is around a subset of a portion of food, wherein the system recommends that the person consume only the subset of the food portion. In an example, a system can display a virtual border, perimeter, and/or outline is around a subset of a portion of food in an image of the food on a screen, wherein the system recommends that the person consume only the subset of the food portion. In an example, a system can display two borders, perimeters, and/or outlines around a portion of one type of food in a meal in an image of the meal, wherein a first border, perimeter, and/or outline is around the entire food portion, wherein a second border, perimeter, and/or outline is around a subset of the food portion, and wherein the system recommends that the person consume only the subset of the food portion.

In an example, a device worn by a person can communicate with the person via tactile sensations (e.g. vibration, pressure, mild electrical stimulus, and/or movement on skin). In an example, a device worn by a person can communicate with the person via sounds (e.g. computer-generated speech, previously-recorded messages, tones, and/or music). In an example, a wearable device can provide auditory feedback to a person concerning the types and/or amounts of food near them, wherein the frequency of sound is based on the amount and/or type of that food.

In an example, a person’s cumulative consumption of food can be tracked during an eating event (e.g. a particular meal) and the person can receive feedback when a maximum target amount for the meal has been reached. In an example, a system can track changes in a person’s food consumption following different types of feedback to the person in order to refine the types of feedback provided to the person to make that feedback more effective. In an example, a system can track changes in a person’s food consumption following different types of feedback to the person in order to identify the most effective types of feedback. In an example, a system can analyze patterns of electromagnetic signals received by EEG sensors worn by a person to identify associations between these patterns tailor feedback to the person to improve the person’s nutritional health.

In an example, a system can correlate different types of feedback provided to a person by the system with subsequent changes in the types and/or amounts of food consumed by that person in order to increase use of the most effective types of feedback for that person. In an example, a device and/or system can provide visual and/or display-based feedback (e.g. a virtual image, text message, or flashing light) to a person concerning their eating behavior based on the types and/or amounts of food consumed by the person. In an example, a device and/or system can provide neurostimulation feedback to a person concerning their eating behavior based on the types and/or amounts of food consumed by the person. In an example, a device and/or system can comprise an LED-based display which is used to provide feedback to a person concerning the types and/or amounts of food which they have consumed.

In an example, feedback can be in the form of a voice. In an example, feedback can be in the form of a virtual object displayed in a person’s field of vision. In an example, feedback can be in the form of a text message. In an example, feedback can be in the form of a pre-recorded voice message. In an example, feedback can be in the form of a machine-generated voice. In an example, feedback can comprise one or more lights (e.g. a flashing light). In an example, a system worn by a person can be integrated with a network of communication with other people so that other people can be part of a feedback provided to the person concerning their consumption of selected types and/or amounts of food.

In an example, a device and/or system can provide acoustic, auditory, and/or sound-based (e.g. a tone, tone progression, song, or computer generated voice) feedback to a person concerning their eating behavior based on the types and/or amounts of food consumed by the person. In an example, a system can provide auditory feedback to help a person adjust (e.g. slow down) their pace of food consumption. In an example, a system can provide auditory feedback to help a person adjust (e.g. slow down) the pace, speed, and/or frequency of their eating motions. In an example, a system can provide auditory feedback to help a person adjust (e.g. slow down) the pace, speed, and/or frequency of their hand-to-mouth motions. In an example, a system can provide auditory feedback to help a person adjust (e.g. slow down) the pace, speed, and/or frequency of their chewing motions.

In an example, a device can further comprise a speaker which emits sounds or tones, plays music, and/or speaks verbal messages into a person’s ear as part of feedback on the person’s food consumption. In an example, a device can further comprise a speaker which emits sounds or tones, plays music, and/or speaks verbal messages into a person’s ear during an eating event as part of real-time feedback on the person’s food consumption. In an example, feedback can comprise one or more sounds (e.g. a tone, a beep, a vocal message, or music). In an example, feedback can be in the form of a sound, tone, musical pattern, and/or song.

In an example, a system can provide musical feedback to help a person adjust (e.g. slow down) their pace of food consumption. In an example, a system can provide musical feedback to help a person adjust (e.g. slow down) the pace, speed, and/or frequency of their eating motions. In an example, a system can provide musical feedback to help a person adjust (e.g. slow down) the pace, speed, and/or frequency of their hand-to-mouth motions. In an example, a system can provide musical feedback to help a person adjust (e.g. slow down) the pace, speed, and/or frequency of their chewing motions.

In an example, a wearable device can provide auditory feedback to a person concerning the types and/or amounts of food near them, wherein the magnitude of sound is based on the amount and/or type of that food. In an example, a wearable device can provide auditory feedback to a person concerning the types and/or amounts of food consumed by the person, wherein the magnitude of sound is based on the amount and/or type of that food. In an example, a wearable device can provide auditory feedback to a person concerning the types and/or amounts of food consumed by the person, wherein the frequency of sound is based on the amount and/or type of that food.

For example, a wearable sensor can trigger an alarm, or other response, if it removed from contact with the person’s skin. Skin contact can be monitored using electromagnetic, pressure, motion, and/or sound sensors. In an example, a wearable motion sensor can trigger an alarm, or other response, if there is a lack of motion that is not also accompanied by specific indications of sleeping activity. In an example, a wearable sound sensor can trigger an alarm or other response if there is a lack of sounds (such as pulse or respiration) that are normally associated with proximity to the person’s body. In an example, a wearable camera may trigger an alarm, or other response, if there is a lack of images (such as a view of the person’s hand or face identified by recognition software) that are associated with proper positioning on the person’s body.

In an example, a wearable device can provide vibrational and/or tactile feedback to a person concerning the types and/or amounts of food consumed by the person, wherein the frequency of vibration and/or device movement is based on the amount and/or type of that food. In an example, feedback can be in the form of a vibration. In an example, feedback can be in the form of a vibration, wherein the frequency and/or strength of the vibration depends on the amount and/or type of food consumed. In an example, feedback can comprise a vibration, mild shock, or other tactile or haptic signal. In an example, a device and/or system for nutritional intake management can trigger unpleasant sensory (e.g. olfactory, acoustic, tactile, or visual) feedback when a person eats a type and/or amount of food which is unhealthy.

In an example, a wearable device can provide vibrational and/or tactile feedback to a person concerning the types and/or amounts of food near them, wherein the magnitude of vibration and/or device movement is based on the amount and/or type of that food. In an example, a wearable device can provide vibrational and/or tactile feedback to a person concerning the types and/or amounts of food consumed by the person, wherein the magnitude of vibration and/or device movement is based on the amount and/or type of that food. In an example, a wearable device can provide vibrational and/or tactile feedback to a person concerning the types and/or amounts of food near them, wherein the frequency of vibration and/or device movement is based on the amount and/or type of that food.

In an example, feedback from a device to a person can include audio, visual, or tactile feedback. In an example, a device and/or system can provide tactile and/or haptic feedback (e.g. a vibration, vibration pattern, mild pressure, or mild shock) to a person concerning their eating behavior based on the types and/or amounts of food consumed by the person. In an example, feedback from a device to a person can include information concerning cumulative calories consumed during a time interval (e.g. during a day) via audio (e.g. computer-generated speech), visual (e.g. display screen or augmented reality display), or tactile (e.g. skin vibration) modalities.

In an example, feedback from a device to a person can include information concerning cumulative calories consumed during an eating event and/or meal via audio (e.g. computer-generated speech), visual (e.g. display screen or augmented reality display), or tactile (e.g. skin vibration) modalities. In an example, feedback from a device to a person can include information concerning cumulative amounts of different types of nutrients consumed during a time interval (e.g. during a day) via audio (e.g. computer-generated speech), visual (e.g. display screen or augmented reality display), or tactile (e.g. skin vibration) modalities. In an example, feedback from a device to a person can include information concerning cumulative amounts of different types of nutrients consumed during an eating event and/or meal via audio (e.g. computer-generated speech), visual (e.g. display screen or augmented reality display), or tactile (e.g. skin vibration) modalities.

In an example, a system can change a color filter in a person’s eyewear based on the types and/or amounts of food consumed by the person. In an example, eyewear can include a heads-up display which provides information about the types and/or amounts of food in front of a person. In an example, eyewear can include a heads-up display which provides information about the types and/or amounts of food consumed by a person. In an example, eyewear can include a virtual display which provides information about the types and/or amounts of food in front of a person. In an example, eyewear can include a virtual display which provides information about the types and/or amounts of food consumed by a person. In an example, feedback can be in the form of a virtual object displayed in virtual reality eyewear. In an example, feedback can be in the form of changing the color spectrum of nearby food in a person’s field of view using smart eyewear.

In an example, eyewear can include a reservoir of material with an unappetizing scent and/or odor which is emitted in proximity to a person’s nose when the person consumes unhealthy food. In an example, eyewear can include a reservoir of scented material which is emitted in proximity to a person’s nose based on the types and/or amounts of food consumed by the person. In an example, eyewear can include a reservoir of scented material which is emitted in proximity to a person’s nose when the person consumes unhealthy food.

In an example, a device and/or system can change the colors of unhealthy foods in a person’s field of vision (e.g. via AR eyewear) to make those appear less appetizing. In an example, a device and/or system can change the colors of different types of food in a person’s field of vision (e.g. via AR eyewear) to make foods which are unhealthy for that person appear less appetizing. In an example, a device and/or system can change the colors of different types of food in a person’s field of vision (e.g. via AR eyewear) based on whether those different types of food are relatively healthy or unhealthy for that person. In an example, a device and/or system can change the colors of different types of food in a person’s field of vision (e.g. via AR eyewear) based on the nutritional compositions of those different types of food.

In an example, a device and/or system can estimate and display via AR eyewear the number of calories in each type of food in a meal. In an example, calories associated with a meal or food portions within a meal can be displayed in a person’s field of vision via AR eyewear. In an example, calories associated with a meal or food portions within a meal can be superimposed over the meal or food portions in a person’s field of vision via AR eyewear. In an example, a device can track a person’s cumulative caloric intake (e.g. positive calories), track the person’s cumulative caloric expenditure (e.g. negative calories), and track the maximum amount of additional caloric intake allowed before caloric energy balance becomes a surplus (e.g. positive net balance of calories). In an example, the net balance of calories can be display graphically on a screen or via AR eyewear.

In an example, a device and/or system can estimate and display via AR eyewear the number of each type of nutrient in each type of food in a meal. In an example, AR eyewear can display information about the types and/or amounts of food in front of a person. In an example, AR eyewear can display information about the types and/or amounts of food consumed by a person. In an example, feedback can comprise one or more projected images (e.g. virtual images projected in a person’s field of vision by AR eyewear). In an example, a system can analyze an image of a menu and display via AR eyewear which dishes would be best for a person. In an example, a system can analyze an image of a menu and indicate via AR eyewear which dishes would be best for a person. In an example, a system can analyze options on a menu and display via AR eyewear which dishes would be best for a person. In an example, a system can analyze options on a menu and indicate via AR eyewear which dishes would be best for a person.

In an example, AR eyewear can display a recommended path in space over nearby food along which a person should move their phone to record images of the food from different angles and/or distances for better identification of food types and/or amounts using images from the phone. In an example, AR eyewear can display a recommended path in space over nearby food along which a person should move their phone to record images of the food from different angles and/or distances to create (more accurate) three-dimensional models of the food using images from the phone.

In an example, AR eyewear can identify unhealthy food in a person’s field of view and block the person’s view of that food. In an example, AR eyewear can identify unhealthy food in a person’s field of view and change the color of that food in the person’s field of view. In an example, AR eyewear can identify unhealthy food in a person’s field of view and change the appearance of that food in the person’s field of view to make it less appealing to that person. In an example, AR eyewear can identify unhealthy food in a person’s field of view and superimpose unappealing virtual objects over the food in the person’s field of view to make the food less appealing. In an example, AR eyewear can identify unhealthy food in a person’s field of view and superimpose unappealing virtual objects (e.g. crawling insects, maggots, worms, or, even worse, annoying emojies) over the food in the person’s field of view to make the food less appealing. In an example, AR eyewear can identify food in a person’s field of view which would be unhealthy for that person to consume and block the person’s view of that food.

In an example, AR eyewear worn by a person can analyze a meal in front of the person and recommend which foods in that meal the person should or should not eat. In an example, AR eyewear worn by a person can analyze a meal in front of the person and recommend how much (e.g. what fraction or percentage) of each of the foods in that meal the person should eat. In an example, AR eyewear worn by a person can analyze an image of a menu and display which dishes would be most healthy for the person. In an example, AR eyewear worn by a person can analyze an image of a menu and indicate which dishes would be most healthy for the person. In an example, AR eyewear worn by a person can analyze options on a menu and display which dishes would be most healthy for the person. In an example, AR eyewear worn by a person can analyze options on a menu and indicate which dishes would be most healthy for the person. In an example, feedback can be in the form of changing the color spectrum of nearby food in a person’s field of view using augmented reality eyeglasses and/or eyewear.

In an example, AR eyewear worn by a person can display an actual border, perimeter, and/or outline around a type and/or portion of food in a meal in the person’s field of vision and can also display a recommended border, perimeter, and/or outline around a subset of that food based on a recommended amount of that food for the person to consume. In an example, AR eyewear worn by a person can display an actual border, perimeter, and/or outline around a type and/or portion of food in a meal in the person’s field of vision and can also display a border, perimeter, and/or outline around a subset of that food based on a recommended amount of that food for the person to consume.

In an example, AR eyewear worn by a person can display borders, perimeters, and/or outlines around different types of food in a meal in the person’s field of vision. In an example, a system can display two borders, perimeters, and/or outlines around a portion of one type of food in a meal in the person’s field of vision via AR eyewear, wherein a first border, perimeter, and/or outline is around the entire food portion, wherein a second border, perimeter, and/or outline is around a subset of the food portion, and wherein the system recommends that the person consume only the subset of the food portion. In an example, AR eyewear worn by a person can display a virtual border, perimeter, and/or outline around a subset of (a portion of) food based on a recommended amount of that food for the person to consume.

In an example, feedback in response to detection of consumption of unhealthy types and/or amounts of food by a person can comprise displaying (e.g. via AR eyewear) images of that person with computer-simulated weight gain. In an example, feedback in response to detection of consumption of unhealthy types and/or amounts of food can comprise displaying (e.g. via AR eyewear) images of an overweight person. In an example, feedback in response to detection of consumption of unhealthy types and/or amounts of food by a person can comprise displaying (e.g. via AR eyewear) computer-simulated images of that person after weight gain. In an example, feedback in response to detection of consumption of unhealthy types and/or amounts of food by a person can comprise displaying (e.g. via AR eyewear) computer-simulated images of that person after projected weight gain based on continued consumption of those types and/or amounts of food.

In an example, images of virtual insects or worms (e.g. crawling over or into food) can be selectively superimposed on unhealthy food in a person’s field of vision by AR eyewear in order to make that unhealthy food less visually appealing. In an example, images of virtual insects or worms (e.g. crawling over or into food) can be selectively superimposed on food in a person’s field of vision by AR eyewear in order to make that food less visually appealing when the amount of food that the person has eaten approaches or exceeds a target amount. In an example, if a Klingon has already eaten too much gagh and further consumption of gagh would be unhealthy for them, then AR eyewear can modify the appearance of the gagh in their field of view so that it less appetizing (e.g. the gagh appears immobile rather than wiggling and fresh).

In an example, the colors of food portions within a meal can be selectively altered in a person’s field of vision by AR eyewear in order to make food less visually appealing when the amount of food that the person has eaten approaches or exceeds a target amount. In an example, the colors of food portions within a meal can be selectively altered in a person’s field of vision by AR eyewear in order to increase the visual appeal of healthy food and decrease the visual appeal of unhealthy food. In an example, the colors of food portions within a meal can be selectively altered in a person’s field of vision by AR eyewear in order to give unhealthy food an unappealing tint. In an example, the colors of food portions within a meal can be selectively altered in a person’s field of vision by AR eyewear in order to decrease the visual appeal of food when the amount of food that the person has eaten approaches or exceeds a target amount.

In an example, the colors of food portions within a meal can be selectively altered in a person’s field of vision by AR eyewear. In an example, the colors of food portions within a meal can be selectively altered in a person’s field of vision by AR eyewear when the amount that the person has eaten approaches or exceeds a target amount. In an example, the colors of food portions within a meal can be selectively altered in a person’s field of vision by AR eyewear in order to make unhealthy food less visually appealing. In an example, the colors of food portions within a meal can be selectively altered in a person’s field of vision by AR eyewear in order to make unhealthy food appear less appetizing.

In an example, words with unappetizing meanings can selectively superimposed on unhealthy food in a person’s field of vision by AR eyewear in order to make that unhealthy food less visually appealing. In an example, words with unappetizing meanings can selectively superimposed on food in a person’s field of vision by AR eyewear in order to make that food less visually appealing when the amount of food that the person has eaten approaches or exceeds a target amount. In an example, unappetizing visual objects can be superimposed over unhealthy food in a person’s field of vision by AR eyewear. In an example, unappetizing visual objects can be superimposed over food in a person’s field of vision by AR eyewear when the amount of food that the person has eaten approaches or exceeds a target amount.

In an example, a device can be part of a system for controlling an insulin pump. In an example, a system can determine the amount of insulin to be delivered to a person by an insulin pump based on the types and/or amounts of food consumed by the person. In an example, a system can include a wearable insulin pump. In an example, a system can trigger an insulin pump to deliver insulin to a person based on the types and/or amounts of food consumed by the person. In an example, a system can trigger an insulin pump to deliver insulin to a person based on the person’s food consumption. In an example, a system for blood glucose management can comprise a wearable device to measure a person’s food consumption and a wearable insulin pump to administer insulin to the person based (at least in part) on the types and/or amounts of food consumed by the person.

In an example, a device can be part of a system for delivering insulin. In an example, a device can be part of a system for managing the dispensation of insulin. In an example, a system for blood glucose management can comprise a device to measure a person’s food consumption and an artificial pancreas to administer insulin to the person based (at least in part) on the types and/or amounts of food consumed by the person. In an example, the types and/or amounts of food consumed by a person which are detected by a device can be part of the factors used to determine how much insulin should be delivered to that person. In an example, the types and/or amounts of nutrients from food consumed by a person which are detected by a device can be part of the factors used to determine how much insulin should be delivered to that person. In an example, a device can be part of a system for managing insulin injections.

In an example, a device can be part of a system for management of blood glucose level which also includes a non-invasive optical glucose monitor. In an example, a device can be part of a system for management of blood glucose level which also includes a non-invasive spectroscopic glucose monitor. In an example, a device can be part of a system for management of blood glucose level which also includes a non-invasive impedance-based glucose monitor. In an example, a device can be part of a system for management of blood glucose level which also includes a non-invasive glucose monitor. In an example, a device can be part of a system for management of blood glucose level which also includes a minimally-invasive glucose monitor.

In an example, a device can be part of a system which analyzes the relationship between a person’s net caloric balance (e.g. cumulative caloric intake minus cumulative caloric expenditure) and changes in the person’s blood glucose level. In an example, a device for measuring a person’s food consumption can be part of a system which analyzes the relationship between a person’s food consumption (e.g. the types and/or amounts of food consumed) and the person’s blood glucose level (e.g. changes in blood glucose level following food consumption in the past. In an example, quantification of this relationship can help to predict future changes in blood glucose level following present or future food consumption. In an example, a device can analyze the relationship between a person’s consumption of specific types and/or amounts of food and subsequent changes in the person’s blood glucose level.

In an example, a device can be part of a system which predicts a person’s blood glucose level based on one or more factors selected from the group consisting of: the amount of exercise that the person has done during a recent period of time; the person’s body mass index; the types and/or amounts of food that the person has consumed during a recent period of time; the person’s rate of chewing, swallowing, and/or food consumption; the person’s BMI; the person’s demographic characteristics; the person’s cumulative caloric intake during a recent period of time; the person’s health status; the results of genetic testing; and the types and/or amounts of exercise that the person has done during a recent period of time.

In an example, a device can be part of a system which predicts changes in a person’s blood glucose level based on one or more factors selected from the group consisting of: the amount of exercise that the person has done during a recent period of time; the person’s body mass index; the types and/or amounts of food that the person has consumed during a recent period of time; the person’s rate of chewing, swallowing, and/or food consumption; the person’s BMI; the person’s demographic characteristics; the person’s cumulative caloric intake during a recent period of time; the person’s health status; the results of genetic testing; and the types and/or amounts of exercise that the person has done during a recent period of time.

In an example, a device can comprise and/or include a blood glucose sensor. In an example, a system can include a continuous glucose monitor (CGM). In an example, a system can adjust recommendations for types and/or amounts of food for a person to consume based on data from a continuous glucose monitor worn by the person. In an example, a system can provide recommendations for types and/or amounts of food for a person to consume based on data from a continuous glucose monitor worn by the person.

In an example, a device can using time-series statistical methods to analyze the relationship between a person’s consumption of specific types and/or amounts of food and subsequent changes in the person’s blood glucose level. In an example, a device can be part of a system which analyzes the time-lagged relationship between a person’s net caloric balance (e.g. cumulative caloric intake minus cumulative caloric expenditure) and subsequent changes in the person’s blood glucose level. In an example, a device can be part of a system which uses time-series statistical methods to identify a time-lagged relationship between a person’s net caloric balance (e.g. cumulative caloric intake minus cumulative caloric expenditure) and subsequent changes in the person’s blood glucose level. In an example, a device can using statistical methods to analyze a time-lagged relationship between a person’s consumption of specific types and/or amounts of food and subsequent changes in the person’s blood glucose level.

In an example, a system for blood glucose management can comprise a wearable device to measure a person’s food consumption and a wearable device to administer a therapeutic agent to the person based (at least in part) on the types and/or amounts of food consumed by the person. In an example, a system for blood glucose management can comprise a wearable device to measure a person’s food consumption and an implanted device to administer a therapeutic agent to the person based (at least in part) on the types and/or amounts of food consumed by the person. In an example, a system for blood glucose management can comprise a wearable device to measure a person’s food consumption and an implanted device to deliver neurostimulation to the person based (at least in part) on the types and/or amounts of food consumed by the person.

In an example, a system for blood glucose management can comprise a wearable device to measure a person’s food consumption and an implanted device to deliver neurostimulation to one or more of the person’s gastrointestinal organs based (at least in part) on the types and/or amounts of food consumed by the person. In an example, a system for blood glucose management can comprise a wearable device to measure a person’s food consumption and an implanted device to deliver electrical stimulation to one or more of the person’s gastrointestinal organs based (at least in part) on the types and/or amounts of food consumed by the person.

In an example, a system for blood glucose management can comprise a device to measure a person’s food consumption and an insulin pump to administer insulin to the person based (at least in part) on the types and/or amounts of food consumed by the person. In an example, a system for managing a person’s blood glucose level can include a wearable device for measuring a person’s food consumption and a wearable insulin pump. In an example, a system for managing a person’s blood glucose level can include an insulin pump on a person and eyewear with sensors which collect data which is analyzed to measure a person’s food consumption. In an example, a system for managing a person’s blood glucose level can include an insulin pump on a person and eyewear with acoustic sensors which collect data which is analyzed to measure a person’s food consumption.

In an example, a system for managing a person’s blood glucose level can include an insulin pump on a person and eyewear with a microphone which collects data which is analyzed to measure a person’s food consumption. In an example, a system for managing a person’s blood glucose level can include an insulin pump on a person and eyewear with a microphone and a camera which collect data which is analyzed to measure a person’s food consumption. In an example, a system for managing a person’s blood glucose level can include an insulin pump on a person and eyewear with a microphone and a camera which record sounds and images which are analyzed to measure a person’s food consumption. In an example, a system for managing a person’s blood glucose level can include an insulin pump on a person and eyewear with a microphone and a camera which record sounds and images which are analyzed to measure the types and amounts of food consumed by the person.

In an example, a system for managing a person’s blood glucose level can include an insulin pump on a person and a wrist-worn device (e.g. smart watch) with sensors which collect data which is analyzed to measure a person’s food consumption. In an example, a system for managing a person’s blood glucose level can include an insulin pump on a person and a wrist-worn device (e.g. smart watch) with acoustic sensors which collect data which is analyzed to measure a person’s food consumption. In an example, a system for managing a person’s blood glucose level can include an insulin pump on a person and a wrist-worn device (e.g. smart watch) with a microphone which collects data which is analyzed to measure a person’s food consumption.

In an example, a system for managing a person’s blood glucose level can include an insulin pump on a person and a wrist-worn device (e.g. smart watch) with a microphone and a camera which collect data which is analyzed to measure a person’s food consumption. In an example, a system for managing a person’s blood glucose level can include an insulin pump on a person and a wrist-worn device (e.g. smart watch) with a microphone and a camera which record sounds and images which are analyzed to measure a person’s food consumption. In an example, a system for managing a person’s blood glucose level can include an insulin pump on a person and a wrist-worn device (e.g. smart watch) with a microphone and a camera which record sounds and images which are analyzed to measure the types and amounts of food consumed by the person.

In an example, information on the types and/or amounts of food consumed by a person based on analysis of data from a wearable device can be used to inform the amount of insulin which the person receives from an insulin pump. In an example, information on the types and/or amounts of food consumed by a person based on analysis of data from a wearable device can be incorporated and/or integrated into the person’s insulin pump. In an example, information on the types and/or amounts of food consumed by a person based on analysis of data from a wearable device can be transmitted into the person’s insulin pump.

In an example, a system can emit a scent and/or odor in proximity to a person’s nose based on the types and/or amounts of food consumed by the person. In an example, a system can trigger neurostimulation of a person’s gastrointestinal organs based on the types and/or amounts of food consumed by the person. In an example, a system can dispense flavored material into a person’s mouth based on the types and/or amounts of food consumed by the person. In an example, a system can adjust the size and/or shape of an intragastric balloon based on the types and/or amounts of food consumed by the person. In an example, a system can adjust a person’s gastric processes based on the types and/or amounts of food consumed by the person. In an example, a system can adjust a person’s gastric pH level based on the types and/or amounts of food consumed by the person.

In an example, data from a system for monitoring food intake can be incorporated into a person’s electronic health (e.g. medical) record. In an example, data from a device for measuring a person’s food consumption can be incorporated into the person’s electronic health (e.g. medical) record. In an example, information on the types and/or amounts of food consumed by a person based on analysis of data from a wearable device can be incorporated and/or integrated into the person’s electronic health (e.g. medical) record.

In an example, information on the types and/or amounts of food consumed by a person based on analysis of data from a wearable device can be transmitted into the person’s electronic health (e.g. medical) record. In an example, a system for monitoring food intake can be integrated with a person’s electronic health (e.g. medical) record. In an example, a system can use data from health issues to estimate the types and/or amounts of food consumed by a person wherein this data can include one or more variables selected from the group consisting of: medical condition, recent exercise history, recent food consumption history, recent sleep history, stress level, and weight.

In an example, a device and/or system can estimate the number of each type of nutrient in each type of food in a meal. In an example, a device and/or system can estimate the number of calories in each type of food in a meal. In an example, a device and/or system can analyze eating behavior to identify snacking behavior. In an example, a device can detect one or more types of food to which a person has a food allergy wherein these one or more types of food is selected from the group consisting of: eggs, gluten, lactose, milk, peanuts, processed sugar, seafood, shellfish, soy, tree nuts, and wheat. In an example, this device can be a nutritional intake and/or nutritional consumption monitor. In an example, this device can be a nutritional intake and/or nutritional consumption tracker. In an example, this device can be a food intake and/or food consumption tracker. In an example, this device can be a food intake and/or food consumption monitor. In an example, the term food used herein is understood to include liquid food (e.g. beverages) as well as solid food.

In an example, a device can include a central processing unit (CPU). In an example, a device and/or system can comprise a central processing unit (CPU). In an example, data from a wearable device can be transmitted to, analyzed within, and/or stored in a remote server. In an example, a wearable device for measuring a person’s food consumption can be in communication with a remote server. In an example, a wearable device for measuring a person’s food consumption can be part of a system which also includes a remote server.

In an example, a device can include a data processor. In an example, a device and/or system can comprises a digital signal processor (DSP). In an example, a device can comprise a data processor and memory. In an example, data from a wearable device can be transmitted to, analyzed within, and/or stored in a remote data processor. In an example, device can be in communication with a remote processor via the internet. In an example, device can be in communication with a remote processor via a data transmitter and receiver. In an example, data from a wearable device can be transmitted to, analyzed within, and/or stored in a data processor in the cloud. In an example, a device can transform and/or convert an analog vibration into a digital signal. In an example, signals and/or data from one or more sensors can be fully analyzed within a local data processor within a wearable device. In an example, signals and/or data from one or more sensors can be transmitted to a remote data processor (e.g. In a separate wearable device, handheld device, or remote server) for analysis within the remote data processor.

In an example, a system can include a data processing and transmission unit which analyzes data from one or more sensors. In an example, a data processing and transmission unit receives data from a motion sensor, a microphone, and a video camera. This data processing and transmission unit is also able to communicate data to and from a remote computer. In an example, all data processing tasks can occur within a wearable device. In an example, some of these data processing tasks can occur within the wearable device and other tasks can occur in a remote computer. In an example, data can be transmitted back and forth from the wearable device to a remote computer via a data processing and transmission unit.

In an example, an iterative method for measuring a person’s food consumption and caloric intake can be performed with the assistance of one or more computing units or data processors. A computing unit can be incorporated into a wearable device, into a mobile device, or located in a remote location via data transmission means such as wireless communication or the internet. In an example, estimation of caloric intake can be largely, or entirely, automated. In an example, this estimation of caloric intake in can be done by a data processing device such as a computer. In an example, this estimation process can be performed within a device that is worn in or on a person. In an example, this estimation process can be performed in a mobile device that is carried by the person. In an example, this estimation process can be performed in a computer in a remote location, with data transferred back and forth between a wearable device and a computer in the remote location. In an example, data transferred between a wearable device and a computer in a remote location can be encrypted for the sake of privacy.

In an example, a device can be part of a system which includes wireless communication with a smart phone, wherein movement of the smart phone over a meal records images of the meal from different angles and/or distances in order to more accurately estimate the types and/or amounts of food in the meal using images from the phone. In an example, a device can be part of a system which includes wireless communication with a smart phone, wherein movement of the smart phone over food records images of the food from different angles and/or distances in order to create a three-dimensional model of the food using images from the phone.

In an example, a device can be part of a system which includes wireless communication with a smart phone, wherein movement of the smart phone over food records images of the food from different angles and/or distances in order to more accurately estimate the types and/or amounts of food using images from the phone. In an example, a wearable device for measuring a person’s food consumption can be in wireless communication with a mobile phone. In an example, a wearable device for measuring a person’s food consumption can be in wireless communication with a handheld device. In an example, device can be in communication with a remote processor via a wireless data transmitter and receiver. In an example, a wearable device for measuring a person’s food consumption can be part of a system which also includes a handheld device. In an example, a device and/or system can comprise a smartphone, tablet, or laptop. In an example, a wearable device for measuring a person’s food consumption can be part of a system which also includes a mobile phone. In an example, device can be in communication with a phone via a data transmitter and receiver.

In an example, a device worn by a person can communicate with the person via visual cues (e.g. displayed words, images, light patterns, colors, and/or virtual objects). In an example, a device can comprise a display screen. In an example, a device and/or system can comprise an LED-based display.

In an example, a device and/or system can comprise Random Access Memory (RAM) and/or Read Only Memory (ROM). In an example, a device can comprise and/or include a GPS component. In an example, a device can comprise and/or include a gyroscope. In an example, a device can comprise and/or include a magnetometer. In an example, a device can further comprise an on-off button. In an example, a device can include a sound output device (such as a speaker). In an example, a device can comprise a holographic projector. In an example, a device can include a camera and a light emitter, wherein the device automatically emits shines light on food which a food image recorded by the camera is too dark. In an example a device can shine a light onto food when a food image recorded by the camera is too dark for accurate food identification.

In an example, a device can include an infrared or near-infrared distance finder. In an example, a device can comprise a signal amplifier. In an example, a device can include a local signal pre-amplifier. In an example, a device and/or system can comprise a touchscreen. In an example, a device can comprise a touch screen. In an example, a device can comprise a touch-based user interface. In an example, a sensor can be a MEMS device. In an example, a sensor to detect eating can comprise one or more MEMS components.

1. A device or system for estimating food consumption comprising:
a first wearable sensor which is configured to be worn by a person, wherein a first set of data is recorded by the first sensor, and wherein the first set of data is analyzed to detect when the person is consuming food;
a second wearable sensor which is configured to be worn by the person, wherein a second set of data is recorded by the second sensor, wherein the first set of data and the second set of data are jointly analyzed to estimate the types and amounts of food consumed by the person, and wherein (a) the second sensor is triggered to start recording the second set of data when analysis of the first set of data indicates that the person is consuming food or (b) wherein the second sensor is triggered to increase the amount, level, or scope of data in the second set of data when analysis of the first set of data indicates that the person is consuming food;
a data processor, wherein the first set of data and the second set of data are analyzed by the data processor; and
a feedback mechanism which provides feedback to the person based on the types and amounts of food consumed by the person.

2. The device or system in claim 1: wherein the first wearable sensor is a microphone which is part of, or attached to, an ear-worn device; wherein the second wearable sensor is a camera which is part of, or attached to, the ear-worn device; and wherein the feedback mechanism communicates information concerning the types and amounts of food to the person via the ear-worn device.

3. The device or system in claim 1: wherein the first wearable sensor is a microphone which is part of, or attached to, eyewear; wherein the second wearable sensor is a camera which is part of, or attached to, the eyewear; and wherein the feedback mechanism communicates information concerning the types and amounts of food to the person via the eyewear.

4. The device or system in claim 1: wherein the first wearable sensor is a microphone which is part of, or attached to, eyewear; wherein the second wearable sensor is a camera which is part of, or attached to, the eyewear; and wherein the feedback mechanism changes the appearance of food in the person’s field of vision via eyewear to change the person’s food consumption behavior.

5. The device or system in claim 1: wherein the first wearable sensor is a motion which is part of, or attached to, a wrist-worn device; wherein the second wearable sensor is a camera which is part of, or attached to, the wrist-worn device; and wherein the feedback mechanism makes recommendations concerning food consumption to the person via the wrist-worn device.

6. The device or system in claim 1: wherein the first wearable sensor is a motion which is part of, or attached to, a wrist-worn device; wherein the second wearable sensor is a camera which is part of, or attached to, the wrist-worn device; and wherein the feedback mechanism communicates information concerning the types and amounts of food to the person via the wrist-worn device.

7. The device or system in claim 1: wherein the first wearable sensor is a reflective optical which is part of, or attached to, an ear-worn device; wherein the second wearable sensor is a camera which is part of, or attached to, the ear-worn device; and wherein the feedback mechanism makes recommendations concerning food consumption to the person via the ear-worn device.

8. The device or system in claim 1: wherein the first wearable sensor is a reflective optical which is part of, or attached to, an ear-worn device; wherein the second wearable sensor is a camera which is part of, or attached to, the ear-worn device; and wherein the feedback mechanism communicates information concerning the types and amounts of food to the person via the ear-worn device.

9. The device or system in claim 1: wherein the first wearable sensor is a reflective optical which is part of, or attached to, eyewear; wherein the second wearable sensor is a camera which is part of, or attached to, the eyewear; and wherein the feedback mechanism communicates information concerning the types and amounts of food to the person via the eyewear.

10. The device or system in claim 1: wherein the first wearable sensor is a reflective optical which is part of, or attached to, eyewear; wherein the second wearable sensor is a camera which is part of, or attached to, the eyewear; and wherein the feedback mechanism changes the appearance of food in the person’s field of vision via eyewear to change the person’s food consumption behavior.

11. The device or system in claim 1: wherein the first wearable sensor is a microphone which is part of, or attached to, an ear-worn device; wherein the second wearable sensor is a camera which is part of, or attached to, the ear-worn device; and wherein the feedback mechanism makes recommendations concerning food consumption to the person via the ear-worn device.

12. The device or system in claim 1: wherein the first wearable sensor is a microphone which is part of, or attached to, an ear-worn device; wherein the second wearable sensor is a camera which is part of, or attached to, the ear-worn device; and wherein the feedback mechanism makes recommendations concerning food consumption to the person via a phone.

13. The device or system in claim 1: wherein the first wearable sensor is a microphone which is part of, or attached to, an ear-worn device; wherein the second wearable sensor is a camera which is part of, or attached to, the ear-worn device; and wherein the feedback mechanism communicates information concerning the types and amounts of food to the person via a phone.

14. The device or system in claim 1: wherein the first wearable sensor is a microphone which is part of, or attached to, eyewear; wherein the second wearable sensor is a camera which is part of, or attached to, the eyewear; and wherein the feedback mechanism makes recommendations concerning food consumption to the person via the eyewear.

15. The device or system in claim 1: wherein the first wearable sensor is a motion which is part of, or attached to, a finger-worn device; wherein the second wearable sensor is a camera which is part of, or attached to, the finger-worn device; and wherein the feedback mechanism makes recommendations concerning food consumption to the person via a phone.

16. The device or system in claim 1: wherein the first wearable sensor is a motion which is part of, or attached to, a finger-worn device; wherein the second wearable sensor is a camera which is part of, or attached to, the finger-worn device; and wherein the feedback mechanism communicates information concerning the types and amounts of food to the person via a phone.

17. The device or system in claim 1: wherein the first wearable sensor is a motion which is part of, or attached to, a wrist-worn device; wherein the second wearable sensor is a camera which is part of, or attached to, the wrist-worn device; and wherein the feedback mechanism communicates information concerning the types and amounts of food to the person via a phone.

18. The device or system in claim 1: wherein the first wearable sensor is a optical which is part of, or attached to, an ear-worn device; wherein the second wearable sensor is a camera which is part of, or attached to, the ear-worn device; and wherein the feedback mechanism makes recommendations concerning food consumption to the person via the ear-worn device.

19. The device or system in claim 1: wherein the first wearable sensor is a reflective optical which is part of, or attached to, eyewear; wherein the second wearable sensor is a camera which is part of, or attached to, the eyewear; and wherein the feedback mechanism makes recommendations concerning food consumption to the person via the eyewear.

20. The device or system in claim 1: wherein the first wearable sensor is a optical which is part of, or attached to, an ear-worn device; wherein the second wearable sensor is a camera which is part of, or attached to, the ear-worn device; and wherein the feedback mechanism communicates information concerning the types and amounts of food to the person via the ear-worn device.
